(19) United States
(12) Patent Application Publication
(10) Pub. No.: US 2025/0382341 A1
(43) Pub. Date: Dec. 18, 2025

(54) METHODS AND COMPOSITIONS OF IN VIVO ENGINEERING OF T-CELLS TO ANTI-INFLAMMATORY CELLS AND THERAPEUTIC APPLICATIONS THERE OF

(71) Applicant: The Regents of the University of California, Oakland, CA (US)

(72) Inventors: Adam Oskowitz, San Francisco, CA (US); Melroy Miranda, San Francisco, CA (US); Chandrashekhar Dasari, San Francisco, CA (US); Mehrdad Rajaei, San Francisco, CA (US)

(73) Assignee: The Regents of the University of California, Oakland, CA (US)

(21) Appl. No.: 19/144,796

(22) PCT Filed: Dec. 29, 2023
(86) PCT No.: PCT/US2023/086538

§ 371 Date: Jun. 30, 2025

Related U.S. Application Data
(60) Provisional application No. 63/478,066, filed on Dec. 30, 2022.

(51) Int. Cl. C07K 14/47; A61K 9/00; A61K 9/51; A61K 38/00; A61K 47/68; A61K 47/69; A61P 29/00; C12N 15/88

(52) U.S. Cl. CPC C07K 14/4702; A61K 9/0019; A61K 9/5146; A61K 47/6849; A61K 47/6929; A61P 29/00; C12N 15/88; A61K 38/00

(57) ABSTRACT

Compositions comprising at least one nanoparticle containing a nucleoside RNA molecule encoding FOXP3 and an optional second agent are described herein. In some cases, the RNA molecule is circular and contains one or more IRES. Methods for treating or preventing inflammation are also described herein.

### CROSS-REFERENCES TO RELATED APPLICATIONS

The present application claims benefit of priority to U.S. Provisional Patent Application No. 63/478,066, filed Dec. 30, 2022, which is incorporated by reference for all purposes.

### BACKGROUND OF THE INVENTION

Inflammation is an evolutionarily conserved process characterized by the activation of immune and non-immune cells that protect the host of pathogens and stimuli. Shifts in the inflammatory response towards uncontrolled acute or chronic inflammation can cause a breakdown of immune tolerance and lead to major alterations in all tissues and organs leading to various pathological conditions and non-communicable diseases.

Regulatory T-cells (Tregs) are immune regulators that provide potent anti-inflammatory activity via direct contact and paracrine actions, showing great potential for treating different inflammatory diseases and balancing immune tolerance. They depend on a continuous expression of the transcription factor FOXP3 which plays a critical role in the regulation of tissue inflammation. FOXP3 expression can also be induced in T cells in the periphery, converting T cells into a regulatory phenotype. This event is thought to aid in the balance of pro-inflammatory signals and anti-inflammatory signals during both acute and chronic inflammatory conditions.

Additional factors also play a significant role in the ability for Tregs to regulate inflammation. The transcription factor Helios has been shown to stabilize the functional capacity of Tregs, while the Interleukin-2 (IL-2) receptor alpha chain (CD 25) has been shown to allow for Treg expansion in vivo. Equally as important as functional capacity is the ability for Tregs to exert their anti-inflammatory potential at sites of tissue injury. Although endogenous Tregs can home to areas of inflammation, once near the tissue, they may not activate to exert anti-inflammatory control on local cells and the microenvironment. Chimeric Antigen Receptors (CARs) are synthetic receptors that can induce T cell and Treg activation. These receptors can be engineered to create highly specific Tregs to recognize distinct inflammatory sites. The development of a CAR-Treg that can target a specific antigen at inflammatory sites has the potential to be used as a potent therapeutic without causing systemic side effects.

At present the ability to utilize the therapeutic potential of Tregs relies on crude techniques including systemic administration of IL-2 or the ex vivo engineering of Tregs and then re-administration in an allogeneic or autologous fashion. These techniques are not titratable and in the case of cell-based therapy not reversible. Furthermore, these techniques cannot be administered rapidly in a targeted fashion precluding their use in a number of critical disease processes where Tregs have a proven benefit, such as sepsis and stroke. Thus, it is important to continue to search for new techniques to promote Treg therapies.

### BRIEF SUMMARY OF THE INVENTION

The present disclosure uses in vivo mRNA-based cell engineering to generate stable engineered Tregs from circulating immune cells. This can be accomplished through the introduction nucleoside modified nucleic acid molecules encoding Forkhead box P3 (FOXP3), either alone or in combination with at least one other agent through targeted delivery to immune cells in vivo. For example, the targeted delivery of nucleoside modified RNA molecules to T cells will induce circulatory inflammatory T cells to function as Tregs in a manner similar to the conversion of T cells that naturally express FOXP3 in the periphery. This conversion of T cells to Tregs will provide potent anti-inflammatory response at sites of inflammation. It will also tip the balance of pro-inflammatory and anti-inflammatory signals toward a more anti-inflammatory state thereby enhancing tissue healing. For example, the delivery of nucleoside modified nucleic acid molecules encoding FOXP3 with a FOXP3 stabilizing protein Helios, and a CAR targeting injured myocardial tissue, can be delivered to T cells immediately following a myocardial infarction. This would then transiently increase the number of circulating Tregs while reducing the number of circulating pro-inflammatory T cells. Furthermore, the induced Tregs would target injured myocardial tissue and therefore activate at areas of myocardial injury and enhance the rapid repair of injured cardiac tissue.

The present disclosure provides nucleoside modified nucleic acid molecules (both coding and non-coding and combinations thereof) which have structural and/or chemical features that avoid one or more of the problems in the art, for example, features which are useful for optimizing nucleic acid-based therapeutics while retaining structural and functional integrity, overcoming the threshold of expression, improving activity, stability, half-life, optimizing target cell localization, and avoiding deleterious bio-responses such as the immune response and/or degradation pathways.

In some embodiments, compositions provided herein comprise at least one nanoparticle conjugated to a targeting domain that specifically binds to a cell surface antigen of a T cell, a progenitor cell, or a precursor to a T cell, wherein the nanoparticle contains a nucleoside-modified RNA molecule comprising a coding sequence for a human FOXP3 polypeptide. In some embodiments, the RNA is a circular RNA and an IRES is operably linked to the coding sequence.

In some embodiments, the human FOXP3 polypeptide comprises an amino acid sequence at least 95% identical to any one of SEQ ID NOS: 8-11. In some embodiments, the nucleoside-modified RNA molecule comprises any one of SEQ ID NOS: 1-7. In some embodiments, the nucleoside-modified RNA molecule further comprises a polyA tail. In other embodiments, the nucleoside-modified RNA molecule further comprises at least one 1-methylpseudouridine.

In some embodiments, the nanoparticle is selected from the group consisting of a lipid carrier, a liposome, a lipid nanoparticle, and a micelle. In some embodiments, the nanoparticle is an ionizable lipid nanoparticle. In some embodiments, the nanoparticle comprises a PEG-lipid conjugated to the targeting domain. Optionally, the targeting domain is selected from the group consisting of a nucleic acid molecule, a peptide, an antibody, and a small molecule. In some embodiments, the targeting domain is an anti-CD4 antibody. In other embodiments, the targeting domain binds IL1R1.

In some embodiments, the cell surface antigen of the T cell is selected from the group consisting of CD1, CD2, CD3, CD4, CD5, CD7, CD8, CD16, CD25, CD26, CD27, CD28, CD30, CD38, CD39, CD40L, CD44, CD45, CD62L, CD69, CD71, CD73, CD80, CD83, CD86, CD95, CD103, CD119, CD126, CD150, CD152, CD153, CD154, CD161, CD183, CD223, CD254, CD275, CD45RA, CXCR3, CXCR5, FasL, IL18RI, CTLA-4, OX40, GITR, LAG3, ICOS, PD-1, leu-12, TCR, TLRI, TLR2, TLR3, TLR4, TLR6, NKG2D, CCR, CCRI, CCR2, CCR4, CCR6, and CCR7.

In some embodiments, the nanoparticle further contains at least a second agent. Optionally, the second agent is selected from the group consisting of a therapeutic agent, a stabilizing agent, an imaging agent, diagnostic agent, a contrast agent, a labeling agent and a detection agent. In some embodiments, the second agent comprises a nucleoside modified nucleic acid molecule encoding a stabilizing agent to stabilize FOXP3. Optionally, the stabilizing agent is selected from the group consisting of IKZF2, PP1, NLK, OGT, OGA, SIRT1, RORγt, USP7, USP21, RNF31, TRAF6, PRMT1, PRMT5, NFAT, LAG-3, GITR, NRP1, c-REL, ALPK1, CREB, STAT5, SMAD3, RXR, ICOS, PHD3, FOXO1, IL-2R, IDO, TIGIT, GARP, CD98, CD28, CD73 and CD39.

In some embodiments, the nanoparticle further contains a nucleoside modified nucleic acid molecule encoding a human IKZF2 Helios polypeptide comprising one of SEQ ID NOS: 12-15. Optionally, the nucleoside modified RNA encoding a modified human IKZF2 Helios polypeptide is at least 95% identical to one of SEQ ID NOS: 12-15.

In some embodiments, the second agent comprises a nucleoside modified nucleic acid molecule encoding a therapeutic agent to target inflammation. In some embodiments, the nanoparticle further contains a nucleoside modified RNA encoding a CAR. Optionally, the CAR is expressed in combination with at least one RNA molecule encoding at least one protein with a secretory signal. Optionally, the nucleoside modified RNA comprises SEQ ID NO: 19, 20, 21, or 22.

In some embodiments, the second agent comprises a receptor linked to a downstream effector or at least one component for gene editing. Optionally, the at least one component for gene editing is a Cas9 mRNA, a guide RNA, or both of a Cas9 mRNA and a guide RNA.

In some embodiments, compositions provided herein comprise a circular RNA comprising at least a first IRES operably linked to a first coding sequence and a second IRES operably linked to a second coding sequence. In some embodiments, there is an intron separating the first IRES and second IRES. In some embodiments, the first coding sequence or second coding sequence or both the first and second coding sequence encodes a human FOXP3 polypeptide at least 95% identical to SEQ ID NOS: 8-11. In other embodiments, the first coding sequence or second coding sequence or both the first and second coding sequence comprises any one of SEQ ID NOS: 1-7. In some embodiments, the first coding sequence or second coding sequence or both the first and second coding sequence encodes a human Helios polypeptide and is at least 95% identical to SEQ ID NOS: 12-15.

Optionally, one of the first coding sequence and the second coding sequence encodes a human FOXP3 polypeptide at least 95% identical to SEQ ID NOS: 8-11 and one of the first coding sequence and the second coding sequence encodes a human Helios polypeptide and is at least 95% identical to SEQ ID NOS: 12-15. Optionally, the first coding sequence encodes the human FOXP3 polypeptide and the second coding sequence encodes the human Helios polypeptide. Optionally, the first coding sequence encodes the human Helios polypeptide and the second coding sequence encodes the human FOXP3 polypeptide.

Optionally, the second coding sequence comprises any one of SEQ ID NOS: 1-7. Optionally, the second coding sequence comprises any one of SEQ ID NOS: 12-15.

In some embodiments, the first IRES comprises any one of SEQ ID NOS: 23-62. Optionally, the second IRES comprises any one of SEQ ID NOS: 23-62. In some embodiments, the circular RNA further comprises a third IRES operably linked to a third coding sequence.

Optionally, circular RNA comprises at least 5% N6-methyladenosine (m⁶A) residues.

In some embodiments, the composition provided herein comprises A nucleoside-modified RNA molecule encoding FOXP3, wherein the nucleoside modified RNA molecule has a sequence comprising Formula I,

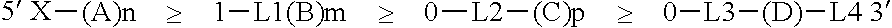

wherein, at least one of regions A, B, or C is positionally modified; at least one of the regions A, B or C is polynucleotide encoding the human FOXP3 polypeptide;
at least one of the regions A, B or C is a polynucleotide encoding at least one therapeutic agent;
n is an integer between 1 and 5;
m is an integer between 0 and 5;
p is an integer between 0 and 5;
X is a UTR between 1 base and 1 kilobase in size;
D is an optional region of linked nucleosides;
L1, L2 and L3 are independently optional linker moieties, said linker moieties being either nucleic acid based, or non-nucleic acid based; and
L4 is an optional conjugate or an optional linker moiety, said linker moiety being either nucleic acid based, or non-nucleic acid based. Optionally, the nucleoside modified RNA molecule is a circular RNA. Optionally, an IRES is operably linked to at least one, two or all three of the regions A, B, or C. Optionally, D is a polyA tail. Optionally, an intron is present between regions A and B or regions B and C or between both regions A and B and regions B and C.

In some embodiments, the region A encodes the human FOXP3 polypeptide, optionally wherein the human FOXP3 polypeptide is at least 95% identical to one of SEQ ID NOS: 8-11. In some embodiments, the region A comprises any one of SEQ ID NOS: 1-7. In other embodiments, the region A encodes a human Helios polypeptide, optionally wherein the nucleic acid encoding the human Helios polypeptide is at least 95% identical to one of SEQ ID NOS: 12-15.

In some embodiments, the region B encodes the human FOXP3 polypeptide, optionally wherein the human FOXP3 polypeptide is at least 95% identical to one of SEQ ID NOS: 8-11. In some embodiments, the region B comprises any one of SEQ ID NOS: 1-7. In other embodiments, the region B encodes a human Helios polypeptide, optionally wherein the nucleic acid encoding the human Helios polypeptide is at least 95% identical to one of SEQ ID NOS: 12-15.

In some embodiments, the region C encodes the human FOXP3 polypeptide, optionally wherein the human FOXP3 polypeptide is at least 95% identical to one of SEQ ID NOS: 8-11. In some embodiments, the region C comprises any one of SEQ ID NOS: 1-7. In other embodiments, the region C encodes a human Helios polypeptide, optionally wherein the nucleic acid encoding the human Helios polypeptide is at least 95% identical to one of SEQ ID NOS: 12-15.

In some embodiments, methods of treating or preventing inflammation or a disease or disorder associated with inflammation in a subject in need thereof are provided. In some embodiments, the method comprises administering to the subject a composition provided herein, a circular RNA provided herein, or a nucleoside-RNA molecule provided herein. Optionally, the composition is administered by a delivery route selected from the group consisting of intradermal, subcutaneous, intracranial, inhalation, intranasal, oral, peroral, and intramuscular.

In some embodiments, the disease or disorder is selected from the group consisting of: an age-related disease or disorder, mitochondrial disease or disorder, metabolic disorder, neurodegenerative disease, polyglutamine disease, anticoagulation condition, antithrombotic condition, allergy, respiratory condition, autoimmune disease, vision impairment, dyslipidemia, hyperlipidemia, diabetes, metabolic syndrome, inflammation, sepsis, apoptosis, autoimmunity, neurodegeneration, Alzheimer's disease, Parkinson's disease, Huntington's disease, oxidative stress, hypercholesterolemia, atherosclerosis, cardiovascular disease (CVD), steatohepatitis (fatty liver disease), pancreatitis, renal lipid deposition, depression, an elevated hCRP level, and cancer.

The present invention relates to are compositions, methods, processes, kits and devices for the design, preparation, manufacture and/or formulation of developing engineered T-regulatory cells from T-cells for immunotherapy and more specifically to methods for modifying T-cells by introducing nucleoside modified nucleic acid molecules encoding Forkhead box P3 (“FoxP3”), either alone or in combination with at least one other agent (“other agent”).

In one nonlimiting embodiment the nucleoside modified nucleic acid molecules encoding “FoxP3” either alone or in combination with “other agents” will be delivered in vivo.

In some embodiments the FoxP3 sequence comprises a nucleotide sequence at least 70%, 75%, 80%, 85%, 90%, 95%, 96%, 97%, 98%, 99%, 99.5% or 100% identical to SEQ ID NOs. 1-7.

In some embodiments the FoxP3 sequence comprises an amino acid sequence at least 70%, 75%, 80%, 85%, 90%, 95%, 96%, 97%, 98%, 99%, 99.5% or 100% identical to SEQ ID NOs. 8-11.

In one embodiment, provided herein a nucleoside modified nucleic acid molecules eg. Chimeric polynucleotides encoding FoxP3, wherein the nucleoside modified nucleic acid molecule has a sequence comprising Formula I,

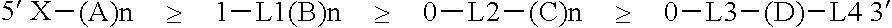

Wherein,
At least one of regions A, B, or C is positionally modified;
At least one of the regions A, B or C is polynucleotide encoding FoxP3
At least one of the regions A, B or C is a polynucleotide encoding at least one therapeutic agent
n is independently an integer between 0 and 1000;
X is a UTR with at least 1 to 20 kb in size
D is an optional region of linked nucleosides
L1, L2 and L3 are independently optional linker moieties, said linker moieties being either nucleic acid based, or non-nucleic acid based; and
L4 is an optional conjugate or an optional linker moiety, said linker moiety being either nucleic acid based, or non-nucleic acid based.

In one nonlimiting embodiment, such nucleoside modified nucleic acid molecules may include one or more agents encoding therapeutic agents such as a CAR and/or a stabilizing agents.

In one nonlimiting embodiment, such nucleoside modified nucleic acid molecules take the form of or function as modified mRNA molecules which encode one or more peptides or polypeptides of interest.

In one embodiment, at least one of the regions of the nucleoside modified nucleic acid molecules at least an open reading frame of a nucleic acid sequence such as, but not limited to SEQ1-15.

In one embodiment, at least one of the regions of the nucleoside modified nucleic acid molecules may be codon optimized for expression in human cells.

In another embodiment, the overall G:C content of the codon optimization region may be no greater than the G:C content prior to codon optimization.

In another embodiment, at least one of the regions of linked nucleoside modified nucleic acid molecules is a cap region. The cap region may comprise at least one cap such as, but not limited to, ARCA, CapO, Capl, Cap2, Cap4, 8-oxo-guanosine, 2-azido-guanosine, Nl-methyl-guanosine, LNA-guanosine, 2′ fluoro-guanosinc, 7-deaza-guanosine, 2-amino-guanosine, and inosine.

In one embodiment, at least one of the regions of nucleoside modified nucleic acid molecules is a polyA tail region.

In another embodiment, the nucleoside modified nucleic acid molecules described herein may also be circular.

In one embodiment, the nucleoside modified nucleic acid molecules for FoxP3 may be encoded across two regions.

In one embodiment, the composition relates to having a delivery vehicle conjugated to an activated T-cell targeting domain, wherein the delivery vehicle comprises at least one agent encoding FoxP3

In one nonlimiting embodiment, the composition relates to having a delivery vehicle conjugated to an activated T-cell targeting domain, wherein the delivery vehicle comprises at least one agent encoding FoxP3 with a CAR.

In one embodiment, the composition relates to having a delivery vehicle comprises at least one agent that directs the activated T-cell to a T-regulatory cell.

In other embodiments, the delivery vehicle directs the target immune cell to express FoxP3 and convert to a cell with T-regulatory properties.

In other embodiments, the delivery vehicle directs the target immune cell to express a CAR or TCR which is specific for binding to the following suitable inflammatory markers: VCAM-1, I-CAM-1 GFAP, ADRP, PECAM-1 CD14, IL-1R1, IL-1R2, MDA-LDL, CLDN7, CCR1/CCRL1, CCR2, CCRL2, CCR3, CCR4, CCR5, CCR6, CCR7, CCR8, CCR9, CCR10, TNFR1, TNFR2, LTBR, CD134, CD40, Fas receptor, DcR3, CD27, CD30, CD137, DR4, DR5, DcR1, DcR2, RANK, Osteoprotegerin, TweakR, TAC, BAFFR, HVEM, NGFR, BCMA, GITR, TAJ/TROY, IL2R, IL15R, IL4R, IL13R, IL7R, IL7RA, IL9R, IL21R, IL3R, IL3RA, IL5R, IL5RA, IL6RA, IL11R, IL27R, OSMR, LIFR, CNTFR, IL12R, IL23R, IL10R, IL22R, IL20R, IL28R, IFNAR1, IFNAR2, -γ/IFNGR1, IFNGR2, IL18R, IL17R, TGFBR1, TGFBR2, MOG, CEA, MBP, FVIII, CD19, Myosin heavy chain alpha including peptide fragments and Myosin Heavy chain alpha peptide 614-628.

In certain embodiments the agent associated therewith comprises a nucleoside modified nucleic acid molecules encoding FoxP3 and a chimeric antigen receptor (CAR) molecule specific for binding to a protein expressed on the surface of an inflammatory cell.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a schematic showing in vivo reprogramming of T cells into Tregs using the compositions provided herein.

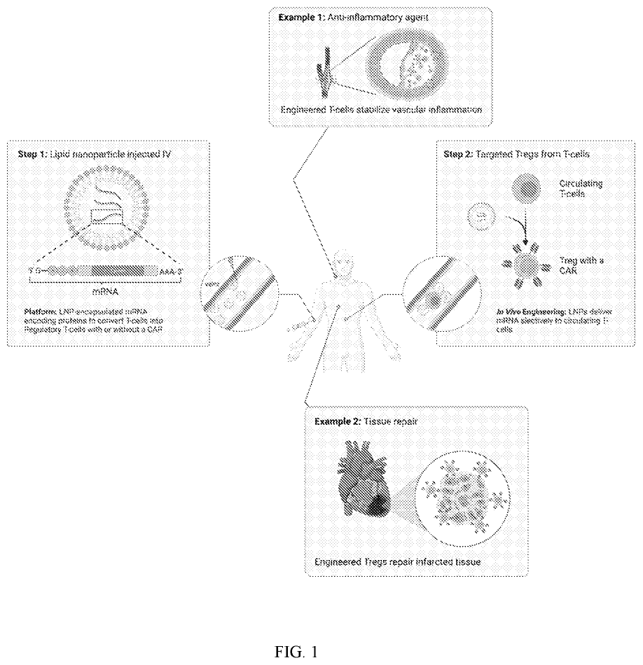

FIG. 2 depicts various exemplary constructs of the nucleoside-modified RNA molecule and the optional second agent, IRES, linker, UTR, 5′ cap, and polyA tail contained in the compositions provided herein.

FIG. 3 depicts additional exemplary constructs of the nucleoside-modified RNA molecule and the optional second agent, IRES, linker, UTR, 5′ cap, and polyA tail contained in the compositions provided herein.

FIG. 4 depicts an exemplary circular RNA construct containing a nucleic acid encoding a therapeutic agent, a nucleic acid encoding a FOXP3 polypeptide, an IRES, a 5′ UTR, a 3′ UTR, and a stop codon.

FIG. 5 is a western blot showing expression of CircRNA GFP, CircRNA GFP V_2, CircRNA-IRES1-NeoG-IRES2-RFP, and CircRNA FoxP3 is maintained in the presence and absence of RNAse R nuclease.

FIG. 6 shows GFP expression is maintained in vitro in a circRNA GFP construct in the presence of RNAse R.

FIG. 7 is a schematic showing the experimental design of a Circular RNA FoxP3 and linear RNA FoxP3 murine study.

FIG. 8 shows FoxP3 expression in lymphocytes in mice treated with either linear RNA GFP, linear RNA foxP3, circular RNA GFP, or circular RNA FoxP3.

FIG. 9 shows FoxP3 expression in splenocytes in mice treated with either linear RNA GFP, linear RNA foxP3, circular RNA GFP, or circular RNA FoxP3.

FIG. 10 is a schematic showing the experimental design of a Circular RNA FoxP3 and linear RNA FoxP3 murine study in an LPS-induced inflammation model.

FIG. 11 shows FoxP3 expression in lymphocytes in mice treated with either linear RNA GFP, linear RNA foxP3, circular RNA GFP, circular RNA FoxP3, LPS only, or untreated.

FIG. 12 shows FoxP3 expression in splenocytes in mice treated with either linear RNA GFP, linear RNA foxP3, circular RNA GFP, circular RNA FoxP3, LPS only, or untreated.

FIG. 13 shows FoxP3 expression in circular RNA FoxP3-treated lymphocytes and splenocytes treated at 12- and 72-hours post LPS injection.

FIG. 14 shows FoxP3 expression in circular RNA FoxP3-treated lymphocytes 12-hours post LPS injection.

FIG. 15 shows IL-17a and IFNg expression in circular RNA FoxP3-treated splenocytes post LPS injection.

FIG. 16 shows serum IL-10 levels in mice injected with circular RNA encoding FoxP3.

### DEFINITIONS

As used in this specification and the appended claims, the singular forms “a,” “an,” and “the” include plural reference unless the context clearly dictates otherwise.

The use of any and all examples or exemplary language (e.g., “for example” or “such as”) provided herein, is intended merely to better illustrate the invention, and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of” those certain elements. As used herein, “and/or” refers to and encompasses any and all possible combinations of one or more of the associated listed items, as well as the lack of combinations where interpreted in the alternative (“or”).

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “antibody” refers to a protein having antigen binding activity and an amino acid sequence from or derived from the framework region of an immunoglobulin encoding gene of an animal producing antibodies. The term includes but is not limited to polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompasses conjugates, including but not limited to fusion proteins containing an immunoglobulin moiety (e.g., chimeric or bispecific antibodies or scFv's), and fragments, such as Fab, F(ab′)2, Fv, single chain variable fragment (scFv), Fd, single domain (dAb) and other compositions.

As used herein, “chimeric antigen receptor” or “CAR” is a recombinant receptor for antigens that redirect the specificity for T cell binding. CARs typically consist of an extracellular antigen-binding domain, for example a scFv, a spacer, a transmembrane domain, and one or more cytoplasmic domains.

As used herein, the term “nanoparticle” refers to a polymeric particle in the nanometer range.

The term “nucleic acid” or “nucleotide” refers to deoxyribonucleic acids (DNA) or ribonucleic acids (RNA) and polymers thereof, for example, “polynucleotides,” in either single- or double-stranded form. The nucleic acid molecule may be derived from a variety of sources, including DNA, cDNA, synthetic DNA, RNA, or combinations thereof. Such nucleic acid sequences may comprise genomic DNA which may or may not include naturally occurring introns. Moreover, such genomic DNA may be obtained in association with promoter regions, introns, or poly A sequences. The nucleic acid molecule, for example RNA, may be linear or circular.

An “internal ribosome entry site” (IRES) refers to a cis-acting polynucleotide sequence, which when present in an RNA, mediates internal entry of the 40S ribosomal subunit upstream of a translation initiation codon in eukaryotic and viral mRNAs.

A polynucleotide component, for example an IRES, is “operably linked” when it is placed into a functional relationship with another nucleic acid sequence, for example, a polynucleotide. For example, an IRES is operably linked to a coding sequence if it affects the translation of the sequence.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The term “amino acid” refers to naturally occurring and synthetic amino acids, as well as amino acid analogs and amino acid mimetics that function in a manner similar to naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) as well as pyrrolysine, pyrroline-carboxy-lysine, and selenocysteine.

A “small molecule” refers to a molecule, for example a lipid, weighing less than 1.5 kilodalton.

“Percentage of sequence identity” is determined by comparing two optimally aligned sequences over a comparison window, wherein the portion of the amino acid sequence or polynucleotide sequence in the comparison window may comprise additions or deletions (i.e., gaps) as compared to the reference sequence (e.g., SEQ ID NO: 1 or 6), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 95% identity, optionally 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. For an amino acid sequence, optionally, identity exists over a region that is at least about 50 amino acids in length, or more preferably over a region that is 100 to 150 or 200 or more amino acids in length, or where not indicated over the entire length of the reference sequence.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 50 to 600, usually about 75 to about 200, more usually about 100 to about 150 in which a sequence may be compared to a reference sequence of the same number of contiguous positions after the two sequences are optimally aligned. Methods of alignment of sequences for comparison are well known in the art.

An algorithm for determining percent sequence identity and sequence similarity is the BLAST 2.0 algorithms, e.g., as described in, and Altschul et al. (1990) J. Mol. Biol. 215:403-410 (see also Altschul et al. (1977) Nuc. Acids Res. 25:3389-3402). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, and N=−4.

### DETAILED DESCRIPTION OF THE INVENTION

The inventors have discovered that the administration of a nanoparticle containing a nucleoside modified RNA molecule encoding Forkhead box P3 (“FOXP3”), either alone or in combination with at least one other agent, can be used for in vivo generation of stable Tregs from circulating immune cells.

This disclosure provides for compositions comprising at least one nanoparticle conjugated to a targeting domain, wherein the nanoparticle contains at least one nucleoside-modified RNA molecule encoding a human FOXP3 polypeptide. The nanoparticle may be selected from the group consisting of a lipid carrier, a liposome, a lipid nanoparticle, and a micelle. Optionally, the nanoparticle is another delivery vehicle.

In some embodiments, the nanoparticle is a lipid nanoparticle. The lipids can be made from one or a mixture of different lipids. Lipids are formed from one or more lipids, which can be neutral, anionic, cationic, non-cationic, or ionizable. Optionally, the nanoparticle comprises an ionizable lipid. Ionizable lipids can be positively charged during production, neutral in storage and in the blood, and revert to positive charge in vivo. In some embodiments, ionizable lipids may be composed of an amine moiety and a lipid moiety, and a cationic amine moiety and a polyanion nucleic acid interact electrostatically to form a positively charged liposome or lipid membrane structure. For example, the ionizable lipid can be C12-200, C12-E1, C12-A1, C12-E2, C12-A2, C12-E3, C12-A3, C14-E1, C14-A1, C14-E2, C14-A2, C14-E3, C14-A3, C16-E1, C16-A1, C16-E2, C16-A2, C16-E3, or C16-A3.

Optionally, the nanoparticle comprises a lipid nanoparticle with a polyethylene glycol (PEG)-lipid conjugated to the targeting domain. Optionally, the nanoparticle comprises one or more cationic lipids, one or more non-cationic lipids and PEG or other lipid excipients like dioleoylphosphoethanolamine (DOPE), or cholesterol. Optionally, the nanoparticle has a 35%, 16%, 42.5%, and 2.5% (e.g., 2-45%, 5-45%, 5-30%) molar ratio of ionizable lipids, DOPE, cholesterol, and PEG, respectively.

The lipids may comprise one or more lipids or amphiphilic compounds. For example, the nanoparticles can be liposomes, lipid micelles, solid lipid particles, or lipid-stabilized polymeric particles. The liposomes can be unilamellar and/or multilamellar liposomes. By way of example, cationic liposomes can be formed from a composition of cationic lipids and phospholipids to form aggregates with macromolecules such as DNA and RNA.

Other exemplary nanoparticles and their manufacture are described in the art, for example in U.S. patent Ser. No. 11/712,481B2, U.S. Application Publication No. 2023/0090515, U.S. Application Publication No. 2023/0364024, U.S. Application Publication No. 2022/0062175, and U.S. Application Publication No. 2023/0090515.

The nanoparticles described herein are conjugated to a targeting domain that specifically binds to a cell surface antigen of a T-cell, a progenitor cell or a precursor to a T cell. The targeting domain may be selected from the group consisting of a nucleic acid molecule, a peptide, an antibody, and a small molecule. The targeting domain may be conjugated to the nanoparticle by methods known in the art, for example, the method contained in Tombacz, et al., Mol. Ther., 29(11): 3932-3304 (2021). In some embodiments, the targeting domain is an anti-CD4 antibody. In some embodiments, the targeting domain is an interleukin 1 receptor type 1 (IL1R1) antagonist. For example, the IL1R1 antagonist can be a protein (e.g., anakinra, isunakinra, or rilonacept) or an antibody (e.g., canakinumab).

The targeting domain binds to a cell surface antigen of a T cell, a progenitor cell or a precursor to a T cell. Optionally, the target antigen is on an activated T cell. The target antigen may be selected from the group consisting of CD1, CD2, CD3, CD4, CD5, CD7, CD8, CD16, CD25, CD26, CD27, CD28, CD30, CD38, CD39, CD40L, CD44, CD45, CD62L, CD69, CD71, CD73, CD80, CD83, CD86, CD95, CD103, CD119, CD126, CD150, CD152, CD153, CD154, CD161, CD183, CD223, CD254, CD275, CD45RA, CXCR3, CXCR5, FasL, IL18RI, CTLA-4, OX40, GITR, LAG3, ICOS, PD-1, leu-12, TCR, TLRI, TLR2, TLR3, TLR4, TLR6, NKG2D, CCR, CCRI, CCR2, CCR4, CCR6, and CCR7.

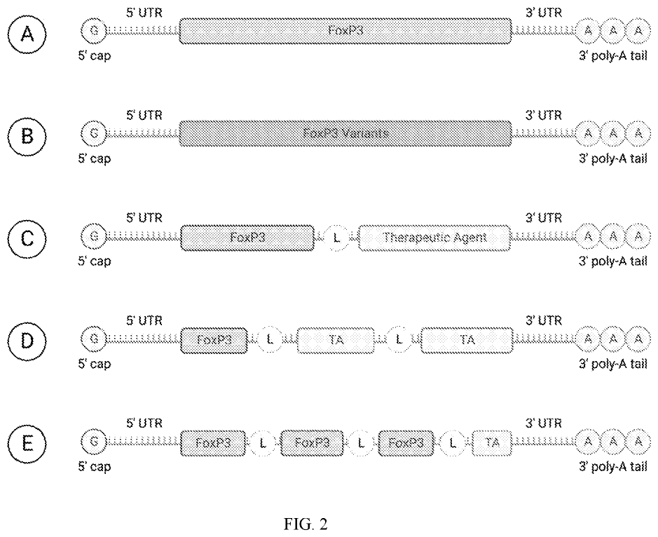

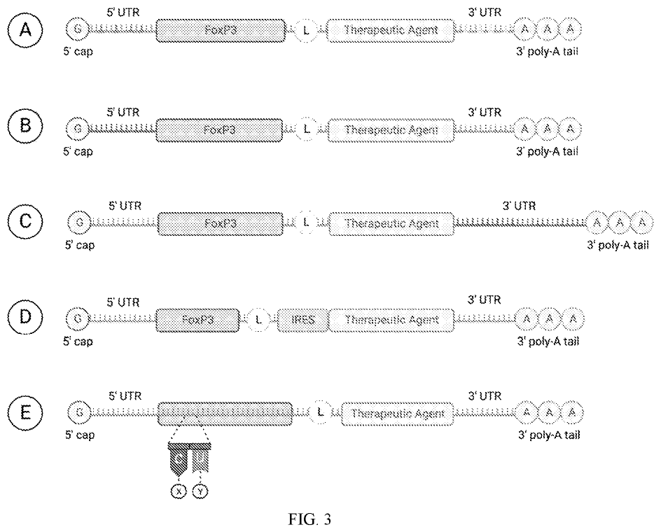

The nanoparticles described herein contain at least one (optionally nucleoside-modified) RNA molecule comprising a coding sequence for a human FOXP3 polypeptide. As shown in FIGS. 2-3, the nanoparticles described herein may contain more than one nucleoside-modified RNA molecules comprising a coding sequence for a human FOXP3 polypeptide. In some embodiments, the nanoparticles described herein contain 1, 2, 3, 4, 5, 6, 7, 8, 9, or 10 such RNA molecules. In some embodiments, the human FOXP3 polypeptide comprises an amino acid sequence at least 80, 85, 90, 95, 98, 99, or 100% identical to any one of SEQ ID NOS: 8-11. In some embodiments, the nucleoside-modified RNA molecule has the sequence of any one of SEQ ID NOS: 1-7. Optionally, the nucleoside-modified RNA sequence is codon optimized.

The RNA molecule contained in the nanoparticles described herein can contain at least one nucleoside modification. In certain embodiments, the nucleoside modification does not change the nucleoside function. In certain embodiments, the nucleoside modification optionally improves stability, for example by preventing degradation. In some embodiments, the nucleoside modification is the addition of a long chain of adenine nucleotides at the 3′ end of RNA (i.e., a polyA tail).

In some embodiments, a truncated FOXP3 polypeptide is encoded by the RNA molecule. Optionally, the nucleoside modification results in the forkhead (FKH) domain being partially or completely deleted. See, e.g., Lopes, et al., J. Immunol., 177(5): 3133-3142 (2006). In other cases, the nucleoside modification results in a FOXP3 polypeptide consisting only of the central region of the protein, including the leucine zipper and zinc finger (ZnF/Zip), see, e.g., Lopes, et al., J. Immunol., 177(5): 3133-3142 (2006). In other embodiments, the nucleoside modification results in a FOXP3 polypeptide consisting of amino acids, beginning at the N-terminus, 1-105, 67-132, 101-198 or 106-198 of SEQ ID NO: 8.

In certain embodiments, the nucleoside modification enhances translation. Optionally, the RNA molecule encoding FOXP3 or other RNA described herein comprises one or more (e.g., 2, 3, 4, 5, 6, 7, 8, 9 or 10 or more) modified uridines. Exemplary modifications can include but are not limited to pseudouridine, 1-methylpseudouridine (i.e., N1-methyl pseudouridine), N1-ethyl pseudouridine, 2-thiouridine, 4′-thiouridine, 5-Methyluridine, 1-methyluridine, 2-thio-1-methyl-1-deaza-pseudouridine, 2-thio-T-methyl-pseudouridine, 2-thio-5-aza-uridine glycosides, 2-thio-dihydropseudouridine, 2-sulfanyl-dihydrouridine, 2-sulfanyl-pseudouridine, 4-methoxy-2-sulfanyl-pseudouridine, 4-methyl Oxy-pseudouridine, 4-thio-1-methyl-pseudouridine, 4-thio-pseudouridine, 5-aza-uridine, 5-carboxyhydroxyuridine, 5-hydroxyuridine, dihydropseudouridine, 5-carbamoylmethyluridine, or 5-methoxyuridine.

In other embodiments, the RNA molecule encoding FOXP3 or other RNA molecule described herein comprises a modified cytidine. Exemplary modifications can include but are not limited to 5-methylcytosine, N4-acetylcytidine, 5-hydroxymethylcytosine, 5-formylcytosine, 1-methyladenosine, 2-thiocytidine, 1-methylinosine, 5-carboxylcytidine, 3-methylcytidine, and 2′-O-methylcytidine.

In other embodiments, the RNA molecule encoding FOXP3 or other RNA molecule described herein comprises a modified guanosine. Exemplary modifications can include but are not limited to N7-methylguanosine, 2′-O-methylguanosine, N2,N2-dimethylguanosine, N2-methylguanosine, 7-methylguanosine, 1-methylguanosine, 7-deazaguanosine, queuosine, or a guanosine hypermodification (e.g., archaeosine, wybutosine and imG).

In other embodiments, the RNA molecule encoding FOXP3 or other RNA molecule described herein comprises a modified adenosine, N6-methyladenosine, N6,2′-O-dimethyladenosine, N6-isopentenyladenosine, N6-threonylcarbamoyladenosine, 2-methylthio-N6-threonylcarbamoyladenosine, N6-hydroxynorleucylcarbamoyladenosine, and 2-methyladenosine, N6-acetyllysyladenosine.

Optionally, the RNA molecule comprises a 5′ cap region. The 5′ cap structure is involved in nuclear export and molecule stability. Exemplary caps include, but are not limited to, Anti-Reverse Cap Analog (ARCA), Cap-0, Cap-1, Cap-2, Cap-4, 8-oxo-guanosine, 2-azido-guanosine, N1-methyl-guanosine, LNA-guanosine, 2-Flouro-2′-deoxygyanosine, 7-deazaguanosine, 2-amino-guanosine, and inosine.

In certain embodiments, the nucleoside modification comprises a positional modification wherein at least two nucleosides are chemically modified. Any of adenosine, guanosine, cytidine, or uridine may be modified in the manner described above or known in the art, for example in Canadian Patent Application No. 2923029. The at least two chemical modifications may be the same or different. Positional modifications may include 2, 3, 4, 5, 6, 7, 8, 9, 10, or more chemical modifications.

In certain embodiments, the RNA molecule comprises an untranslated region (UTR) (see, e.g., FIGS. 2-3). A UTR is a section of nucleic acids that are transcribed but not translated. As shown in FIGS. 2-4, the UTR may be located at either the 5′ or 3′ end of the RNA molecule, or both the 5′ and 3′ end. The UTR optionally comprises one or more mutations or modifications, for example an AU-rich sequence. The UTR is optionally from a different gene than is encoded by the RNA molecule, for example but not limited to, a UTR from the beta-globin gene. In some embodiments, the UTR is from a histone, a cyclin, a heat shock protein (HSP), or a cytokine (e.g., Interleukin, for example but not limited to, IL-2 or IL-6). In other embodiments, the UTR is from actin, elongation factor 1-alpha (EF1A), collagen, glyceraldehyde 3-phosphate dehydrogenase (GAPDH), ferritin, ovalbumin, albumin, insulin, epidermal growth factor (EGF), or transferrin receptor (TfR).

In certain embodiments, the RNA molecule comprises a linker moiety. See, for example, FIGS. 2-3. The linker moiety can be used to attach coding regions. Linker moieties may be nucleic-acid based (e.g., Poly-A tail, Poly-UG/UA repeat, (GU)n, or aptamers) or non-nucleic acid based (e.g., Gly-Gly-Gly-Gly-Ser)n, (Ala-Gly-Ala-Gly)n, or (Proline)n where n is an integer). In other embodiments, linker moieties may be derived from viral or phage peptides (e.g., thosea asigna virus 2A (T2A) peptide or porcine teschovirus-1 2A (P2A) peptide), or from hammerhead ribozymes.

In some embodiments, the RNA molecules comprise a conjugate. Optionally, the RNA molecule is conjugated to one or more other polynucleotides, dyes, intercalating agents (e.g., acridines), cross-linker (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug.

The nanoparticles described herein optionally comprise a second agent or a nucleic acid encoding the second agent. Optionally, more than one second agents are present in the nanoparticles described herein. In some embodiments, the second agents is encoded by an RNA. In some embodiments, the second agent is encoded by the RNA that encodes the FOXP3 protein. The arrangement of the optional second agent and the at least one nucleoside-modified RNA molecule comprising a coding sequence for a human FOXP3 polypeptide may vary. For example, the RNA molecule encoding FOXP3 may be at the 5′ end and the second agent at the 3′ end or the RNA, as shown in FIGS. 2-3. Optionally, the second agent is at the 5′ end of the RNA followed by the sequence encoding FOXP3. Additionally, the ratio of the optional second agent and RNA molecule encoding FOXP3 may vary. In some embodiments, there are more RNA molecules encoding FOXP3 than there are second agents (FIG. 2). In other embodiments, there are more second agents than there are RNA molecules encoding FOXP3 (FIG. 2). In embodiments in which both FOXP3 and the second agent are encoded by the same RNA, the first coding sequence can in some embodiments be translated at a higher rate than the subsequent coding sequence on the RNA, thereby also controlling the ratio of expression between the first and second encoded protein in the RNA. The second agent may be in some embodiments a therapeutic agent, a stabilizing agent, an imaging agent, a diagnostic agent, a labeling agent, or a detection agent. In some embodiments, the second agent is a nucleic acid.

In some embodiments, the nanoparticles comprise a therapeutic agent, as shown in FIGS. 2-4. The therapeutic agent optionally targets, i.e., causes reduction in, inflammation. In some embodiments, the therapeutic agent is a nucleoside modified RNA encoding a CAR or a T-cell receptor (TCR). Optionally, the nucleoside modified RNA encoding a CAR comprises SEQ ID NOS: 19, 20, 21, or 22. In these embodiments, CAR is delivered to and expressed in the T cell, a progenitor cell or a precursor to a T cell, along with expression of the FOXP3. The CAR will assist in targeting the T cell, a progenitor cell or a precursor to a T cell to a source of inflammation.

In some cases, the CAR or TCR comprises an extracellular binding domain that binds to inflammatory markers, for example, VCAM-1, I-CAM-1 GFAP, ADRP, PECAM-1 CD14, IL-1R1, IL-1R2, MDA-LDL, CLDN7, CCR1/CCRL1, CCR2, CCRL2, CCR3, CCR4, CCR5, CCR6, CCR7, CCR8, CCR9, CCR10, TNFR1, TNFR2, LTBR, CD134, CD40, Fas receptor, DcR3, CD27, CD30, CD137, DR4, DR5, DcR1, DcR2, RANK, Osteoprotegerin, TweakR, TAC, BAFFR, HVEM, NGFR, BCMA, GITR, TAJ/TROY, IL2R, IL15R, IL4R, IL13R, IL7R, IL7RA, IL9R, IL21R, IL3R, IL3RA, IL5R, IL5RA, IL6RA, IL11R, IL27R, OSMR, LIFR, CNTFR, IL12R, IL23R, IL10R, IL22R, IL20R, IL28R, IFNAR1, IFNAR2, γ/IFNGR1, IFNGR2, IL18R, IL17R, TGFBR1, TGFBR2, MOG, CEA, MBP, FVIII, CD19, Myosin heavy chain alpha including peptide fragments and Myosin Heavy chain alpha peptide 614-628.

Chimeric antigen receptors (CARs) are recombinant receptor constructs comprising an extracellular antigen-binding domain joined to a transmembrane domain, and further linked to an intracellular signaling domain (e.g., an intracellular T cell signaling domain of a T cell receptor) that transduces a signal to elicit a function.

In some standard CAR embodiments, the components include an extracellular targeting domain, e.g., as described herein, a transmembrane domain and intracellular signaling/activation domain, which are typically linearly constructed as a single fusion protein. The “transmembrane domain” is the portion of the CAR that links the extracellular binding portion and intracellular signaling domain and anchors the CAR to the plasma membrane of the host cell that is modified to express the CAR. The intracellular region may contain a signaling domain of TCR complex, and/or one or more costimulatory signaling domains, such as those from CD28, 4-1BB (CD137) and OX-40 (CD134). For example, a “first-generation CAR” generally has a CD3-zeta signaling domain. Additional costimulatory intracellular domains may also be introduced (e.g., second and third generation CARS) and further domains including homing and suicide domains may be included in CAR constructs. CAR components are further described below.

A CAR construct encoding a CAR may also comprise a sequence that encodes a signal peptide to target the extracellular domain to the cell surface.

In some embodiments, the CAR may contain one or more hinge domains that link the antigen binding domain and the transmembrane domain for positioning the antigen binding domain. Such a hinge domain may be derived either from a natural, synthetic, semi-synthetic, or recombinant source. The hinge domain can include the amino acid sequence of a naturally occurring immunoglobulin hinge region, e.g., a naturally occurring human immunoglobulin hinge region, or an altered immunoglobulin hinge region. Illustrative hinge domains suitable for use in the CARs described herein include the hinge region derived from the extracellular regions of type 1 membrane proteins such as CD8 alpha, CD4, CD28, PD1, CD 152, and CD7, which may be wild-type hinge regions from these molecules or may be altered.

Any transmembrane suitable for use in a CAR construct may be employed. Such transmembrane domains, include, but are not limited to, all or part of the transmembrane domain of the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB 1, CD29, ITGB2, CD 18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100, (SEMA4D), SLAM1F6 (NTB-A, Lyl08), SLAM (SLAMF1, CD150, IPO-3), BLAME, (SLAMF8), SELPLG (CD162), LTBR, PAG/Cbp, NKG2D, or NKG2C.

A transmembrane domain incorporated into a CAR construct may be derived either from a natural, synthetic, semi-synthetic, or recombinant source.

A CAR construct can include one or more intracellular signaling domains, also referred to herein as co-stimulatory domains, or cytoplasmic domains that activate or otherwise modulate an immune cell. The intracellular signaling domain is generally responsible for activation of at least one of the normal effector functions of the immune cell in which the CAR has been introduced. In one embodiment, a co-stimulatory domain is used that increases CAR immune T cell cytokine production. In another embodiment, a co-stimulatory domain is used that facilitates immune cell (e.g., T cell) replication. In still another embodiment, a co-stimulatory domain is used that prevents CAR immune cell (e.g., T cell) exhaustion. In another embodiment, a co-stimulatory domain is used that increases immune cell (e.g., T cell) antitumor activity. In still a further embodiment, a co-stimulatory domain is used that enhances survival of CAR immune cells (e.g., T cells) (e.g., post-infusion into patients).

Examples of intracellular signaling domains for use in a CAR include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains include those of CD3 zeta, common FcR gamma, Fc gamma Rlla, FcR beta (Fc Epsilon Rib), CD3 gamma, CD3 delta, CD3 epsilon, CD79a, CD79b, DAP10, and DAP12. In one embodiment, a CAR comprises an intracellular signaling domain, e.g., a primary signaling domain of CD3-zeta.

An intracellular signaling domain of a CAR can comprise a primary intracellular signaling domain only, or may comprise additional desired intracellular signaling domain(s) useful in the context of a CAR. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that binds to CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD 160, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB 1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAMl, CRTAM, Ly9 (CD229), CD160 (BY55), PSGL1, CD100 (SEMA4D), CD69, SLAMF6 (NTB-A, Lyl08), SLAM, (SLAMFl, CD150, IPO-3), BLAME (SLAMF8), SELPLG (CD162), LTBR, LAT, GADS, SLP-76, PAG/Cbp, and CD 19a.

In some embodiments, the CAR is expressed in combination with at least one RNA molecule encoding at least one protein with a secretory signal. Exemplary proteins with a secretory signal may include albumin or an IgG antibody.

In other embodiments, the therapeutic agent is a receptor linked to a downstream effector. For example, a CAR targeting a specific tissue or disease state. In some embodiments, the CAR targets cardiac tissue, vascular tissue, intestinal tissue, skin including the dermis and epidermis, lung tissue, skeletal muscle tissue, tendons, ligaments, bones and synovium, renal tissue including glomeruli, nervous tissue including peripheral nerves and central nervous tissue including the brain and spinal cord, and mesenchymal tissue. In other embodiments, the CAR targets an antigen associated with myocarditis, myocardial infarction, vasculitis, aneurysms, atherosclerosis, encephalomyelitis, multiple sclerosis, amyotrophic lateral sclerosis, Alzheimer's disease, Crohn's disease, ulcerative colitis, esophagitis, gastritis, chronic obstructive pulmonary disease, emphysema, arthritis, rheumatoid arthritis, diabetes, thyroiditis, glomerulonephritis, hepatitis, systemic lupus erythematosus or another type of lupus, Hashimoto's disease, Graves disease, Sjogren's syndrome, psoriasis, or another autoimmune disease.

In some embodiments, the therapeutic agent is an RNA molecule encoding at least one component for gene editing, for example an RNA-guided endonuclease, which may be an active endonuclease, or an endonuclease engineered to lack endonuclease activity by introduction of 1, 2, or more mutations (e.g., dCas9). The at least one component for gene editing may be a CRISPR associated protein 9 (Cas9), a guide RNA, or both of a Cas9 and a guide RNA. Cas9 mRNA encodes the Cas9 protein, which is an endonuclease capable of binding and cutting DNA at specific targets useful for gene editing. A guide RNA sequence directs Cas9 to the target DNA site.

In some embodiments, the nanoparticles provided herein contain a stabilizing agent. The stabilizing agent optionally stabilizes FOXP3 expression in Tregs. The function of the stabilizing agent is optionally measured by FOXP3 expression, as assessed by one of the known methods in the art, e.g., flow cytometry or immunohistochemistry. The stabilizing agent may be, for example, IKZF2, PP1, NLK, OGT, OGA, SIRT1, RORγt, USP7, USP21, RNF31, TRAF6, PRMT1, PRMT5, NFAT, LAG-3, GITR, NRP1, c-REL, ALPK1, CREB, STAT5, SMAD3, RXR, ICOS, PHD3, FOXO1, IL-2R, IDO, TIGIT, GARP, CD98, CD28, CD73 and CD39. In some embodiments, the stabilizing agent is IKZF2 (Helios) polypeptide. In some embodiments, IKZF2 is a polypeptide at least 95% identical to SEQ ID NO: 16. In some embodiments, IKZF2 is encoded by, one of SEQ ID NOS: 12-15 or a sequence at least 95, 98, or 99% identical to one of SEQ ID NOS: 12-15.

In some embodiments, the nanoparticles provided herein contain an imaging agent. Optionally, the imaging agent is a fluorescent dye, for example a fluorescein, a rhodamine, a cyanine, or quantum dots. Optionally, the imaging agent is a magnetic resonance imaging (MRI) contrast agent, for example, a gadolinium-based agent, a manganese-based agent, or an iron oxide nanoparticle. Optionally, the imaging agent is a positron emission tomography (PET) radiotracer, for example, Fluorine-18 (¹⁸F), Copper-65 (⁶⁴Cu), or Zirconium-89 (⁸⁹Zr). Optionally, the imaging agent is a single-photon emission computerized tomography (SPECT) radiotracer, for example Technetium-99m (^{99m}Tc) or Indium-111 (¹¹¹In).

In some embodiments, the nanoparticles provided herein contain a diagnostic agent. Optionally, the diagnostic agent is an antibody conjugated to target a disease marker. In other embodiments, the diagnostic agent is an aptamer specific to a disease marker. In some embodiments, the antibody or aptamer targets a marker associated with myocarditis, myocardial infarction, vasculitis, aneurysms, atherosclerosis, encephalomyelitis, multiple sclerosis, amyotrophic lateral sclerosis, Alzheimer's disease, Crohn's disease, ulcerative colitis, esophagitis, gastritis, chronic obstructive pulmonary disease, emphysema, arthritis, rheumatoid arthritis, diabetes, thyroiditis, glomerulonephritis, hepatitis, systemic lupus erythematosus or another type of lupus, Hashimoto's disease, Graves disease, Sjogren's syndrome, psoriasis, or another autoimmune disease. In other embodiments, the diagnostic agent is an enzyme (see, for example, Singh, et al., Advances in Enzyme Technology 225-271 (2019)).

In some embodiments, the nanoparticles provided herein contain a labeling agent. Optionally, the labeling agent is a fluorescent protein such as green fluorescent protein (GFP), mCherry, or near-infrared fluorescent protein (iRFP). Optionally, the labeling agent is an isotope label, for example Tritium (3H), Carbon-14 (¹⁴C), or Sulfur-35 (³⁵S).

In some embodiments, the nanoparticles provided herein contain a detection agent. Optionally, the detection agent is a luminescent dot, for example quantum dots or firefly luciferase. Optionally, the detection agent is an electron-dense marker, for example colloidal gold nanoparticles used in electron microscopy. Optionally, the detection agent is a photoacoustic marker, for example indocyanine green (ICG) or methylene blue.

Optionally, the nanoparticles provided herein contain at least one (optionally nucleoside-modified, and optionally circular) RNA molecule comprising a coding sequence for a human FOXP3 polypeptide and a second agent (e.g., second polypeptide) selected from the group of: IKZF2, NFAT, CREB, IRF4, Cbfβ, and SATB1.

The RNA molecules contained in the nanoparticles described herein may be linear or circular. Optionally, the circular RNA molecules comprise at least 5% N⁶-methyladenosine (m⁶A) relative to total nucleoside bases. m⁶A is a methylation at the adenosine nitrogen-6 position and is one of the most abundant RNA modifications. On mRNAs, m⁶A has been demonstrated to regulate different functions including splicing, translation, and degradation, which can have cell- and tissue-wide effects (see, for example, Zhang et al., J. Exp. Clin. Cancer Rsch., 39: 192 (2020)). m⁶A is also present on circRNA and has the potential to initiate cap-independent translation (see, for example, Qin et al., Mol. Med., 28: 79 (2022)). In some embodiments, the circular RNA molecules described herein may contain 5-20% or more m⁶A relative to total nucleoside bases. The percentage of m⁶A may be confirmed by methods known in the art, for example liquid chromatography coupled with tandem mass spectrometry or enzyme-linked immunosorbent assay (ELISA).

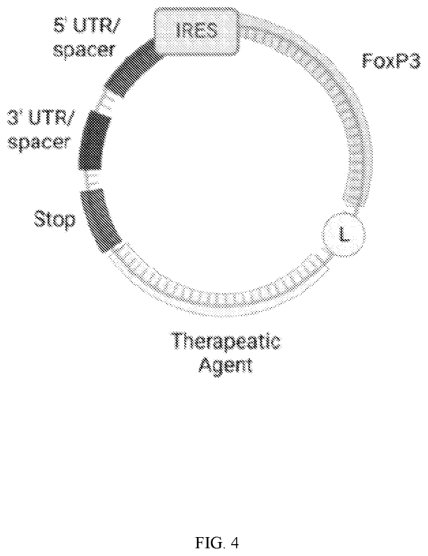

Circular RNAs lack both a 5′ and 3′ end since they are covalently joined head to tail. Conventional eukaryotic translation initiation relies on a 5⁷G cap at the 5′ end of linear RNA for ribosome recruitment. Without the translation initiation sites in the 5′ end, naturally-occurring circular RNA is generally non-coding. See, for example, Chen, et al., Nature Biotechnology, 41: 262-272 (2023). IRES play a role in initiating protein synthesis in the absence of the 5′ cap structure in the circular RNA. Some viruses containing circular RNA utilize IRES. An IRES may act as the sole ribosome binding site or may serve as one of the multiple binding sites. RNA molecules described herein may contain one or more IRES operably linked to the same or different coding sequence (FIGS. 3-4).

In some embodiments, the IRES is derived from a virus. Examples of viruses from which IRES can be derived are known in the art and include picornaviruses, hepatitis viruses, herpesviruses, and pestiviruses. In some embodiments, the IRES is any one of the following IRES: iEMCV, iSimianEV-A, iCovid19, iHCV, iCVB5, iCVA20, iSwineVesicular, iHRV-A2, iHRV-C3, iHRV-C11, iCVB1, iPV2, iHRV-B17, iEchoV-E15, iEchoV-E11, iEchoVll, iCrPV, iHRV-A89, iHRV-B26, iBEVI, iEchoV1, iHRV-A21, iPV1, iEV71, iHRV-A9, iSiminanV4, iEV-D94, iSimianA5, iPV3, iHRV-C54, iHRV-AlOO, iHRV-B37, iHRV-B4, iHRV-B92, iHRV-A1, iEV-B107, iHRV-A57, iHRV-B4, iHRV-B97, iHRVB-B14, or a fragment or derivative thereof. In other embodiments, the IRES is derived from a protein (e.g., fibroblast growth factor (FGF), platelet-derived growth factor (PDGF), or vascular endothelial growth factor (VEGF).

In some embodiments, circular RNA is synthesized from an expression vector comprising self-splicing introns, a 5′ spacer, a 3′ untranslated region (UTR), and an IRES. Expression vector refers to a DNA structure used to express, for example, a polynucleotide encoding a desired polypeptide. As described herein, expression vectors may include, for example, a collection of genetic elements that have a regulatory effect on gene expression, such as promoters and enhancers; structures or coding sequences that are transcribed into mRNA and translated into proteins; one or more IRES; a 5′ spacer of varying length; a 3′ UTR of varying length; and appropriate initiation and termination sequences. Any vector can be used, including plasmids, viruses, phages, and transposons. Optionally, the expression vector comprises a chromosomal, non-chromosomal and synthetic DNA sequence, such as a viral plasmid, bacterial plasmid, phage DNA, yeast plasmid, and a vector derived from combinations of plasmids and phage DNA, such as lentivirus, DNA of viruses such as retrovirus, vaccinia, adenovirus, fowlpox, baculovirus, SV40 and pseudorabies.

Also described herein is a circular RNA molecule (optionally comprising one or more nucleoside-modified uracil or other nucleotide as described elsewhere herein) comprising a first IRES operably linked to a first coding sequence and a second IRES operably linked to a second coding sequence. In some embodiments, the first IRES operably linked to the first coding sequence and the second IRES operably linked to the second coding sequence are separated by an intron. The term “intron,” as used herein, refers to a nucleic acid sequence present in a given gene which is removed by RNA splicing during maturation of the final RNA product. Introns are generally found between exons. During transcription, introns are removed from precursor messenger RNA (pre-mRNA), and exons are joined via RNA splicing. The intron used in the circular RNA molecule described herein may be, for example, a self-splicing group I intron, a self-splicing group II intron, a spliceosomal intron, or a tRNA intron. In some embodiments, the intron is a self-splicing group I or self-splicing group II intron. Group I and group II introns are advantageous in that they can be readily used for production of circular RNAs in vitro as well as in vivo because of their ability to undergo self-splicing due to their autocatalytic ribozyme activity.

In some embodiments, the circular RNA molecule comprises a third IRES operably linked to a third coding sequence. The first, second, and third coding sequences can optionally be chosen from the group consisting of a nucleic acid encoding a human Helios polypeptide (e.g., a polypeptide at least 95% identical to SEQ ID NO: 16) or a nucleic acid encoding a FOXP3 polypeptide (e.g., a polypeptide at least 95% identical to any one of SEQ ID NO: 8-11). In some embodiments, the third coding sequence is a CAR, a TCR, or another synthetic receptor (e.g., a synthetic Notch receptor) that can be used to activate a cell signaling pathway.

In other embodiments, the circular RNA molecule comprises 1, 2, 3, 4, 5, 6, 7, 8, 9, or 10 IRES each operably linked to a coding sequence. In some embodiments, the coding sequence for the human Helios polypeptide comprises one of SEQ ID NOS: 12-15. In some embodiments, the coding sequence for the human FOXP3 polypeptide comprises one of SEQ ID NOS: 1-7. In some cases, the circular RNA molecule is contained in a nanoparticle, e.g., as described herein. In further embodiments, the circular RNA further comprises a coding sequence for a CAR, e.g., as described herein. Thus in some embodiments, the circular RNA comprises an IRES and coding sequence for each of a FOXP3 protein and a Helios protein; or FOXP3 protein and a CAR protein; or FOXP3 protein and a Helios protein and a CAR protein.

Optionally, the circular RNA molecules comprise at least 5% m⁶A relative to total nucleoside bases. The circular RNA molecules described herein may contain 5-20% m⁶A relative to total nucleoside bases. The percentage of m⁶A may be confirmed by methods known in the art, for example liquid chromatography coupled with tandem mass spectrometry or enzyme-linked immunosorbent assay (ELISA).

Also provided herein is a method of treating or preventing inflammation or a disease or disorder associated with inflammation. Expression of at least FOXP3 in the target cell, for example a T-cell or other immune cell, can result in conversion of the cell to a helper T-cell phenotype, which in turn will reduce inflammation. Expression of further components described herein, e.g., the CAR, can direct the resulting cell to sites of inflammation. In some embodiments, the method comprises administering to the subject in need thereof an effective amount of any of the compositions described herein, for example a nanoparticle comprising an RNA (optionally comprising one or more nucleoside-modified uracil or other nucleotide as described elsewhere herein) encoding a FOXP3 protein and optionally a Helios protein or other stabilizing protein, a CAR protein, or both.

A disease or disorder associated with inflammation may be, for example, an age-related disease or disorder, mitochondrial disease or disorder, metabolic disorder, neurodegenerative disease, polyglutamine disease, anticoagulation condition, antithrombotic condition, allergy, respiratory condition, autoimmune disease, vision impairment, dyslipidemia, hyperlipidemia, diabetes, metabolic syndrome, inflammation, sepsis, apoptosis, autoimmunity, neurodegeneration, Alzheimer's disease, Parkinson's disease, Huntington's disease, oxidative stress, hypercholesterolemia, atherosclerosis, cardiovascular disease (CVD), steatohepatitis (fatty liver disease), pancreatitis, renal lipid deposition, depression, an elevated hCRP level, and cancer.

The subject may be a vertebrate, specifically a mammal. The term does not denote a particular age or sex. Thus, adult, newborn, and pediatric subjects, whether male or female, are intended to be covered. As used herein, patient or subject may be used interchangeably and can refer to a subject diagnosed with or at risk of developing a disorder. The term patient or subject includes human and veterinary subjects. In any of the methods provided herein, the subject can be a subject diagnosed with inflammation or a disease or disorder associated therewith, or at risk of developing inflammation or a disease or disorder associated therewith.

Administration is introducing, injecting, or otherwise physically delivering a substance as it exists outside of the body (e.g., the compositions provided herein) into a subject. Administration may be intradermal, subcutaneous, intracranial, inhalation, intranasal, oral, peroral, and intramuscular. Administration may occur in vivo, in vitro, or ex vivo. Preferably, the composition is administered in vivo.

An effective amount is an amount of any of the compositions described herein that, when administered to a subject, is effective, alone or in combination with additional agents, to treat a disease or disorder either by one dose or over the course of multiple doses. A suitable dose can depend on a variety of factors including the particular composition used and whether it is used concomitantly with other therapeutic agents. Other factors affecting the dose administered to the subject include, e.g., the type or severity of the disease.

Optionally, the composition is a unit dosage form having a dosage of 15 micrograms to 800 micrograms of the RNA molecule for a dose of up to 2.5 mg/kg in a human subject. Other exemplary effective amounts of the compositions described herein can be determined by one of ordinary skill in the art. Factors that influence dosage can include, e.g., other medical disorders concurrently or previously affecting the subject, the general health of the subject, the genetic disposition of the subject, diet, time of administration, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

In certain embodiments at least one nucleoside modified nucleic acid molecules encoding FoXP3, or a nucleoside modified nucleic acid molecule encoding FoxP3 and at least one therapeutic agent, for use in a method of treatment, amelioration, mitigation, slowing, arresting or reversing or prevention of an age-related disease or of a condition selected from the group consisting of: mitochondrial-related diseases/disorders, metabolic disorders, neurodegenerative diseases, polyglutamine diseases, anticoagulation and antithrombotic conditions, allergies and respiratory conditions, autoimmune diseases, vision impairment, dyslipidemia, hyperlipidemia, diabetes, metabolic syndrome, inflammation, apoptosis, neurodegeneration, oxidative stress and cancer, hypercholesterolemia, autoimmunity, atherosclerosis, cardiovascular disease (CVD), steatohepatitis (fatty liver disease), pancreatitis, renal lipid deposition, depression, and obesity-related conditions. Preferably diseases related to dyslipidemia, diabetes, insulin resistance, tatty liver disease, neurodegeneration, preferably Parkisons and Alzheimers, and cancer, preferably pancreatic cancer, are treated.

As for age related diseases these include conditions such as: frailty; bone density loss; bone mineral density loss; weight loss; muscular atrophy; muscular degeneration; decline in muscle mass; decline in muscle strength; decline in hand strength; decline in leg strength; decline in physical fitness; decline in movement; decline in freedom of movement; decline in quality of life assessment; decline in ejection fraction; decline in exercise capacity; decline in learning; decline in learning capacity; decline in memory; decline in intellectual quotient; cognitive deterioration; forgetfulness; decline in cognitive capacity; decline in cognitive function; decline in synaptic plasticity; decline in synaptic function; cellular senescence; chronic kidney disease (CKD); chronic kidney disease-mineral and bone disorder (CKD-MBD); polycystic kidney disease (PKD); autosomal dominant polycystic kidney disease (ADPKD); acute kidney injury (AKI); acute tubular necrosis (ATN); acute allergic interstitial nephritis (AAIN); glomerulonephritis; kidney disease; renal failure; Alport Syndrome; nonoliguric renal failure; alcoholism; hyperphosphatemia; muscular dystrophy (MS); type 1 diabetes; type 2 diabetes; cardiovascular disease (CVD); cardiovascular calcification; cerebrovascular insufficiency; vascular calcification; coronary artery disease; heart failure; left ventricular hypertrophy; uremic cardiomyopathy; abnormalities in blood pressure; salt-sensitive hypertension; tissue calcification; calcific atherosclerotic plaque burden; calcinosis; familial tumoral calcinosis; cancer; one or more tumors; myelin-related diseases; demyelinating diseases; neurodegenerative disease; neurovascular diseases; progressive supranuclear palsy (PSP); Pompe disease; Niemann-Pick disease; microgliosis; Farber disease (FD); bone mass diseases; osteoporosis; osteopenia; osteopenia (particularly loss of BMD of cortical bone); pulmonary emphysema; pulmonary fibrosis; cystic fibrosis, idiopathic (i.e., cause unknown) pulmonary fibrosis, radiation-induced lung injury, cirrhosis, biliary atresia, atrial fibrosis, endomyocardial fibrosis, (old) myocardial infarction, glial scar, arterial stiffness, arthrofibrosis, Crohn's disease, Dupuytren's contracture, keloid, mediastinal fibrosis, myelofibrosis, Peyronie's disease, nephrogenic systemic fibrosis, progressive massive fibrosis, retroperitoneal fibrosis, scleroderma/systemic sclerosis, adhesive capsulitis, skin atrophy; thymic atrophy; accumulation of renal interstitial matrix; glomerulosclerosis; anemia; albuminuria; proteinuria; infertility; Alzheimer's disease; Parkinson's Disease; dementia; vascular dementia; amyotrophic lateral sclerosis (ALS); motor neuron disease (MND); atrial fibrillation; chronic obstructive pulmonary disease (COPD); fibromyalgia; adult onset diabetes; arthritis; rheumatoid arthritis; osteoarthritis; glaucoma; cataracts; macular degeneration; multiple sclerosis (MS); lupus; ulcerative colitis; cachexia; obesity; vitamin D-related conditions; bone diseases; bone diseases through bone remodeling; stem cell depletion; sea sickness; space adaptation syndrome (SAS); nausea; vertigo; nonalcoholic steatohepatitis (NASH), cirrhosis of the liver and alcoholic steatohepatitis.

More generally speaking the proposed system is for use in a method of treatment, amelioration, mitigation, slowing, arresting or reversing or prevention of at least one of the following conditions.

A “mitochondrial-related diseases/disorders” characterized by malfunction of the mitochondria. A mitochondrial-related disease or disorder includes a muscle structure disorder, a neuronal activation disorder, a muscle fatigue disorder, a muscle mass disorder, a metabolic disease, a cancer, a vascular disease, an ocular vascular disease, a muscular eye disease, or a renal disease. In some embodiments, a “mitochondrial-related disease or disorder” is selected from non-alcoholic fatty liver disease (NAFLD), non-alcoholic steatohepatitis (NASH), renal ischemia/reperfusion injury (IRI), Duchenne & Becker muscular dystrophy, diabetes (type 1 or type II), obesity, and sarcopenia. In another embodiment, a “mitochondrial-related disease or disorder” is selected from Alpers's Disease, CPEO-Chronic progressive external ophthalmoplegia, Kearns-Sayra Syndrome (KSS), Leber Hereditary Optic Neuropathy (LHON), MELAS—Mitochondrial myopathy, encephalomyopathy, lactic acidosis, and stroke-like episodes, MERRF—Myoclonic epilepsy and ragged-red fiber disease, NARP—neurogenic muscle weakness, ataxia, and retinitis pigmentosa, Pearson Syndrome, platinum-based chemotherapy induced ototoxicity, Cockayne syndrome, xeroderma pigmentosum A, Wallerian degeneration, and HIV-induced lipodystrophy and peroxisomal diseases like X-linked adrenoleukodystrophy. In certain embodiments, the agent may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external ophthalmoplegia, the Kearns-Sayre syndrome (with ophthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and Fatal).

Sports performance refers to the ability of the athlete's muscles to perform when participating in sports activities. Enhanced sports performance, strength speed and endurance are measured by an increase in muscular contraction strength, increase in amplitude of muscle contraction, shortening of muscle reaction time between stimulation and contraction. Athlete refers to an individual who participates in sports at any level and who seeks to achieve an improved level of strength, speed and endurance in their performance, such as, for example, body builders, bicyclists, long distance runners, short distance runners, etc. Enhanced sports performance in manifested by the ability to overcome muscle fatigue, ability to maintain activity for longer periods of time, and have a more effective workout.

Fat-related metabolic disorders amenable to treatment with the proposed modified nucleic acid molecules which directs the target immune cell to express a FoxP3 and/or a therapeutic agents include disorders in which (i) increased fat storage, reduced fat mobilization, and/or reduced fat burning is desired, and (ii) other disorders in which reduced fat storage, increased fat mobilization and/or increased fat burning is desired. Examples of the first category of disorders include, e.g., anorexia nervosa, wasting, AIDS-related weight loss, bulimia, cachexia. Examples of the latter category include, e.g., obesity, cardiovascular disease, osteoarthritis. The classification of other disorders (e.g., infertility, increased surgical risk, pregnancy complications) may depend on the weight of the subject, e.g., whether the subject is over- or underweight.

Obesity-related disease” and “Fat-related metabolic disorder” include, but are not limited to, anorexia nervosa, wasting, AIDS-related weight loss, bulimia, cachexia, lipid disorders including hyperlipidemia and hyperuricemia, insulin resistance, noninsulin dependent diabetes mellitus (NIDDM, or Type II diabetes), insulin dependent diabetes mellitus (IDDM or Type I diabetes), diabetes-related complications including microangiopathic lesions, ocular lesions, retinopathy, neuropathy, and renal lesions (including diabetic nephropathy), cardiovascular disease (including cardiac insufficiency, coronary insufficiency, and high blood pressure), atherosclerosis, atheromatous disease, stroke, hypertension, Syndrome X, gallbladder disease, osteoarthritis, sleep apnea, forms of cancer such as uterine, breast, colon, colorectal, pancreatic, kidney, and gallbladder, high cholesterol levels, complications of pregnancy, menstrual irregularities, hirsutism, muscular dystrophy, infertility, a weight-related disorder (characterized by a subject being over or under weight, e.g., being within the top or bottom 25th percentile of body mass index) and increased surgical risk. In preferred embodiments, a treated or diagnosed subject is a mammal, preferably a human.

Examples of neurodegenerative and/or neuroinflammation diseases amenable to treatment with the proposed modified nucleic acid molecules which directs the target immune cell to express a FoxP3 and/or a therapeutic agents, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies, Friedreich's ataxia, dementia (including Lewy Body disease, mild cognitive impairment (MCI), Primary Senile Degenerative Dementia, Alzheimer Type Senile Dementia and Alzheimer Type Dementia), Parkinsonian disorders (including Lewy Body disease and Parkinsonism-linked to chromosome 17 (FTDP-17)), progressive supranuclear palsy (also known as Steele-Richardson-Olszewski Syndrome or Disease, Progressive Supranuclear Ophthalmoplegia), Pick's disease and corticobasal degeneration. Multiple sclerosis (MS), including relapsing MS and monosymptomatic MS, and other demyelinating conditions, such as, for example, chronic inflammatory demyelinating polyneuropathy (CIDP), or symptoms associated therewith.

Diabetic neuropathies may be amenable to treatment with the proposed modified nucleic acid molecules. Diabetic neuropathies are neuropathic disorders that are associated with diabetes mellitus. Relatively common conditions which may be associated with peripheral neuropathy, diabetic neuropathy include third nerve palsy; mononeuropathy; mononeuritis multiplex; diabetic amyotrophy; a painful polyneuropathy; autonomic neuropathy; and thoracoabdominal neuropathy.

Peripheral nervous system diseases treatable with the proposed modified nucleic acid molecules include: diabetes, leprosy, Charcot-Marie-Tooth disease, Guillain-Barre syndrome and Brachial Plexus Neuropathies (diseases of the cervical and first thoracic roots, nerve trunks, cords, and peripheral nerve components of the brachial plexus.

In another embodiment, the proposed agent target immune cell to express a CAR is used to treat or prevent a polyglutamine disease. Exemplary polyglutamine diseases include Spinobulbar muscular atrophy (Kennedy disease), Huntington's Disease (FID), Dentatorubral-pallidoluysian atrophy (Haw River syndrome), Spinocerebellar ataxia type 1, Spinocerebellar ataxia type 2, Spinocerebellar ataxia type 3 (Machado-Joseph disease), Spinocerebellar ataxia type 6, Spinocerebellar ataxia type 7, and Spinocerebellar ataxia type 17.

Accordingly, the present invention provides anticoagulation and antithrombotic treatments with the proposed modified nucleic acid molecules which directs the target immune cell to express a FoxP3 and/or a therapeutic agents aiming at inhibiting inflammation and the formation of blood clots in order to prevent or treat blood coagulation disorders such as myocardial infarction, stroke, loss of a limb by peripheral artery disease or pulmonary embolism.

In another embodiment, the proposed modified nucleic acid molecules which directs the target immune cell to express a FoxP3 and/or a therapeutic agents is used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, the proposed modified nucleic acid molecules which directs the target immune cell to express a FoxP3 and/or a therapeutic agents can be used to treat autoimmune diseases and/or inflammation associated with autoimmune diseases such as organ-tissue autoimmune diseases (e.g., Raynaud's syndrome), inflammatory bowel disease, scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain aspects of the invention, the vision impairment is amenable to the delivery vehicle carrying modified nucleic acid molecules which directs the target immune cell to express a FoxP3 and/or a therapeutic agents caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis.

In certain aspects of the invention, the vision impairment amenable to treatment with the modified nucleic acid molecules which directs the target immune cell to express a FoxP3 and/or a therapeutic agent is caused by retinal damage or inflammation or the retina. In particular embodiments, retinal damage is caused by disturbances in blood flow to the eye (e.g., arteriosclerosis, vasculitis). In particular embodiments, retinal damage is caused by disruption of the macula (e.g., exudative or non-exudative macular degeneration).

Exemplary retinal diseases amenable to treatment with the delivery vehicle carrying modified nucleic acid molecules directs the target immune cell to express a FoxP3 and/or a therapeutic agent include Exudative Age Related Macular Degeneration, Nonexudative Age Related Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion. Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis, Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes.

Other exemplary diseases amenable to treatment with the delivery vehicle carrying modified nucleic acid molecules directs the target immune cell to express a FoxP3 and/or a therapeutic agent include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis.

Muscular dystrophy amenable to treatment with the delivery vehicle carrying modified nucleic acid molecules which directs the target immune cell to express a FoxP3 and/or a therapeutic agent refers to a family of diseases involving deterioration of neuromuscular structure and function, often resulting in atrophy of skeletal muscle and myocardial dysfunction, such as Duchenne muscular dystrophy.

In certain embodiments, the delivery vehicle carrying modified nucleic acid molecules directs the target immune cell to express a FoxP3 and/or a therapeutic agent may be used for reducing the rate of decline in T-regulatory cells in the body and for improving reducing human C-reactive protein level status in patients with diseases.

### EXAMPLES

### Methods

Cell culture. HEK293T (CRL-11268) and Jurkat cells obtained from American Type Culture Collection were routinely cultured. Cells were maintained in Dulbecco's Modified Eagle Medium (DMEM; Thermo Fisher Scientific) supplemented with 10% fetal bovine serum (FBS; Gibco) and 1% penicillin-streptomycin (Gibco) at 37° C. with 5% C02. Cell lines were not authenticated. For subculture, cells were detached from flasks using 0.25% TrypLE (Thermo Fisher Scientific) and resuspended in fresh culture medium. Cells were passaged 2-3 times per week and used for experiments in logarithmic growth phase. Jurkat cells were routinely cultured in RPMI 1640 medium (Gibco) supplemented with 10% fetal bovine serum (FBS), 1 mM sodium pyruvate, 10 mM HEPES buffer, 100 units/mL penicillin, 4.5 g/L glucose and 100 μg/mL streptomycin. Cells were maintained in sterile 24-well tissue culture dishes at 37° C. with 5% C02. When cells reached 80% confluence approximately every 3 days, they were passaged by gently resuspending with fresh, pre-warmed medium. Cell viability and concentration were determined using trypan blue exclusion and a hemocytometer, respectively. Only cells exhibiting greater than 90% viability were used for experiments.

Circular RNA synthesis. To construct the GFP, FoxP3 GFP-RFP target genes containing different elements, gene synthesis and cloning was performed. The DNA vector used to construct the circular RNA includes T7 promoter, 5′ homology arm, IRES element, GFP or FoxP3 coding region, 3′ homology arm, the T7 terminator, and Poly A tail with restriction sites for linearization. The resulting gene fragment was ligated into the pUC57 vector. For the GFP-RFP target gene, a second IRES element was added before the RFP coding element separated with and without an intron. For the GFP-FoxP3 target gene, a second IRES element was added before the FoxP3 coding element separated with and without an intron. The linear plasmid was used for in vitro transcription (IVT).

IVT was performed using the HiScribe™ T7 High Yield RNA Synthesis Kit (NEB). One microgram of the purified circRNA template was used in 20 μl IVT reactions. Reactions were incubated overnight at 37° C. with shaking at 1000 rpm. To remove the DNA template, 2 μl of DNase I was added to each reaction and incubated for 20 minutes at 37° C. with shaking. The RNA was then purified using column purification. To isolate the circRNAs, RNase R digestion was performed on the purified RNA. One unit of RNase R per microgram of RNA was added and reactions were incubated for 60 minutes at 37° C. with shaking. RNase R selectively degrades linear RNAs but not circRNAs. This allowed isolation of circRNAs from the reaction. Samples were column purified and quantified using a NanoDrop One spectrophotometer. Verification of complete RNase R digestion was confirmed on agarose gel to distinguish circRNAs from any remaining linear RNA. If linear RNA was still present, the RNase R digestion step was repeated. This process generated purified circRNA transcripts from PCR-amplified templates using IVT and RNase R treatment to isolate the circular forms.

mRNA synthesis. Templates for in vitro transcription (IVT) of mRNA were generated by PCR amplification of the desired mRNA sequence. Oligonucleotides were used to amplify the mRNA template for 30 cycles. The PCR product was then purified using column purification. IVT reactions were carried out using the Takara IVTpro T7 mRNA Synthesis Kit with modifications to synthesize modified mRNA. One microgram of the purified mRNA template was used per 20 μl reaction. CleanCap AG was added to a final concentration of 4 mM to add a 5′ cap structure. N1-methylpseudouridine (N1Ψ) was fully substituted for uridine triphosphate (UTP) during transcription. Reactions were incubated for 2 hours at 37° C. with shaking at 1000 rpm. To remove DNA templates, 2 μl of DNase I was added per reaction and further incubated for 20 minutes.

The resulting mRNA transcripts containing N1Ψ modifications were purified from the reaction using column purification. This generated milligram quantities of modified mRNA for downstream applications. The template-driven IVT using T7 RNA polymerase, along with capping and N1′ modifications, allowed production of defined mRNA sequences with enhanced stability.

RNA transfection. For RNA delivery, three transfection methods were compared: TransIT-mRNA (Mirus Bio), polyethyleneimine (PEI), and Lipofectamine (Thermo Fisher Scientific). Equimolar amounts of crude GFP circRNA (RNAse R untreated), pure GFP circRNA (RNAse R treated), or GFP mRNA were transfected using standardized conditions for each agent. Using TransIT-mRNA, 5 μl reagent was used per 1 μg RNA following the manufacturer's recommendations. Experiments were performed using equimolar quantity of RNA. Within each experiment, the same transfection method was used to deliver all RNA samples in parallel for comparative analyses. This optimization of cell culture conditions and defined transfection protocols ensured consistency across experiments evaluating functional outcomes of mRNA versus circRNA delivery.

Microscopy. Cells were seeded onto 8-well Chamber slides (Nunc™ Lab-Tek™, Cat #177402) and incubated at 37° C. for the cells to settle or adhere to the bottom. This allowed consistent attachment across the samples. After 24 hours of transfection, fluorescence images were captured using the 10× magnification lens on the Keyence BZ-X710 microscope. To ensure data integrity, exposure time, light intensity, and other imaging parameters were kept constant, matching those used for the negative control. For each well, at least 3 images were acquired at different locations to capture diverse regions of the cell population. These images were then used for downstream analysis, quantifying features like cell number and fluorescence intensity.

Flow cytometry. Single-cell suspensions were generated from spleens and lymph nodes. Tissues were gently disrupted between glass slides to release cells which were passed through a 70 μm cell strainer into RPMI 1640+10% FBS. Cells were harvested and pelleted by centrifugation at 300×g for 5 minutes and resuspended in fresh medium. This was followed by surface staining at 4° C. for 30 minutes. Subsequently, the cells were incubated with a viability dye for 10 minutes at room temperature. After incubation, the cells were washed twice with a flow wash buffer (DPBS with 1% BSA). Fixation and permeabilization were then performed according to the manufacturer's recommendations (TONBO biosciences, TNB-1022-L160). Following this, intracellular staining was carried out for 30 minutes at room temperature. Cells were stained with a cocktail of fluorophore-conjugated anti-mouse antibodies targeting surface markers, shown in Table 1, for 30 minutes on ice while protected from light. The unbound antibodies were washed off twice with permeabilization buffer. Finally, the cells were analyzed using a BD Fusion flow cytometer. Stained cells were acquired on a BD LSR II flow cytometer, collecting 50,000 total events per sample. Fluorophore signal compensation was performed using ArC Amine Reactive beads and Compbead particles. GFP was measured in live cells, prior to fixing. Data were analyzed with FlowJo software to characterize immune cell populations and expression of transgenic proteins.

TABLE 1
Antibodies for flow cytometry

| | | | | Catalogue | Concentration/100 ul |
|---|---|---|---|---|---|
| Antibody | Clone | Fluorochrome | Company | number | cells (<1M cells) |
| Anti mouse | FJK-16S | Alexa Fluor | Invitrogen | 53-5773-82 | 1 ul |
| FoxP3 | | 488 | | | |
| Anti mouse | 150D/E4 | PE | Invitrogen | 12-4774-72 | 1 ul |
| FoxP3 | | | | | |
| Anti | 17A2 | APC/Fire 750 | BioLegend | 100248 | 1 ul |
| mouseCD3 | | | | | |
| Anti | RM4-5 | BV711 | BioLegend | 100248 | 1 ul |
| mouseCD4 | | | | | |
| Anti mouse | TC11- | BV421 | BioLegend | 100248 | 1 ul |
| IL-17A | 18H10.1 | | | | |
| Anti mouse | XMG1.2 | PE/Dazzle 594 | BioLegend | 100248 | 1 ul |
| IFN-γ | | | | | |
| Anti mouse | 22F6 | eFluor 450 | BioLegend | 100248 | 1 ul |
| Helios | | | | | |
| Anti Myc- | 9811 | Alexa 647 | Cell | 2233S | 1 ul |
| tag | | | signaling | | |
| Fixable | | eFluor 780 | eBioscience | 65-0865-14 | 1 ul |
| viability dye | | | | | |
| 780 | | | | | |

In vitro cell transfection studies. For cell transfection studies using FOXP3 mRNA, human CD4+ T cells were plated in 16-well plates. After 24 h, LNPs carrying reporter FOXP3 mRNA were added at increasing concentrations to the cells and incubated for 1.5 h. Plates were then washed three times with PBS, and complete medium was added to the cells. After culturing for 24 h in complete media, cells were washed with PBS and protein expression was measured by flow cytometry. Briefly cells were stained with Live/Dead Aqua (Thermo Fisher Scientific, L34966) and antibodies against CD3, CD4 and CD8 and the percentage of cells-expressing FOXP3 were determined using flow cytometry. For stability studies cells were transfected with LNPs encapsulating nucleosides of FOXP3 and Helios. Transfections were performed in triplicate.

In vitro suppression assay. To assay for in vitro suppression of polyclonally activated cells, CD4+CD25− responder cells from spleens of mice were isolated, labeled with 3-5 μmol/l CellTrace Violet (Invitrogen, Carlsbad, CA) and cultured with CD4− total splenocytes. T-cells were cocultured with each cell iTreg variant at a 1:1 ratio with or without stimulation at 5×105 cells/mL. Cells were stimulated with anti-CD3 and anti-CD28 coated DYNAL Dynabeads (Thermo Fisher Scientific) (1:16 bead:target cell ratio). CD4+ T cells treated with anti-CD4/FOXP3 mRNA-LNP or control IgG/mRNA-LNP formulations were added at various Treg:T responder ratios and cells were cultured for 72 h at 37° C. Dilution of the CellTrace Violet label in FOXP3-proliferating responder cells were quantified. Proliferation was determined by quantifying CellTrace Violet fluorescence intensity relative to a parent population of unstimulated responder cells (0% proliferation) and stimulated cells incubated without Treg (100% proliferation). Percentage of CD4+ responder T cell proliferation was also determined using flow cytometry. Percent suppression was calculated by the following equation: [(percent responder proliferation alone)−(percent responder proliferation with transduced cells)]/(percent responder proliferation alone)×100.

CD4+CD25− Treg Expression ex vivo. CD4+ T-cells were prepared from pooled spleen and total lymph nodes (inguinal, axillary, brachial, superficial cervical, and lumbar) of C57BL/6 mice treated with anti-CD4/FOXP3 mRNA-LNP or control IgG/mRNA-LNP formulations. Cell suspensions were stained with anti-CD4-PerCP-Cy5.5, anti-CD25-FITC, and enriched for CD25+ cells using anti-FITC beads (Miltenyi). Cells were then sorted for CD4+CD25hi on a BD FACSAria. Treg expression was assessed using intracellular staining for FOXP3 using flow cytometry.

In vivo delivery of circRNA and mRNA. Two-month old male C57B16 mice obtained from The Jackson Laboratory were used for all in vivo studies. Mice were injected via tail vein with liposomes containing: Circular GFP (circGFP), Circular FoxP3 (circFoxP3), mRNA GFP, mRNA FoxP3. For RNA formulations, 10 μg of either mRNA or circular RNA were complexed at equimolar ratios with polyethyleneimine (PEI). Each RNA-PEI complex was diluted in 250 μl of injection buffer prior to administration via the tail vein. Mice received a single intravenous injection of either the GFP mRNA, circGFP, FoxP3 mRNA or FoxP3 circRNA nanoparticles. Mice were culled at 72 h post-injection.

Biodistribution of anti-CD4/FOXP3 mRNA-LNPs in C57BL/6J mice: FOXP3 translation at tissue and cellular level. C57BL/6J mice were injected via tail vein (i.v.) with anti-CD4/FOXP3 mRNA-LNP or control IgG/mRNA-LNP formulations. Both groups contained reporter Luciferase mRNA to assess transfection efficiency. After 4 h of injection, mice were euthanized, and selected organs (liver, lymph nodes, spleen, lung, kidney and heart) were harvested, rinsed with PBS. Tissue samples were homogenized in appropriate volumes of cell lysis buffer (Promega Corp, Madison, WI, USA) containing protease inhibitor cocktail. Protein expression in different tissues were assessed by flow cytometry and luciferase activity. We analyzed the mRNA expression in the CD3+ cell population. CD3+ cells were isolated from the spleens or lymph nodes using the immune-magnetic positive selection using for Mouse CD3+ Selection Kit (Thermo Fisher Scientific) following manufacturer's protocol. Viability was confirmed with Live/Dead Aqua flow cytometric assays. Cells were then evaluated for expression T-cell and Treg markers using flow cytometry including CD4, CD8, CD25, FOXP3, Helios, CTLA-4. FOXP3 expression levels was assessed by flow cytometric analysis.

Lymph node harvest and iTreg assays. C57BL/6J mice were injected i.v. with anti-CD4/FOXP3 mRNA-LNP variants or control IgG/mRNA-LNP formulations as described above. 24 hours after injection mice were euthanized and LNs harvested. T-cells were isolated and CD4+ T-cells were enriched using immunomagnetic beads and FACS as previously described. CD4+ T-cells were then further sorted for GFP expression using FACS to generate GFP+CD4+ T-cells and GFP-CD4+ cells. Cells were then evaluated for expression T-cell and Treg markers including CD4, CD8, CD25, FOXP3, Helios, CTLA-4, CD69. RNA expression analysis was also preformed on selected subsets. In some cases, the isolated cells were cultured in the presence or absence of immobilized MDA-ApoB and evaluated for markers of activation, including CD69 and cytokine expression. T-cell suppression assays were performed as described above using different ratios of iTregs to CD4+ cells.

LPS sepsis inflammation model. Two-month old male C57B16 mice obtained from The Jackson Laboratory were used for all in vivo studies. Mice were injected i.p. with 80 μg lipopolysaccharide (LPS) or vehicle to induce inflammation. After 4 hours, mice were injected via tail vein with liposomes containing: Circular GFP (circGFP), Circular FoxP3 (circFoxP3), mRNA GFP, mRNA FoxP3. For RNA formulations, 10 g of mRNA or circular RNA circRNA were complexed at equimolar ratios with polyethyleneimine (PEI). Each RNA-PEI complex was diluted in 250 μl of injection buffer prior to administration via the tail vein. Mice received a single intravenous injection of either the GFP mRNA, circGFP, FoxP3 mRNA or FoxP3 circRNA nanoparticles. Blood glucose from fasted mice was measured via tail vein sampling using Contour glucometers. Mice were fasted and culled at 12 h, 36 h and 72 h timepoint post-glucose measurements. Spleen and lympnode tissues were harvest and plasma samples were clotted and collected for cytokine analysis by enzyme-linked immunosorbent assay (ELISA). ELISA kits for murine interleukin-6 (IL-6) and murine interleukin-1 (IL-1β), were purchased from Abcam. IL-6 and IL-10 in the sera of mice were measured by ELISA according to the manufacturer's instructions.

### Results

### Example 1. Anti-CD4/mRNA-LNPs Target CD4+ T Cells In Vitro

To assess internalization and functional activity of the targeted mRNA-LNPs, anti-CD4 antibody- or control IgG-conjugated LNPs encapsulated with FOXP3-encoding mRNA were incubated with human CD4+ T cells at different doses. Flow cytometric analysis showed a dose-dependent increase in intracellular expression of FOXP3 protein in cells targeted by anti-CD4/FOXP3-mRNA-LNPs compared to control IgG/mRNA-LNPs suggesting an increase in translation of FOXP3 mRNA. Anti-CD4/FOXP3-mRNA-LNPs treated CD4+ T cells acquired a Treg-like phenotype, with high expression of CD25 and GITR and low expression of IL-7Ra chain (CD127). Functional activity was assessed by CD4+CD25− T-cells suppression assay. Anti-CD4/FOXP3-mRNA-LNPs formulation treated CD4+ T cells potently suppressed the proliferation of responder CD4+CD25− T cells. Suppression was still strongly evident at a Treg:Teff (responder) cell ratio of 1:16, indicating that FOXP3 alone is capable of producing cells with Treg function. The control IgG/mRNA-LNPs treated CD4+ T-cells did not suppress at any ratio.

### Example 2. Biodistribution of Anti-CD4 FOXP3 mRNA-LNPs

We next analyzed the biodistribution of anti-CD4/FOXP3-mRNA-LNPs in mice after tail vein based intravenous (i.v.) administration using luciferase mRNA. The spleen, lymph nodes kidney, lungs, heart and liver were harvested the next day, and single-cell suspensions were prepared from each tissue. The expression pattern of FOXP3 mRNA showed a marked difference between anti-CD4/FOXP3-Luc mRNA-LNP and control IgG/Luc mRNA-LNP-treated mice. Only mice treated with anti-CD4/FOXP3-Luc mRNA-LNP showed an increase in FOXP3 mRNA which decreased significantly in liver with CD4 targeting. Interestingly, luciferase activity for anti-CD4/FOXP3-Luc-mRNA-LNPs was significantly higher compared to the control IgG/Luc mRNA-LNPs in the spleen compared to other organs like kidney, lungs, heart, and liver.

### Example 3. FOXP3-Helios-CAR-mRNA LNPs Reprogram T-Cells to Become Stable Induced Tregs (iTregs)

We next analyzed the functional capacity of iTregs in mice treated with intravenous administration of various forms of anti-CD4/FOXP3-mRNA-LNPs using a Green Fluorescent Protein (GFP) Reporter. Mice were injected with 4 different groups of LNP formulations. 1) anti-CD4/FOXP3-GFP-mRNA-LNPs, 2) anti-CD4/FOXP3-Helios-GFP-mRNA-LNPs 3) anti-CD4/FOXP3-Helios-CAR-GFP-mRNA-LNPs 4) negative control LNPs. In this case the CAR was a chimeric antigen receptor targeting malondialdehyde modified apolipoprotein B (MDA-ApoB). 24 hours after injection mice were sacrificed and lymph nodes (LN) harvested. CD4+ T-cells were isolated and then GFP+ cells separated from GFP negative CD4+ cells. The cells were then evaluated for expression of FOXP3, Helios and the MDA-ApoB CAR. Cells were further evaluated for activation potential when cultured with MDA-ApoB and compared to control conditions. Finally, cells were evaluated for the ability to suppress the proliferation of responder CD4+CD25− T cells in a T-cell suppression assay. GFP positive cells showed the appropriate protein expression of co-administered mRNA. GFP positive cells showed significant T-cells suppression, while GFP negative cells had no effect on T-cell suppression. GFP+ cells from mice injected with anti-CD4/FOXP3-Helios-CAR-GFP-mRNA-LNPs showed increased activation markers after culture with MDA-ApoB and had the most robust T-cell suppression of any cell type tested.

### Example 4. Stability of Circular RNA In Vitro

Conventional eukaryotic translation initiation relies on a 5⁷G cap at the 5′ end of linear mRNA for ribosome recruitment. However, some viruses utilize internal ribosome entry sequences (IRES) for cap-independent initiation. IRES-driven translation typically has lower efficiency than cap-dependent mechanisms. Introduction of an internal ribosome entry sequence (IRES) into a circular RNA allows translation of a protein encoded by a circRNA. This study evaluated viral and synthetic IRES elements for cap-independent FoxP3 expression in T cells. A circular RNA encoding FoxP3 with an IRES was synthesized to independently titrate FoxP3 levels without altering RNA dose in T-cells compared to mRNA. IRES strength varies between elements and cell types. This allows expression tuning from polycistronic transcripts. The described IRES elements address the need for controlled multi-gene delivery. Evaluation of IRES-driven FoxP3 expression profiling demonstrates that optimized RNA-engineered regulatory T cell generation for modulating immune responses via a non-canonical translation mechanism is possible. This fine-tuned expression offers advantages over conventional dosing of mRNA for stable genetic reprogramming applications of T-cells.

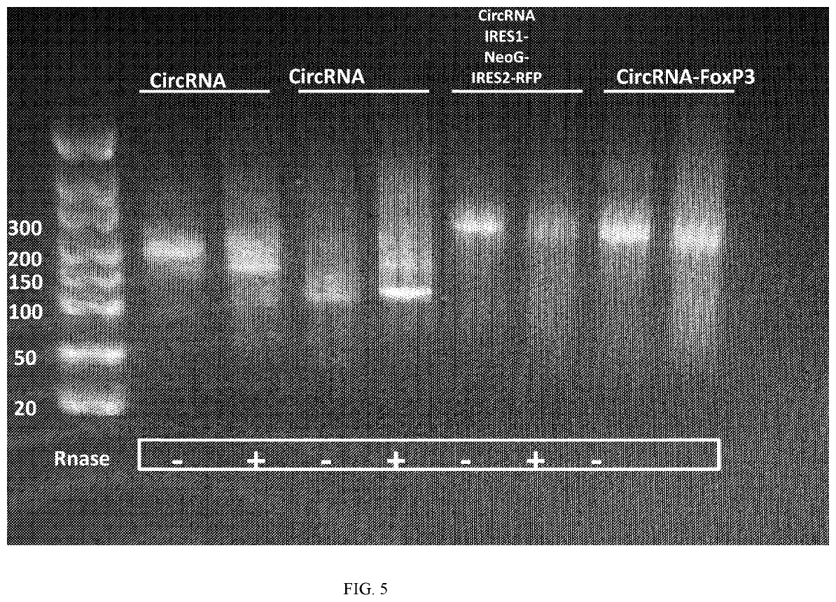

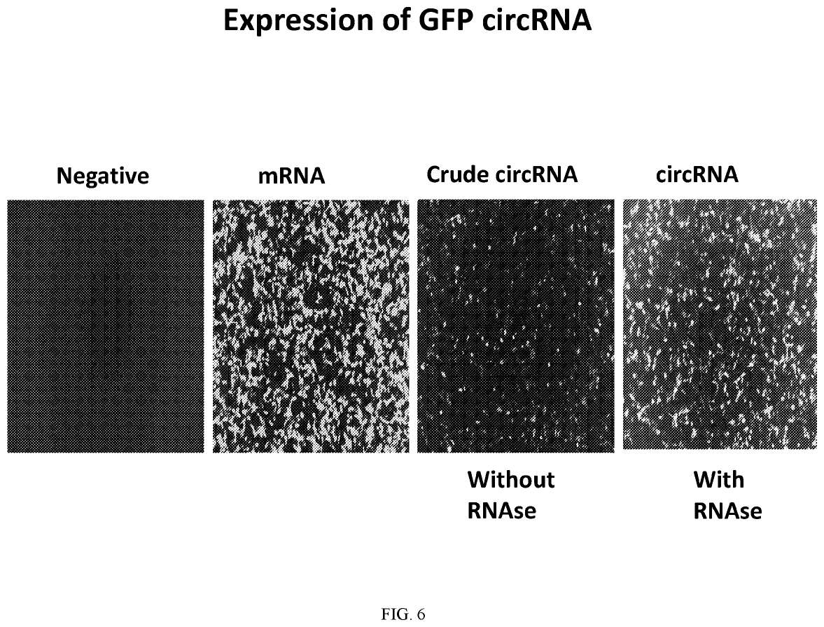

FoxP3 expression was achieved using a circular RNA containing an internal ribosome entry site (IRES) for cap-independent translation. IRES sequences facilitate recruitment of ribosomes internally within mRNAs, allowing expression of multiple proteins from a single transcript. Viral and synthetic IRES elements were evaluated for expressing FoxP3 in T-cells. Compared to cap-dependent translation, IRES-driven expression has lower but tunable strength depending on the IRES chosen. A circular RNA encoding both FoxP3 or GFP with an IRES sequence was synthesized. The IRES independently controlled FoxP3 and GFP expression levels without altering RNA dose. This dual-expression strategy utilized the RNA structure for stability while titrating FoxP3 production for optimal Treg conversion from conventional T-cells. The IRES thus enabled defined FoxP3 expression profiling to efficiently generate induced Tregs for modulating immune responses. As shown in FIGS. 5-6, the circular RNA GFP product is resistant to nuclease degradation and has improved functionality in vitro.

### Example 4. Functionality of Circular RNA In Vitro

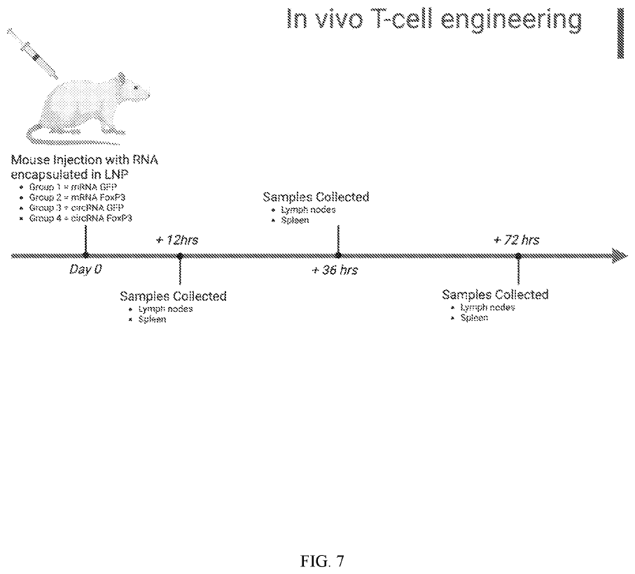

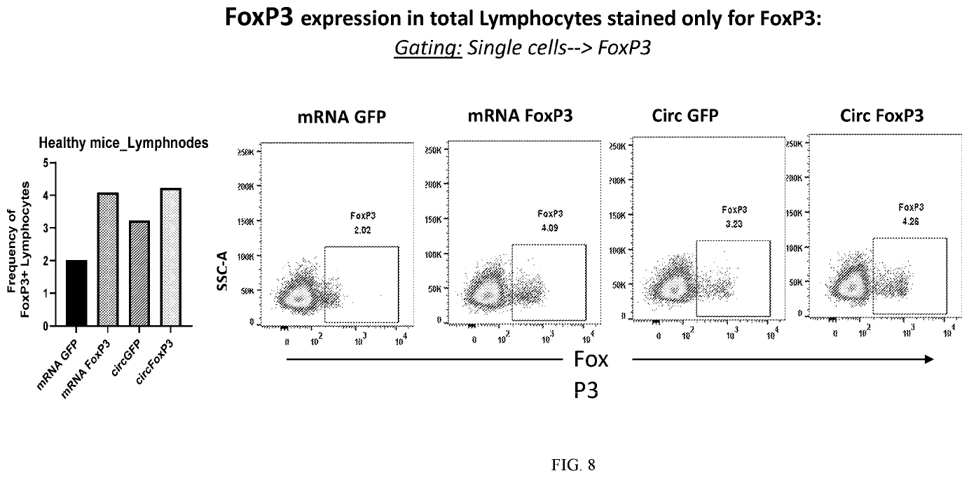

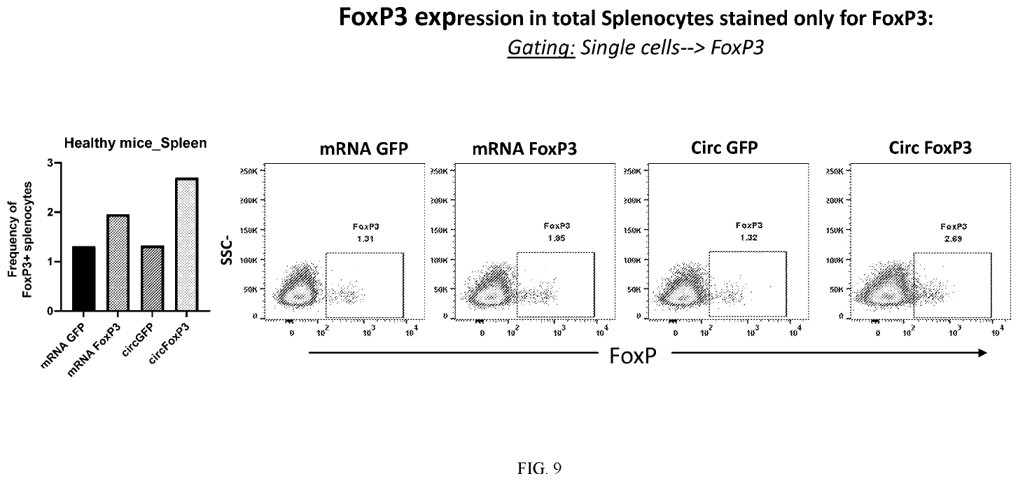

Functionality of circular RNA encoding FoxP3 and linear RNA encoding FOXP3 was assessed as shown in FIG. 7. Mice were injected with one of four groups of lipid nanoparticles containing either mRNA GFP, mRNA FoxP3, circular RNA GFP, or circular RNA FoxP3. The lymph nodes and spleen were collected 12-, 36-, and 72-hours post injection. In lymph nodes, non-integrating FoxP3 mRNA therapy caused reprogramming of cells (FIG. 8). The same result was found in the spleen (FIG. 9).

### Example 5. Circular RNA in a LPS-Induced Inflammatory Model

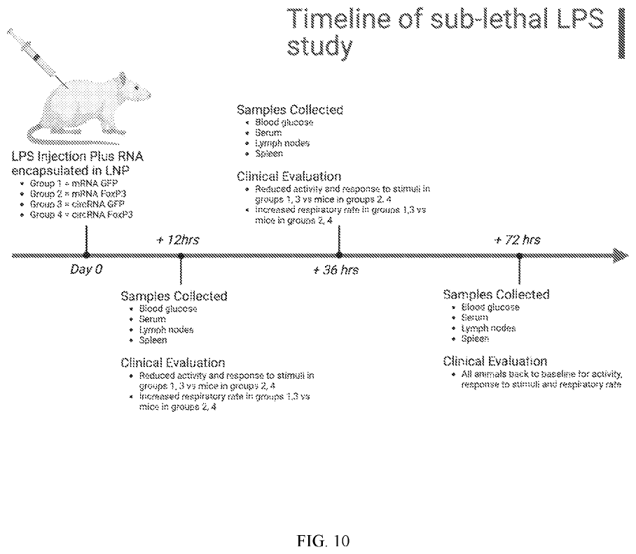

Next, circRNA FoxP3 and mRNA FoxP3 were assessed in a LPS-induced inflammatory murine model (FIG. 10). Mice were injected with LPS and one of four groups of lipid nanoparticles containing either mRNA GFP, mRNA FoxP3, circular RNA GFP, or circular RNA FoxP3. Serum, lymph nodes, and the spleen were collected 12-, 36-, and 72-hours post injection, and blood glucose assessed at these time points. Also at these time points, the mice were clinically evaluated for activity level and response to stimuli, and respiratory rate.

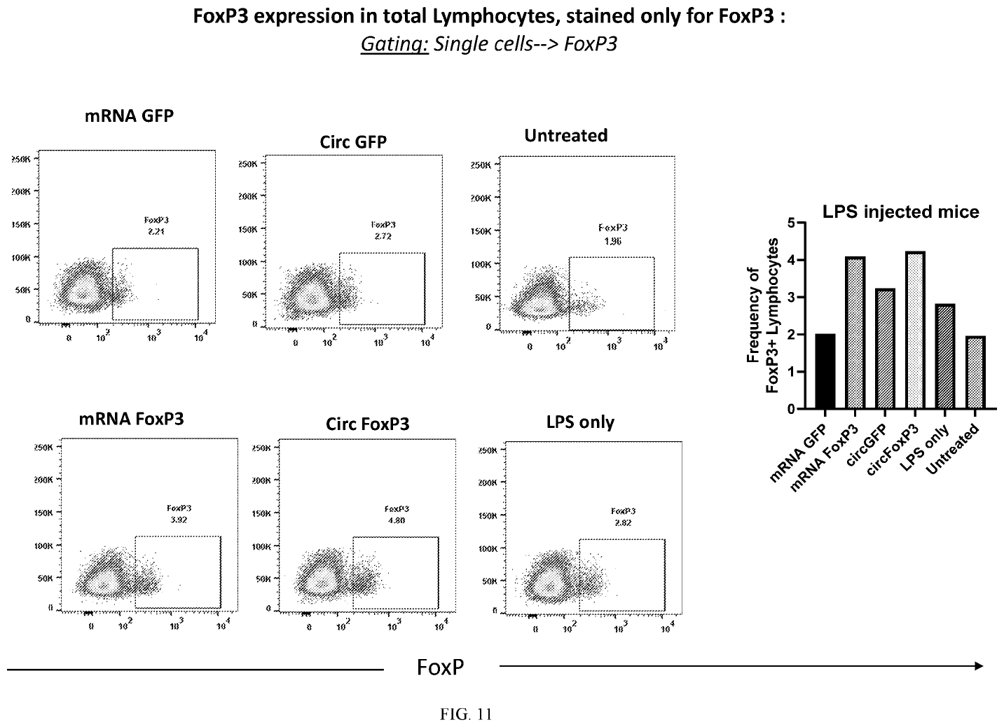

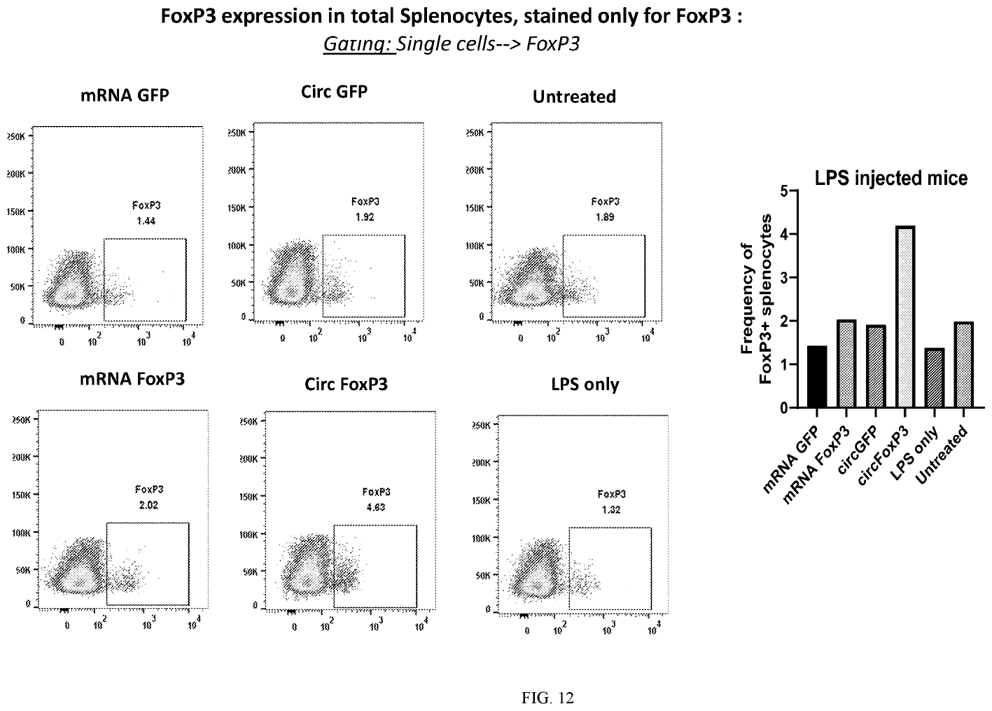

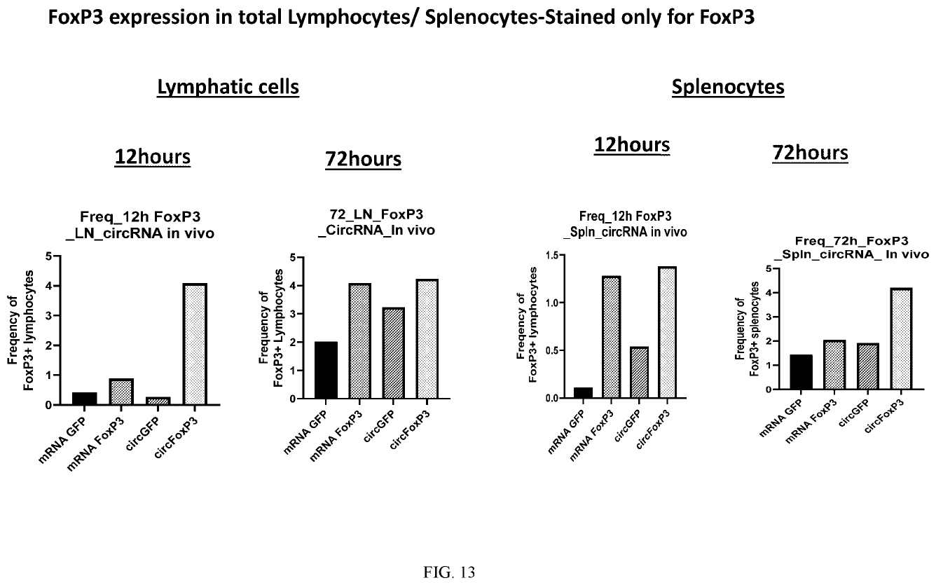

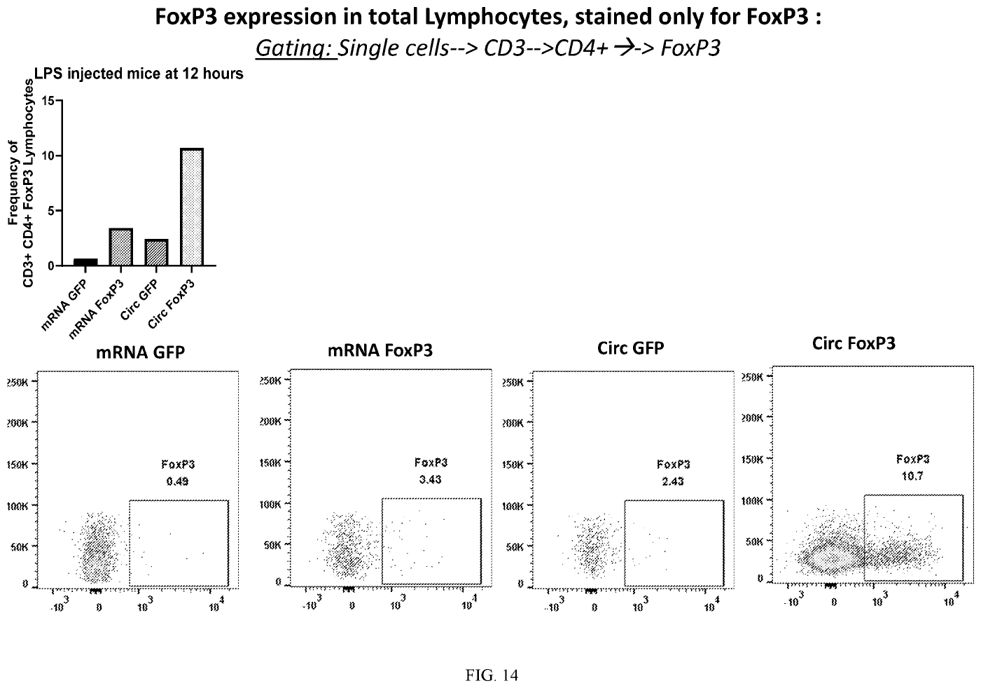

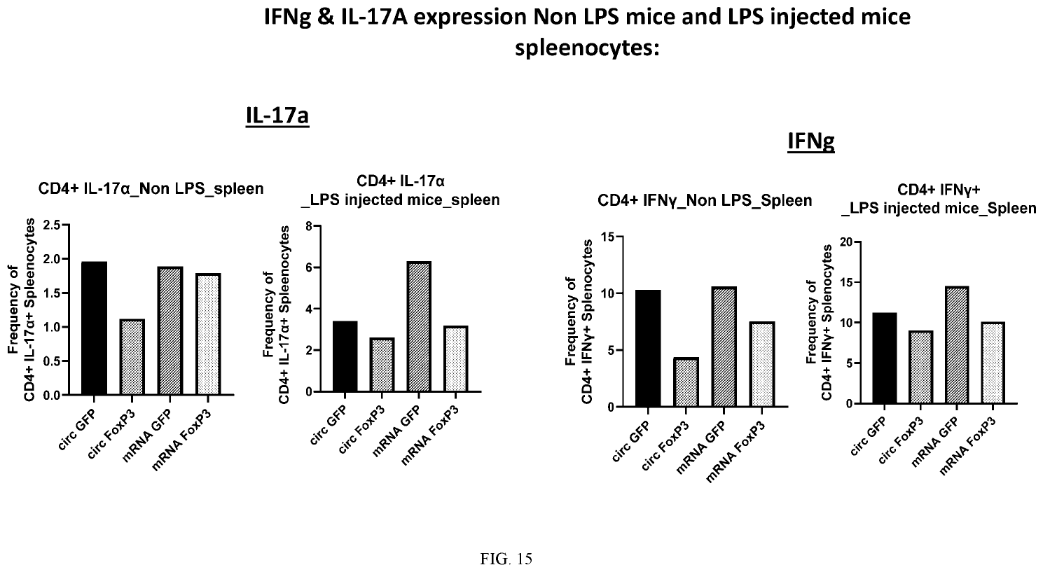

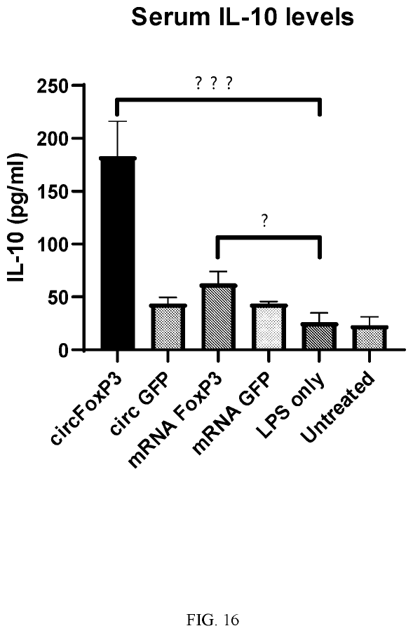

As shown in FIG. 11, non-integrating FoxP3 RNA therapy causes reprogramming of cells in lymph nodes during LPS-induced systemic inflammation. The same result was found in the spleens, as shown in FIG. 12. Circular RNA-injected groups showed longer FoxP3 ex[ression in both the spleen and lymph nodes (FIG. 13). Circular RNA FoxP3 reprogrammed of CD4+ T-cells into stable inducible Tregs under inflammatory conditions in vivo in lymphocytes (FIG. 14). IL-17a and IFNg expression was measured to assess the anti-inflammatory Treg profile post-injection (FIG. 15). For both markers, circular RNA FoxP3 treatment maintained an anti-inflammatory Treg profile in LPS-induced inflammation models (FIG. 15). Likewise, IL-10 secretion was increased in mice injected with circular RNA FoxP3 (FIG. 16).

All publications, patents, patent applications or other documents cited herein are hereby incorporated by reference in their entirety for all purposes to the same extent as if each individual publication, patent, patent application, or other document was individually indicated to be incorporated by reference for all purposes. Where a conflict exists between the instant application and a reference provided herein, the instant application shall dominate.

SEQUENCE LISTING
DNA sequence for human FOXP3 transcript
variant 1 mRNA cDNA
SEQ ID NO: 1
AGTTTCCCACAAGCCAGGCTGATCCTTTTCTGTCAGTCCACTTCACCAAG
CCTGCCCTTGGACAAGGACCCGATGCCCAACCCCAGGCCTGGCAAGCCCT
CGGCCCCTTCCTTGGCCCTTGGCCCATCCCCAGGAGCCTCGCCCAGCTGG
AGGGCTGCACCCAAAGCCTCAGACCTGCTGGGGGCCCGGGGCCCAGGGGG
AACCTTCCAGGGCCGAGATCTTCGAGGCGGGGCCCATGCCTCCTCTTCTT
CCTTGAACCCCATGCCACCATCGCAGCTGCAGCTGCCCACACTGCCCCTA
GTCATGGTGGCACCCTCCGGGGCACGGCTGGGCCCCTTGCCCCACTTACA
GGCACTCCTCCAGGACAGGCCACATTTCATGCACCAGCTCTCAACGGTGG
ATGCCCACGCCCGGACCCCTGTGCTGCAGGTGCACCCCCTGGAGAGCCCA
GCCATGATCAGCCTCACACCACCCACCACCGCCACTGGGGTCTTCTCCCT
CAAGGCCCGGCCTGGCCTCCCACCTGGGATCAACGTGGCCAGCCTGGAAT
GGGTGTCCAGGGAGCCGGCACTGCTCTGCACCTTCCCAAATCCCAGTGCA
CCCAGGAAGGACAGCACCCTTTCGGCTGTGCCCCAGAGCTCCTACCCACT
GCTGGCAAATGGTGTCTGCAAGTGGCCCGGATGTGAGAAGGTCTTCGAAG
AGCCAGAGGACTTCCTCAAGCACTGCCAGGCGGACCATCTTCTGGATGAG
AAGGGCAGGGCACAATGTCTCCTCCAGAGAGAGATGGTACAGTCTCTGGA
GCAGCAGCTGGTGCTGGAGAAGGAGAAGCTGAGTGCCATGCAGGCCCACC
TGGCTGGGAAAATGGCACTGACCAAGGCTTCATCTGTGGCATCATCCGAC
AAGGGCTCCTGCTGCATCGTAGCTGCTGGCAGCCAAGGCCCTGTCGTCCC
AGCCTGGTCTGGCCCCCGGGAGGCCCCTGACAGCCTGTTTGCTGTCCGGA
GGCACCTGTGGGGTAGCCATGGAAACAGCACATTCCCAGAGTTCCTCCAC
AACATGGACTACTTCAAGTTCCACAACATGCGACCCCCTTTCACCTACGC
CACGCTCATCCGCTGGGCCATCCTGGAGGCTCCAGAGAAGCAGCGGACAC
TCAATGAGATCTACCACTGGTTCACACGCATGTTTGCCTTCTTCAGAAAC
CATCCTGCCACCTGGAAGAACGCCATCCGCCACAACCTGAGTCTGCACAA
GTGCTTTGTGCGGGTGGAGAGCGAGAAGGGGGCTGTGTGGACCGTGGATG
AGCTGGAGTTCCGCAAGAAACGGAGCCAGAGGCCCAGCAGGTGTTCCAAC
CCTACACCTGGCCCCTGACCTCAAGATCAAGGAAAGGAGGATGGACGAAC
AGGGGCCAAACTGGTGGGAGGCAGAGGTGGTGGGGGCAGGGATGATAGGC
CCTGGATGTGCCCACAGGGACCAAGAAGTGAGGTTTCCACTGTCTTGCCT
GCCAGGGCCCCTGTTCCCCCGCTGGCAGCCACCCCCTCCCCCATCATATC
CTTTGCCCCAAGGCTGCTCAGAGGGGCCCCGGTCCTGGCCCCAGCCCCCA
CCTCCGCCCCAGACACACCCCCCAGTCGAGCCCTGCAGCCAAACAGAGCC
TTCACAACCAGCCACACAGAGCCTGCCTCAGCTGCTCGCACAGATTACTT
CAGGGCTGGAAAAGTCACACAGACACACAAAATGTCACAATCCTGTCCCT
CACTCAACACAAACCCCAAAACACAGAGAGCCTGCCTCAGTACACTCAAA
CAACCTCAAAGCTGCATCATCACACAATCACACACAAGCACAGCCCTGAC
AACCCACACACCCCAAGGCACGCACCCACAGCCAGCCTCAGGGCCCACAG
GGGCACTGTCAACACAGGGGTGTGCCCAGAGGCCTACACAGAAGCAGCGT
CAGTACCCTCAGGATCTGAGGTCCCAACACGTGCTCGCTCACACACACGG
CCTGTTAGAATTCACCTGTGTATCTCACGCATATGCACACGCACAGCCCC
CCAGTGGGTCTCTTGAGTCCCGTGCAGACACACACAGCCACACACACTGC
CTTGCCAAAAATACCCCGTGTCTCCCCTGCCACTCACCTCACTCCCATTC
CCTGAGCCCTGATCCATGCCTCAGCTTAGACTGCAGAGGAACTACTCATT
TATTTGGGATCCAAGGCCCCCAACCCACAGTACCGTCCCCAATAAACTGC
AGCCGAGCTCCCCA
DNA sequence for human FOXP3 transcript
variant 1 mRNA coding sequence cDNA
SEQ ID NO: 2
ATGCCCAACCCCAGGCCTGGCAAGCCCTCGGCCCCTTCCTTGGCCCTTGG
CCCATCCCCAGGAGCCTCGCCCAGCTGGAGGGCTGCACCCAAAGCCTCAG
ACCTGCTGGGGGCCCGGGGCCCAGGGGGAACCTTCCAGGGCCGAGATCTT
CGAGGCGGGGCCCATGCCTCCTCTTCTTCCTTGAACCCCATGCCACCATC
GCAGCTGCAGCTGCCCACACTGCCCCTAGTCATGGTGGCACCCTCCGGGG
CACGGCTGGGCCCCTTGCCCCACTTACAGGCACTCCTCCAGGACAGGCCA
CATTTCATGCACCAGCTCTCAACGGTGGATGCCCACGCCCGGACCCCTGT
GCTGCAGGTGCACCCCCTGGAGAGCCCAGCCATGATCAGCCTCACACCAC
CCACCACCGCCACTGGGGTCTTCTCCCTCAAGGCCCGGCCTGGCCTCCCA
CCTGGGATCAACGTGGCCAGCCTGGAATGGGTGTCCAGGGAGCCGGCACT
GCTCTGCACCTTCCCAAATCCCAGTGCACCCAGGAAGGACAGCACCCTTT
CGGCTGTGCCCCAGAGCTCCTACCCACTGCTGGCAAATGGTGTCTGCAAG
TGGCCCGGATGTGAGAAGGTCTTCGAAGAGCCAGAGGACTTCCTCAAGCA
CTGCCAGGCGGACCATCTTCTGGATGAGAAGGGCAGGGCACAATGTCTCC
TCCAGAGAGAGATGGTACAGTCTCTGGAGCAGCAGCTGGTGCTGGAGAAG
GAGAAGCTGAGTGCCATGCAGGCCCACCTGGCTGGGAAAATGGCACTGAC
CAAGGCTTCATCTGTGGCATCATCCGACAAGGGCTCCTGCTGCATCGTAG
CTGCTGGCAGCCAAGGCCCTGTCGTCCCAGCCTGGTCTGGCCCCCGGGAG
GCCCCTGACAGCCTGTTTGCTGTCCGGAGGCACCTGTGGGGTAGCCATGG
AAACAGCACATTCCCAGAGTTCCTCCACAACATGGACTACTTCAAGTTCC
ACAACATGCGACCCCCTTTCACCTACGCCACGCTCATCCGCTGGGCCATC
CTGGAGGCTCCAGAGAAGCAGCGGACACTCAATGAGATCTACCACTGGTT
CACACGCATGTTTGCCTTCTTCAGAAACCATCCTGCCACCTGGAAGAACG
CCATCCGCCACAACCTGAGTCTGCACAAGTGCTTTGTGCGGGTGGAGAGC
GAGAAGGGGGCTGTGTGGACCGTGGATGAGCTGGAGTTCCGCAAGAAACG
GAGCCAGAGGCCCAGCAGGTGTTCCAACCCTACACCTGGCCCCTGA
DNA sequence for human FOXP3 transcript
variant 1 mRNA coding sequence cDNA
codon optimized version 1
SEQ ID NO: 3
ATGGGCCCCAACCCCCGTCCGGGCAAACCCTCTGCTCCATCACTCGCGCT
GGGCCCTTCGCCTGGAGCTTCCCCCAGTTGGCGGGCCGCTCCCAAGGCCA
GCGATTTGCTGGGTGCCCGGGGACCCGGAGGTACGTTCCAGGGTCGCGAC
CTGAGAGGCGGCGCGCATGCCTCTTCGTCCTCCCTCAATCCCATGCCTCC
AAGCCAGCTCCAACTGCCCACCCTGCCTCTGGTGATGGTCGCCCCATCTG
GGGCTCGCCTGGGTCCGCTTCCTCATCTGCAAGCCCTGCTGCAGGACAGG
CCTCACTTCATGCACCAGCTGAGCACCGTGGACGCCCACGCCCGCACCCC
CGTGCTGCAGGTGCACCCCCTGGAATCTCCGGCCATGATCTCTCTGACTC
CACCTACCACAGCCACCGGCGTTTTCAGCCTGAAAGCCCGTCCTGGCCTG
CCTCCTGGCATCAACGTGGCCTCCCTCGAGTGGGTCTCGCGGGAACCGGC
GCTTCTGTGCACTTTTCCCAATCCTTCCGCTCCTCGGAAGGATTCCACAC
TGAGCGCCGTGCCTCAGAGTAGCTACCCCTTGCTGGCCAACGGCGTGTGC
AAGTGGCCCGGGTGCGAGAAGGTGTTCGAGGAGCCCGAGGACTTCCTGAA
GCACTGTCAGGCCGACCACCTGCTGGACGAGAAGGGCAGAGCCCAGTGCC
TGCTTCAGCGCGAGATGGTGCAGAGCTTGGAACAGCAGCTGGTATTAGAA
AAAGAAAAGCTGTCTGCCATGCAGGCCCATCTGGCTGGCAAGATGGCCCT
GACCAAGGCAAGCTCCGTGGCTTCGAGCGACAAGGGCAGCTGTTGTATCG
TGGCCGCCGGATCTCAGGGCCCTGTGGTCCCTGCTTGGTCTGGCCCACGC
GAGGCACCCGACTCCCTGTTCGCCGTGAGAAGACACCTGTGGGGCAGCCA
CGGCAACAGCACCTTTCCAGAGTTTCTGCACAACATGGATTATTTCAAGT
TCCACAACATGAGACCTCCCTTCACCTACGCCACGCTGATCAGATGGGCC
ATCCTGGAGGCCCCTGAAAAGCAGAGAACCCTGAACGAGATCTACCACTG
GTTCACTCGGATGTTTGCGTTCTTCCGCAATCACCCTGCTACCTGGAAGA
ACGCCATTCGCCACAACCTGAGCCTGCACAAGTGCTTTGTCAGAGTGGAG
AGCGAGAAAGGCGCTGTGTGGACCGTTGATGAGCTGGAGTTCAGAAAGAA
GAGGAGCCAGAGACCATCCAGGTGTAGCAACCCCACACCTGGCCCCTGA
DNA sequence for human FOXP3 transcript
variant 1 mRNA coding sequence cDNA
codon optimized version 2
SEQ ID NO: 4
ATGGGACCCAATCCCAGACCCGGGAAGCCTAGCGCCCCGTCTCTTGCCTT
GGGGCCCTCTCCCGGTGCCTCACCTAGTTGGAGAGCCGCGCCTAAGGCTT
CTGATCTGCTGGGCGCTAGAGGCCCTGGGGGGACGTTCCAGGGCCGGGAC
CTGAGAGGCGGAGCCCACGCCTCCAGTTCCTCCCTGAACCCAATGCCTCC
ATCGCAGCTGCAGCTGCCCACACTGCCCTTAGTGATGGTGGCTCCTAGCG
GAGCTCGGCTGGGACCCCTGCCTCATCTGCAAGCCCTACTGCAGGACAGG
CCGCACTTTATGCACCAGCTGTCCACCGTCGACGCACATGCCAGGACCCC
TGTGCTCCAAGTGCACCCTCTGGAGAGTCCTGCTATGATCAGCCTGACTC
CGCCTACCACAGCCACAGGCGTGTTCTCCCTGAAGGCCAGACCCGGCCTG
CCTCCTGGCATCAACGTGGCTTCCCTGGAGTGGGTCTCTCGGGAGCCCGC
TCTGCTGTGCACTTTCCCTAACCCTAGCGCCCCGAGAAAGGACAGCACAC
TGAGCGCCGTTCCTCAGAGCTCGTACCCCCTGCTGGCCAACGGAGTTTGT
AAATGGCCCGGGTGCGAGAAAGTGTTCGAAGAGCCGGAGGACTTCTTGAA
GCACTGTCAGGCCGATCACCTGCTCGACGAAAAGGGACGTGCCCAGTGCC
TGTTGCAGCGGGAGATGGTGCAGTCCCTAGAACAGCAGCTCGTGCTGGAA
AAGGAAAAGCTGAGCGCCATGCAGGCCCATCTGGCAGGTAAGATGGCCCT
TACAAAAGCCTCTTCCGTGGCCAGTAGCGACAAAGGCTCCTGCTGCATCG
TCGCTGCTGGCTCCCAGGGCCCGGTGGTGCCTGCCTGGTCTGGCCCACGA
GAGGCTCCTGATAGCCTCTTTGCTGTGCGGCGCCATCTCTGGGGCAGCCA
CGGCAATTCCACCTTCCCCGAGTTCCTGCACAACATGGATTATTTCAAGT
TTCACAACATGCGTCCCCCTTTCACCTACGCTACACTGATCCGGTGGGCC
ATCCTGGAAGCACCTGAGAAACAGAGAACACTGAACGAGATCTACCACTG
GTTCACCCGGATGTTTGCGTTCTTCCGCAACCACCCTGCTACCTGGAAGA
ACGCTATTCGCCACAATCTGTCTCTGCACAAATGCTTTGTCCGAGTAGAG
AGCGAGAAGGGCGCCGTGTGGACCGTGGATGAACTGGAGTTCAGAAAGAA
GAGATCCCAGAGACCATCGAGGTGTAGCAACCCAACTCCGGGCCCC
RNA sequence for human FOXP3 transcript
variant 1, mRNA coding sequence cDNA
codon optimized version 2
SEQ ID NO: 5
AUGGGACCCAAUCCCAGACCCGGGAAGCCUAGCGCCCCGUCUCUUGCCUU
GGGGCCCUCUCCCGGUGCCUCACCUAGUUGGAGAGCCGCGCCUAAGGCUU
CUGAUCUGCUGGGCGCUAGAGGCCCUGGGGGGACGUUCCAGGGCCGGGAC
CUGAGAGGCGGAGCCCACGCCUCCAGUUCCUCCCUGAACCCAAUGCCUCC
AUCGCAGCUGCAGCUGCCCACACUGCCCUUAGUGAUGGUGGCUCCUAGCG
GAGCUCGGCUGGGACCCCUGCCUCAUCUGCAAGCCCUACUGCAGGACAGG
CCGCACUUUAUGCACCAGCUGUCCACCGUCGACGCACAUGCCAGGACCCC
UGUGCUCCAAGUGCACCCUCUGGAGAGUCCUGCUAUGAUCAGCCUGACUC
CGCCUACCACAGCCACAGGCGUGUUCUCCCUGAAGGCCAGACCCGGCCUG
CCUCCUGGCAUCAACGUGGCUUCCCUGGAGUGGGUCUCUCGGGAGCCCGC
UCUGCUGUGCACUUUCCCUAACCCUAGCGCCCCGAGAAAGGACAGCACAC
UGAGCGCCGUUCCUCAGAGCUCGUACCCCCUGCUGGCCAACGGAGUUUGU
AAAUGGCCCGGGUGCGAGAAAGUGUUCGAAGAGCCGGAGGACUUCUUGAA
GCACUGUCAGGCCGAUCACCUGCUCGACGAAAAGGGACGUGCCCAGUGCC
UGUUGCAGCGGGAGAUGGUGCAGUCCCUAGAACAGCAGCUCGUGCUGGAA
AAGGAAAAGCUGAGCGCCAUGCAGGCCCAUCUGGCAGGUAAGAUGGCCCU
UACAAAAGCCUCUUCCGUGGCCAGUAGCGACAAAGGCUCCUGCUGCAUCG
UCGCUGCUGGCUCCCAGGGCCCGGUGGUGCCUGCCUGGUCUGGCCCACGA
GAGGCUCCUGAUAGCCUCUUUGCUGUGCGGCGCCAUCUCUGGGGCAGCCA
CGGCAAUUCCACCUUCCCCGAGUUCCUGCACAACAUGGAUUAUUUCAAGU
UUCACAACAUGCGUCCCCCUUUCACCUACGCUACACUGAUCCGGUGGGCC
AUCCUGGAAGCACCUGAGAAACAGAGAACACUGAACGAGAUCUACCACUG
GUUCACCCGGAUGUUUGCGUUCUUCCGCAACCACCCUGCUACCUGGAAGA
ACGCUAUUCGCCACAAUCUGUCUCUGCACAAAUGCUUUGUCCGAGUAGAG
AGCGAGAAGGGCGCCGUGUGGACCGUGGAUGAACUGGAGUUCAGAAAGAA
GAGAUCCCAGAGACCAUCGAGGUGUAGCAACCCAACUCCGGGCCCC
DNA sequence for human FOXP3 transcript
variant 2 mRNA cDNA
SEQ ID NO: 6
AGTTTCCCACAAGCCAGGCTGATCCTTTTCTGTCAGTCCACTTCACCAAG
CCTGCCCTTGGACAAGGACCCGATGCCCAACCCCAGGCCTGGCAAGCCCT
CGGCCCCTTCCTTGGCCCTTGGCCCATCCCCAGGAGCCTCGCCCAGCTGG
AGGGCTGCACCCAAAGCCTCAGACCTGCTGGGGGCCCGGGGCCCAGGGGG
AACCTTCCAGGGCCGAGATCTTCGAGGCGGGGCCCATGCCTCCTCTTCTT
CCTTGAACCCCATGCCACCATCGCAGCTGCAGCTCTCAACGGTGGATGCC
CACGCCCGGACCCCTGTGCTGCAGGTGCACCCCCTGGAGAGCCCAGCCAT
GATCAGCCTCACACCACCCACCACCGCCACTGGGGTCTTCTCCCTCAAGG
CCCGGCCTGGCCTCCCACCTGGGATCAACGTGGCCAGCCTGGAATGGGTG
TCCAGGGAGCCGGCACTGCTCTGCACCTTCCCAAATCCCAGTGCACCCAG
GAAGGACAGCACCCTTTCGGCTGTGCCCCAGAGCTCCTACCCACTGCTGG
CAAATGGTGTCTGCAAGTGGCCCGGATGTGAGAAGGTCTTCGAAGAGCCA
GAGGACTTCCTCAAGCACTGCCAGGCGGACCATCTTCTGGATGAGAAGGG
CAGGGCACAATGTCTCCTCCAGAGAGAGATGGTACAGTCTCTGGAGCAGC
AGCTGGTGCTGGAGAAGGAGAAGCTGAGTGCCATGCAGGCCCACCTGGCT
GGGAAAATGGCACTGACCAAGGCTTCATCTGTGGCATCATCCGACAAGGG
CTCCTGCTGCATCGTAGCTGCTGGCAGCCAAGGCCCTGTCGTCCCAGCCT
GGTCTGGCCCCCGGGAGGCCCCTGACAGCCTGTTTGCTGTCCGGAGGCAC
CTGTGGGGTAGCCATGGAAACAGCACATTCCCAGAGTTCCTCCACAACAT
GGACTACTTCAAGTTCCACAACATGCGACCCCCTTTCACCTACGCCACGC
TCATCCGCTGGGCCATCCTGGAGGCTCCAGAGAAGCAGCGGACACTCAAT
GAGATCTACCACTGGTTCACACGCATGTTTGCCTTCTTCAGAAACCATCC
TGCCACCTGGAAGAACGCCATCCGCCACAACCTGAGTCTGCACAAGTGCT
TTGTGCGGGTGGAGAGCGAGAAGGGGGCTGTGTGGACCGTGGATGAGCTG
GAGTTCCGCAAGAAACGGAGCCAGAGGCCCAGCAGGTGTTCCAACCCTAC
ACCTGGCCCCTGACCTCAAGATCAAGGAAAGGAGGATGGACGAACAGGGG
CCAAACTGGTGGGAGGCAGAGGTGGTGGGGGCAGGGATGATAGGCCCTGG
ATGTGCCCACAGGGACCAAGAAGTGAGGTTTCCACTGTCTTGCCTGCCAG
GGCCCCTGTTCCCCCGCTGGCAGCCACCCCCTCCCCCATCATATCCTTTG
CCCCAAGGCTGCTCAGAGGGGCCCCGGTCCTGGCCCCAGCCCCCACCTCC
GCCCCAGACACACCCCCCAGTCGAGCCCTGCAGCCAAACAGAGCCTTCAC
AACCAGCCACACAGAGCCTGCCTCAGCTGCTCGCACAGATTACTTCAGGG
CTGGAAAAGTCACACAGACACACAAAATGTCACAATCCTGTCCCTCACTC
AACACAAACCCCAAAACACAGAGAGCCTGCCTCAGTACACTCAAACAACC
TCAAAGCTGCATCATCACACAATCACACACAAGCACAGCCCTGACAACCC
ACACACCCCAAGGCACGCACCCACAGCCAGCCTCAGGGCCCACAGGGGCA
CTGTCAACACAGGGGTGTGCCCAGAGGCCTACACAGAAGCAGCGTCAGTA
CCCTCAGGATCTGAGGTCCCAACACGTGCTCGCTCACACACACGGCCTGT
TAGAATTCACCTGTGTATCTCACGCATATGCACACGCACAGCCCCCCAGT
GGGTCTCTTGAGTCCCGTGCAGACACACACAGCCACACACACTGCCTTGC
CAAAAATACCCCGTGTCTCCCCTGCCACTCACCTCACTCCCATTCCCTGA
GCCCTGATCCATGCCTCAGCTTAGACTGCAGAGGAACTACTCATTTATTT
GGGATCCAAGGCCCCCAACCCACAGTACCGTCCCCAATAAACTGCAGCCG
AGCTCCCCA
DNA sequence for human FOXP3 transcript
variant 2 mRNA coding sequence cDNA
SEQ ID NO: 7
ATGCCCAACCCCAGGCCTGGCAAGCCCTCGGCCCCTTCCTTGGCCCTTGG
CCCATCCCCAGGAGCCTCGCCCAGCTGGAGGGCTGCACCCAAAGCCTCAG
ACCTGCTGGGGGCCCGGGGCCCAGGGGGAACCTTCCAGGGCCGAGATCTT
CGAGGCGGGGCCCATGCCTCCTCTTCTTCCTTGAACCCCATGCCACCATC
GCAGCTGCAGCTCTCAACGGTGGATGCCCACGCCCGGACCCCTGTGCTGC
AGGTGCACCCCCTGGAGAGCCCAGCCATGATCAGCCTCACACCACCCACC
ACCGCCACTGGGGTCTTCTCCCTCAAGGCCCGGCCTGGCCTCCCACCTGG
GATCAACGTGGCCAGCCTGGAATGGGTGTCCAGGGAGCCGGCACTGCTCT
GCACCTTCCCAAATCCCAGTGCACCCAGGAAGGACAGCACCCTTTCGGCT
GTGCCCCAGAGCTCCTACCCACTGCTGGCAAATGGTGTCTGCAAGTGGCC
CGGATGTGAGAAGGTCTTCGAAGAGCCAGAGGACTTCCTCAAGCACTGCC
AGGCGGACCATCTTCTGGATGAGAAGGGCAGGGCACAATGTCTCCTCCAG
AGAGAGATGGTACAGTCTCTGGAGCAGCAGCTGGTGCTGGAGAAGGAGAA
GCTGAGTGCCATGCAGGCCCACCTGGCTGGGAAAATGGCACTGACCAAGG
CTTCATCTGTGGCATCATCCGACAAGGGCTCCTGCTGCATCGTAGCTGCT
GGCAGCCAAGGCCCTGTCGTCCCAGCCTGGTCTGGCCCCCGGGAGGCCCC
TGACAGCCTGTTTGCTGTCCGGAGGCACCTGTGGGGTAGCCATGGAAACA
GCACATTCCCAGAGTTCCTCCACAACATGGACTACTTCAAGTTCCACAAC
ATGCGACCCCCTTTCACCTACGCCACGCTCATCCGCTGGGCCATCCTGGA
GGCTCCAGAGAAGCAGCGGACACTCAATGAGATCTACCACTGGTTCACAC
GCATGTTTGCCTTCTTCAGAAACCATCCTGCCACCTGGAAGAACGCCATC
CGCCACAACCTGAGTCTGCACAAGTGCTTTGTGCGGGTGGAGAGCGAGAA
GGGGGCTGTGTGGACCGTGGATGAGCTGGAGTTCCGCAAGAAACGGAGCC
AGAGGCCCAGCAGGTGTTCCAACCCTACACCTGGCCCCTGA
amino acid sequence for human FOXP3
isoform A
SEQ ID NO: 8
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQLVLEK
EKLSAMQAHLAGKMALTKASSVASSDKGSCCIVAAGSQGPVVPAWSGPRE
APDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVES
EKGAVWTVDELEFRKKRSQRPSRCSNPTPGP
amino acid sequence for human FOXP3
isoform B
SEQ ID NO: 9
MPNPRPGKPSAPSLALGPSPGASPSWRAAPKASDLLGARGPGGTFQGRDL
RGGAHASSSSLNPMPPSQLQLSTVDAHARTPVLQVHPLESPAMISLTPPT
TATGVFSLKARPGLPPGINVASLEWVSREPALLCTFPNPSAPRKDSTLSA
VPQSSYPLLANGVCKWPGCEKVFEEPEDFLKHCQADHLLDEKGRAQCLLQ
REMVQSLEQQLVLEKEKLSAMQAHLAGKMALTKASSVASSDKGSCCIVAA
GSQGPVVPAWSGPREAPDSLFAVRRHLWGSHGNSTFPEFLHNMDYFKFHN
MRPPFTYATLIRWAILEAPEKQRTLNEIYHWFTRMFAFFRNHPATWKNAI
RHNLSLHKCFVRVESEKGAVWTVDELEFRKKRSQRPSRCSNPTPGP
amino acid sequence for human FOXP3
isoform C
SEQ ID NO: 10
MPNPRPGKPSAPSLALGPSPGASPSWRAAPKASDLLGARGPGGTFQGRDL
RGGAHASSSSLNPMPPSQLQLSTVDAHARTPVLQVHPLESPAMISLTPPT
TATGVFSLKARPGLPPGINVASLEWVSREPALLCTFPNPSAPRKDSTLSA
VPQSSYPLLANGVCKWPGCEKVFEEPEDFLKHCQADHLLDEKGRAQCLLQ
REMVQSLEQQLVLEKEKLSAMQAHLAGKMALTKASSVASSDKGSCCIVAA
GSQGPVVPAWSGPREAPDSLFAVRRHLWGSHGNSTFPEFLHNMDYFKFHN
MRPPFTYATLIRWAILEAPEKQRTLNEIYHWFTRMFAFFRNHPATWKVSS
SEVAVTGMASSAIAAQSGQAWVWAHRHIGEERDVGCWWWLLASEVDAHLL
PVPGLPQNAIRHNLSLHKCFVRVESEKGAVWTVDELEFRKKRSQRPSRCS
NPTPGP
amino acid sequence for human FOXP3
isoform D
SEQ ID NO: 11
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQASSDK
GSCCIVAAGSQGPVVPAWSGPREAPDSLFAVRRHLWGSHGNSTFPEFLHN
MDYFKFHNMRPPFTYATLIRWAILEAPEKQRTLNEIYHWFTRMFAFFRNH
PATWKNAIRHNLSLHKCFVRVESEKGAVWTVDELEFRKKRSQRPSRCSNP
TPGP
DNA sequence of human IKZF2, transcript
variant 2, cDNA
SEQ ID NO: 12
ATGGAAACAGAGGCTATTGATGGCTATATAACGTGTGACAATGAGCTTTC
ACCCGAAAGGGAGCACTCCAATATGGCAATTGACCTCACCTCAAGCACAC
CCAATGGACAGCATGCCTCACCAAGTCACATGACAAGCACAAATTCAGTA
AAGCTAGAAATGCAGAGTGATGAAGAGTGTGACAGGAAACCCCTGAGCCG
TGAAGATGAGATCAGGGGCCATGATGAGGGTAGCAGCCTAGAAGAACCCC
TAATTGAGAGCAGCGAGGTGGCTGACAACAGGAAAGTCCAGGAGCTTCAA
GGCGAGGGAGGAATCCGGCTTCCGAATGGTGAACGCCCCTTCCACTGTAA
CCAGTGTGGAGCTTCTTTTACTCAGAAGGGCAACCTTCTGAGACACATAA
AGTTACACTCTGGAGAGAAGCCGTTCAAATGTCCTTTCTGTAGCTACGCC
TGTAGAAGAAGGGACGCCCTCACAGGACACCTCAGGACCCATTCTGTGGG
TAAACCTCACAAGTGCAACTACTGTGGACGAAGCTACAAGCAGCGCAGTT
CACTGGAGGAGCACAAGGAACGCTGCCACAACTATCTCCAGAATGTCAGC
ATGGAGGCTGCTGGGCAGGTCATGAGTCACCATGTACCTCCTATGGAAGA
TTGTAAGGAACAAGAGCCTATTATGGACAACAATATTTCTCTGGTGCCTT
TTGAGAGACCTGCTGTCATAGAGAAGCTCACGGGGAATATGGGAAAACGT
AAAAGCTCCACTCCACAAAAGTTTGTGGGGGAAAAGCTCATGCGATTCAG
CTACCCAGATATTCACTTTGATATGAACTTAACATATGAGAAGGAGGCTG
AGCTGATGCAGTCTCATATGATGGACCAAGCCATCAACAATGCAATCACC
TACCTTGGAGCTGAGGCCCTTCACCCTCTGATGCAGCACCCGCCAAGCAC
AATCGCTGAAGTGGCCCCAGTTATAAGCTCAGCTTATTCTCAGGTCTATC
ATCCAAATAGGATAGAAAGACCCATTAGCAGGGAAACTGCTGATAGTCAT
GAAAACAACATGGATGGCCCCATCTCTCTCATCAGACCAAAGAGTCGACC
CCAGGAAAGAGAGGCCTCTCCCAGCAATAGCTGCCTGGATTCCACTGACT
CAGAAAGCAGCCATGATGACCACCAGTCCTACCAAGGACACCCTGCCTTA
AATCCCAAGAGGAAACAAAGCCCAGCTTACATGAAGGAGGATGTCAAAGC
TTTGGATACTACCAAGGCTCCTAAGGGCTCTCTGAAGGACATCTACAAGG
TCTTCAATGGAGAAGGAGAACAGATTAGGGCCTTCAAGTGTGAGCACTGC
CGAGTCCTTTTCCTAGACCATGTCATGTACACCATTCACATGGGTTGCCA
TGGCTACCGGGACCCACTGGAATGCAACATCTGTGGCTACAGAAGCCAGG
ACCGTTATGAGTTTTCATCACACATTGTTCGAGGGGAGCACACATTCCAC
TAG
DNA sequence of human IKZF2, transcript
variant 2, CDS cDNA codon optimized
version 1
SEQ ID NO: 13
ATGGAAACCGAGGCTATCGACGGCTACATCACCTGCGACAACGAGCTTTC
TCCTGAGAGAGAACACTCAAATATGGCCATCGACCTGACGTCTAGCACTC
CGAACGGACAGCACGCGAGCCCCAGCCATATGACCAGCACCAACAGCGTT
AAGCTGGAGATGCAGAGCGATGAAGAGTGTGATAGGAAGCCTCTGAGTCG
CGAGGACGAGATCCGGGGACACGACGAGGGCTCCTCGTTGGAAGAACCTC
TTATTGAGTCTAGCGAGGTGGCGGACAACAGAAAAGTGCAGGAGCTGCAG
GGCGAGGGCGGCATTCGCCTACCTAACGGTGAGCGGCCTTTCCACTGTAA
CCAGTGTGGCGCTAGCTTCACCCAGAAAGGCAACCTGTTGCGCCACATCA
AACTGCACTCTGGCGAGAAGCCGTTTAAATGTCCCTTCTGCAGCTACGCC
TGCCGGCGCCGCGACGCCCTGACCGGCCACTTGAGAACACACAGCGTGGG
CAAGCCTCACAAGTGCAACTACTGCGGCAGAAGCTACAAGCAAAGATCTT
CCCTTGAGGAGCACAAAGAGAGGTGTCATAACTACCTGCAGAATGTGTCC
ATGGAAGCAGCCGGCCAAGTAATGAGCCACCACGTCCCACCCATGGAAGA
CTGCAAGGAGCAGGAGCCCATCATGGATAACAATATCTCTCTGGTGCCCT
TCGAGCGTCCAGCCGTGATCGAGAAACTCACTGGTAATATGGGAAAGCGC
AAGAGCTCTACACCTCAGAAGTTCGTGGGGGAGAAGCTGATGCGCTTTTC
ATACCCCGACATCCATTTCGATATGAACCTGACCTATGAGAAAGAAGCTG
AGCTGATGCAGAGTCATATGATGGACCAGGCCATCAACAACGCCATTACC
TACCTGGGCGCCGAGGCCCTGCACCCCCTGATGCAGCACCCTCCATCCAC
GATCGCCGAAGTCGCCCCTGTGATCTCCAGCGCCTACAGCCAAGTCTACC
ACCCCAACCGGATCGAACGGCCGATTTCCAGGGAGACAGCCGACAGCCAC
GAGAACAACATGGACGGGCCCATCTCTCTGATCCGGCCCAAGTCCCGCCC
TCAGGAGAGGGAAGCTTCCCCCAGCAACTCATGCCTGGACAGTACCGACT
CTGAGAGCAGCCATGATGACCACCAGAGCTATCAGGGACATCCTGCTCTC
AATCCAAAGCGCAAGCAGTCCCCAGCGTACATGAAGGAAGATGTGAAGGC
TCTGGATACAACCAAGGCTCCTAAGGGCTCCCTGAAGGACATCTACAAGG
TGTTCAACGGCGAAGGCGAACAGATCAGAGCCTTCAAGTGCGAGCACTGC
AGAGTGCTGTTCCTGGACCACGTGATGTACACCATCCACATGGGCTGCCA
CGGTTACAGAGATCCACTGGAGTGTAACATCTGCGGCTATCGTTCTCAGG
ACAGGTACGAGTTCAGCTCGCACATCGTGCGGGGTGAGCATACCTTTCAC
TGA
DNA sequence of human IKZF2, transcript
variant 2, CDS cDNA codon optimized
version 2
SEQ ID NO: 14
ATGGAAACGGAGGCTATTGACGGCTACATCACCTGCGACAACGAACTATC
TCCTGAAAGAGAGCACTCCAATATGGCGATCGACCTAACCAGCTCCACCC
CTAACGGACAGCATGCCTCTCCGTCCCACATGACCAGCACCAACAGCGTC
AAACTGGAGATGCAGTCCGACGAGGAGTGTGACCGCAAGCCCCTGTCTCG
CGAGGACGAGATAAGAGGTCATGACGAGGGATCTAGCCTCGAGGAACCAC
TCATTGAGAGCTCCGAGGTGGCCGACAATCGGAAGGTGCAGGAACTACAA
GGCGAAGGCGGCATCCGTCTGCCTAATGGCGAGAGACCTTTCCACTGTAA
CCAGTGTGGCGCGAGCTTCACCCAGAAGGGCAATCTGCTGAGACACATCA
AGCTGCACTCTGGTGAGAAGCCTTTTAAATGCCCATTCTGCTCTTACGCC
TGCCGACGGCGCGACGCCCTGACCGGCCACCTCCGCACCCACTCTGTGGG
CAAACCTCACAAGTGCAACTACTGCGGCCGCTCCTACAAGCAGAGAAGCA
GCTTGGAAGAGCACAAGGAGCGCTGTCACAACTATCTGCAGAACGTGTCC
ATGGAAGCCGCCGGCCAGGTGATGAGCCACCACGTGCCGCCCATGGAAGA
TTGCAAGGAGCAGGAGCCCATCATGGACAACAACATCTCACTGGTCCCTT
TCGAGAGACCGGCGGTGATTGAGAAGCTGACAGGAAACATGGGCAAGCGC
AAAAGCTCGACGCCCCAGAAGTTCGTCGGCGAGAAGCTGATGCGTTTCAG
CTATCCCGACATCCACTTCGACATGAACCTGACTTACGAGAAAGAGGCTG
AACTGATGCAAAGTCACATGATGGACCAGGCCATCAATAACGCCATCACT
TACCTGGGCGCCGAGGCCCTGCACCCCCTGATGCAGCATCCCCCGTCTAC
CATCGCCGAGGTGGCCCCCGTCATTAGCAGCGCCTACTCCCAGGTGTACC
ACCCCAACCGCATCGAGCGGCCCATCTCTCGTGAGACTGCGGATTCTCAC
GAGAACAATATGGATGGTCCCATCAGCCTCATCAGACCTAAGAGCCGCCC
TCAAGAGAGAGAGGCCTCCCCAAGCAACAGCTGCCTGGATTCTACCGACA
GCGAATCGAGCCACGATGACCACCAGAGCTATCAGGGTCACCCTGCCCTC
AACCCGAAGAGAAAGCAGAGCCCCGCCTACATGAAGGAAGACGTGAAGGC
CCTGGACACCACCAAGGCCCCTAAGGGCTCCCTGAAGGACATCTACAAGG
TGTTCAACGGCGAGGGCGAGCAAATCCGCGCATTCAAGTGCGAGCACTGT
AGAGTGCTGTTCCTGGACCACGTGATGTACACCATCCATATGGGCTGTCA
TGGCTACCGCGACCCACTTGAGTGCAACATCTGTGGCTACAGATCTCAGG
ATCGCTACGAGTTTTCGAGCCACATCGTGCGCGGAGAGCACACGTTTCAC
TGA
RNA sequence of human IKZF2, transcript
variant 2, CDS codon optimized
version 2
SEQ ID NO: 15
AUGGAAACGGAGGCUAUUGACGGCUACAUCACCUGCGACAACGAACUAUC
UCCUGAAAGAGAGCACUCCAAUAUGGCGAUCGACCUAACCAGCUCCACCC
CUAACGGACAGCAUGCCUCUCCGUCCCACAUGACCAGCACCAACAGCGUC
AAACUGGAGAUGCAGUCCGACGAGGAGUGUGACCGCAAGCCCCUGUCUCG
CGAGGACGAGAUAAGAGGUCAUGACGAGGGAUCUAGCCUCGAGGAACCAC
UCAUUGAGAGCUCCGAGGUGGCCGACAAUCGGAAGGUGCAGGAACUACAA
GGCGAAGGCGGCAUCCGUCUGCCUAAUGGCGAGAGACCUUUCCACUGUAA
CCAGUGUGGCGCGAGCUUCACCCAGAAGGGCAAUCUGCUGAGACACAUCA
AGCUGCACUCUGGUGAGAAGCCUUUUAAAUGCCCAUUCUGCUCUUACGCC
UGCCGACGGCGCGACGCCCUGACCGGCCACCUCCGCACCCACUCUGUGGG
CAAACCUCACAAGUGCAACUACUGCGGCCGCUCCUACAAGCAGAGAAGCA
GCUUGGAAGAGCACAAGGAGCGCUGUCACAACUAUCUGCAGAACGUGUCC
AUGGAAGCCGCCGGCCAGGUGAUGAGCCACCACGUGCCGCCCAUGGAAGA
UUGCAAGGAGCAGGAGCCCAUCAUGGACAACAACAUCUCACUGGUCCCUU
UCGAGAGACCGGCGGUGAUUGAGAAGCUGACAGGAAACAUGGGCAAGCGC
AAAAGCUCGACGCCCCAGAAGUUCGUCGGCGAGAAGCUGAUGCGUUUCAG
CUAUCCCGACAUCCACUUCGACAUGAACCUGACUUACGAGAAAGAGGCUG
AACUGAUGCAAAGUCACAUGAUGGACCAGGCCAUCAAUAACGCCAUCACU
UACCUGGGCGCCGAGGCCCUGCACCCCCUGAUGCAGCAUCCCCCGUCUAC
CAUCGCCGAGGUGGCCCCCGUCAUUAGCAGCGCCUACUCCCAGGUGUACC
ACCCCAACCGCAUCGAGCGGCCCAUCUCUCGUGAGACUGCGGAUUCUCAC
GAGAACAAUAUGGAUGGUCCCAUCAGCCUCAUCAGACCUAAGAGCCGCCC
UCAAGAGAGAGAGGCCUCCCCAAGCAACAGCUGCCUGGAUUCUACCGACA
GCGAAUCGAGCCACGAUGACCACCAGAGCUAUCAGGGUCACCCUGCCCUC
AACCCGAAGAGAAAGCAGAGCCCCGCCUACAUGAAGGAAGACGUGAAGGC
CCUGGACACCACCAAGGCCCCUAAGGGCUCCCUGAAGGACAUCUACAAGG
UGUUCAACGGCGAGGGCGAGCAAAUCCGCGCAUUCAAGUGCGAGCACUGU
AGAGUGCUGUUCCUGGACCACGUGAUGUACACCAUCCAUAUGGGCUGUCA
UGGCUACCGCGACCCACUUGAGUGCAACAUCUGUGGCUACAGAUCUCAGG
AUCGCUACGAGUUUUCGAGCCACAUCGUGCGCGGAGAGCACACGUUUCAC
UGA
amino acid sequence of human IKZF2
Helios isoform 2
SEQ ID NO: 16
METEAIDGYITCDNELSPEREHSNMAIDLTSSTPNGQHASPSHMTSTNSV
KLEMQSDEECDRKPLSREDEIRGHDEGSSLEEPLIESSEVADNRKVQELQ
GEGGIRLPNGERPFHCNQCGASFTQKGNLLRHIKLHSGEKPFKCPFCSYA
CRRRDALTGHLRTHSVGKPHKCNYCGRSYKQRSSLEEHKERCHNYLQNVS
MEAAGQVMSHHVPPMEDCKEQEPIMDNNISLVPFERPAVIEKLTGNMGKR
KSSTPQKFVGEKLMRFSYPDIHFDMNLTYEKEAELMQSHMMDQAINNAIT
YLGAEALHPLMQHPPSTIAEVAPVISSAYSQVYHPNRIERPISRETADSH
ENNMDGPISLIRPKSRPQEREASPSNSCLDSTDSESSHDDHQSYQGHPAL
NPKRKQSPAYMKEDVKALDTTKAPKGSLKDIYKVFNGEGEQIRAFKCEHC
RVLFLDHVMYTIHMGCHGYRDPLECNICGYRSQDRYEFSSHIVRGEHTFH
DNA sequence of signal peptide-myc-HGL
CAR19 (vH-3Gx4-vL-CD8 tm spacer-CD28-
CD3z) cDNA
SEQ ID NO: 17
ATGGCTAGCCCCCTGACACGCTTCCTGTCCCTGAACCTGCTGCTGCTGGG
AGAGAGCATCATCCTGGGCTCCGGCGAAGCCGAACAGAAACTGATTTCTG
AGGAAGACCTGGACGTGCAGCTGCAAGAGTCTGGGCCTGGGCTGGTGAAG
CCTAGCCAGAGCCTGTCTCTAACCTGCAGCGTCACAGGCTACTCCATCAC
AAGCGGCTACTACTGGACCTGGATCCGCCAGTTTCCGGGCAACAAGCTGG
AATGGATGGGGTCGATTGGCTACGACGGCACCAACTACTACAACCCCAGT
CTCAAGAACAGAATCAGCATCACCCGTGACACCAGCAAGAACCAGTTCTT
CTTGAAGCTGCATTCCGTGACCACCGAGGATACCGCTACCTATTACTGCG
CCAAGCGGGGCATCACCACTGGCGACTATTGGGGCCAGGGAACCACACTC
ACAGTTAGCTCTGGCGGGGGAGGCTCCGGTGGCGGCGGTAGCGGAGGTGG
TGGGTCCCAGGCCGTGGTGACGCAGGAGAGCGCCCTGACGACATCTCCCG
GCGAGACCGTGACCCTGACCTGTCGCAGCAGCACCGGCGCCGTCACTACC
TCCAACTACGCGAATTGGGTGCAGGAAAAACCCGACCACCTGTTCATCGG
ACTGATAGGTGGGACCAATAAGCGTACACCTGGCGTACCAGCTCGGTTTT
CTGGTTCCCTTATTGGGGACAAGGCCGCTCTGACCATCACTGGCACACAG
ACTGAGGACGAGGCCATCTACTTCTGCGCTCTGTGGTACAGCAACCACCT
GGTGTTCGGCGGCGGCACCAAGCTGACCGTCCTGTCGACCACAACCAAAC
CTGTGCTGAGAACTCCGTCGCCAGTGCACCCCACGGGCACCAGCCAGCCT
CAGAGACCTGAGGATTGTCGCCCTAGAGGGTCTGTGAAAGGCACGGGACT
GGACTTCGCCTGCGACATCTACATCTGGGCCCCTCTGGCTGGTATCTGTG
TGGCCCTGCTGCTCTCGCTGATCATCACCCTGATCTGCTACAACAGCAGA
CGAAACCGGCTCTTGCAGAGCGACTACATGAACATGACCCCCAGAAGGCC
TGGCCTAACTCGCAAGCCCTATCAGCCTTACGCGCCGGCCCGCGACTTTG
CGGCCTACCGGCCAAGAGCCAAGTTCAGCCGGAGAGCAGAAACAGCCGCC
AACCTGCAGGACCCCAACCAGCTGTACAACGAGCTAAACCTCGGCAGAAG
AGAGGAGTACGACGTGCTGGAGAAGAAGCGCGCTCGCGACCCAGAGATGG
GCGGTAAGCAGCAGCGCCGTCGGAATCCTCAAGAGGGCGTGTACAACGCC
CTCCAGAAGGATAAGATGGCCGAGGCCTATAGCGAGATCGGCACAAAGGG
CGAGAGAAGGCGCGGCAAAGGCCACGATGGCCTGTACCAGGGCCTGTCCA
CCGCCACCAAGGATACCTACGATGCCCTGCACATGCAGACGCTTGCACCC
CGGTGA
amino acid sequence of signal peptide-
myc-HGL CAR19 (vH-3Gx4-vL-CD8 tm
spacer-CD28-CD3z)
SEQ ID NO: 18
MASPLTRFLSLNLLLLGESIILGSGEAEQKLISEEDLDVQLQESGPGLVK
PSQSLSLTCSVTGYSITSGYYWTWIRQFPGNKLEWMGSIGYDGTNYYNPS
LKNRISITRDTSKNQFFLKLHSVTTEDTATYYCAKRGITTGDYWGQGTTL
TVSSGGGGSGGGGSGGGGSQAVVTQESALTTSPGETVTLTCRSSTGAVTT
SNYANWVQEKPDHLFIGLIGGTNKRTPGVPARFSGSLIGDKAALTITGTQ
TEDEAIYFCALWYSNHLVFGGGTKLTVLSTTTKPVLRTPSPVHPTGTSQP
QRPEDCRPRGSVKGTGLDFACDIYIWAPLAGICVALLLSLIITLICYNSR
RNRLLQSDYMNMTPRRPGLTRKPYQPYAPARDFAAYRPRAKFSRRAETAA
NLQDPNQLYNELNLGRREEYDVLEKKRARDPEMGGKQQRRRNPQEGVYNA
LQKDKMAEAYSEIGTKGERRRGKGHDGLYQGLSTATKDTYDALHMQTLAP
R
DNA sequence of 5′kozak-FOXP3-p2a-IKZF2-
spacer-ires-kozak-signal peptide-myc
tag-HGL scFV-CAR19 cDNA sequence
SEQ ID NO: 19
ATAGTAATAGCCGCCACCATGGGACCCAATCCCAGACCCGGGAAGCCTAG
CGCCCCGTCTCTTGCCTTGGGGCCCTCTCCCGGTGCCTCACCTAGTTGGA
GAGCCGCGCCTAAGGCTTCTGATCTGCTGGGCGCTAGAGGCCCTGGGGGG
ACGTTCCAGGGCCGGGACCTGAGAGGCGGAGCCCACGCCTCCAGTTCCTC
CCTGAACCCAATGCCTCCATCGCAGCTGCAGCTGCCCACACTGCCCTTAG
TGATGGTGGCTCCTAGCGGAGCTCGGCTGGGACCCCTGCCTCATCTGCAA
GCCCTACTGCAGGACAGGCCGCACTTTATGCACCAGCTGTCCACCGTCGA
CGCACATGCCAGGACCCCTGTGCTCCAAGTGCACCCTCTGGAGAGTCCTG
CTATGATCAGCCTGACTCCGCCTACCACAGCCACAGGCGTGTTCTCCCTG
AAGGCCAGACCCGGCCTGCCTCCTGGCATCAACGTGGCTTCCCTGGAGTG
GGTCTCTCGGGAGCCCGCTCTGCTGTGCACTTTCCCTAACCCTAGCGCCC
CGAGAAAGGACAGCACACTGAGCGCCGTTCCTCAGAGCTCGTACCCCCTG
CTGGCCAACGGAGTTTGTAAATGGCCCGGGTGCGAGAAAGTGTTCGAAGA
GCCGGAGGACTTCTTGAAGCACTGTCAGGCCGATCACCTGCTCGACGAAA
AGGGACGTGCCCAGTGCCTGTTGCAGCGGGAGATGGTGCAGTCCCTAGAA
CAGCAGCTCGTGCTGGAAAAGGAAAAGCTGAGCGCCATGCAGGCCCATCT
GGCAGGTAAGATGGCCCTTACAAAAGCCTCTTCCGTGGCCAGTAGCGACA
AAGGCTCCTGCTGCATCGTCGCTGCTGGCTCCCAGGGCCCGGTGGTGCCT
GCCTGGTCTGGCCCACGAGAGGCTCCTGATAGCCTCTTTGCTGTGCGGCG
CCATCTCTGGGGCAGCCACGGCAATTCCACCTTCCCCGAGTTCCTGCACA
ACATGGATTATTTCAAGTTTCACAACATGCGTCCCCCTTTCACCTACGCT
ACACTGATCCGGTGGGCCATCCTGGAAGCACCTGAGAAACAGAGAACACT
GAACGAGATCTACCACTGGTTCACCCGGATGTTTGCGTTCTTCCGCAACC
ACCCTGCTACCTGGAAGAACGCTATTCGCCACAATCTGTCTCTGCACAAA
TGCTTTGTCCGAGTAGAGAGCGAGAAGGGCGCCGTGTGGACCGTGGATGA
ACTGGAGTTCAGAAAGAAGAGATCCCAGAGACCATCGAGGTGTAGCAACC
CAACTCCGGGCCCCGGGTCGGGCGCTACCAACTTCAGCCTGCTGAAGCAG
GCCGGCGATGTGGAAGAAAACCCTGGGCCCATGGAAACGGAGGCTATTGA
CGGCTACATCACCTGCGACAACGAACTATCTCCTGAAAGAGAGCACTCCA
ATATGGCGATCGACCTAACCAGCTCCACCCCTAACGGACAGCATGCCTCT
CCGTCCCACATGACCAGCACCAACAGCGTCAAACTGGAGATGCAGTCCGA
CGAGGAGTGTGACCGCAAGCCCCTGTCTCGCGAGGACGAGATAAGAGGTC
ATGACGAGGGATCTAGCCTCGAGGAACCACTCATTGAGAGCTCCGAGGTG
GCCGACAATCGGAAGGTGCAGGAACTACAAGGCGAAGGCGGCATCCGTCT
GCCTAATGGCGAGAGACCTTTCCACTGTAACCAGTGTGGCGCGAGCTTCA
CCCAGAAGGGCAATCTGCTGAGACACATCAAGCTGCACTCTGGTGAGAAG
CCTTTTAAATGCCCATTCTGCTCTTACGCCTGCCGACGGCGCGACGCCCT
GACCGGCCACCTCCGCACCCACTCTGTGGGCAAACCTCACAAGTGCAACT
ACTGCGGCCGCTCCTACAAGCAGAGAAGCAGCTTGGAAGAGCACAAGGAG
CGCTGTCACAACTATCTGCAGAACGTGTCCATGGAAGCCGCCGGCCAGGT
GATGAGCCACCACGTGCCGCCCATGGAAGATTGCAAGGAGCAGGAGCCCA
TCATGGACAACAACATCTCACTGGTCCCTTTCGAGAGACCGGCGGTGATT
GAGAAGCTGACAGGAAACATGGGCAAGCGCAAAAGCTCGACGCCCCAGAA
GTTCGTCGGCGAGAAGCTGATGCGTTTCAGCTATCCCGACATCCACTTCG
ACATGAACCTGACTTACGAGAAAGAGGCTGAACTGATGCAAAGTCACATG
ATGGACCAGGCCATCAATAACGCCATCACTTACCTGGGCGCCGAGGCCCT
GCACCCCCTGATGCAGCATCCCCCGTCTACCATCGCCGAGGTGGCCCCCG
TCATTAGCAGCGCCTACTCCCAGGTGTACCACCCCAACCGCATCGAGCGG
CCCATCTCTCGTGAGACTGCGGATTCTCACGAGAACAATATGGATGGTCC
CATCAGCCTCATCAGACCTAAGAGCCGCCCTCAAGAGAGAGAGGCCTCCC
CAAGCAACAGCTGCCTGGATTCTACCGACAGCGAATCGAGCCACGATGAC
CACCAGAGCTATCAGGGTCACCCTGCCCTCAACCCGAAGAGAAAGCAGAG
CCCCGCCTACATGAAGGAAGACGTGAAGGCCCTGGACACCACCAAGGCCC
CTAAGGGCTCCCTGAAGGACATCTACAAGGTGTTCAACGGCGAGGGCGAG
CAAATCCGCGCATTCAAGTGCGAGCACTGTAGAGTGCTGTTCCTGGACCA
CGTGATGTACACCATCCATATGGGCTGTCATGGCTACCGCGACCCACTTG
AGTGCAACATCTGTGGCTACAGATCTCAGGATCGCTACGAGTTTTCGAGC
CACATCGTGCGCGGAGAGCACACGTTTCACTGATTAACAACAAGAGGGCC
CGGAAACCTGGCCCTGTCTTCTTGACGAGCATTCCTAGGGGTCTTTCCCC
TCTCGCCAAAGGAATGCAAGGTCTGTTGAATGTCGTGAAGGAAGCAGTTC
CTCTGGAAGCTTCTTGAAGACAAACAACGTCTGTAGCGACCCTTTGCAGG
CAGCGGAACCCCCCACCTGGCGACAGGTGCCTCTGCGGCCAAAAGCCACG
TGTATAAGATACACCTGCAAAGGCGGCACAACCCCAGTGCCACGTTGTGA
GTTGGATAGTTGTGGAAAGAGTCAAATGGCTCTCCTCAAGCGTATTCAAC
AAGGGGCTGAAGGATGCCCAGAAGGTACCCCATTGTATGGGATCTGATCT
GGGGCCTCGGTGCACATGCTTTACATGTGTTTAGTCGAGGTTAAAAAAAC
GTCTAGGCCCCCCGAACCACGGGGACGTGGTTTTCCTTTGAAAAACACGA
TGATAACATAGCATACATAGCCGCCACCATGGCTTCCCCCCTGACCCGCT
TCCTGTCTCTGAACCTGCTACTCCTCGGAGAGTCCATCATCCTGGGCTCT
GGCGAGGCAGAACAGAAACTGATTTCTGAGGAAGATCTGGATGTGCAGCT
GCAAGAATCTGGGCCCGGCCTGGTGAAGCCCTCTCAGAGCTTGTCACTGA
CCTGTTCCGTGACCGGATACAGCATCACCTCCGGCTACTATTGGACCTGG
ATCCGGCAGTTTCCTGGCAACAAGCTGGAGTGGATGGGATCCATTGGCTA
TGATGGCACCAACTACTACAATCCATCTCTTAAGAACCGCATCTCTATCA
CCAGAGATACATCCAAGAACCAGTTCTTTCTGAAGCTGCACAGCGTCACC
ACCGAGGACACCGCCACTTACTACTGCGCCAAGCGCGGCATCACCACTGG
CGACTACTGGGGCCAGGGTACAACCCTGACCGTGTCCAGCGGCGGGGGCG
GGTCCGGAGGCGGAGGATCCGGCGGCGGTGGCAGCCAGGCCGTGGTGACG
CAAGAGAGCGCTCTGACCACCAGCCCCGGCGAGACAGTGACCCTGACCTG
TAGAAGCAGCACCGGTGCGGTAACGACAAGCAACTACGCCAACTGGGTCC
AGGAGAAGCCCGACCATCTGTTCATCGGGCTGATCGGTGGCACCAACAAG
CGGACTCCGGGTGTGCCTGCTCGTTTCAGCGGGTCGTTAATTGGCGACAA
GGCCGCGCTCACCATTACAGGTACCCAGACCGAAGACGAGGCTATCTACT
TCTGTGCCCTGTGGTACAGCAACCACCTGGTGTTCGGCGGGGGCACCAAG
CTAACAGTGCTGTCCACCACGACCAAGCCTGTCCTGAGAACACCTAGCCC
TGTGCACCCCACGGGCACTAGCCAACCTCAGAGACCCGAGGACTGCCGAC
CCCGTGGATCTGTTAAGGGCACCGGCCTGGACTTCGCCTGCGATATCTAC
ATCTGGGCCCCTCTGGCCGGCATCTGCGTGGCCCTCCTGCTGAGCCTGAT
CATCACTCTGATCTGCTACAACAGCAGACGGAATCGTCTGCTGCAGAGCG
ATTACATGAATATGACCCCTAGGCGGCCTGGCCTTACGAGAAAACCCTAT
CAGCCATACGCTCCTGCCCGCGACTTTGCGGCCTACCGGCCGAGAGCCAA
ATTCTCCCGCCGAGCCGAGACTGCAGCCAACCTGCAGGACCCCAACCAGC
TCTACAACGAGCTGAACCTGGGCAGACGCGAGGAGTACGACGTGCTGGAA
AAGAAGAGGGCTCGGGACCCTGAGATGGGCGGCAAGCAGCAGAGAAGACG
GAATCCACAGGAGGGCGTGTACAACGCCTTGCAGAAGGACAAGATGGCCG
AGGCTTATTCCGAGATCGGTACAAAAGGTGAGAGAAGGCGCGGCAAAGGC
CACGACGGCCTCTACCAGGGCCTGAGCACAGCCACCAAGGACACCTACGA
CGCCCTGCACATGCAGACACTGGCGCCGCGCTGA
RNA sequence of 5′kozak-FOXP3-p2a-IKZF2-
spacer-ires-kozak-signal peptide-myc
tag-HGL scFV-CAR19 cDNA sequence
SEQ ID NO: 20
AUAGUAAUAGCCGCCACCAUGGGACCCAAUCCCAGACCCGGGAAGCCUAG
CGCCCCGUCUCUUGCCUUGGGGCCCUCUCCCGGUGCCUCACCUAGUUGGA
GAGCCGCGCCUAAGGCUUCUGAUCUGCUGGGCGCUAGAGGCCCUGGGGGG
ACGUUCCAGGGCCGGGACCUGAGAGGCGGAGCCCACGCCUCCAGUUCCUC
CCUGAACCCAAUGCCUCCAUCGCAGCUGCAGCUGCCCACACUGCCCUUAG
UGAUGGUGGCUCCUAGCGGAGCUCGGCUGGGACCCCUGCCUCAUCUGCAA
GCCCUACUGCAGGACAGGCCGCACUUUAUGCACCAGCUGUCCACCGUCGA
CGCACAUGCCAGGACCCCUGUGCUCCAAGUGCACCCUCUGGAGAGUCCUG
CUAUGAUCAGCCUGACUCCGCCUACCACAGCCACAGGCGUGUUCUCCCUG
AAGGCCAGACCCGGCCUGCCUCCUGGCAUCAACGUGGCUUCCCUGGAGUG
GGUCUCUCGGGAGCCCGCUCUGCUGUGCACUUUCCCUAACCCUAGCGCCC
CGAGAAAGGACAGCACACUGAGCGCCGUUCCUCAGAGCUCGUACCCCCUG
CUGGCCAACGGAGUUUGUAAAUGGCCCGGGUGCGAGAAAGUGUUCGAAGA
GCCGGAGGACUUCUUGAAGCACUGUCAGGCCGAUCACCUGCUCGACGAAA
AGGGACGUGCCCAGUGCCUGUUGCAGCGGGAGAUGGUGCAGUCCCUAGAA
CAGCAGCUCGUGCUGGAAAAGGAAAAGCUGAGCGCCAUGCAGGCCCAUCU
GGCAGGUAAGAUGGCCCUUACAAAAGCCUCUUCCGUGGCCAGUAGCGACA
AAGGCUCCUGCUGCAUCGUCGCUGCUGGCUCCCAGGGCCCGGUGGUGCCU
GCCUGGUCUGGCCCACGAGAGGCUCCUGAUAGCCUCUUUGCUGUGCGGCG
CCAUCUCUGGGGCAGCCACGGCAAUUCCACCUUCCCCGAGUUCCUGCACA
ACAUGGAUUAUUUCAAGUUUCACAACAUGCGUCCCCCUUUCACCUACGCU
ACACUGAUCCGGUGGGCCAUCCUGGAAGCACCUGAGAAACAGAGAACACU
GAACGAGAUCUACCACUGGUUCACCCGGAUGUUUGCGUUCUUCCGCAACC
ACCCUGCUACCUGGAAGAACGCUAUUCGCCACAAUCUGUCUCUGCACAAA
UGCUUUGUCCGAGUAGAGAGCGAGAAGGGCGCCGUGUGGACCGUGGAUGA
ACUGGAGUUCAGAAAGAAGAGAUCCCAGAGACCAUCGAGGUGUAGCAACC
CAACUCCGGGCCCCGGGUCGGGCGCUACCAACUUCAGCCUGCUGAAGCAG
GCCGGCGAUGUGGAAGAAAACCCUGGGCCCAUGGAAACGGAGGCUAUUGA
CGGCUACAUCACCUGCGACAACGAACUAUCUCCUGAAAGAGAGCACUCCA
AUAUGGCGAUCGACCUAACCAGCUCCACCCCUAACGGACAGCAUGCCUCU
CCGUCCCACAUGACCAGCACCAACAGCGUCAAACUGGAGAUGCAGUCCGA
CGAGGAGUGUGACCGCAAGCCCCUGUCUCGCGAGGACGAGAUAAGAGGUC
AUGACGAGGGAUCUAGCCUCGAGGAACCACUCAUUGAGAGCUCCGAGGUG
GCCGACAAUCGGAAGGUGCAGGAACUACAAGGCGAAGGCGGCAUCCGUCU
GCCUAAUGGCGAGAGACCUUUCCACUGUAACCAGUGUGGCGCGAGCUUCA
CCCAGAAGGGCAAUCUGCUGAGACACAUCAAGCUGCACUCUGGUGAGAAG
CCUUUUAAAUGCCCAUUCUGCUCUUACGCCUGCCGACGGCGCGACGCCCU
GACCGGCCACCUCCGCACCCACUCUGUGGGCAAACCUCACAAGUGCAACU
ACUGCGGCCGCUCCUACAAGCAGAGAAGCAGCUUGGAAGAGCACAAGGAG
CGCUGUCACAACUAUCUGCAGAACGUGUCCAUGGAAGCCGCCGGCCAGGU
GAUGAGCCACCACGUGCCGCCCAUGGAAGAUUGCAAGGAGCAGGAGCCCA
UCAUGGACAACAACAUCUCACUGGUCCCUUUCGAGAGACCGGCGGUGAUU
GAGAAGCUGACAGGAAACAUGGGCAAGCGCAAAAGCUCGACGCCCCAGAA
GUUCGUCGGCGAGAAGCUGAUGCGUUUCAGCUAUCCCGACAUCCACUUCG
ACAUGAACCUGACUUACGAGAAAGAGGCUGAACUGAUGCAAAGUCACAUG
AUGGACCAGGCCAUCAAUAACGCCAUCACUUACCUGGGCGCCGAGGCCCU
GCACCCCCUGAUGCAGCAUCCCCCGUCUACCAUCGCCGAGGUGGCCCCCG
UCAUUAGCAGCGCCUACUCCCAGGUGUACCACCCCAACCGCAUCGAGCGG
CCCAUCUCUCGUGAGACUGCGGAUUCUCACGAGAACAAUAUGGAUGGUCC
CAUCAGCCUCAUCAGACCUAAGAGCCGCCCUCAAGAGAGAGAGGCCUCCC
CAAGCAACAGCUGCCUGGAUUCUACCGACAGCGAAUCGAGCCACGAUGAC
CACCAGAGCUAUCAGGGUCACCCUGCCCUCAACCCGAAGAGAAAGCAGAG
CCCCGCCUACAUGAAGGAAGACGUGAAGGCCCUGGACACCACCAAGGCCC
CUAAGGGCUCCCUGAAGGACAUCUACAAGGUGUUCAACGGCGAGGGCGAG
CAAAUCCGCGCAUUCAAGUGCGAGCACUGUAGAGUGCUGUUCCUGGACCA
CGUGAUGUACACCAUCCAUAUGGGCUGUCAUGGCUACCGCGACCCACUUG
AGUGCAACAUCUGUGGCUACAGAUCUCAGGAUCGCUACGAGUUUUCGAGC
CACAUCGUGCGCGGAGAGCACACGUUUCACUGAUUAACAACAAGAGGGCC
CGGAAACCUGGCCCUGUCUUCUUGACGAGCAUUCCUAGGGGUCUUUCCCC
UCUCGCCAAAGGAAUGCAAGGUCUGUUGAAUGUCGUGAAGGAAGCAGUUC
CUCUGGAAGCUUCUUGAAGACAAACAACGUCUGUAGCGACCCUUUGCAGG
CAGCGGAACCCCCCACCUGGCGACAGGUGCCUCUGCGGCCAAAAGCCACG
UGUAUAAGAUACACCUGCAAAGGCGGCACAACCCCAGUGCCACGUUGUGA
GUUGGAUAGUUGUGGAAAGAGUCAAAUGGCUCUCCUCAAGCGUAUUCAAC
AAGGGGCUGAAGGAUGCCCAGAAGGUACCCCAUUGUAUGGGAUCUGAUCU
GGGGCCUCGGUGCACAUGCUUUACAUGUGUUUAGUCGAGGUUAAAAAAAC
GUCUAGGCCCCCCGAACCACGGGGACGUGGUUUUCCUUUGAAAAACACGA
UGAUAACAUAGCAUACAUAGCCGCCACCAUGGCUUCCCCCCUGACCCGCU
UCCUGUCUCUGAACCUGCUACUCCUCGGAGAGUCCAUCAUCCUGGGCUCU
GGCGAGGCAGAACAGAAACUGAUUUCUGAGGAAGAUCUGGAUGUGCAGCU
GCAAGAAUCUGGGCCCGGCCUGGUGAAGCCCUCUCAGAGCUUGUCACUGA
CCUGUUCCGUGACCGGAUACAGCAUCACCUCCGGCUACUAUUGGACCUGG
AUCCGGCAGUUUCCUGGCAACAAGCUGGAGUGGAUGGGAUCCAUUGGCUA
UGAUGGCACCAACUACUACAAUCCAUCUCUUAAGAACCGCAUCUCUAUCA
CCAGAGAUACAUCCAAGAACCAGUUCUUUCUGAAGCUGCACAGCGUCACC
ACCGAGGACACCGCCACUUACUACUGCGCCAAGCGCGGCAUCACCACUGG
CGACUACUGGGGCCAGGGUACAACCCUGACCGUGUCCAGCGGCGGGGGCG
GGUCCGGAGGCGGAGGAUCCGGCGGCGGUGGCAGCCAGGCCGUGGUGACG
CAAGAGAGCGCUCUGACCACCAGCCCCGGCGAGACAGUGACCCUGACCUG
UAGAAGCAGCACCGGUGCGGUAACGACAAGCAACUACGCCAACUGGGUCC
AGGAGAAGCCCGACCAUCUGUUCAUCGGGCUGAUCGGUGGCACCAACAAG
CGGACUCCGGGUGUGCCUGCUCGUUUCAGCGGGUCGUUAAUUGGCGACAA
GGCCGCGCUCACCAUUACAGGUACCCAGACCGAAGACGAGGCUAUCUACU
UCUGUGCCCUGUGGUACAGCAACCACCUGGUGUUCGGCGGGGGCACCAAG
CUAACAGUGCUGUCCACCACGACCAAGCCUGUCCUGAGAACACCUAGCCC
UGUGCACCCCACGGGCACUAGCCAACCUCAGAGACCCGAGGACUGCCGAC
CCCGUGGAUCUGUUAAGGGCACCGGCCUGGACUUCGCCUGCGAUAUCUAC
AUCUGGGCCCCUCUGGCCGGCAUCUGCGUGGCCCUCCUGCUGAGCCUGAU
CAUCACUCUGAUCUGCUACAACAGCAGACGGAAUCGUCUGCUGCAGAGCG
AUUACAUGAAUAUGACCCCUAGGCGGCCUGGCCUUACGAGAAAACCCUAU
CAGCCAUACGCUCCUGCCCGCGACUUUGCGGCCUACCGGCCGAGAGCCAA
AUUCUCCCGCCGAGCCGAGACUGCAGCCAACCUGCAGGACCCCAACCAGC
UCUACAACGAGCUGAACCUGGGCAGACGCGAGGAGUACGACGUGCUGGAA
AAGAAGAGGGCUCGGGACCCUGAGAUGGGCGGCAAGCAGCAGAGAAGACG
GAAUCCACAGGAGGGCGUGUACAACGCCUUGCAGAAGGACAAGAUGGCCG
AGGCUUAUUCCGAGAUCGGUACAAAAGGUGAGAGAAGGCGCGGCAAAGGC
CACGACGGCCUCUACCAGGGCCUGAGCACAGCCACCAAGGACACCUACGA
CGCCCUGCACAUGCAGACACUGGCGCCGCGCUGA
DNA sequence of 5′Kozak-IKZF2-p2a-FOXP3-
spacer-ires-kozak-signal peptide-myc
tag-HGL scFV cDNA sequence
SEQ ID NO: 21
ATAGTAATAGCCGCCACCATGGAAACCGAGGCCATCGATGGCTACATCAC
CTGCGACAACGAGCTGAGCCCGGAGAGAGAGCACTCAAATATGGCCATTG
ACCTGACCAGCAGCACACCTAATGGTCAGCACGCCTCTCCGTCTCATATG
ACCTCCACCAACTCCGTAAAGCTGGAGATGCAGAGCGATGAGGAGTGTGA
TCGCAAGCCTCTGTCGCGGGAAGACGAGATCCGGGGCCATGACGAAGGCA
GCTCCCTGGAAGAGCCTTTGATCGAGAGCTCTGAAGTCGCGGACAACCGC
AAAGTGCAGGAGCTGCAGGGGGAGGGCGGCATCAGGCTGCCCAACGGTGA
GCGACCGTTCCACTGTAATCAGTGTGGGGCTTCCTTCACCCAGAAGGGTA
ACCTGCTGCGCCACATCAAGCTGCACAGCGGCGAGAAACCCTTCAAGTGC
CCATTCTGCTCTTATGCCTGCAGACGACGCGACGCTCTGACCGGCCACCT
CCGCACCCACAGCGTGGGCAAGCCCCACAAGTGCAACTACTGCGGGCGTT
CCTACAAGCAGAGATCTTCTCTGGAAGAACACAAGGAGCGGTGCCACAAC
TACCTGCAGAACGTGAGCATGGAGGCTGCGGGACAGGTGATGAGCCACCA
CGTGCCACCTATGGAAGATTGCAAGGAACAGGAGCCCATCATGGATAACA
ACATCTCCCTCGTGCCTTTCGAGCGCCCTGCCGTGATTGAGAAACTTACC
GGCAACATGGGCAAGCGCAAGTCCTCTACTCCGCAGAAGTTCGTGGGGGA
GAAGCTGATGCGGTTCAGCTACCCCGACATCCATTTCGACATGAACCTGA
CCTACGAGAAAGAGGCCGAGCTGATGCAGAGCCACATGATGGACCAGGCT
ATCAACAACGCCATTACCTATCTGGGCGCTGAGGCCCTGCACCCCCTGAT
GCAGCACCCCCCTAGCACAATCGCCGAGGTGGCTCCGGTGATCTCATCCG
CCTACAGCCAAGTGTACCACCCTAACAGAATCGAACGGCCCATCTCCCGG
GAGACCGCCGACAGCCACGAGAACAACATGGACGGCCCTATATCCCTGAT
CCGCCCTAAGAGCCGCCCTCAGGAGAGAGAGGCTAGCCCATCTAATTCAT
GCCTGGATTCTACCGACTCCGAGAGTAGCCACGATGACCACCAGAGCTAC
CAGGGCCACCCCGCTCTGAACCCAAAGCGGAAGCAGAGCCCCGCCTACAT
GAAGGAGGATGTGAAGGCCCTAGACACCACCAAGGCCCCGAAAGGGTCCC
TGAAAGACATCTACAAGGTGTTTAACGGCGAGGGCGAGCAAATCAGAGCT
TTTAAGTGCGAGCACTGCCGGGTGCTGTTCCTGGATCATGTGATGTACAC
CATCCACATGGGTTGTCACGGCTATAGAGACCCACTGGAATGTAACATCT
GTGGCTACAGATCGCAAGACAGATATGAGTTCAGCAGCCACATCGTGCGC
GGCGAACATACCTTCCATGGCTCCGGCGCTACCAACTTCTCCCTACTGAA
GCAGGCTGGCGATGTGGAAGAGAATCCAGGGCCGATGCCTAATCCTCGAC
CCGGCAAACCTTCCGCACCGTCTCTGGCCCTGGGACCCAGCCCTGGAGCT
TCTCCAAGTTGGCGGGCCGCGCCCAAGGCTTCGGACCTGTTGGGCGCGAG
AGGACCGGGCGGCACCTTCCAGGGCCGGGATCTGCGCGGGGGAGCGCACG
CCAGCAGCTCCTCGCTGAACCCCATGCCTCCATCGCAGCTGCAATTGCCA
ACCCTGCCCCTGGTGATGGTGGCCCCTAGCGGTGCCCGCCTGGGCCCTCT
GCCTCATCTTCAGGCCCTGCTCCAGGACAGGCCTCATTTTATGCACCAGC
TGAGTACGGTTGACGCCCATGCAAGAACACCTGTGTTACAGGTCCACCCT
CTGGAGTCCCCCGCCATGATCTCTCTTACCCCGCCTACAACTGCTACGGG
TGTGTTCAGTCTGAAGGCCCGTCCTGGCCTACCCCCAGGCATCAACGTGG
CTAGTTTGGAATGGGTTAGCCGGGAACCAGCCCTGCTGTGCACTTTCCCC
AATCCAAGCGCTCCTAGAAAGGACTCCACACTGTCTGCCGTCCCTCAGTC
TAGCTACCCTCTGCTGGCCAACGGCGTGTGCAAATGGCCTGGATGCGAGA
AAGTGTTTGAGGAGCCTGAGGACTTCCTGAAGCACTGTCAGGCCGACCAC
CTGCTCGACGAGAAGGGCCGCGCCCAGTGCCTGCTCCAGCGCGAGATGGT
GCAGAGCCTGGAACAGCAGCTGGTGCTCGAAAAAGAGAAACTTAGCGCCA
TGCAGGCCCATCTGGCAGGAAAGATGGCTCTCACAAAGGCAAGCAGCGTG
GCCTCCTCTGACAAGGGGAGCTGTTGTATTGTCGCCGCCGGCTCACAGGG
ACCGGTGGTCCCCGCTTGGTCTGGCCCCCGCGAAGCCCCCGACTCCCTGT
TCGCCGTACGTCGACACCTTTGGGGCTCTCACGGCAACTCCACATTTCCT
GAATTCCTGCACAACATGGACTACTTCAAATTTCACAACATGAGACCACC
CTTCACCTACGCCACGCTGATCAGATGGGCCATCCTGGAGGCGCCCGAGA
AGCAGCGGACTCTGAACGAGATCTACCACTGGTTCACACGCATGTTTGCG
TTCTTCCGGAACCACCCCGCAACCTGGAAGAACGCCATCAGACACAACCT
GAGCCTCCACAAATGTTTCGTCAGAGTGGAGAGCGAGAAGGGCGCCGTGT
GGACCGTGGACGAGCTGGAATTCAGAAAGAAGCGGAGCCAAAGACCTTCG
AGGTGTAGTAACCCCACCCCCGGACCTTGATTAACAACAAGAGGGCCCGG
AAACCTGGCCCTGTCTTCTTGACGAGCATTCCTAGGGGTCTTTCCCCTCT
CGCCAAAGGAATGCAAGGTCTGTTGAATGTCGTGAAGGAAGCAGTTCCTC
TGGAAGCTTCTTGAAGACAAACAACGTCTGTAGCGACCCTTTGCAGGCAG
CGGAACCCCCCACCTGGCGACAGGTGCCTCTGCGGCCAAAAGCCACGTGT
ATAAGATACACCTGCAAAGGCGGCACAACCCCAGTGCCACGTTGTGAGTT
GGATAGTTGTGGAAAGAGTCAAATGGCTCTCCTCAAGCGTATTCAACAAG
GGGCTGAAGGATGCCCAGAAGGTACCCCATTGTATGGGATCTGATCTGGG
GCCTCGGTGCACATGCTTTACATGTGTTTAGTCGAGGTTAAAAAAACGTC
TAGGCCCCCCGAACCACGGGGACGTGGTTTTCCTTTGAAAAACACGATGA
TAACATAGCATACATAGCCGCCACCATGGCCTCCCCACTGACCAGATTCT
TGTCTCTCAACCTGCTGCTGCTGGGCGAAAGCATCATCCTGGGCTCCGGA
GAGGCTGAACAGAAGCTGATCAGCGAGGAAGACCTGGACGTGCAGCTGCA
AGAGTCTGGACCTGGTCTAGTGAAGCCTTCCCAGAGCCTGTCTCTGACCT
GTAGCGTGACCGGCTATAGCATCACCTCGGGCTACTATTGGACCTGGATC
CGCCAGTTTCCTGGCAACAAACTGGAGTGGATGGGAAGTATCGGATACGA
CGGCACCAACTACTACAACCCTAGCCTGAAGAACCGGATCTCTATCACCC
GCGACACTTCCAAGAATCAGTTCTTCCTGAAACTGCACAGCGTTACCACC
GAGGACACTGCCACCTACTACTGCGCCAAACGCGGTATCACCACAGGTGA
TTATTGGGGCCAGGGCACCACACTGACTGTGTCTTCTGGCGGAGGGGGTT
CCGGAGGGGGAGGTTCCGGCGGGGGCGGCAGCCAGGCCGTGGTGACCCAG
GAGAGCGCCCTGACCACCAGCCCTGGCGAGACAGTAACACTGACCTGCAG
ATCCTCGACCGGCGCGGTCACCACATCTAATTACGCCAATTGGGTCCAAG
AGAAGCCCGACCATCTGTTCATCGGCCTCATTGGCGGCACCAACAAGCGG
ACTCCTGGCGTGCCTGCACGATTTTCGGGTTCTCTGATTGGCGACAAGGC
CGCCCTCACAATTACTGGCACCCAGACTGAGGACGAGGCGATCTACTTCT
GTGCTCTGTGGTACAGCAACCACCTGGTGTTCGGCGGTGGAACCAAGCTG
ACCGTGCTGAGCACCACAACCAAGCCGGTCCTGAGAACACCTTCTCCTGT
GCACCCCACGGGGACCTCCCAGCCTCAGCGCCCGGAAGATTGCAGACCAA
GGGGCAGTGTGAAAGGCACCGGTCTTGACTTTGCCTGCGACATCTACATC
TGGGCCCCTCTGGCCGGCATCTGTGTGGCCCTGCTGCTTTCCCTCATCAT
CACCCTCATCTGCTACAACAGCCGCCGGAACCGCCTGCTGCAAAGCGACT
ACATGAACATGACCCCCAGACGGCCCGGCCTCACAAGAAAGCCCTATCAG
CCATATGCCCCGGCGCGGGACTTCGCCGCTTACAGGCCCAGAGCCAAGTT
CAGCAGACGCGCTGAGACCGCGGCCAACCTGCAGGACCCCAACCAGCTAT
ACAACGAGCTGAATCTCGGCAGACGCGAGGAGTACGATGTGCTGGAAAAG
AAGCGCGCGAGAGATCCTGAGATGGGCGGCAAGCAGCAGAGACGTCGTAA
CCCCCAGGAGGGCGTGTACAACGCCTTGCAGAAGGACAAGATGGCTGAAG
CCTACTCCGAGATCGGGACAAAGGGCGAGCGGCGTAGAGGCAAAGGCCAC
GATGGCCTGTACCAGGGCCTCAGCACAGCTACCAAGGATACATACGACGC
CCTGCACATGCAGACGTTGGCTCCTCGTTGA
RNA sequence of 5′Kozak-IKZF2-p2a-FOXP3-
spacer-ires-kozak-signal peptide-myc
tag-HGL scFV cDNA sequence
SEQ ID NO: 22
AUAGUAAUAGCCGCCACCAUGGAAACCGAGGCCAUCGAUGGCUACAUCAC
CUGCGACAACGAGCUGAGCCCGGAGAGAGAGCACUCAAAUAUGGCCAUUG
ACCUGACCAGCAGCACACCUAAUGGUCAGCACGCCUCUCCGUCUCAUAUG
ACCUCCACCAACUCCGUAAAGCUGGAGAUGCAGAGCGAUGAGGAGUGUGA
UCGCAAGCCUCUGUCGCGGGAAGACGAGAUCCGGGGCCAUGACGAAGGCA
GCUCCCUGGAAGAGCCUUUGAUCGAGAGCUCUGAAGUCGCGGACAACCGC
AAAGUGCAGGAGCUGCAGGGGGAGGGCGGCAUCAGGCUGCCCAACGGUGA
GCGACCGUUCCACUGUAAUCAGUGUGGGGCUUCCUUCACCCAGAAGGGUA
ACCUGCUGCGCCACAUCAAGCUGCACAGCGGCGAGAAACCCUUCAAGUGC
CCAUUCUGCUCUUAUGCCUGCAGACGACGCGACGCUCUGACCGGCCACCU
CCGCACCCACAGCGUGGGCAAGCCCCACAAGUGCAACUACUGCGGGCGUU
CCUACAAGCAGAGAUCUUCUCUGGAAGAACACAAGGAGCGGUGCCACAAC
UACCUGCAGAACGUGAGCAUGGAGGCUGCGGGACAGGUGAUGAGCCACCA
CGUGCCACCUAUGGAAGAUUGCAAGGAACAGGAGCCCAUCAUGGAUAACA
ACAUCUCCCUCGUGCCUUUCGAGCGCCCUGCCGUGAUUGAGAAACUUACC
GGCAACAUGGGCAAGCGCAAGUCCUCUACUCCGCAGAAGUUCGUGGGGGA
GAAGCUGAUGCGGUUCAGCUACCCCGACAUCCAUUUCGACAUGAACCUGA
CCUACGAGAAAGAGGCCGAGCUGAUGCAGAGCCACAUGAUGGACCAGGCU
AUCAACAACGCCAUUACCUAUCUGGGCGCUGAGGCCCUGCACCCCCUGAU
GCAGCACCCCCCUAGCACAAUCGCCGAGGUGGCUCCGGUGAUCUCAUCCG
CCUACAGCCAAGUGUACCACCCUAACAGAAUCGAACGGCCCAUCUCCCGG
GAGACCGCCGACAGCCACGAGAACAACAUGGACGGCCCUAUAUCCCUGAU
CCGCCCUAAGAGCCGCCCUCAGGAGAGAGAGGCUAGCCCAUCUAAUUCAU
GCCUGGAUUCUACCGACUCCGAGAGUAGCCACGAUGACCACCAGAGCUAC
CAGGGCCACCCCGCUCUGAACCCAAAGCGGAAGCAGAGCCCCGCCUACAU
GAAGGAGGAUGUGAAGGCCCUAGACACCACCAAGGCCCCGAAAGGGUCCC
UGAAAGACAUCUACAAGGUGUUUAACGGCGAGGGCGAGCAAAUCAGAGCU
UUUAAGUGCGAGCACUGCCGGGUGCUGUUCCUGGAUCAUGUGAUGUACAC
CAUCCACAUGGGUUGUCACGGCUAUAGAGACCCACUGGAAUGUAACAUCU
GUGGCUACAGAUCGCAAGACAGAUAUGAGUUCAGCAGCCACAUCGUGCGC
GGCGAACAUACCUUCCAUGGCUCCGGCGCUACCAACUUCUCCCUACUGAA
GCAGGCUGGCGAUGUGGAAGAGAAUCCAGGGCCGAUGCCUAAUCCUCGAC
CCGGCAAACCUUCCGCACCGUCUCUGGCCCUGGGACCCAGCCCUGGAGCU
UCUCCAAGUUGGCGGGCCGCGCCCAAGGCUUCGGACCUGUUGGGCGCGAG
AGGACCGGGCGGCACCUUCCAGGGCCGGGAUCUGCGCGGGGGAGCGCACG
CCAGCAGCUCCUCGCUGAACCCCAUGCCUCCAUCGCAGCUGCAAUUGCCA
ACCCUGCCCCUGGUGAUGGUGGCCCCUAGCGGUGCCCGCCUGGGCCCUCU
GCCUCAUCUUCAGGCCCUGCUCCAGGACAGGCCUCAUUUUAUGCACCAGC
UGAGUACGGUUGACGCCCAUGCAAGAACACCUGUGUUACAGGUCCACCCU
CUGGAGUCCCCCGCCAUGAUCUCUCUUACCCCGCCUACAACUGCUACGGG
UGUGUUCAGUCUGAAGGCCCGUCCUGGCCUACCCCCAGGCAUCAACGUGG
CUAGUUUGGAAUGGGUUAGCCGGGAACCAGCCCUGCUGUGCACUUUCCCC
AAUCCAAGCGCUCCUAGAAAGGACUCCACACUGUCUGCCGUCCCUCAGUC
UAGCUACCCUCUGCUGGCCAACGGCGUGUGCAAAUGGCCUGGAUGCGAGA
AAGUGUUUGAGGAGCCUGAGGACUUCCUGAAGCACUGUCAGGCCGACCAC
CUGCUCGACGAGAAGGGCCGCGCCCAGUGCCUGCUCCAGCGCGAGAUGGU
GCAGAGCCUGGAACAGCAGCUGGUGCUCGAAAAAGAGAAACUUAGCGCCA
UGCAGGCCCAUCUGGCAGGAAAGAUGGCUCUCACAAAGGCAAGCAGCGUG
GCCUCCUCUGACAAGGGGAGCUGUUGUAUUGUCGCCGCCGGCUCACAGGG
ACCGGUGGUCCCCGCUUGGUCUGGCCCCCGCGAAGCCCCCGACUCCCUGU
UCGCCGUACGUCGACACCUUUGGGGCUCUCACGGCAACUCCACAUUUCCU
GAAUUCCUGCACAACAUGGACUACUUCAAAUUUCACAACAUGAGACCACC
CUUCACCUACGCCACGCUGAUCAGAUGGGCCAUCCUGGAGGCGCCCGAGA
AGCAGCGGACUCUGAACGAGAUCUACCACUGGUUCACACGCAUGUUUGCG
UUCUUCCGGAACCACCCCGCAACCUGGAAGAACGCCAUCAGACACAACCU
GAGCCUCCACAAAUGUUUCGUCAGAGUGGAGAGCGAGAAGGGCGCCGUGU
GGACCGUGGACGAGCUGGAAUUCAGAAAGAAGCGGAGCCAAAGACCUUCG
AGGUGUAGUAACCCCACCCCCGGACCUUGAUUAACAACAAGAGGGCCCGG
AAACCUGGCCCUGUCUUCUUGACGAGCAUUCCUAGGGGUCUUUCCCCUCU
CGCCAAAGGAAUGCAAGGUCUGUUGAAUGUCGUGAAGGAAGCAGUUCCUC
UGGAAGCUUCUUGAAGACAAACAACGUCUGUAGCGACCCUUUGCAGGCAG
CGGAACCCCCCACCUGGCGACAGGUGCCUCUGCGGCCAAAAGCCACGUGU
AUAAGAUACACCUGCAAAGGCGGCACAACCCCAGUGCCACGUUGUGAGUU
GGAUAGUUGUGGAAAGAGUCAAAUGGCUCUCCUCAAGCGUAUUCAACAAG
GGGCUGAAGGAUGCCCAGAAGGUACCCCAUUGUAUGGGAUCUGAUCUGGG
GCCUCGGUGCACAUGCUUUACAUGUGUUUAGUCGAGGUUAAAAAAACGUC
UAGGCCCCCCGAACCACGGGGACGUGGUUUUCCUUUGAAAAACACGAUGA
UAACAUAGCAUACAUAGCCGCCACCAUGGCCUCCCCACUGACCAGAUUCU
UGUCUCUCAACCUGCUGCUGCUGGGCGAAAGCAUCAUCCUGGGCUCCGGA
GAGGCUGAACAGAAGCUGAUCAGCGAGGAAGACCUGGACGUGCAGCUGCA
AGAGUCUGGACCUGGUCUAGUGAAGCCUUCCCAGAGCCUGUCUCUGACCU
GUAGCGUGACCGGCUAUAGCAUCACCUCGGGCUACUAUUGGACCUGGAUC
CGCCAGUUUCCUGGCAACAAACUGGAGUGGAUGGGAAGUAUCGGAUACGA
CGGCACCAACUACUACAACCCUAGCCUGAAGAACCGGAUCUCUAUCACCC
GCGACACUUCCAAGAAUCAGUUCUUCCUGAAACUGCACAGCGUUACCACC
GAGGACACUGCCACCUACUACUGCGCCAAACGCGGUAUCACCACAGGUGA
UUAUUGGGGCCAGGGCACCACACUGACUGUGUCUUCUGGCGGAGGGGGUU
CCGGAGGGGGAGGUUCCGGCGGGGGCGGCAGCCAGGCCGUGGUGACCCAG
GAGAGCGCCCUGACCACCAGCCCUGGCGAGACAGUAACACUGACCUGCAG
AUCCUCGACCGGCGCGGUCACCACAUCUAAUUACGCCAAUUGGGUCCAAG
AGAAGCCCGACCAUCUGUUCAUCGGCCUCAUUGGCGGCACCAACAAGCGG
ACUCCUGGCGUGCCUGCACGAUUUUCGGGUUCUCUGAUUGGCGACAAGGC
CGCCCUCACAAUUACUGGCACCCAGACUGAGGACGAGGCGAUCUACUUCU
GUGCUCUGUGGUACAGCAACCACCUGGUGUUCGGCGGUGGAACCAAGCUG
ACCGUGCUGAGCACCACAACCAAGCCGGUCCUGAGAACACCUUCUCCUGU
GCACCCCACGGGGACCUCCCAGCCUCAGCGCCCGGAAGAUUGCAGACCAA
GGGGCAGUGUGAAAGGCACCGGUCUUGACUUUGCCUGCGACAUCUACAUC
UGGGCCCCUCUGGCCGGCAUCUGUGUGGCCCUGCUGCUUUCCCUCAUCAU
CACCCUCAUCUGCUACAACAGCCGCCGGAACCGCCUGCUGCAAAGCGACU
ACAUGAACAUGACCCCCAGACGGCCCGGCCUCACAAGAAAGCCCUAUCAG
CCAUAUGCCCCGGCGCGGGACUUCGCCGCUUACAGGCCCAGAGCCAAGUU
CAGCAGACGCGCUGAGACCGCGGCCAACCUGCAGGACCCCAACCAGCUAU
ACAACGAGCUGAAUCUCGGCAGACGCGAGGAGUACGAUGUGCUGGAAAAG
AAGCGCGCGAGAGAUCCUGAGAUGGGCGGCAAGCAGCAGAGACGUCGUAA
CCCCCAGGAGGGCGUGUACAACGCCUUGCAGAAGGACAAGAUGGCUGAAG
CCUACUCCGAGAUCGGGACAAAGGGCGAGCGGCGUAGAGGCAAAGGCCAC
GAUGGCCUGUACCAGGGCCUCAGCACAGCUACCAAGGAUACAUACGACGC
CCUGCACAUGCAGACGUUGGCUCCUCGUUGA
DNA sequence of iEMCV
SEQ ID NO: 23
CCCCTCTCCCTCCCCCCCCCCTAACGTTACTGGCCGAAGCCGCTTGGAAT
AAGGCCGGTGTGCGTTTGTCTATATGTTATTTTCCACCATATTGCCGTCT
TTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATG
TCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCT
CTGCGGCCAAAAGCCACGTGTATAAGATACACCTGCAAAGGCGGCACAAC
CCCAGTGCCACGTTGTGAGTTGGATAGTTGTGGAAAGAGTCAAATGGCTC
TCCTCAAGCGTATTCAACAAGGGGCTGAAGGATGCCCAGAAGGTACCCCA
TTGTATGGGATCTGATCTGGGGCCTCGGTACACATGCTTTACATGTGTTT
AGTCGAGGTTAAAAAAACGTCTAGGCCCCCCGAACCACGGGGACGTGGTT
TTCCTTTGAAAAACACGATGATAATATGGCCACAACC
DNA sequence of iSimianEV-A
SEQ ID NO: 24
GAGTGTTCCCACCCAACAGGCCCACTGGGTGTTGTACTCTGGTATTACGG
TACCTTTGTACGCCTATTTTATTTCCCCCCCCTTTTTGAAACTTAGAAGT
TAATAATAAACACGCTCACTAGGTGCACTACATCCAGTAGTGTAATGAGC
AAGCACTTCTGTCTTCCCCGGGAGGGATATATGGTACGCTGTGCAAACGG
CGGAAATTAATCCTACCGTTAACCGCCCACCTACTCCGAGAAGCCTAGTA
CCTAATTGGATTTATCAATGGAGTTGCGCTCAGCAGGTGACCCTGACCTG
CCAGCTCCGGCTGATGGACCTGGGCTTTCCCCACAGGCGACTGTGGCCCA
GGTCGCGTGGCGGCCGGCCCACCCCCCTGGGTGGGACGCCTTGATAATGA
CAAGGTGGGAAGAGCCTATTGGGCTAGCTGGTTTCCTCCGGCCTCCTGAA
TGCGGCTAACCTTAACCCCAGAGCATATGGTAGCAACCCAGCTACTAGTA
TGTCATAATGCGTAAGTCTGGGATGGGACCGACTACTTTGGAGAGTCCGT
GTTTCTATTGTTTCTTTAATCAATCTTATGGTGACAATTTATAGTGCCCT
GAGTATTGATTGGTTGTTGCTTTTGACAATTATTGAGACATCACATAGAC
ATA
DNA sequence of iCovid19
SEQ ID NO: 25
ATTAAAGGTTTATACCTTCCCAGGTAACAAACCAACCAACTTTCGATCTC
TTGTAGATCTGTTCTCTAAACGAACTTTAAAATCTGTGTGGCTGTCACTC
GGCTGCATGCTTAGTGCACTCACGCAGTATAATTAATAACTAATTACTGT
CGTTGACAGGACACGAGTAACTCGTCTATCTTCTGCAGGCTGCTTACGGT
TTCGTCCGTGTTGCAGCCGATCATCAGCACATCTAGGTTTCGTCCGGGTG
TGACCGAAAGGTAAG
DNA sequence of iHCV
SEQ ID NO: 26
GCCAGCCCCCGATTGGGGGCGACACTCCACCATAGATCACTCCCCTGTGA
GGAACTACTGTCTTCACGCAGAAAGCGTCTAGCCATGGCGTTAGTATGAG
AGTCGTGCAGCCTCCAGGACCCCCCCTCCCGGGAGAGCCATAGTGGTCTG
CGGAACCGGTGAGTACACCGGAATTGCCAGGACGACCGGGTCCTTTCTTG
GATCAACCCGCTCAATGCCTGGAGATTTGGGCGTGCCCCCGCAAGACTGC
TAGCCGAGTAGTGTTGGGTCGCGAAAGGCCTTGTGGTACTGCCTGATAGG
GTGCTTGCGAGTGCCCCGGGAGGTCTCGTAGACCGTGCACCATGAGCACG
AATCCTAAACCTCAAAGAAAAACCAAACGTAAC
DNA sequence of iCVB5
SEQ ID NO: 27
TTAAAACAGCCTGTGGGTTGTTCCCACCCGCAGGGCCCACTGGGCGCCAG
CACTCTGGTATCACGGTACCTTTGTGCGCCTGTTTTAAAACCCTCTCCCC
AATTTGAAACTTAGAAGCAATACACCTCGATCAATAGTAGGCATGACACG
CCAGCCATGTCTTGATCAAGCACTTCTGTTTCCCCGGACTGAGTATCAAT
AAACTGCTTGCGCGGTCGAAGGAGAAAACGTCCGTTACCCGACTAACTAC
TTCGAGAAACCCAGTAACACCATGGAAATTGCGGAGTGTTTCACTCAGCA
CATTCCCAGTGTAGATCAGGTCGATGAGTCACCGCATTCCCCACGGGTGA
CCGTGGCGGTGGCTGCGCTGGCGGCCTGCCCATGGGGCAACCCATGGGAC
GCTTCAATATGGACATGGTGTGAAGAGTCTATTGAGCTAGTTAGTAGTCC
TCCGGCCCCTGAATGCGGCTAATCCTAACTGCGGAGCACGTGCCTCCATT
CCAGGGGGTGGCGTGTCGTAACGGGCAACTCTGCAGCGGAACCGACTACT
TTGGGTGTCCGTGTTTCTTTTAATTTTATACTGGCTGCTTATGGTGACAA
TTGAAAGATTGTTGCCATATAGCTATTGGATTGGCCATCCGGTATCCAAC
AGAGCAATTGTGTACCTTTTTGTTGGATTTGTACCACTTACCAGAACAAG
TTTTCATACACTGTGTTACATTATTAGACTAAACACAGAAAA
DNA sequence of iCVA20
SEQ ID NO: 28
TTAAAACAGCTCTGGGGTTGTACCCACCCCAGAGGCCCACGTGGCGGCTA
GTACTCCGGTATTACGGTACCCTTGTACGCCTGTTTTATACTCCCTCCCT
CGTAACTTAGAAGCACAAAACCAAGTTCAATAGAAGGGGGTGCAAACCAG
TACCACCACGAACAAGCATTTCTGTTTCCCCGGTGATGTTGTATAGACTG
CCCGCGCGGTTGAAAGCAACGGTACCGTTACCCGCTCAAGTACTTCGAGA
AGCCTAGTATTACCTTGGAATCTTCGATGCGTTGCGTTCAGCACTCGACC
CTGGAGTGTAGCTTAGGCTGATGAGTCTGGACGTCCCTCACCGGTGACGG
TGGTCCAGGCTGCGTTGGCGGCCTACCTATGGCTAACGCCATAGGACGCT
AGTTGTGAACAAGGTGTGAAGAGCCTATTGAGCTACTTGAGAGTCCTCCG
GCCCCTGAATGCGGCTAATCCTAACCATGGAGCAGGCGGTCACAGACCAG
TGACTAGCTTGTCGTAATGCGCAAGTCTATGGCGGAACCGACTACTTTGG
GTGTCCGTGTTTCCTTTTATTTTTATTATGGCTGCTTATGGTGACAATCA
TTGATTGTTATCATAAAGCGAATTGGATTGGCCATCCGGTGAAAGCGAGA
CTTACTATTTACTTACTTGTTGGACTTACCACACTTAATACATTTATTCT
AGGTGTCACTTGTATAGCAATTAGAATCAAACAGTTGCATCATA
DNA sequence of iSwine Vesicular
SEQ ID NO: 29
TTAAAACAGCCTGTGGGTTGTTCCCACCCACAGGGCCCACTGGGCGCTAG
CACACTGGTATCACGGTACCTTTGTGCGCCTGTTTGACTTACCCTCCCCA
AACGCAACTTAGAAGCACAACTTAAATGGTCAATAGGCGGCTCAGTATGC
CAACTGAGTCTCGATCAAGCACTTCTGTTACCCCGGACTGAGTACCAATA
GGCTGCTCACGCGGCTGAAGGGGAAACCGTTCGTTACCCGACTAACTACT
TCGAGAAACCTAGTACCACCATGAAAGTTGCGCAGCGTTTCGCTCCGCAC
AACCCCAGTGTAGATCAGGTCGATGAGTCACCGCAAACCCCACGGGCGAC
CGTGGCGGTGGCTGCGCTGGCGGCCTGCCCATGGGGCAACTCATGGGACG
CTTCAATACTGACATGGTGCGAAGAGTCTATTGAGCTAGTTGGTAGTCCT
CCGGCCCCTGAATGCGGCTAATCCTAACTGCGGAGCAGATACCCACGCAC
CAGTGGGCAGTCTGTCGTAATGGGCAACTCTGCAGCGGAACCGACTACTT
TGGGTGTCCGTGTTTCCTTTTGTTCTTATACTGGCTACTTATGGTGACAA
TTGAGAGATTGTAACCATATTGCTATTGGATTGGCCACCCGGCGACGAAT
AGAACAGTTGCTTACCTGTTTGTTAGTCTCGTATCACTGAACTACAAATC
CTTAAACACCCTTTAATTTCGTCATAACGCTCAATACGTTAAA
DNA sequence of iHRV-A2
SEQ ID NO: 30
ATCCAGGTTGTTCCCACCTGGATTTCCCACAGGGAGTGGTACTCTGTTAT
TACGGTAACTTTGTACGCCAGTTTTATCTCCCTTCCCCCATGTAACTTAG
AAGTTTTTCACAAAGACCAATAGCCGGTAATCAGCCAGATTACTGAAGGT
CAAGCACTTCTGTTTCCCCGGTCAATGTTGATATGCTCCAACAGGGCAAA
AACAACTGCGATCGTTAACCGCAAAGCGCCTACGCAAAGCTTAGTAGCAT
CTTTGAAATCGTTTGGCTGGTCGATCCGCCATTTCCCCTGGTAGACCTGG
CAGATGAGGCTAGAAATACCCCACTGGCGACAGTGTTCTAGCCTGCGTGG
CTGCCTGCACACCCTATGGGTGTGAAGCCAAACAATGGACAAGGTGTGAA
GAGCCCCGTGTGCTCGCTTTGAGTCCTCCGGCCCCTGAATGTGGCTAACC
TTAACCCTGCAGCTAGAGCACGTAACCCAATGTGTATCTAGTCGTAATGA
GCAATTGCGGGATGGGACCAACTACTTTGGGTGTCCGTGTTTCACTTTTT
CCTTTATATTTGCTTATGGTGACAATATATACAATATATATATTGGCACC
DNA sequence of iHRV-C3
SEQ ID NO: 31
TTAAAGCTGGATCATGGTTGTTCCCACCATGATTACCCACGCGGTGCAGT
GGTCTTGTATTACGGTACATTTCCATACCAGTTTTATACACCCCACCCCG
AAACTCATAGAAGTTTGTACACAATGACCAATAGGTGGTGGCCATCCAGG
TCGCTAATGGTCAAGCACTTCTGTTTCCCCGGCACCCTTGTATACGCTTC
ACCCGAGGCGAAAAATGAGGTTGTCGTTATCCGCAAAGTGCCTACGAAAA
GCCTAGTAACACTTTGAAAACCCATGGTTGGTCGCTCAGCTGTTTACCCA
ACAGTAGACCTGGCAGATGAGGCTAGACATTCCCCACCAGCGATGGTGGT
CTAGCCTGCGTGGCTGCCTGCACACCCTGCCGGGTGTGAAGCCAGAAAGT
GGACAAGGTGTGAAGAGCCTATTGTGCTCACTTTGAGTCCTCCGGCCCCT
GAATGTGGCTAACCCTAACCCCGTAGCTGTTGCATGTAACCCAACATGTA
TGCAGTCGTAATGGGCAACTATGGGATGGGACCAACTACTTTGGGTGTCC
GTGTTTCCTGTTTTACTTTTTCATTGCTTATGGTGACAATTGTATCTGAT
ACACTTGTTACC
DNA sequence of iHRV-CII
SEQ ID NO: 32
TTAAAACTGGATACAGGTTGTTCCCACCTGTATCACCCAAGTGGTGTGGT
GCTCTTGTATTTCGGTACGTTTGCACGCCAGTTTGCTACCCCTTCCCTTT
TACGTAACTTAGAAGTTTACACAAAGACCAATAGGCGGTGGTAAATCCAT
ACCACTAACGGTCAAATACTTCTGTTTCCCCGGCATGCGAGGAATAGGCT
CCAAAAGGGCTGAAGCCACTAGTGTCGTTATCCGCATTGGTACTACGCAA
AGCCTAGTATTACCTTGAAAATTTCTTGGCTGGTCGCTCCACCAGATACC
CCACTGGTAGACCTGGCAGATGAGGCAGGACTTACCCCACTGGCGACAGT
GGTCCTGCCTGCGTGGCTGCCTGCACACCCCTTACGGGGTGTGAAGCCCA
GAAACAGACAAGGTGTGAAGAGCCCCGTGTGCTACTAGTGAGTCCTCCGG
CCCCTGAATGCGGCTAATCTTACCCCACAGCTGTTGCACGCAAACCAGCG
TGTATGCAGTCGTAATGAGCAATTGTGGGATGGAACCGACTACTTTGGGT
GTCCGTGTTTCTTTTTATTCCTATTATTTGCTTATGGTGACAATATTGAT
ATTATCAGTGTTGTCATC
DNA sequence of iCVBI
SEQ ID NO: 33
TTAAAACAGCCTGTGGGTTGTTCCCACCCACAGGCCCATTGGGCGCTAGC
ACTCTGGTATCACGGTACCTTTGTGCGCCTGTTTTACATCCCCTCCCCAA
ATTGTAATTTAGAAGTTTCACACACCGATCATTAGCAAGCGTGGCACACC
AGCCATGTTTTGATCAAGCACTTCTGTTACCCCGGACTGAGTATCAATAG
ACCGCTAACGCGGTTGAAGGAGAAAACGTTCGTTACCCGGCCAACTACTT
CGAAAAACCTAGTAACACCATGGAAGTTGCGGAGTGTTTCGCTCAGCACT
ACCCCAGTGTAGATCAGGTCGATGAGTCACCGCGTTCCCCACGGGCGACC
GTGGCGGTGGCTGCGTTGGCGGCCTGCCTACGGGGAAACCCGTAGGACGC
TCTAATACAGACATGGTGCGAAGAGTCTATTGAGCTAGTTGGTAATCCTC
CGGCCCCTGAATGCGGCTAATCCTAACTGCGGAGCACATACCCTCAAACC
AGGGGGCAGTGTGTCGTAACGGGCAACTCTGCAGCGGAACCGACTACTTT
GGGTGTCCGTGTTTCATTTTATTCCTATACTGGCTGCTTATGGTGACAAT
TGACAGGTTGTTACCATATAGTTATTGGATTGGCCATCCGGTGACTAACA
GAGCAATTATATATCTCTTTGTTGGGTTTATACCACTTAGCTTGAAAGAG
GTTAAAACACTACATCTCATCATTAAACTAAATACAACAAA
DNA sequence of iPV2
SEQ ID NO: 34
TTAAAACAGCTCTGGGGTTGTTCCCACCCCAGAGGCCCACGTGGCGGCCA
GTACACTGGTATCGCGGTACCTTTGTACGCCTGTTTTATACTCCCTTCCC
CCGTAACTTAGAAGCACAACGTCCAAGTTCAATAGAAGGGGGTACAAACC
AGTACCACCACGAACAAGCACTTCTGTTCCCCCGGTGAGGCTGTATAGGC
TGTTTCCACGGCTAAAAGCGGCTGATCCGTTATCCGCTCATGTACTTCGA
GAAGCCTAGTATCACCTTGGAATCTTCGATGCGTTGCGCTCAACACTCAA
CCCCAGAGTGTAGCTTAGGTCGATGAGTCTGGACGTTCCTCACCGGCGAC
GGTGGTCCAGGCTGCGTTGGCGGCCTACCTGTGGCCCAAAGCCACAGGAC
GCTAGTTGTGAACAAGGTGTGAAGAGCCTATTGAGCTACCTGAGAGTCCT
CCGGCCCCTGAATGCGGCTAATCCTAACCACGGAGCAGGCAGTGGCAATC
CAGCGACCAGCCTGTCGTAACGCGCAAGTTCGTGGCGGAACCGACTACTT
TGGGTGTCCGTGTTTCCTTTTATTTTTACAATGGCTGCTTATGGTGACAA
TTATTGATAGTTATCATAAAGCAAATTGGATTGGCCATCCGGTGAGAATT
TGATTATTAAATTACTCTCTTGTTGGGATTGCTCCTTTGAAATCCTGTGC
ACTCACACCTATTGGAATTACCTCATTGTTGAGATATTATTACCACT
DNA sequence of iHRV-B17
SEQ ID NO: 35
TTTAAACAGCGGATGGGTTCCCCACCATCCGACCCACTGGGTGTAGTACT
CTGGTATTTTGTACCTTTGTACGCCTGTTTCTCCCCTACCTCCCAACCTA
AACAATCCTGGTAACTTAGAAGACTTAAATCATCGTACAATAGGTGCTGT
CACATCCAGTGACGGCTAGTACAAGCACTTCTGTTTCCCCGGAGCGGAGT
ATAAATGGCCACCGCTGTCAAAAGCTCTTAACCGTTATCCGCCAATTAAC
TACGCAACGGCTAGTAACATCTTGTTATTTTTAGGGCGTTCGATCAGGTG
AGTAAACCCCTCACTAGTCTGGTCGATGAGGCTGAGAATTCCCCACGGGC
GACCGTGTCTCAGCCTGCGTGGCGGCCAGCCCAGCTAATGCTGGGACGCC
TTAATTGTGACATGGTGTGAAGACCCACGTGTGCTTAATTGTGAGTCCTC
CGGCCCCTGAATGCGGCTAACCTAAACCCTGGAGCCTTGAGACACAATCC
AGTGTTGGCAAGGTCGTAATGAGTAATTCCGGGACGGGACCGACTACTTT
GGGTGTCCGTGTTTCCTTTTATTTTCAAATTGTTCTTATGGTCACAATAT
AAGTAATATATTGTGATC
DNA sequence of iEchoV-E15
SEQ ID NO: 36
TTAAAACAGCCTGTGGGTTGTACCCACCCACAGGGCCCACTGGGCGCTAG
CACTCTGGTATTACGGTACCCTTGTGCGCCTGTTTTATATACCCCGCCCC
AAGCAAACGCTAGATGTAACGCACTTATGATCAATAGCAGGCGTGGCACT
CCAGCCACGTTATGATCAAGCACTTCTGTCTCCCCGGACCGAGTATCAAT
AGACTGCTCACGCGGTCGAAGGAGAAAACGTTCGTTACCCGACCAGCTAC
TTCGAGAAACCTAGTAACTCCATGGAGGTTGCAGAGTGTTTCGTTCAGCA
CTTCCCCCGTGTAGATCAGGCTGATGAGTCACCGCGTTCCTCACGGGCGA
CCGTGGCGGTGGCTGCGTTGGCGGCCTGCCTATGGGTATACCCATAGGAC
GCTCTAATACTGACATGGTGCGAAGAGTCTATTGAGCTAGTTGGTAGTCC
TCCGGCCCCTGAATGCGGCTAATCCTAACTGCGGAGCACATACCCCCAAC
CCAGGGGGCAGTGTGTCGTAACGGGTAACTCTGCAGCGGAACCGACTACT
TTGGGTGTCCGTGTTTCCTTTTATTCTTACATTGGCTGCTTATGGTGACA
ATTGAGAGATTGTTACCATATAGCTATTGGATTGGCCATCCAGTGACTAA
CAGAGCTATTATTTACCTTTTTGTTGGCTTCGTATCACTTGGTTTAAAAG
AGGTTAGTACTTTATATTGCATTATATTACTAAACACGAGAAA
DNA sequence of iEchoV-E11
SEQ ID NO: 37
TTAAAACAGCCTGTGGGTTGTACCCACCCACAGGGCCCACTGGGCGCTAG
CACACTGGTATCACGGTACCTTTGTGCGCCTGTTTTATACCCCCTTCCCG
CAACCGCAAATTTAGAAGCAAAGCTAACCCGATCGATAGCGGATGCGCAT
GCCAGCCGCATTTTGATCAAGTACTTCTGTTTCCCCGGACCGAGTATCAA
TAGACTGCTCACGCGGTTGAAGGAGAAAACGTCCGTTACCCGACCAACTA
CTTCGAGAAACCTAGTAACATCATGAATGTTGCAGGGCGTTTCGATCAGC
ACGACCCTGGTGTAGATCAGGCTGATGAGTCACCGCATTCCCCACGGGTG
ACCGTGGCGGTGGCTGCGTTGGCGGCCTGCCTATGGGGTGACCCATAGGA
CGCTCTAATACGGACATGGTGCGAAGAGTCTATTGAGCTAGTTGGTAGTC
CTCCGGCCCCTGAATGCGGTTAATCCTAACTGCGGACGACATACCCCTAA
TCCAAGGGGCAGTGTGTCGTAACGGGCAACTCTGCAGCGGAACCGACTAC
TTTGGGTGTCCGTGTTTCCTTTTATTTTTATACTGGCTGCTTATGGTGAC
AATCTCAGAGTTGTTACCATATAGCTATTGGATTGGCCATCCGGTGAGCA
ACAGAGCTGTCATTTATCAGTTTGTTGGCTTTATACCTCTAAATCACACG
GTTTTTTTTTTTTGGAACGCTTGTATTCATCTTAACCCTCAATAAGGCAA
A
DNA sequence of iEchoV11
SEQ ID NO: 38
TTAAAACAGCCTGTGGGTTGTACCCACCCACAGGGCCCACTGGGCGCTAG
CACACTGGTATCACGGTACCTTTGTGCGCCTGTTTTATACCCCCTTCCCG
CAACCGCAAATTTAGAAGCAAAGCTAACCCGATCGATAGCGGATGCGCAT
GCCAGCCGCATTTTGATCAAGTACTTCTGTTTCCCCGGACCGAGTATCAA
TAGACTGCTCACGCGGTTGAAGGAGAAAACGTCCGTTACCCGACCAACTA
CTTCGAGAAACCTAGTAACATCATGAATGTTGCAGGGCGTTTCGATCAGC
ACGACCCTGGTGTAGATCAGGCTGATGAGTCACCGCATTCCCCACGGGTG
ACCGTGGCGGTGGCTGCGTTGGCGGCCTGCCTATGGGGTGACCCATAGGA
CGCTCTAATACGGACATGGTGCGAAGAGTCTATTGAGCTAGTTGGTAGTC
CTCCGGCCCCTGAATGCGGTTAATCCTAACTGCGGACGACATACCCCTAA
TCCAAGGGGCAGTGTGTCGTAACGGGCAACTCTGCAGCGGAACCGACTAC
TTTGGGTGTCCGTGTTTCCTTTTATTTTTATACTGGCTGCTTATGGTGAC
AATCTCAGAGTTGTTACCATATAGCTATTGGATTGGCCATCCGGTGAGCA
ACAGAGCTGTCATTTATCAGTTTGTTGGCTTTATACCTCTAAATCACACG
GTTTTTTTTTTTTGGAACGCTTGTATTCATCTTAACCCTCAATAAGGCAA
A
DNA sequence of iCrPV
SEQ ID NO: 39
TTCTAGCACTAGTAAAGCAAAAATGTGATCTTGCTTGTAAATACAATTTT
GAGAGGTTAATAAATTACAAGTAGTGCTATTTTTGTATTTAGGTTAGCTA
TTTAGCTTTACGTTCCAGGATGCCTAGTGGCAGCCCCACAATATCCAGGA
AGCCCTCTCTGCGGTTTTTCAGATTAGGTAGTCGAAAAACCTAAGAAATT
TACCTGCTACATTTCAAGATAATCTAGCCACC
DNA sequence of iHRV-A89
SEQ ID NO: 40
TTAAAACTGGGAGTGGGTTGTTCCCACTCACTCCACCCATGCGGTGTTGT
ACTCTGTTATTACGGTAACTTTGTACGCCAGTTTTTCCCACCCTTCCCCA
TAATGTAACTTAGAAGTTTGTACAATATGACCAATAGGTGACAATCATCC
AGACTGTCAAAGGTCAAGCACTTCTGTTTCCCCGGTCAATGAGGATATGC
TTTACCCAAGGCAAAAACCTTAGAGATCGTTATCCCCACACTGCCTACAC
AGAGCCCAGTACCATTTTTGATATAATTGGGTTGGTCGCTCCCTGCAAAC
CCAGCAGTAGACCTGGCAGATGAGGCTGGACATTCCCCACTGGCGACAGT
GGTCCAGCCTGCGTGGCTGCCTGCTCACCCTTCTTGGGTGAGAAGCCTAA
TTATTGACAAGGTGTGAAGAGCCGCGTGTGCTCAGTGTGCTTCCTCCGGC
CCCTGAATGTGGCTAACCTTAACCCTGCAGCCGTTGCCCATAATCCAATG
GGTTTGCGGTCGTAATGCGTAAGTGCGGGATGGGACCAACTACTTTGGGT
GTCCGTGTTTCCTGTTTTTCTTTTGATTGCATTTTATGGTGACAATTTAT
AGTGTATAGATTGTCATC
DNA sequence of iHRV-B26
SEQ ID NO: 41
TTAAAACAGCGGGTGGGTATCCCACCACCCGACCCACTGGGTGTTGTACA
CTGGTATTTTGTACCTTTGTATGCCTGTTTGCACCTCCCCACCCCTTCCA
ATTACCCTTACCCGAATTGTATTATGCGGTAACATTAGAAGAAGTGAACA
CAGTGCAATAGGACGTATCACATCCAGTGATATAAAGCACAAGCAATTCT
TGTTCCCCGGAGCTGGATATAGACTGCTAACGTGGTTGAAGGTCCTTAAC
CGTTATCCGCCAACCAACTACGACACGGCTAGTAATATCATGTTTGTCTT
TGAGCGTTCGATCAGGTGAATTCCCCATTCACTAGTTTGGTCGATGAGGC
TGAGAACTCCCCACAGGTGACTGTGTCTCAGCCTGCGTGGCGGCCAACCC
AGCCACGGCTGGGACGCCCACTGATAGACATGGTGTGAAGACCCAATTGT
GCTTGGTTGTGACTCCTCCGGCCCCTGAATGCGGCTAACCTCAACCCCGG
AGCCTTGTAGTGTAAGCCAACACATACAAGGTCGTAATGGGCAACTCTGG
GACGGGACCGACTACTTTGGGTGTCCGTGTTTCCTTTATTTTTATCTTTT
GTGTCTTATGGTTACAAGTATTGATTGTAACC
DNA sequence of iBEV1
SEQ ID NO: 42
TTAAAACAGCCTGGGGGTTGTACCCACCCCTGGGGCCCACGCGGCGCCAG
TACTCTGGTACGCTAGTACCTTTGTACGCCTGTTTTCCCCACCCTTAAAT
AAATTAAGATTACCACTGCTGTGGGGAGTAGTCCGACTCCGCACCGATAC
GTCGCACCAGTGAACTGGTTCGCTTAGAACCTTTGCACGGAGCAGCTGGT
ATCCCCCCCCGTAACTTAGAAGCCTGGACAAACCGACCAATAGAGGCGTT
GTAGCCAGCAGCGCAACGGTCAAGCACTTCTGTTTCCCCGGCGCCATGCG
TCGTTACCCGCCCGGCCTACTGCGGGAAGCCTAGTAGGACCCCAATCGGA
CGCGCGGTTGCGTTCAGCCACAACCCCAGTGGTAGCTCTGAGGAATGGGA
CTCGCCTACCCCCCACAGCAATGTGGTGGCTTGTCCGCGTGTGTCCTCGG
GTTCGCTCGTTGAGCGATCACCGCAACTCTGAGTAAGGTCTCAAGAGCCT
ACTGCGCTAGGTCGGTTCTCCTCCGGAGCCGTGAATGCTGCTAATCCCAA
CCTCCGAGCGTGTGCGCACAACCCAGTGTTGCTACGTCGTAATGCGTAAG
TTGGAGGCGGAACAGACTACTTTCGGTACTCCGTGTTTCCTTAATTTTTG
TTCATTATTACATGGTGACATTGACTGACATTTGTGAATTTGCCCGCCTG
CCCTTGAACATAGCTCTCTATTACTTGATTGCATTTCACAAAACAACCCA
ACTCACTGTTGAACTTGTTGACTTCGCAGTCTACTTGAACTTACAGTACA
ATCACATTCACA
DNA sequence of iEchoV1
SEQ ID NO: 43
TTAAAACAGCCTGTGGGTTGTTCCCACCCACAGGGCCCACTGGGCGTTAG
CACACTGGTATCACGGTACCTTTGTGCGCCTGTTTTATACTCCCCCCCCT
AAGGAAACTTTAGAAGCAAAGCAATTGTGATCAATAGTGGGTATGGCACA
CCAGTCATATCTTGATCAAGCACTTCTGTTCCCCCGGACTTAGTACCAAT
AGACTGCTCAAGCGGTTGAAGGGGAAAACGTTCGTTATCCGGCCAACTAC
TTCGAGAAACCTAGTAGCACCATGAAAGTTGCGGAGTGTTTCGCTCAGCA
CTTCCCCCGTGTAGATCAGGCTGATGAGTCACCGTATTCCCCACGGGCGA
CCGTGACGGTGGCTGCGTTGGCGGCCTGCCCATGGGGTAACCCATGGGAC
GCTCTAAAACAGACACGGTGCGAAGAGTCTATTGAGCTAGTTGGTAGTCC
TCCGGCCCCTGAATGCGGCCAATCCTAACTGCGGAGCACATACTCCCAAT
CCAGGGAGCAGTGTGTCGTAATGGGTAACTCTGCAGCGGAACCGACTACT
TTGGGTGTCCGTGTTTCCTTTTATTCTCACATTGACTGCTTATGGTGACA
ATTGAAAGATTGTTACCATATAGCTATTGGATTGGTCATCCGGTGAGCAA
TAGAGCTATTGTTTATCAATTTGTTGGATTTGTACCACTCAACTTTTCTG
TTTTGAGAACACTCAACTACATCTTACTGCTAAACACATCAAA
DNA sequence of iHRV-A21
SEQ ID NO: 44
TTAAAACTGGGTCCAGGTTGTTCCCACCTGGATCTCCTATTGGGAGTTGT
ACTCTATTATTCCGGTAATTTTGTACGCCAGTTTTATCTCCCCTTCCCCA
ATTGCAACTTAGAAGTTTATCAATATGACCAATAGGCGGTAGTTAGCCAA
ACTACCAGAGGTCAAGCACTTCTGTTTCCCCGGTCAAAGTTGATATGCTC
CAACAGGGCAAAAACAACTGAGATCGTTATCCGCAAAGTGCCTACGCAAA
GCCTAGTAACACCTTTGAAGATTTATGGTTGGTCGTTCCGCTATTTCCCA
TAGTAGACCTGGCAGATGAGGCTAGAAATTCCCCACTGGCGACAGTGTTC
TAGCCTGCGTGGCTGCCTGCGCACCCCTTGGGTGCGAAGCCATACATTGG
ACAAGGTGTGAAGAGCCCCGTGTGCTCACTTTGAGTCCTCCGGCCCCTGA
ATGTGGCTAACCTTAACCCTGCAGCTAGTGCATGTAATCCAACATGTTGC
TAGTCGTAATGAGCAATTGCGGGACGGGACCAACTACTTTGGGTGTCCGT
GTTTCACTTTTTCCTTTTAATATTGCTTATGGTGACAATATATATAAACA
TATATATTGACACC
DNA sequence of iPV1
SEQ ID NO: 45
TTAAAACAGCTCTGGGGTTGTACCCACCCCAGAGGCCCACGTGGCGGCTA
GTACTCCGGTATTGCGGTACCCTTGTACGCCTGTTTTATACTCCCTTCCC
GTAACTTAGACGCACAAAACCAAGTTCAATAGAAGGGGGTACAAACCAGT
ACCACCACGAACAAGCACTTCTGTTTCCCCGGTGATGTCGTATAGACTGC
TTGCGTGGTTGAAAGCGACGGATCCGTTATCCGCTTATGTACTTCGAGAA
GCCCAGTACCACCTCGGAATCTTCGATGCGTTGCGCTCAGCACTCAACCC
CAGAGTGTAGCTTAGGCTGATGAGTCTGGACATCCCTCACCGGTGACGGT
GGTCCAGGCTGCGTTGGCGGCCTACCTATGGCTAACGCCATGGGACGCTA
GTTGTGAACAAGGTGTGAAGAGCCTATTGAGCTACATAAGAATCCTCCGG
CCCCTGAATGCGGCTAATCCCAACCTCGGAGCAGGTGGTCACAAACCAGT
GATTGGCCTGTCGTAACGCGCAAGTCCGTGGCGGAACCGACTACTTTGGG
TGTCCGTGTTTCCTTTTATTTTATTGTGGCTGCTTATGGTGACAATCACA
GATTGTTATCATAAAGCGAATTGGATTGGCCATCCGGTGAAAGTGAGACT
CATTATCTATCTGTTTGCTGGATCCGCTCCATTGAGTGTGTTTACTCTAA
GTACAATTTCAACAGTTATTTCAATCAGACAATTGTATCACC
DNA sequence of iEV71
SEQ ID NO: 46
TTAAAACAGCCTGTGGGTTGCACCCACTCACAGGGCCCACTGGGCGCAAG
CACTCTGGTACCTCGGTACCTTTGTGCGCCTGTTTTACACCCCCCCCCCA
GTGAAACTTAGAAGCAGCAAACCACGATCAATAGCGGGCATAACGCTCCA
GTTATGTCTTGATCAAGCACTTCTGTTTCCCCGGACTGAGTATCAATAGA
CTGCTCGCGCGGTTGAAGGAGAAAACGTTCGTTATCCGGCTAGCTACTTC
GGGAAACCTAGTAACACCATGAAAGTTGCGGAGAGCTTCGTTCAGCACTC
CCCCAGTGTAGATCAGGTCGATGAGTCACCGCATTCCCCACGGGCGACCG
TGGCGGTGGCTGCGTTGGCGGCCTGCCCATGGGGTAACCCATGGGGCGCT
CTAATACGGACATGGTGTGAAGAGTCTACTGAGCTAGTTAGTAGTCCTCC
GGCCCCTGAATGCGGCTAATCCCAACTGCGGAGCACACGCCCACAAGCCA
GCGGGTAGCGTGTCGTAACGGGTAACTCTGCAGCGGAACCGACTACTTTG
GGTGTCCGTGTTTCTTTTTATCTTTATACTGGCTGCTTATGGTGACAATT
AAAGAATTGTTACCATATAGCTATTGGATTGGCCATCCGGTGTGCAACAG
AGCAATTGTTTACCTATTCATTGGTTTCGTACCATTAACCTTGAAGTCTG
TGACCACCCTTAACTACATCTTGACCCTTAACACAGCTAAAC
DNA sequence of iHRV-A9
SEQ ID NO: 47
TTAAAACTGGATCTGGGTTGTTCCCACTCAGATCTCCCACGTGGAGTAGT
ACACTATTATTACGGTAATCTTGTACGCCAGTTTTATAATCCCCTTACCC
AAGTAACTTAGAAGATAAACACAAAGACCAATAGGAGATGATCATCCAGA
TCATCATAGGTCAAGCACTTCTGTTTCCCCGGTCAAGGTTGATATGCTCT
AACAGGGCAAAAACAGCTGAGATCGTTATCCGCAAAGCGCCTACGCAAAG
CCTAGTAACTATCTGGAAGTTGCTTGGTTGGTCGCTCCGCCATATCCCAT
GGTAGACCTGGCAGATGAGGCTAGAAATTCCCCACTGGTGACAGTGTTCT
AGCCTGCGTGGCTGCCTGCACACCCTCTGGGTGTGAAACCAAGTAATGGA
CAGGGTGTGAAGAGCCCCGTGTGCTCGCTTTGAGTCCTCCGGCCCCTGAA
TGTGGCTAACCTTAACCCTGCAGCTAGGGCACACAATCCAGTGTGTATCT
AGTCGTAATGAGCAATTGCGGGATGGGACCAACTACTTTGGGTGTCCGTG
TTTCTTGTTTTTCTTTTATGTTTGCTTATGGTGACAATATATAGTAATAT
ATATTGGCATC
DNA sequence of iSiminan V4
SEQ ID NO: 48
TTAAAATAGCTGCTGGGTTGTTCCCATCCAGCAGGCCCACTGGGCGTGAT
ACTCTGGTATTGCGGTACCTTTGTATGCCTATTTTACCTCCCTTCCCCCT
GAAACTTAGAAGAAAGAAACAAACACGCTCACTAGGTACACTGCATCCAG
CAGTGTAATGAGCAAGCACTTCTGTTTCCCCGGAAGGGATATATGGTACG
CTGTGCAAACGGCGGAAATTAATCCTACCGTTAACCGCCCATCTACTCCG
AGAAGCCTAGTACCTAATTGAACTTATCAATGGAGTTGCGCTCAGCAGGT
GACCCTGACCTGCCAGCTCCGGCTGATGGACCTGGGCATCCCCCACAGGC
GACTGTGGCCCAGGTCGCGTGGCGGCCGGCCCACTCTTTTGAGTGGGACG
CCTTGATAATGACAAGGTGGGAAGAGCCTATTGGGCTAGCTGGTTTCCTC
CGGCCTCCTGAATGCGGCTAACCCTAACCCCGGAGCATATGGTGGCAACC
CAGCCACTGGTATGTCGTAACGCGTAAGTCTGGGATGGGACCGACTACTT
TGGAGAGTCCGTGTTTCCATTATTTTCTTTATTGAATCTTATGGTGACAA
ATTGGGAGTTACTCGGGTGACGATTGATTATTACTTCTGAAAACGTAGAG
ATATAGAATCAACACA
DNA sequence of iEV-D94
SEQ ID NO: 49
TTAAAACAGCCTGTGGGTTGTTCCCACCCCAGAGGCCCACGTGGCGGCCA
GTACTCTGGTATCACGGTACCTTTGTACGCCTGTTTTATATCCCCTTCCC
CCGCAACTTAGAAGAAAACAAATCAAGTTCACTAGGAGGGGGTACAAACC
AGTACCACCACGAACAAGCACTTCTGTTTCCCCGGTGATGTCGTATAGAC
TGTAACCACGGTTGAAAACGATTGATCCGTTATCCGCTCTTGTACTTCGA
AAAGCCCAGTATCACCTTGGAATCTTCGATGCGTTGCGCTCAGCACTCAA
CCCCAGAGTGTAGCTTAGGTCGATGAGTCTGGACACTCCTCACCGGCGAC
GGTGGTCCAGGCTGCGTTGGCGGCCTACCTGTGGTCCAAAGCCACAGGAC
GCTAGTTGTGAACAAGGTGTGAAGAGCCTATTGAGCTACAAGAGAATCCT
CCGGCCCCTGAATGCGGCTAATCCTAACCACGGAGCAAGGGTACACAAAC
CAGTGTATATCTTGTCGTAACGCGCAAGTCTGTGGCGGAACCGACTACTT
TGGGTGTCCGTGTTTCCTTTTGTTTTTATCATGGCTGCTTATGGTGACAA
TCTAAGATTGTTATCATATAGCTGTTGGATTGGCCATCCGGTAATTTATT
GAGATTTGAGCATTTGCTTGTTTCTTCAACAATTTCACCTATTCATTGCA
TTTCAGCAGTCAAA
DNA sequence of iSimianA5
SEQ ID NO: 50
TTAAAATAGCCTGTGGGTTGCTCCCACCCACAGGGCCCAAGTGGCGTAGT
ACTTGGTATTCCGGTACCTTTGTACACCTATTTACAAACCCTACCCCTTG
TAACCTTAGAAGCAATTATTTAACCGCTCACTAGGGGGTGTGCTATCCAA
GCACATCAAGAGCAAGCACTTCTGTCTCCCCGGGAGGGGCTAATGGTACG
CTGTGCCCACGGCGGAAATGAGCCCTACCGTTAACCGGCAGTCTACTTCG
GGAAGCCCAGTAACTACATTGAAACTTTGAGGCGTTACACTCAGCACATA
ACCCCAATGTGTAGTTCTGGTCGATGAGCCTTGGCATCCCCCACAGGCGA
CTGTGGCCAAGGCTGCGTTGGCGGCCAGCCTGCGGACCAAAAGTCCGTAG
GACGCCTAATTGTGGACATGGTGTGAAGAGCCTACTGAGCTAGACTGTAG
TCCTCCGGCCCCTGAATGCGGCTAATCCTAACCCTGGAGCATCCGCGTGC
AACCCAGTACGTAGGGTGTCGTAATGCGTAAGTCTGGGATGGAACCGACT
ACTTTGGGTGTCCGTGTTTCTTGTTTTTCATACTGGGTCGCTTATGGTTA
CAACTAATTGTTGTAATCATTGGCAGTGCGCGCTGACCACGCGATTATTG
ATATTTCCATTTGTTGGATACTCCAATAGTGTCAACTCATATACACAACT
TTTACCACTGATCAAGATAAAA
DNA sequence of iPV3
SEQ ID NO: 51
TTAAAACAGCTCTGGGGTTGCTCCCACCCCAGAGGCCCACGTGGCGGCCA
GTACTCTGGTATTGCGGTACCTTTGTACGCCTGTTTTATACTCCCTCCCC
CCGTGCAACTTTAGAAGAAATCCACAAAGTTCAATAGAGGGGGTGCAAAC
CAGCACCACCACGAACAAGCACTTCTGTTTCCCCGGTGATGTCGTATAAG
CTGTACCCACGGCTGAAGGCGACGGATCCGTTATCCGCTTGAGTACTTCG
AGAAGCCTAGTATTACCTTGGAATCTTCGACGCGTTGCGCTCAGCACTCT
ACCCCGAGTGTAGCTTAGGTCGATGAGTCTGGGCACGCCCCACCGGCGAC
GGTGGCCCAGGCTGCGTTGGCGGCCTACCCATGGCTATCACCATGGGACG
CTAGTTGTGAACAAGGTGTGAAGAGCCTATTGAGCTACCCAAGAGTCCTC
CGGCCCCTGAATGCGGCTAATCCTAACCACGGAGCAAGTGTCCTCAACCC
AGGGGATGGCTTGTCGTAACGCGAAAGTCTGTGGCGGAACCGACTACTTT
GGGTGTCCGTGTTTCCTTTTATTTTTATGTATGGCTGCTTATGGTGACAA
TCAAAGGTTGTTACCATAAAGCAATTTGGATTGGCCATCCGGTGAGAATC
AAACATATTATCTACCTGTTTGTTGGGTTTTCTTCTTTTACTTGAACAAT
ACCTCTAATAATAACTGCTATATTGTCAATAAGACATTATCATCACA
DNA sequence of iHRV-C54
SEQ ID NO: 52
TTAAAACAGCTGTGTGGTTGTTCCCACCACCAGGCACACTGTGCGTTGTA
CACTGGGATTCCGGTCACTTTGTACGCCTGTTTGCTATCCCCCCCAACTT
ATGTAATTTAGAAGATGTACACAACGCCCATTAGGATGCGGCCAAACCAG
GTCCGCTTAGGGCAAGCACTTCTGTTTCCCCGGGTGTGTGAATAGACTCT
AACAGGGTTGAAGCTGTAGCACTCGTTATCCGCGCAACTACTACGCGAAT
GTTAGTAGCATCCTGTGTTGCATTTGGGATTTCGCTCCGCAGAAAACCCC
ATCTGTAGATTAGGGCAATGAGGCTACACATACCCCACTGGCGACAGTGG
TGTAGCCTGCGTGGTGCCCTACCCAGGCCATCTTGGCCTGGGATTCCACT
TACAAGACAGGGTGTGAAGGCACTAGTGTGCTAGTTGTGAGTCCTCCGGC
CCCTGAATGCGGCTAATCTTAACCCCGTAGCCCCCGCAAGTAAACCAACT
TGTAGGTGGTCGTAATGAGTAATTACGGGATGGAACCGACTACTTTGGGT
GTCCGTGTTTCCTTTTATTCTTTATATTTGCATCCTATGGTTACAACATA
AGTAATC
DNA sequence of iHRV-A100
SEQ ID NO: 53
TTAAAACTGAATCCAGATTGTTCCCATCTGGATTTCCTACATGGAGTTGT
ACTCTATTATTCCGGTAATTTTGTACGCCAGTTTTATCACCCCTTCCCCC
GTAACTTAGAAGTTTGAAACAAAAGACCAATAGGAGGTAACTATCCAAGT
TACTATAGGTCAAGCACTTCTGTTTCCCCGGTCAAAGTTGATATGCTCCA
CCAGGGCAAAAACAATTGAGATCGTTATCCGCAAAGTGCCTACGCAAAGC
CTAGTAGTATCTTGAAAAGCATGTGGTTGGTTGCTCCGCTGTACCCCACA
GTAAACCTGGCAGATGAGGCTAGAAGTTCCCCACTGGTGACAGTGTTCTA
GCCTGCGTGGCTGCCTGCGCACTCTTTGAGTGCGAAGCCATATGTTTGAC
AAGGTGTGAAGAGCCCCGTGTGCTCACTTTGAGTCCTCCGGCCCCTGAAT
GTGGCTAACCTTAACCCTGCAGCTAGTGCATGCAATCCAGCATGTGGCTA
GTCGTAATGAGCAATTGCGGGATGGGACCAACTACTTTGGGTGTCCGTGT
TTCACTTTTTTCCTTTTATAATTGCTTATGGTGACAATATATAGTGATAT
ATATTGACACC
DNA sequence of iHRV-B37
SEQ ID NO: 54
TTTAAACAGCGGATGGGTATCCCACCATCCGACCCACTGGGTGTAGTACT
CTGGTATTTTGTACCTTTGTACGCCTGTTGTTCCTAATGTACCCACCCTA
AAACTTCCTACCCAAGTAACGTTAGAAGTTTCATCAACAAGTACAATAGG
AAGCATCACATCCAGTGGTGTTTTGTACAAGCACTTCTGTTTCCCCGGAG
CGAGGTATAGGCTGTACCCACTGCCGAAAGCCTTTAACCGTTATCCGCCA
ACCAACTACGTAAAAGCTAGTATCATCATGTTTTAAAATAGGCGTTCGAT
CAGGTGGTACCCCCCTCCACTAGTTTGGTCGATGAGGCTAGGAACTCCCC
ACGGGTGACCGTGTCCTAGCCTGCGTGGCGGCCAACCCAGCTTCTGCTGG
GACGCCTTTTTATGGACATGGTGTGAAGACTCGCATGTGCTTGGTTGTGA
CTCCTCCGGCCCCTGAATGCGGCTAACCTTAACCCCGGAGCCCTGTGTTG
CAATCCAGTAACATTAGGGTCGTAATGAGCAATTTCGGGACGGGACCGAC
TACTTTGGGTGTCCGTGTTTCTCATTTTTCTTATTATTGTCTTATGGTCA
CAGCATATATATAACGTATATACTGTGATC
DNA sequence of iHRV-B4
SEQ ID NO: 55
TTAAAACAGCGGATGGGTTTCCCACCATCCGACCCACTGGGTGTAGTGCT
CTGGTATTTTGTACCTTTGCACGCCTGTTTCCCATTTGTACCCTTCCTTA
ATCTCCTTCCCCCGTAACGTTAGAAGTTTTGGAATTTTAAAGTACAATAG
GAAGCGCCACATCCAGTGGTGTTGCGTACAAATACTTCTGTTTACCCGGA
GCGAGGTATAGGTTGTACCCACGGCCAAAAGCCTTTAACCGTTATCCGCC
AATCAACTACGTAACGGCTAGTATCATCTTGCTTTTGATTTGGTGTTCGA
TCAGGTGGTATCCCCCACTAGTCTGGTCGATGAGGCTAGGAATTCCCCAC
GGGCGACCGTGTCCTAGCCTGCGTGGCGGCCAGCCCAGCTTTTGCTGGGA
CGCCTTTTCAAAGACATGGTGTGAAGACCTGCATGTGCTTGGTTGTGAGT
CCTCCGGCCCCTGAATGCGGCTAACCTTAACCCTGGAGCCCAGCAGCATA
ATCCAATGTTGTTTGGGTCGTAATGAGCAATTCCGGGACGGGACCGACTA
CTTTGGGTGTCCGTGTTTCCTTTTATTCTTACATTGTCTTATGGTCACAG
CATATATATTATATATACTGTGATC
DNA sequence of iHRV-B92
SEQ ID NO: 56
TTAAAACAGCGGATGGGTATCCCACCATCCGGCCCACTGGGTGTAGTACT
CTGGTACATTGTACCTTTGTACGCCTGTTTTCCCCCTCTTGTACCCGCCC
TTCAAGCTCCTTGCCCAAGTAACGTTAGAAGTTTGAACATTGGTACAATA
GGAAGCATCACATCCAGTGGTGTACTGTACAAACACTTCTGTTGCCCCGG
AGCGAGGTATAGATGGTCCCCACCGTCAAAAGCCTTTAACCGTTATCCGC
CAATCAACTACGTAATGGCTAGTAGCACCTTGGATTTAAGTTGGCGTTCG
ATCAGGTGGTAACCCCCACTAGTTTGGTCGATGAGGCTAGGAATTCCCCA
CGGGTGACCGTGTCCTAGCCTGCGTGGCGGCCAACCCAGCATCCGCTGGG
ACGCCAATTTAATGACATGGTGTGAAGACCTGCATGTGCTTGATTGTGAG
TCCTCCGGCCCCTGAATGCGGCTAACCCTAACCCCGGAGCCTTGCAGCAC
AATCCAGTGTTGTTAAGGTCGTAATGAGCAATTCTGGGATGGGACCGACT
ACTTTGGGTGTCCGTGTTTCTTATTTTTCTTGAATTTTTCTTATGGTCAC
AGCATATATACATTATATACTGTGATC
DNA sequence of iHRV-Al
SEQ ID NO: 57
TTAAAACTGGGTGTGGGTTGTTCCCACCCACACCACCCAATGGGTGTTGT
ACTCTGTTATTCCGGTAACTTTGTACGCCAGTTTTTCCCTCCCCTCCCCA
TCCTTTTACGTAACTTAGAAGTTTTAAATACAAGACCAATAGTAGGCAAC
TCTCCAGGTTGTCTAAGGTCAAGCACTTCTGTTTCCCCGGTTGATGTTGA
TATGCTCCAACAGGGCAAAAACAACAGATACCGTTATCCGCAAAGTGCCT
ACACAGAGCTTAGTAGGATTCTGAAAGATCTTTGGTTGGTCGTTCAGCTG
CATACCCAGCAGTAGACCTTGCAGATGAGGCTGGACATTCCCCACTGGTA
ACAGTGGTCCAGCCTGCGTGGCTGCCTGCGCACCTCTCATGAGGTGTGAA
GCCAAAGATCGGACAGGGTGTGAAGAGCCGCGTGTGCTCACTTTGAGTCC
TCCGGCCCCTGAATGCGGCTAACCTTAAACCTGCAGCCATGGCTCATAAG
CCAATGAGTTTATGGTCGTAACGAGTAATTGCGGGATGGGACCGACTACT
TTGGGTGTCCGTGTTTCACTTTTTCCTTTATTAATTGCTTATGGTGACAA
TATATATATTGATATATATTGGCATC
DNA sequence of iEV-B107
SEQ ID NO: 58
TTAAAACAGCCTGTGGGTTGTTCCCACCCGCAGGGCCCACTGGGCGCTAG
CACACTGGTATCCCGGTACCCTTGTGCGCCTGTTTTATATACCCTCCCCC
TTATGTAACTTAGAAGTATGATTCAAACGGTCGACAGGCGGCTCAGTGCA
CCAACTGAGTCATGACCAAGCACTTCTGTTACCCCGGACTGAGTATCAAT
AAGCTGTTCACACGGCTGAAGGAGAAAACGTTCGTTACCCGGCCAATTAC
TTCGAGAAACCTAGTACCACCATGAAGGTTGCGCAGTGTTTCGCTCCACA
CAACCCCAGTGTAGATCAGGTCGATGAGTCACCGCATTCCCCACGGGCGA
CCGTGGCGGTGGCTGCGTTGGCGGCCTGCCCATGGGGCAACCCATGGGAC
GCTTCAATACTGACATGGTGTGAAGAGTCTATTGAGCTAATTGGTAGTCC
TCCGGCCCCTGAATGCGGCTAATCCCAACTGTGGAGCAGATACTCACAAA
CCAGTGAGCGGTCTGTCGTAACGGGCAACTCCGCAGCGGAACCGACTACT
TTGGGTGTCCGTGTTTCTTTTTATTCTTACATTGGCTGCTTATGGTGACA
ATTGACAAATTGTTACCATATAGCTATTGGATTGGCCATCCGGTGACAAA
CAGAGCTATTGTTTACTTGTTTGTTGGTTTCATACCATTAAATTACAAGG
TCTTAGAAACTCTCAACTTTATTTTGACACTCAATACAGCAAA
DNA sequence of iHRV-A57
SEQ ID NO: 59
TTGTTTGATGCCAGTTTTATCTCCCCCCCCAATTGCAACTTAGAAGATGT
ACACAAAGACCAATAGGCAGTGGTCACCCAGACCACTGAAGGTCAAGCAC
TTCTGTCTCCCCGGTCAAAGTTGATATGCTCCAACAGGGCAAAAACAACT
TAGATCGTTATCCGCAAAGTGCCTACGCAAAGCTTAGTACCATCTTTGAG
AGCCTATGGTTGGTCGCTCCACTGAACCCCACAGTAGACCTGGCAGATGA
GGCTAGAAGACCCCCACTGGCGACAGTGTTCTAGCCTGCGTGGCTGCCTG
CACACCCTTACGGGTGTGAAGCCATATGTTTGACAAGGTGCGAAGAGCCC
CGTGTGCTCACTTTGAGTCCTCCGGCCCCTGAATGTGGCTAACCTTAACC
CTGCAGCTAGTGCACACAAGCCAGTGTGTTGCTAGTCGTAATGAGCAATT
GCGGGATGGGACCAACTACTTTGGGTGTCCGTGTTTCACTTTTTACCTTT
ATTTTGCTTATGGTGACAATATATATAGTATATATATTGGCACC
DNA sequence of human XIAP
SEQ ID NO: 60
CTTGTAAAAACAACTTTGATGCCTTGAATATATAATGATTCATTATAACA
ATTATGCATAGATTTTAATAATCTGCATATTTTATGCTTTCATGTTTTTC
CTAATTAATGATTTGACATGGTTAATAATTATAATATATTCTGCATCACA
GTTTACATATTTATGTAAAATAAGCATTTAAAAATTATTAGTTTTATTCT
GCCTGCTTAAATATTACTTTCCTCAAAAAGAGAAAACAAAAATGCTAGAT
TTTACTTTATGACTTGAATGATGTGGTAATGTCGAACTCTAGTATTTAGA
ATTAGAATGTTTCTTAGCGGTCGTGTAGTTATTTTTATGTCATAAGTGGA
TAATTTGTTAGCTCCTATAACAAAAGTCTGTTGCTTGTGTTTCACATTTT
GGATTTCCTAATATAATGTTCTCTTTTTAGAAAAGGTGGACAAGTCCTAT
TTTCAAGAGA
DNA sequence of iHRV-B97
SEQ ID NO: 61
TTAAAACAGCGGATGGGTTTCCCACCATCCGACCCACTGGGTGTAGTGCT
CTGGTATTTTGTACCTTTGCACGCCTGTTTCCCCTTTGTACCCATCCTGA
ATTTCCTCCCTCTGCAACGTTAGAAGTTTGTGAAATTAAAAGTACAATAG
GAAGCATCACATCCAGTGGTGTTCAGTACAAGCACTCCTGTTTCCCCGGA
GCGAGGTATAGGTTGTACCCACGACCGAAAGCCTTTAACCGTTATCCGCC
AATCAACTACATAACGGCTAGTATCATCATGTTTTTGATCTGGCGTTCGA
TCAGGTGGTTTCCCCCACTAGTCTGGTCGATGAGGCTAGGATTTCCCCAC
GGGCGACCGTGTCCTAGCCTGCGTGGCGGCCAGCCCAGCTTATGCTGGGA
CGCCTTTTTAAAGACATGGTGTGAAGACCTGCATGTGCTTGATTGTGAGT
CCTCCGGCCCCTGAATGCGGCTAACCTTAACCCTGGAGCCCGACAGCATA
ATCCAATGTTGTTTGGGTCGTAATGAGCAATTCCGGGATGGGACCGACTA
CTTTGGGTGTCCGTGTTTCTTTTTATTCTTATATTGTCTTATGGTCACAG
CATATATAGTATATATACTGTGATC
DNA sequence of iHRVB-B14
SEQ ID NO: 62
TACTCTGGTATTATGTACCTTTGTACGCCTGTTTCTTCCCTACAACCCCT
TCCTAAAACTCCCACCCATGAAACGTTAGAAGCTTGACATTTAAGTACAA
TAGGTGGCACCACATCCAGTGGTGTCTACGTACAAGCACTTCTGTTTCCC
CGGAGCGAGGTATAGGCTGTACCCACTGCCAAAAGCCTTTAACCGTTATC
CGCCAACCAACTACGTAACAGCTAGTATCATCTTGTTCTTCACTGGACGT
TCGATCAGGTGGATTCCCCCTCCACTAGTTTGGTCGATGAGGCTAGGAGC
TCCCCACGGGTGACCGTGTCCTAGCCTGCGTGGCGGCCAACCCAGCTCAT
GCTGGGACGCCCTTTTAAGGACATGGTGTGAAGACTCGCATGTGCTTGGT
TGTGAGTCCTCCGGCCCCTGAATGCGGCTAACCTTAACCCTGGAGCCTTA
TGCCACGAACCAGTGGTTGTAAGGTCGTAACGAGCAATTCCGGGATGGGA
CCGACTACTTTGGGTGTCCGTGTTTCCTATTTTTCTTTATATTGTCTTAT
GGTCACAGCATATATATAAGTATATACTGTGATC
DNA sequence of EMCV IRES-NeonGreen
(T7 promoter (nucleotides 1-19) T4
td upstream (nucleotides 21-275)
5′UTR (PABPv3) (nucleotides 277-326)
EMCV IRES (nucleotides 327-894)
NeonGreen (nucleotides 1034-1751)
3′UTR (HBA1 full length) (nucleotides
1752-1852) T4 td Downstream
(nucleotides 1853-2059))
SEQ ID NO: 63
TAATACGACTCACTATAGGGGGGAATTCTAGAGAAAATTTCGTCTGGATT
AGTTACTTATCGTGTAAAATCTGATAAATGGAATTGGTTCTACATAAATG
CCTAACGACTATCCCTTTGGGGAGTAGGGTCAAGTGACTCGAAACGATAG
ACAACTTGCTTTAACAAGTTGGAGATATAGTCTGCTCTGCATGGTGACAT
GCAGCTGGATATAATTCCGGGGTAAGATTAACGACCTTATCTGAACATAA
CGCTACCGTTTAATATTGCGTCATATAAAAAAAAAAAACCAAAAAAAAAA
AACAAAAAAAAAAAATAATTGACTAATCCCTCCCCCCCCCCTAACGTTAC
TGGCCGAAGCCGCTTGGAATAAGGCCGGTGTGCGTTTGTCTATATGTTAT
TTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGG
AATGCAAGGTCTGTTGAATGTCGTGAAGGAAGCAGTTCCTCTGGAAGCTT
CTTGAAGACAAACAACGTCTGTAGCGACCCTTTGCAGGCAGCGGAACCCC
CCACCTGGCGACAGGTGCCTCTGCGGCCAAAAGCCACGTGTATAAGATAC
ACCTGCAAAGGCGGCACAACCCCAGTGCCACGTTGTGAGTTGGATAGTTG
TGGAAAGAGTCAAATGGCTCTCCTCAAGCGTATTCAACAAGGGGCTGAAG
GATGCCCAGAAGGTACCCCATTGTATGGGATCTGATCTGGGGCCTCGGTG
CACATGCTTTACATGTGTTTAGTCGAGGTTAAAAAAACGTCTAGGCCCCC
CGAACCACGGGGACGTGGTTTTCCTTTGAAAAACACGATGATAAGCTTGC
CACAACCCGGGATCCTCTAGGAGTACTGAGCTCGTTTAGTGAACCGTCAG
ATCGCCTGGAGACGCCATCCACGCTGTTTTGACCTCCATAGAAGACACCG
GGACCGATCCAGCCTCCGCGGCCCCGAATTCACCATGGGCTCTGTGTCTA
AAGGAGAAGAAGACAACATGGCGAGTCTGCCGGCGACACATGAGCTGCAC
ATCTTTGGATCCATCAACGGTGTCGACTTTGACATGGTTGGCCAAGGCAC
CGGAAATCCAAATGATGGCTATGAGGAGCTGAACCTGAAGAGTACGAAAG
GAGACCTGCAGTTCTCCCCCTGGATCCTGGTGCCTCACATTGGCTATGGC
TTCCACCAGTATCTGCCCTACCCAGACGGCATGAGCCCTTTCCAGGCTGC
CATGGTGGATGGATCAGGCTACCAGGTGCACAGGACCATGCAGTTTGAGG
ATGGGGCTTCGTTGACTGTGAACTACAGATACACATATGAAGGGTCTCAT
ATTAAAGGAGAGGCCCAGGTGAAGGGGACAGGCTTCCCTGCTGACGGTCC
TGTCATGACCAACAGCCTGACTGCAGCTGACTGGTGCCGCAGCAAAAAGA
CGTACCCCAATGACAAGACCATCATAAGCACTTTCAAGTGGAGCTACACC
ACTGGGAATGGCAAGAGGTACCGCTCCACTGCCCGTACCACCTATACCTT
TGCCAAGCCAATGGCTGCCAATTACCTGAAGAACCAGCCCATGTACGTCT
TCCGGAAGACAGAGTTGAAGCACAGCAAAACTGAACTCAACTTCAAAGAG
TGGCAGAAGGCCTTCACAGATGTGATGGGGATGGATGAGCTCTACAAATG
AGCTGGAGCCTCGGTGGCCATGCTTCTTGCCCCTTGGGCCTCCCCCCAGC
CCCTCCTCCCCTTCCTGCACCCGTACCCCCGTGGTCTTTGAATAAAGTCT
GACTCAGTAGATGTTTTCTTGGGTTAATTGAGGCCTGAGTATAAGGTGAC
TTATACTTGTAATCTATCTAAACGGGGAACCTCTCTAGTAGACAATCCCG
TGCTAAATTGTAGGACTGCCCTTTAATAAATACTTCTATATTTAAAGAGG
TATTTATGAAAAGCGGAATTTATCAGATTAAAAATACTTTCTCTAGAGTC
GACCTGCAG
DNA sequence of EMCV IRES-RFP ((T7
promoter (nucleotides 1-19) T4 td
upstream (nucleotides 21-275) 5′UTR
(PABPv3) (nucleotides 277-326) EMCV
IRES (nucleotides 327-894) RFP
(nucleotides 930-1607) 3′UTR (HBA1
full length) (nucleotides 1608-1708) T4
td Downstream (nucleotides 1709-1915))
SEQ ID NO: 64
TAATACGACTCACTATAGGGGGGAATTCTAGAGAAAATTTCGTCTGGATT
AGTTACTTATCGTGTAAAATCTGATAAATGGAATTGGTTCTACATAAATG
CCTAACGACTATCCCTTTGGGGAGTAGGGTCAAGTGACTCGAAACGATAG
ACAACTTGCTTTAACAAGTTGGAGATATAGTCTGCTCTGCATGGTGACAT
GCAGCTGGATATAATTCCGGGGTAAGATTAACGACCTTATCTGAACATAA
CGCTACCGTTTAATATTGCGTCATATAAAAAAAAAAAACCAAAAAAAAAA
AACAAAAAAAAAAAATAATTGACTAATCCCTCCCCCCCCCCTAACGTTAC
TGGCCGAAGCCGCTTGGAATAAGGCCGGTGTGCGTTTGTCTATATGTTAT
TTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGG
AATGCAAGGTCTGTTGAATGTCGTGAAGGAAGCAGTTCCTCTGGAAGCTT
CTTGAAGACAAACAACGTCTGTAGCGACCCTTTGCAGGCAGCGGAACCCC
CCACCTGGCGACAGGTGCCTCTGCGGCCAAAAGCCACGTGTATAAGATAC
ACCTGCAAAGGCGGCACAACCCCAGTGCCACGTTGTGAGTTGGATAGTTG
TGGAAAGAGTCAAATGGCTCTCCTCAAGCGTATTCAACAAGGGGCTGAAG
GATGCCCAGAAGGTACCCCATTGTATGGGATCTGATCTGGGGCCTCGGTG
CACATGCTTTACATGTGTTTAGTCGAGGTTAAAAAAACGTCTAGGCCCCC
CGAACCACGGGGACGTGGTTTTCCTTTGAAAAACACGATGATAAGCTTGC
CACAACCCGGGATAATTCCTGCAGCCAATATGGCTTCATCGGAAGACGTC
ATCAAAGAATTCATGAGGTTCAAGGTTCGCATGGAGGGCAGTGTGAACGG
CCATGAGTTTGAAATCGAGGGTGAGGGGGAGGGCCGCCCCTACGAAGGGA
CACAGACCGCCAAACTAAAGGTCACCAAAGGGGGCCCGTTGCCCTTTGCC
TGGGACATCTTATCCCCCCAGTTCCAGTACGGGAGCAAGGCCTATGTAAA
ACACCCTGCTGACATTCCAGACTACCTTAAACTGAGTTTCCCAGAAGGCT
TCAAGTGGGAGAGGGTGATGAACTTTGAGGATGGCGGAGTGGTGACAGTG
ACTCAAGACTCCTCCCTGCAGGATGGGGAGTTCATCTACAAAGTGAAGCT
CCGCGGCACTAACTTCCCTTCTGATGGCCCGGTCATGCAGAAGAAAACCA
TGGGCTGGGAAGCCAGCACGGAGAGAATGTACCCTGAGGACGGTGCCCTG
AAAGGTGAGATAAAGATGCGGCTGAAGTTGAAGGACGGAGGGCACTATGA
TGCTGAGGTCAAGACCACCTATATGGCGAAGAAGCCTGTCCAGCTGCCAG
GAGCCTACAAGACAGACATTAAACTTGATATCACCTCTCACAATGAAGAC
TATACCATTGTGGAGCAGTATGAGCGTGCAGAGGGACGGCACAGCACTGG
GGCGTGAGCTGGAGCCTCGGTGGCCATGCTTCTTGCCCCTTGGGCCTCCC
CCCAGCCCCTCCTCCCCTTCCTGCACCCGTACCCCCGTGGTCTTTGAATA
AAGTCTGACTCAGTAGATGTTTTCTTGGGTTAATTGAGGCCTGAGTATAA
GGTGACTTATACTTGTAATCTATCTAAACGGGGAACCTCTCTAGTAGACA
ATCCCGTGCTAAATTGTAGGACTGCCCTTTAATAAATACTTCTATATTTA
AAGAGGTATTTATGAAAAGCGGAATTTATCAGATTAAAAATACTTTCTCT
AGAGTCGACCTGCAG
DNA sequence of EMCV IRES-NeonGreen-
chimeric Intron-EMCV IRES- RFP ((T7
promoter (nucleotides 1-19) T4 td
upstream (nucleotides 21-275) 5′UTR
(PABPv3) (nucleotides 276-326) EMCV
IRES (nucleotides 327-894) NeonGreen
(nucleotides 1035-1751) Chimeric intron
(nucleotides 1837-2066) RFP (nucleotides
2748-3425) 3′UTR (HBA1 full length)
(nucleotides 3426-3526) T4 td Downstream
(nucleotides 3527-3733))
SEQ ID NO: 65
TAATACGACTCACTATAGGGGGGAATTCTAGAGAAAATTTCGTCTGGATT
AGTTACTTATCGTGTAAAATCTGATAAATGGAATTGGTTCTACATAAATG
CCTAACGACTATCCCTTTGGGGAGTAGGGTCAAGTGACTCGAAACGATAG
ACAACTTGCTTTAACAAGTTGGAGATATAGTCTGCTCTGCATGGTGACAT
GCAGCTGGATATAATTCCGGGGTAAGATTAACGACCTTATCTGAACATAA
CGCTACCGTTTAATATTGCGTCATATAAAAAAAAAAAACCAAAAAAAAAA
AACAAAAAAAAAAAATAATTGACTAATCCCTCCCCCCCCCCTAACGTTAC
TGGCCGAAGCCGCTTGGAATAAGGCCGGTGTGCGTTTGTCTATATGTTAT
TTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGG
AATGCAAGGTCTGTTGAATGTCGTGAAGGAAGCAGTTCCTCTGGAAGCTT
CTTGAAGACAAACAACGTCTGTAGCGACCCTTTGCAGGCAGCGGAACCCC
CCACCTGGCGACAGGTGCCTCTGCGGCCAAAAGCCACGTGTATAAGATAC
ACCTGCAAAGGCGGCACAACCCCAGTGCCACGTTGTGAGTTGGATAGTTG
TGGAAAGAGTCAAATGGCTCTCCTCAAGCGTATTCAACAAGGGGCTGAAG
GATGCCCAGAAGGTACCCCATTGTATGGGATCTGATCTGGGGCCTCGGTG
CACATGCTTTACATGTGTTTAGTCGAGGTTAAAAAAACGTCTAGGCCCCC
CGAACCACGGGGACGTGGTTTTCCTTTGAAAAACACGATGATAAGCTTGC
CACAACCCGGGATCCTCTAGGAGTACTGAGCTCGTTTAGTGAACCGTCAG
ATCGCCTGGAGACGCCATCCACGCTGTTTTGACCTCCATAGAAGACACCG
GGACCGATCCAGCCTCCGCGGCCCCGAATTCACCATGGGCTCTGTGTCTA
AAGGAGAAGAAGACAACATGGCGAGTCTGCCGGCGACACATGAGCTGCAC
ATCTTTGGATCCATCAACGGTGTCGACTTTGACATGGTTGGCCAAGGCAC
CGGAAATCCAAATGATGGCTATGAGGAGCTGAACCTGAAGAGTACGAAAG
GAGACCTGCAGTTCTCCCCCTGGATCCTGGTGCCTCACATTGGCTATGGC
TTCCACCAGTATCTGCCCTACCCAGACGGCATGAGCCCTTTCCAGGCTGC
CATGGTGGATGGATCAGGCTACCAGGTGCACAGGACCATGCAGTTTGAGG
ATGGGGCTTCGTTGACTGTGAACTACAGATACACATATGAAGGGTCTCAT
ATTAAAGGAGAGGCCCAGGTGAAGGGGACAGGCTTCCCTGCTGACGGTCC
TGTCATGACCAACAGCCTGACTGCAGCTGACTGGTGCCGCAGCAAAAAGA
CGTACCCCAATGACAAGACCATCATAAGCACTTTCAAGTGGAGCTACACC
ACTGGGAATGGCAAGAGGTACCGCTCCACTGCCCGTACCACCTATACCTT
TGCCAAGCCAATGGCTGCCAATTACCTGAAGAACCAGCCCATGTACGTCT
TCCGGAAGACAGAGTTGAAGCACAGCAAAACTGAACTCAACTTCAAAGAG
TGGCAGAAGGCCTTCACAGATGTGATGGGGATGGATGAGCTCTACAAATG
ATCGAGGTTAATTAATGAGCGGCCGCATAGATAACTGATCCAGTGTGCTG
GAATTAATTCGCTGTCTGCGAGGGCCAGCTGTTGGGGTGAGTACTCCCTC
TCAAAAGCGGGCATGACTTCTGCGCTAAGATTGTCAGTTTCCAAAAACGA
GGAGGATTTGATATTCACCTGGCCCGCGGTGATGCCTTTGAGGGTGGCCG
CGTCCATCTGGTCAGAAAAGACAATCTTTTTGTTGTCAAGCTTGAGGTGT
GGCAGGCTTGAGATCTGGCCATACACTTGAGTGACAATGACATCCACTTT
GCCTTTCTCTCCACAGGTGTCCACTCCCAGGTCCAACTGCAGGTCGAGCA
TGCATCTAGGGCGGCCAATTCCGCCCCTCTCCCCCCCCCCCTTTTCCCTC
CCCCCCCCCTAACGTTACTGGCCGAAGCCGCTTGGAATAAGGCCGGTGTG
CGTTTGTCTATATGTTATTTTCCACCATATTGCCGTCTTTTGGCAATGTG
AGGGCCCGGAAACCTGGCCCTGTCTTCTTGACGAGCATTCCTAGGGGTCT
TTCCCCTCTCGCCAAAGGAATGCAAGGTCTGTTGAATGTCGTGAAGGAAG
CAGTTCCTCTGGAAGCTTCTTGAAGACAAACAACGTCTGTAGCGACCCTT
TGCAGGCAGCGGAACCCCCCACCTGGCGACAGGTGCCTCTGCGGCCAAAA
GCCACGTGTATAAGATACACCTGCAAAGGCGGCACAACCCCAGTGCCACG
TTGTGAGTTGGATAGTTGTGGAAAGAGTCAAATGGCTCTCCTCAAGCGTA
TTCAACAAGGGGCTGAAGGATGCCCAGAAGGTACCCCATTGTATGGGATC
TGATCTGGGGCCTCGGTGCACATGCTTTACATGTGTTTAGTCGAGGTTAA
AAAAACGTCTAGGCCCCCCGAACCACGGGGACGTGGTTTTCCTTTGAAAA
ACACGATGATAAGCTTGCCACAACCCGGGATAATTCCTGCAGCCAATATG
GCTTCATCGGAAGACGTCATCAAAGAATTCATGAGGTTCAAGGTTCGCAT
GGAGGGCAGTGTGAACGGCCATGAGTTTGAAATCGAGGGTGAGGGGGAGG
GCCGCCCCTACGAAGGGACACAGACCGCCAAACTAAAGGTCACCAAAGGG
GGCCCGTTGCCCTTTGCCTGGGACATCTTATCCCCCCAGTTCCAGTACGG
GAGCAAGGCCTATGTAAAACACCCTGCTGACATTCCAGACTACCTTAAAC
TGAGTTTCCCAGAAGGCTTCAAGTGGGAGAGGGTGATGAACTTTGAGGAT
GGCGGAGTGGTGACAGTGACTCAAGACTCCTCCCTGCAGGATGGGGAGTT
CATCTACAAAGTGAAGCTCCGCGGCACTAACTTCCCTTCTGATGGCCCGG
TCATGCAGAAGAAAACCATGGGCTGGGAAGCCAGCACGGAGAGAATGTAC
CCTGAGGACGGTGCCCTGAAAGGTGAGATAAAGATGCGGCTGAAGTTGAA
GGACGGAGGGCACTATGATGCTGAGGTCAAGACCACCTATATGGCGAAGA
AGCCTGTCCAGCTGCCAGGAGCCTACAAGACAGACATTAAACTTGATATC
ACCTCTCACAATGAAGACTATACCATTGTGGAGCAGTATGAGCGTGCAGA
GGGACGGCACAGCACTGGGGCGTGAGCTGGAGCCTCGGTGGCCATGCTTC
TTGCCCCTTGGGCCTCCCCCCAGCCCCTCCTCCCCTTCCTGCACCCGTAC
CCCCGTGGTCTTTGAATAAAGTCTGACTCAGTAGATGTTTTCTTGGGTTA
ATTGAGGCCTGAGTATAAGGTGACTTATACTTGTAATCTATCTAAACGGG
GAACCTCTCTAGTAGACAATCCCGTGCTAAATTGTAGGACTGCCCTTTAA
TAAATACTTCTATATTTAAAGAGGTATTTATGAAAAGCGGAATTTATCAG
ATTAAAAATACTTTCTCTAGAGTCGACCTGCAG
DNA sequence of EMCV IRES-mFOXP3-
himeric intron-EMCV IRES-Thy 1.1
(T7 promoter (nucleotides 1-19)
T4 td upstream (nucleotides 21-275)
5′UTR (PABPv3) (nucleotides 277-326)
EMCV IRES (nucleotides 327-894)
mFOXP3 (nucleotides 1035-2327)
Chimeric intron (nucleotides
2413-2642) THY1.1 (nucleotides
3324-3812) 3′UTR (HBA1 full length)
(nucleotides 3813-3913) T4 td
Downstream (nucleotides 3914-4120)
SEQ ID NO: 66
TAATACGACTCACTATAGGGGGGAATTCTAGAGAAAATTTCGTCTGGATT
AGTTACTTATCGTGTAAAATCTGATAAATGGAATTGGTTCTACATAAATG
CCTAACGACTATCCCTTTGGGGAGTAGGGTCAAGTGACTCGAAACGATAG
ACAACTTGCTTTAACAAGTTGGAGATATAGTCTGCTCTGCATGGTGACAT
GCAGCTGGATATAATTCCGGGGTAAGATTAACGACCTTATCTGAACATAA
CGCTACCGTTTAATATTGCGTCATATAAAAAAAAAAAACCAAAAAAAAAA
AACAAAAAAAAAAAATAATTGACTAATCCCTCCCCCCCCCCTAACGTTAC
TGGCCGAAGCCGCTTGGAATAAGGCCGGTGTGCGTTTGTCTATATGTTAT
TTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGG
AATGCAAGGTCTGTTGAATGTCGTGAAGGAAGCAGTTCCTCTGGAAGCTT
CTTGAAGACAAACAACGTCTGTAGCGACCCTTTGCAGGCAGCGGAACCCC
CCACCTGGCGACAGGTGCCTCTGCGGCCAAAAGCCACGTGTATAAGATAC
ACCTGCAAAGGCGGCACAACCCCAGTGCCACGTTGTGAGTTGGATAGTTG
TGGAAAGAGTCAAATGGCTCTCCTCAAGCGTATTCAACAAGGGGCTGAAG
GATGCCCAGAAGGTACCCCATTGTATGGGATCTGATCTGGGGCCTCGGTG
CACATGCTTTACATGTGTTTAGTCGAGGTTAAAAAAACGTCTAGGCCCCC
CGAACCACGGGGACGTGGTTTTCCTTTGAAAAACACGATGATAAGCTTGC
CACAACCCGGGATCCTCTAGGAGTACTGAGCTCGTTTAGTGAACCGTCAG
ATCGCCTGGAGACGCCATCCACGCTGTTTTGACCTCCATAGAAGACACCG
GGACCGATCCAGCCTCCGCGGCCCCGAATTCACCATGCCAAACCCCCGTC
CTGCCAAGCCCATGGCTCCTTCTCTTGCACTGGGACCCTCTCCTGGGGTG
CTTCCATCCTGGAAAACAGCCCCCAAAGGCTCGGAATTACTCGGCACTCG
AGGCTCTGGAGGGCCTTTCCAGGGCAGAGATCTCCGCAGTGGTGCTCACA
CAAGTAGTAGCTTAAATCCACTCCCGCCCAGCCAGCTACAACTGCCCACT
GTTCCACTGGTGATGGTCGCACCCAGTGGTGCCAGGTTGGGCCCCTCACC
ACATTTGCAAGCACTTCTGCAGGACAGGCCTCACTTCATGCACCAGCTGT
CGACGGTGGACGCTCATGCCCAGACACCTGTCCTTCAGGTTCGCCCCCTG
GACAACCCTGCTATGATCTCTCTACCGCCCCCATCAGCAGCCACAGGAGT
CTTCTCTCTCAAAGCTCGGCCAGGGCTGCCTCCAGGAATTAATGTTGCTT
CCCTTGAGTGGGTATCACGTGAGCCCGCCTTGCTGTGTACATTTCCTCGG
TCAGGAACTCCCCGGAAAGACAGCAACTTGTTAGCTGCACCTCAAGGCTC
CTATCCACTGCTTGCCAACGGTGTCTGCAAGTGGCCTGGCTGTGAGAAGG
TGTTTGAAGAGCCTGAGGAGTTCCTGAAGCACTGCCAGGCTGACCACCTG
CTGGATGAAAAAGGGAAAGCTCAGTGTCTGCTCCAAAGGGAAGTGGTGCA
GAGCTTGGAGCAGCAGTTGGAACTGGAAAAGGAGAAACTTGGTGCCATGC
AAGCCCATCTCGCAGGCAAGATGGCCTTAGCCAAAGCACCGAGCGTGGCC
TCCATGGACAAGTCCTCCTGCTGCATTGTAGCCACCAGTACCCAGGGAAG
CGTACTGCCAGCGTGGAGCGCGCCGCGGGAAGCACCTGATGGGGGCCTGT
TTGCCGTCAGACGCCATCTGTGGGGTAGCCATGGGAATTCTTCCTTCCCA
GAATTCTTCCACAACATGGATTACTTCAAGTATCATAATATGAGGCCACC
CTTCACCTATGCCACTCTCATCCGCTGGGCAATATTGGAAGCCCCAGAGA
GACAGAGGACGCTGAATGAGATTTACCACTGGTTTACCAGGATGTTTGCA
TACTTTAGAAACCACCCAGCTACCTGGAAGAATGCCATCCGACATAACCT
CTCCCTGCACAAGTGCTTTGTTCGAGTGGAGTCAGAAAAAGGAGCTGTGT
GGACAGTGGATGAGTTTGAGTTCAGAAAGAAGAGATCCCAGCGGCCCAAC
AAGTGTAGCAATCCATGTCCATGATAATCGAGGTTAATTAATGAGCGGCC
GCATAGATAACTGATCCAGTGTGCTGGAATTAATTCGCTGTCTGCGAGGG
CCAGCTGTTGGGGTGAGTACTCCCTCTCAAAAGCGGGCATGACTTCTGCG
CTAAGATTGTCAGTTTCCAAAAACGAGGAGGATTTGATATTCACCTGGCC
CGCGGTGATGCCTTTGAGGGTGGCCGCGTCCATCTGGTCAGAAAAGACAA
TCTTTTTGTTGTCAAGCTTGAGGTGTGGCAGGCTTGAGATCTGGCCATAC
ACTTGAGTGACAATGACATCCACTTTGCCTTTCTCTCCACAGGTGTCCAC
TCCCAGGTCCAACTGCAGGTCGAGCATGCATCTAGGGCGGCCAATTCCGC
CCCTCTCCCCCCCCCCCTTTTCCCTCCCCCCCCCCTAACGTTACTGGCCG
AAGCCGCTTGGAATAAGGCCGGTGTGCGTTTGTCTATATGTTATTTTCCA
CCATATTGCCGTCTTTTGGCAATGTGAGGGCCCGGAAACCTGGCCCTGTC
TTCTTGACGAGCATTCCTAGGGGTCTTTCCCCTCTCGCCAAAGGAATGCA
AGGTCTGTTGAATGTCGTGAAGGAAGCAGTTCCTCTGGAAGCTTCTTGAA
GACAAACAACGTCTGTAGCGACCCTTTGCAGGCAGCGGAACCCCCCACCT
GGCGACAGGTGCCTCTGCGGCCAAAAGCCACGTGTATAAGATACACCTGC
AAAGGCGGCACAACCCCAGTGCCACGTTGTGAGTTGGATAGTTGTGGAAA
GAGTCAAATGGCTCTCCTCAAGCGTATTCAACAAGGGGCTGAAGGATGCC
CAGAAGGTACCCCATTGTATGGGATCTGATCTGGGGCCTCGGTGCACATG
CTTTACATGTGTTTAGTCGAGGTTAAAAAAACGTCTAGGCCCCCCGAACC
ACGGGGACGTGGTTTTCCTTTGAAAAACACGATGATAAGCTTGCCACAAC
CCGGGATAATTCCTGCAGCCAATATGAACCCAGCCATCAGCGTCGCTCTC
CTGCTCTCAGTCTTGCAGGTGTCCCGAGGGCAGAAGGTGACCAGCCTGAC
AGCCTGCCTGGTGAACCAAAACCTTCGCCTGGACTGCCGCCATGAGAATA
ACACCAAGGATAACTCCATCCAGCATGAGTTCAGCCTGACCCGAGAGAAG
AGGAAGCACGTGCTCTCAGGCACCCTTGGGATACCCGAGCACACGTACCG
CTCCCGCGTCACCCTCTCCAACCAGCCCTATATCAAGGTCCTTACCCTAG
CCAACTTCACCACCAAGGATGAGGGCGACTACTTTTGTGAGCTTCGCGTC
TCGGGCGCGAATCCCATGAGCTCCAATAAAAGTATCAGTGTGTATAGAGA
CAAGCTGGTCAAGTGTGGCGGCATAAGCCTGCTGGTTCAGAACACATCCT
GGATGCTGCTGCTGCTGCTTTCCCTCTCCCTCCTCCAAGCCCTGGACTTC
ATTTCTCTGTGAGCTGGAGCCTCGGTGGCCATGCTTCTTGCCCCTTGGGC
CTCCCCCCAGCCCCTCCTCCCCTTCCTGCACCCGTACCCCCGTGGTCTTT
GAATAAAGTCTGACTCAGTAGATGTTTTCTTGGGTTAATTGAGGCCTGAG
TATAAGGTGACTTATACTTGTAATCTATCTAAACGGGGAACCTCTCTAGT
AGACAATCCCGTGCTAAATTGTAGGACTGCCCTTTAATAAATACTTCTAT
ATTTAAAGAGGTATTTATGAAAAGCGGAATTTATCAGATTAAAAATACTT
TCTCTAGAGTCGACCTGCAG
DNA sequence of EVB 107 IRES-NeonGreen
T7 promoter (T7 promoter (nucleotides
1-19) T4 td upstream (nucleotides 21-275)
5′UTR (PABPv3) (nucleotides 277-326)
EVB107 IRES (nucleotides 327-1069)
NeonGreen (nucleotides 1210-1926) 3′UTR
(HBA1 full length) (nucleotides 1927-2027)
T4 td Downstream (nucleotides 2028-2234))
SEQ ID NO: 67
TAATACGACTCACTATAGGGGGGAATTCTAGAGAAAATTTCGTCTGGATT
AGTTACTTATCGTGTAAAATCTGATAAATGGAATTGGTTCTACATAAATG
CCTAACGACTATCCCTTTGGGGAGTAGGGTCAAGTGACTCGAAACGATAG
ACAACTTGCTTTAACAAGTTGGAGATATAGTCTGCTCTGCATGGTGACAT
GCAGCTGGATATAATTCCGGGGTAAGATTAACGACCTTATCTGAACATAA
CGCTACCGTTTAATATTGCGTCATATAAAAAAAAAAAACCAAAAAAAAAA
AACAAAAAAAAAAAATAATTGACTAATTAAAACAGCCTGTGGGTTGTTCC
CACCCGCAGGGCCCACTGGGCGCTAGCACACTGGTATCCCGGTACCCTTG
TGCGCCTGTTTTATATACCCTCCCCCTTATGTAACTTAGAAGTATGATTC
AAACGGTCGACAGGCGGCTCAGTGCACCAACTGAGTCATGACCAAGCACT
TCTGTTACCCCGGACTGAGTATCAATAAGCTGTTCACACGGCTGAAGGAG
AAAACGTTCGTTACCCGGCCAATTACTTCGAGAAACCTAGTACCACCATG
AAGGTTGCGCAGTGTTTCGCTCCACACAACCCCAGTGTAGATCAGGTCGA
TGAGTCACCGCATTCCCCACGGGCGACCGTGGCGGTGGCTGCGTTGGCGG
CCTGCCCATGGGGCAACCCATGGGACGCTTCAATACTGACATGGTGTGAA
GAGTCTATTGAGCTAATTGGTAGTCCTCCGGCCCCTGAATGCGGCTAATC
CCAACTGTGGAGCAGATACTCACAAACCAGTGAGCGGTCTGTCGTAACGG
GCAACTCCGCAGCGGAACCGACTACTTTGGGTGTCCGTGTTTCTTTTTAT
TCTTACATTGGCTGCTTATGGTGACAATTGACAAATTGTTACCATATAGC
TATTGGATTGGCCATCCGGTGACAAACAGAGCTATTGTTTACTTGTTTGT
TGGTTTCATACCATTAAATTACAAGGTCTTAGAAACTCTCAACTTTATTT
TGACACTCAATACAGCAAAGCTTGCCACAACCCGGGATCCTCTAGGAGTA
CTGAGCTCGTTTAGTGAACCGTCAGATCGCCTGGAGACGCCATCCACGCT
GTTTTGACCTCCATAGAAGACACCGGGACCGATCCAGCCTCCGCGGCCCC
GAATTCACCATGGGCTCTGTGTCTAAAGGAGAAGAAGACAACATGGCGAG
TCTGCCGGCGACACATGAGCTGCACATCTTTGGATCCATCAACGGTGTCG
ACTTTGACATGGTTGGCCAAGGCACCGGAAATCCAAATGATGGCTATGAG
GAGCTGAACCTGAAGAGTACGAAAGGAGACCTGCAGTTCTCCCCCTGGAT
CCTGGTGCCTCACATTGGCTATGGCTTCCACCAGTATCTGCCCTACCCAG
ACGGCATGAGCCCTTTCCAGGCTGCCATGGTGGATGGATCAGGCTACCAG
GTGCACAGGACCATGCAGTTTGAGGATGGGGCTTCGTTGACTGTGAACTA
CAGATACACATATGAAGGGTCTCATATTAAAGGAGAGGCCCAGGTGAAGG
GGACAGGCTTCCCTGCTGACGGTCCTGTCATGACCAACAGCCTGACTGCA
GCTGACTGGTGCCGCAGCAAAAAGACGTACCCCAATGACAAGACCATCAT
AAGCACTTTCAAGTGGAGCTACACCACTGGGAATGGCAAGAGGTACCGCT
CCACTGCCCGTACCACCTATACCTTTGCCAAGCCAATGGCTGCCAATTAC
CTGAAGAACCAGCCCATGTACGTCTTCCGGAAGACAGAGTTGAAGCACAG
CAAAACTGAACTCAACTTCAAAGAGTGGCAGAAGGCCTTCACAGATGTGA
TGGGGATGGATGAGCTCTACAAATGAGCTGGAGCCTCGGTGGCCATGCTT
CTTGCCCCTTGGGCCTCCCCCCAGCCCCTCCTCCCCTTCCTGCACCCGTA
CCCCCGTGGTCTTTGAATAAAGTCTGACTCAGTAGATGTTTTCTTGGGTT
AATTGAGGCCTGAGTATAAGGTGACTTATACTTGTAATCTATCTAAACGG
GGAACCTCTCTAGTAGACAATCCCGTGCTAAATTGTAGGACTGCCCTTTA
ATAAATACTTCTATATTTAAAGAGGTATTTATGAAAAGCGGAATTTATCA
GATTAAAAATACTTTCTCTAGAGTCGACCTGCAG
DNA sequence of EVB 107 IRES-RFP
(T7 promoter (nucleotides 1-19)
T4 td upstream (nucleotides 21-275)
5′UTR (PABPv3) (nucleotides 277-326)
EVB107 IRES (nucleotides 327-1069)
RFP (nucleotides 1151-1782) 3′UTR
(HBA1 full length) (nucleotides
1783-1883) T4 td Downstream
(nucleotides 1884-2090))
SEQ ID NO: 68
TAATACGACTCACTATAGGGGGGAATTCTAGAGAAAATTTCGTCTGGATT
AGTTACTTATCGTGTAAAATCTGATAAATGGAATTGGTTCTACATAAATG
CCTAACGACTATCCCTTTGGGGAGTAGGGTCAAGTGACTCGAAACGATAG
ACAACTTGCTTTAACAAGTTGGAGATATAGTCTGCTCTGCATGGTGACAT
GCAGCTGGATATAATTCCGGGGTAAGATTAACGACCTTATCTGAACATAA
CGCTACCGTTTAATATTGCGTCATATAAAAAAAAAAAACCAAAAAAAAAA
AACAAAAAAAAAAAATAATTGACTAATTAAAACAGCCTGTGGGTTGTTCC
CACCCGCAGGGCCCACTGGGCGCTAGCACACTGGTATCCCGGTACCCTTG
TGCGCCTGTTTTATATACCCTCCCCCTTATGTAACTTAGAAGTATGATTC
AAACGGTCGACAGGCGGCTCAGTGCACCAACTGAGTCATGACCAAGCACT
TCTGTTACCCCGGACTGAGTATCAATAAGCTGTTCACACGGCTGAAGGAG
AAAACGTTCGTTACCCGGCCAATTACTTCGAGAAACCTAGTACCACCATG
AAGGTTGCGCAGTGTTTCGCTCCACACAACCCCAGTGTAGATCAGGTCGA
TGAGTCACCGCATTCCCCACGGGCGACCGTGGCGGTGGCTGCGTTGGCGG
CCTGCCCATGGGGCAACCCATGGGACGCTTCAATACTGACATGGTGTGAA
GAGTCTATTGAGCTAATTGGTAGTCCTCCGGCCCCTGAATGCGGCTAATC
CCAACTGTGGAGCAGATACTCACAAACCAGTGAGCGGTCTGTCGTAACGG
GCAACTCCGCAGCGGAACCGACTACTTTGGGTGTCCGTGTTTCTTTTTAT
TCTTACATTGGCTGCTTATGGTGACAATTGACAAATTGTTACCATATAGC
TATTGGATTGGCCATCCGGTGACAAACAGAGCTATTGTTTACTTGTTTGT
TGGTTTCATACCATTAAATTACAAGGTCTTAGAAACTCTCAACTTTATTT
TGACACTCAATACAGCAAAGCTTGCCACAACCCGGGATAATTCCTGCAGC
CAATATGGCTTCATCGGAAGACGTCATCAAAGAATTCATGAGGTTCAAGG
TTCGCATGGAGGGCAGTGTGAACGGCCATGAGTTTGAAATCGAGGGTGAG
GGGGAGGGCCGCCCCTACGAAGGGACACAGACCGCCAAACTAAAGGTCAC
CAAAGGGGGCCCGTTGCCCTTTGCCTGGGACATCTTATCCCCCCAGTTCC
AGTACGGGAGCAAGGCCTATGTAAAACACCCTGCTGACATTCCAGACTAC
CTTAAACTGAGTTTCCCAGAAGGCTTCAAGTGGGAGAGGGTGATGAACTT
TGAGGATGGCGGAGTGGTGACAGTGACTCAAGACTCCTCCCTGCAGGATG
GGGAGTTCATCTACAAAGTGAAGCTCCGCGGCACTAACTTCCCTTCTGAT
GGCCCGGTCATGCAGAAGAAAACCATGGGCTGGGAAGCCAGCACGGAGAG
AATGTACCCTGAGGACGGTGCCCTGAAAGGTGAGATAAAGATGCGGCTGA
AGTTGAAGGACGGAGGGCACTATGATGCTGAGGTCAAGACCACCTATATG
GCGAAGAAGCCTGTCCAGCTGCCAGGAGCCTACAAGACAGACATTAAACT
TGATATCACCTCTCACAATGAAGACTATACCATTGTGGAGCAGTATGAGC
GTGCAGAGGGACGGCACAGCACTGGGGCGTGAGCTGGAGCCTCGGTGGCC
ATGCTTCTTGCCCCTTGGGCCTCCCCCCAGCCCCTCCTCCCCTTCCTGCA
CCCGTACCCCCGTGGTCTTTGAATAAAGTCTGACTCAGTAGATGTTTTCT
TGGGTTAATTGAGGCCTGAGTATAAGGTGACTTATACTTGTAATCTATCT
AAACGGGGAACCTCTCTAGTAGACAATCCCGTGCTAAATTGTAGGACTGC
CCTTTAATAAATACTTCTATATTTAAAGAGGTATTTATGAAAAGCGGAAT
TTATCAGATTAAAAATACTTTCTCTAGAGTCGACCTGCAG
DNA sequence of EVB 107 IRES-NeonGreen-
chimeric intron-EVB 107 IRES-RFP (T7
promoter (nucleotides 1-19) T4 td
upstream (nucleotides 21-275) 5′UTR
(PABPv3) (nucleotides 277-326) EVB107
IRES (nucleotides 327-1069) NeonGreen
(nucleotides 1210-1926) NeonGreen
(nucleotides 1210-1926) chimeric intron
(nucleotides 2012-2241) EVB107 IRES
(nucleotides 2320-3062) RFP (nucleotides
4084-3775) 3′UTR (HBA1 full length)
(nucleotides 3776-3876) T4 td Downstream
(nucleotides 3877-4083))
SEQ ID NO: 69
TAATACGACTCACTATAGGGGGGAATTCTAGAGAAAATTTCGTCTGGATT
AGTTACTTATCGTGTAAAATCTGATAAATGGAATTGGTTCTACATAAATG
CCTAACGACTATCCCTTTGGGGAGTAGGGTCAAGTGACTCGAAACGATAG
ACAACTTGCTTTAACAAGTTGGAGATATAGTCTGCTCTGCATGGTGACAT
GCAGCTGGATATAATTCCGGGGTAAGATTAACGACCTTATCTGAACATAA
CGCTACCGTTTAATATTGCGTCATATAAAAAAAAAAAACCAAAAAAAAAA
AACAAAAAAAAAAAATAATTGACTAATTAAAACAGCCTGTGGGTTGTTCC
CACCCGCAGGGCCCACTGGGCGCTAGCACACTGGTATCCCGGTACCCTTG
TGCGCCTGTTTTATATACCCTCCCCCTTATGTAACTTAGAAGTATGATTC
AAACGGTCGACAGGCGGCTCAGTGCACCAACTGAGTCATGACCAAGCACT
TCTGTTACCCCGGACTGAGTATCAATAAGCTGTTCACACGGCTGAAGGAG
AAAACGTTCGTTACCCGGCCAATTACTTCGAGAAACCTAGTACCACCATG
AAGGTTGCGCAGTGTTTCGCTCCACACAACCCCAGTGTAGATCAGGTCGA
TGAGTCACCGCATTCCCCACGGGCGACCGTGGCGGTGGCTGCGTTGGCGG
CCTGCCCATGGGGCAACCCATGGGACGCTTCAATACTGACATGGTGTGAA
GAGTCTATTGAGCTAATTGGTAGTCCTCCGGCCCCTGAATGCGGCTAATC
CCAACTGTGGAGCAGATACTCACAAACCAGTGAGCGGTCTGTCGTAACGG
GCAACTCCGCAGCGGAACCGACTACTTTGGGTGTCCGTGTTTCTTTTTAT
TCTTACATTGGCTGCTTATGGTGACAATTGACAAATTGTTACCATATAGC
TATTGGATTGGCCATCCGGTGACAAACAGAGCTATTGTTTACTTGTTTGT
TGGTTTCATACCATTAAATTACAAGGTCTTAGAAACTCTCAACTTTATTT
TGACACTCAATACAGCAAAGCTTGCCACAACCCGGGATCCTCTAGGAGTA
CTGAGCTCGTTTAGTGAACCGTCAGATCGCCTGGAGACGCCATCCACGCT
GTTTTGACCTCCATAGAAGACACCGGGACCGATCCAGCCTCCGCGGCCCC
GAATTCACCATGGGCTCTGTGTCTAAAGGAGAAGAAGACAACATGGCGAG
TCTGCCGGCGACACATGAGCTGCACATCTTTGGATCCATCAACGGTGTCG
ACTTTGACATGGTTGGCCAAGGCACCGGAAATCCAAATGATGGCTATGAG
GAGCTGAACCTGAAGAGTACGAAAGGAGACCTGCAGTTCTCCCCCTGGAT
CCTGGTGCCTCACATTGGCTATGGCTTCCACCAGTATCTGCCCTACCCAG
ACGGCATGAGCCCTTTCCAGGCTGCCATGGTGGATGGATCAGGCTACCAG
GTGCACAGGACCATGCAGTTTGAGGATGGGGCTTCGTTGACTGTGAACTA
CAGATACACATATGAAGGGTCTCATATTAAAGGAGAGGCCCAGGTGAAGG
GGACAGGCTTCCCTGCTGACGGTCCTGTCATGACCAACAGCCTGACTGCA
GCTGACTGGTGCCGCAGCAAAAAGACGTACCCCAATGACAAGACCATCAT
AAGCACTTTCAAGTGGAGCTACACCACTGGGAATGGCAAGAGGTACCGCT
CCACTGCCCGTACCACCTATACCTTTGCCAAGCCAATGGCTGCCAATTAC
CTGAAGAACCAGCCCATGTACGTCTTCCGGAAGACAGAGTTGAAGCACAG
CAAAACTGAACTCAACTTCAAAGAGTGGCAGAAGGCCTTCACAGATGTGA
TGGGGATGGATGAGCTCTACAAATGATCGAGGTTAATTAATGAGCGGCCG
CATAGATAACTGATCCAGTGTGCTGGAATTAATTCGCTGTCTGCGAGGGC
CAGCTGTTGGGGTGAGTACTCCCTCTCAAAAGCGGGCATGACTTCTGCGC
TAAGATTGTCAGTTTCCAAAAACGAGGAGGATTTGATATTCACCTGGCCC
GCGGTGATGCCTTTGAGGGTGGCCGCGTCCATCTGGTCAGAAAAGACAAT
CTTTTTGTTGTCAAGCTTGAGGTGTGGCAGGCTTGAGATCTGGCCATACA
CTTGAGTGACAATGACATCCACTTTGCCTTTCTCTCCACAGGTGTCCACT
CCCAGGTCCAACTGCAGGTCGAGCATGCATCTAGGGCGGCCAATTCCGCC
CCTCTCCCCCCCCCCCTTTTTAAAACAGCCTGTGGGTTGTTCCCACCCGC
AGGGCCCACTGGGCGCTAGCACACTGGTATCCCGGTACCCTTGTGCGCCT
GTTTTATATACCCTCCCCCTTATGTAACTTAGAAGTATGATTCAAACGGT
CGACAGGCGGCTCAGTGCACCAACTGAGTCATGACCAAGCACTTCTGTTA
CCCCGGACTGAGTATCAATAAGCTGTTCACACGGCTGAAGGAGAAAACGT
TCGTTACCCGGCCAATTACTTCGAGAAACCTAGTACCACCATGAAGGTTG
CGCAGTGTTTCGCTCCACACAACCCCAGTGTAGATCAGGTCGATGAGTCA
CCGCATTCCCCACGGGCGACCGTGGCGGTGGCTGCGTTGGCGGCCTGCCC
ATGGGGCAACCCATGGGACGCTTCAATACTGACATGGTGTGAAGAGTCTA
TTGAGCTAATTGGTAGTCCTCCGGCCCCTGAATGCGGCTAATCCCAACTG
TGGAGCAGATACTCACAAACCAGTGAGCGGTCTGTCGTAACGGGCAACTC
CGCAGCGGAACCGACTACTTTGGGTGTCCGTGTTTCTTTTTATTCTTACA
TTGGCTGCTTATGGTGACAATTGACAAATTGTTACCATATAGCTATTGGA
TTGGCCATCCGGTGACAAACAGAGCTATTGTTTACTTGTTTGTTGGTTTC
ATACCATTAAATTACAAGGTCTTAGAAACTCTCAACTTTATTTTGACACT
CAATACAGCAAAGCTTGCCACAACCCGGGATAATTCCTGCAGCCAATATG
GCTTCATCGGAAGACGTCATCAAAGAATTCATGAGGTTCAAGGTTCGCAT
GGAGGGCAGTGTGAACGGCCATGAGTTTGAAATCGAGGGTGAGGGGGAGG
GCCGCCCCTACGAAGGGACACAGACCGCCAAACTAAAGGTCACCAAAGGG
GGCCCGTTGCCCTTTGCCTGGGACATCTTATCCCCCCAGTTCCAGTACGG
GAGCAAGGCCTATGTAAAACACCCTGCTGACATTCCAGACTACCTTAAAC
TGAGTTTCCCAGAAGGCTTCAAGTGGGAGAGGGTGATGAACTTTGAGGAT
GGCGGAGTGGTGACAGTGACTCAAGACTCCTCCCTGCAGGATGGGGAGTT
CATCTACAAAGTGAAGCTCCGCGGCACTAACTTCCCTTCTGATGGCCCGG
TCATGCAGAAGAAAACCATGGGCTGGGAAGCCAGCACGGAGAGAATGTAC
CCTGAGGACGGTGCCCTGAAAGGTGAGATAAAGATGCGGCTGAAGTTGAA
GGACGGAGGGCACTATGATGCTGAGGTCAAGACCACCTATATGGCGAAGA
AGCCTGTCCAGCTGCCAGGAGCCTACAAGACAGACATTAAACTTGATATC
ACCTCTCACAATGAAGACTATACCATTGTGGAGCAGTATGAGCGTGCAGA
GGGACGGCACAGCACTGGGGCGTGAGCTGGAGCCTCGGTGGCCATGCTTC
TTGCCCCTTGGGCCTCCCCCCAGCCCCTCCTCCCCTTCCTGCACCCGTAC
CCCCGTGGTCTTTGAATAAAGTCTGACTCAGTAGATGTTTTCTTGGGTTA
ATTGAGGCCTGAGTATAAGGTGACTTATACTTGTAATCTATCTAAACGGG
GAACCTCTCTAGTAGACAATCCCGTGCTAAATTGTAGGACTGCCCTTTAA
TAAATACTTCTATATTTAAAGAGGTATTTATGAAAAGCGGAATTTATCAG
ATTAAAAATACTTTCTCTAGAGTCGACCTGCAG
DNA sequence of EVB 107 IRES-mFOXP3-
chimeric intron-EVB 107 IRES-THY1.1
(T7 promoter (nucleotides 1-19) T4 td
upstream (nucleotides 21-275) 5′UTR
(PABPv3) (nucleotides 277-326) EVB107
IRES (nucleotides 327-1069) mFOXP3
(nucleotides 1210-2502) chimeric
intron (nucleotides 4471-2817) EVB107
IRES (nucleotides 2896-3638) THY1.1
(nucleotides 3674-4162) 3′UTR (HBA1
full length) (nucleotides 4163-4263)
T4 td Downstream (nucleotides 4264-4470))
SEQ ID NO: 70
TAATACGACTCACTATAGGGGGGAATTCTAGAGAAAATTTCGTCTGGATT
AGTTACTTATCGTGTAAAATCTGATAAATGGAATTGGTTCTACATAAATG
CCTAACGACTATCCCTTTGGGGAGTAGGGTCAAGTGACTCGAAACGATAG
ACAACTTGCTTTAACAAGTTGGAGATATAGTCTGCTCTGCATGGTGACAT
GCAGCTGGATATAATTCCGGGGTAAGATTAACGACCTTATCTGAACATAA
CGCTACCGTTTAATATTGCGTCATATAAAAAAAAAAAACCAAAAAAAAAA
AACAAAAAAAAAAAATAATTGACTAATTAAAACAGCCTGTGGGTTGTTCC
CACCCGCAGGGCCCACTGGGCGCTAGCACACTGGTATCCCGGTACCCTTG
TGCGCCTGTTTTATATACCCTCCCCCTTATGTAACTTAGAAGTATGATTC
AAACGGTCGACAGGCGGCTCAGTGCACCAACTGAGTCATGACCAAGCACT
TCTGTTACCCCGGACTGAGTATCAATAAGCTGTTCACACGGCTGAAGGAG
AAAACGTTCGTTACCCGGCCAATTACTTCGAGAAACCTAGTACCACCATG
AAGGTTGCGCAGTGTTTCGCTCCACACAACCCCAGTGTAGATCAGGTCGA
TGAGTCACCGCATTCCCCACGGGCGACCGTGGCGGTGGCTGCGTTGGCGG
CCTGCCCATGGGGCAACCCATGGGACGCTTCAATACTGACATGGTGTGAA
GAGTCTATTGAGCTAATTGGTAGTCCTCCGGCCCCTGAATGCGGCTAATC
CCAACTGTGGAGCAGATACTCACAAACCAGTGAGCGGTCTGTCGTAACGG
GCAACTCCGCAGCGGAACCGACTACTTTGGGTGTCCGTGTTTCTTTTTAT
TCTTACATTGGCTGCTTATGGTGACAATTGACAAATTGTTACCATATAGC
TATTGGATTGGCCATCCGGTGACAAACAGAGCTATTGTTTACTTGTTTGT
TGGTTTCATACCATTAAATTACAAGGTCTTAGAAACTCTCAACTTTATTT
TGACACTCAATACAGCAAAGCTTGCCACAACCCGGGATCCTCTAGGAGTA
CTGAGCTCGTTTAGTGAACCGTCAGATCGCCTGGAGACGCCATCCACGCT
GTTTTGACCTCCATAGAAGACACCGGGACCGATCCAGCCTCCGCGGCCCC
GAATTCACCATGCCAAACCCCCGTCCTGCCAAGCCCATGGCTCCTTCTCT
TGCACTGGGACCCTCTCCTGGGGTGCTTCCATCCTGGAAAACAGCCCCCA
AAGGCTCGGAATTACTCGGCACTCGAGGCTCTGGAGGGCCTTTCCAGGGC
AGAGATCTCCGCAGTGGTGCTCACACAAGTAGTAGCTTAAATCCACTCCC
GCCCAGCCAGCTACAACTGCCCACTGTTCCACTGGTGATGGTCGCACCCA
GTGGTGCCAGGTTGGGCCCCTCACCACATTTGCAAGCACTTCTGCAGGAC
AGGCCTCACTTCATGCACCAGCTGTCGACGGTGGACGCTCATGCCCAGAC
ACCTGTCCTTCAGGTTCGCCCCCTGGACAACCCTGCTATGATCTCTCTAC
CGCCCCCATCAGCAGCCACAGGAGTCTTCTCTCTCAAAGCTCGGCCAGGG
CTGCCTCCAGGAATTAATGTTGCTTCCCTTGAGTGGGTATCACGTGAGCC
CGCCTTGCTGTGTACATTTCCTCGGTCAGGAACTCCCCGGAAAGACAGCA
ACTTGTTAGCTGCACCTCAAGGCTCCTATCCACTGCTTGCCAACGGTGTC
TGCAAGTGGCCTGGCTGTGAGAAGGTGTTTGAAGAGCCTGAGGAGTTCCT
GAAGCACTGCCAGGCTGACCACCTGCTGGATGAAAAAGGGAAAGCTCAGT
GTCTGCTCCAAAGGGAAGTGGTGCAGAGCTTGGAGCAGCAGTTGGAACTG
GAAAAGGAGAAACTTGGTGCCATGCAAGCCCATCTCGCAGGCAAGATGGC
CTTAGCCAAAGCACCGAGCGTGGCCTCCATGGACAAGTCCTCCTGCTGCA
TTGTAGCCACCAGTACCCAGGGAAGCGTACTGCCAGCGTGGAGCGCGCCG
CGGGAAGCACCTGATGGGGGCCTGTTTGCCGTCAGACGCCATCTGTGGGG
TAGCCATGGGAATTCTTCCTTCCCAGAATTCTTCCACAACATGGATTACT
TCAAGTATCATAATATGAGGCCACCCTTCACCTATGCCACTCTCATCCGC
TGGGCAATATTGGAAGCCCCAGAGAGACAGAGGACGCTGAATGAGATTTA
CCACTGGTTTACCAGGATGTTTGCATACTTTAGAAACCACCCAGCTACCT
GGAAGAATGCCATCCGACATAACCTCTCCCTGCACAAGTGCTTTGTTCGA
GTGGAGTCAGAAAAAGGAGCTGTGTGGACAGTGGATGAGTTTGAGTTCAG
AAAGAAGAGATCCCAGCGGCCCAACAAGTGTAGCAATCCATGTCCATGAT
AATCGAGGTTAATTAATGAGCGGCCGCATAGATAACTGATCCAGTGTGCT
GGAATTAATTCGCTGTCTGCGAGGGCCAGCTGTTGGGGTGAGTACTCCCT
CTCAAAAGCGGGCATGACTTCTGCGCTAAGATTGTCAGTTTCCAAAAACG
AGGAGGATTTGATATTCACCTGGCCCGCGGTGATGCCTTTGAGGGTGGCC
GCGTCCATCTGGTCAGAAAAGACAATCTTTTTGTTGTCAAGCTTGAGGTG
TGGCAGGCTTGAGATCTGGCCATACACTTGAGTGACAATGACATCCACTT
TGCCTTTCTCTCCACAGGTGTCCACTCCCAGGTCCAACTGCAGGTCGAGC
ATGCATCTAGGGCGGCCAATTCCGCCCCTCTCCCCCCCCCCCTTTTTAAA
ACAGCCTGTGGGTTGTTCCCACCCGCAGGGCCCACTGGGCGCTAGCACAC
TGGTATCCCGGTACCCTTGTGCGCCTGTTTTATATACCCTCCCCCTTATG
TAACTTAGAAGTATGATTCAAACGGTCGACAGGCGGCTCAGTGCACCAAC
TGAGTCATGACCAAGCACTTCTGTTACCCCGGACTGAGTATCAATAAGCT
GTTCACACGGCTGAAGGAGAAAACGTTCGTTACCCGGCCAATTACTTCGA
GAAACCTAGTACCACCATGAAGGTTGCGCAGTGTTTCGCTCCACACAACC
CCAGTGTAGATCAGGTCGATGAGTCACCGCATTCCCCACGGGCGACCGTG
GCGGTGGCTGCGTTGGCGGCCTGCCCATGGGGCAACCCATGGGACGCTTC
AATACTGACATGGTGTGAAGAGTCTATTGAGCTAATTGGTAGTCCTCCGG
CCCCTGAATGCGGCTAATCCCAACTGTGGAGCAGATACTCACAAACCAGT
GAGCGGTCTGTCGTAACGGGCAACTCCGCAGCGGAACCGACTACTTTGGG
TGTCCGTGTTTCTTTTTATTCTTACATTGGCTGCTTATGGTGACAATTGA
CAAATTGTTACCATATAGCTATTGGATTGGCCATCCGGTGACAAACAGAG
CTATTGTTTACTTGTTTGTTGGTTTCATACCATTAAATTACAAGGTCTTA
GAAACTCTCAACTTTATTTTGACACTCAATACAGCAAAGCTTGCCACAAC
CCGGGATAATTCCTGCAGCCAATATGAACCCAGCCATCAGCGTCGCTCTC
CTGCTCTCAGTCTTGCAGGTGTCCCGAGGGCAGAAGGTGACCAGCCTGAC
AGCCTGCCTGGTGAACCAAAACCTTCGCCTGGACTGCCGCCATGAGAATA
ACACCAAGGATAACTCCATCCAGCATGAGTTCAGCCTGACCCGAGAGAAG
AGGAAGCACGTGCTCTCAGGCACCCTTGGGATACCCGAGCACACGTACCG
CTCCCGCGTCACCCTCTCCAACCAGCCCTATATCAAGGTCCTTACCCTAG
CCAACTTCACCACCAAGGATGAGGGCGACTACTTTTGTGAGCTTCGCGTC
TCGGGCGCGAATCCCATGAGCTCCAATAAAAGTATCAGTGTGTATAGAGA
CAAGCTGGTCAAGTGTGGCGGCATAAGCCTGCTGGTTCAGAACACATCCT
GGATGCTGCTGCTGCTGCTTTCCCTCTCCCTCCTCCAAGCCCTGGACTTC
ATTTCTCTGTGAGCTGGAGCCTCGGTGGCCATGCTTCTTGCCCCTTGGGC
CTCCCCCCAGCCCCTCCTCCCCTTCCTGCACCCGTACCCCCGTGGTCTTT
GAATAAAGTCTGACTCAGTAGATGTTTTCTTGGGTTAATTGAGGCCTGAG
TATAAGGTGACTTATACTTGTAATCTATCTAAACGGGGAACCTCTCTAGT
AGACAATCCCGTGCTAAATTGTAGGACTGCCCTTTAATAAATACTTCTAT
ATTTAAAGAGGTATTTATGAAAAGCGGAATTTATCAGATTAAAAATACTT
TCTCTAGAGTCGACCTGCAG

### LIST OF FURTHER EMBODIMENTS

The following Items are illustrative of various embodiments of the present disclosure.

Item 1. A composition comprising at least one delivery vehicle conjugated to a targeting domain (“delivery vehicle”)_wherein the targeting domain specifically binds to a cell surface antigen of a T-cell, a progenitor cell or a precursor to a T-cell, and further wherein the delivery vehicle cargo includes a nucleoside modified nucleic acid molecule encoding at least one agent for expressing forkhead box P3 (“FoxP3”), either alone or in combination with at least one other agent (“other agent”).

Item 2. The composition of Item 1, wherein the nucleic acid molecule comprises a nucleoside modified RNA molecule.

Item 3. The composition of Item 1 comprising a nucleoside modified RNA molecule having an open reading frame encoding a polypeptide comprising at least one human FoxP3 isoform (SEQ ID NOs: 1-7).

Item 4. The composition of Item 1 comprising an nucleoside modified RNA molecule having an open reading frame encoding a polypeptide comprising a modified version of least one human FoxP3 isoform

Item 5. In one iteration of Item 4 the modified version of the FoxP3 isoform is truncated to improve stability

Item 6. In another iteration of Item 4 the modified version of FoxP3 with modified nucleoside sequences to enhance stability

Item 7. In another iteration of Item 4 the modified version of FoxP3 contains a polyA tail to prevent degradation

Item 8. In another iteration of Item 4 the modified version of FoxP3 contains 1 methylpseudouridine in order to enhance translation

Item 9. The composition of Item 1, wherein the other agent is selected from the group consisting of a therapeutic agent, a stabilizing agent, an imaging agent, diagnostic agent, a contrast agent, a labeling agent and a detection agent.

Item 10. The composition of Item 1, wherein the other agent is a therapeutic agent.

Item 11. The composition of Item 10, wherein the therapeutic agent comprises a nucleoside modified nucleic acid molecule encoding an agent for the expression of Helios (SEQ ID NOs. 12-15) or a modified version of Helios

Item 12. The composition of Item 11 comprising a nucleoside modified RNA molecule having an open reading frame encoding a polypeptide comprising at least one human “Human” isoform (SEQ ID NOs: 12-15).

Item 13. The composition of Item 10, wherein the therapeutic agent comprises a nucleoside modified nucleic acid molecule encoding a chimeric antigen receptor (“CAR”).

Item 14. The composition of Item 13, wherein the CAR is expressed in combination with at least one agent for expressing forkhead box P3 (SEQ ID NOs: 18-22).

Item 15. The composition of Item 13, wherein the CAR is expressed in combination with at least one isolated RNA molecule encoding at least one protein with a secretory signal.

Item 16. The composition of Item 10 the therapeutic agent comprises a nucleoside modified nucleic acid molecule encoding a receptor linked to a downstream effector.

Item 17. The composition of Item 10, wherein the therapeutic agent comprises at least one isolated RNA molecule encoding at least one component for gene editing.

Item 18. The composition of Item 10, wherein the therapeutic agent comprises at least one selected from the group consisting of a Cas9 mRNA and a guide RNA.

Item 19. The composition of Item 10, wherein the therapeutic agent comprises a nucleoside modified nucleic acid molecule encoding a stabilizing agent to stabilize FoxP3.

Item 20. The composition of Item 19, wherein the stabilizing agent is selected from the group consisting of IKZF2, PP1, NLK, OGT, OGA, SIRT1, RORγt, USP7, USP21, RNF31, TRAF6, PRMT1, PRMT5, NFAT, LAG-3, GITR, NRP1, c-REL, ALPK1, CREB, STAT5, SMAD3, RXR, ICOS, PHD3, FOXO1, IL-2R, IDO, TIGIT, GARP, CD98, CD28, CD73 and CD39.

Item 21. The composition of Item 1, wherein the cell surface antigen of the T cell is selected from the group consisting of CD1, CD2, CD3, CD4, CDS, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD152, CD153, CD154, CD161, CD183, CD223, CD254, CD275, CD45RA, CXCR3, CXCR5, FasL, IL18RI, CTLA-4, OX40, GITR, LAG3, ICOS, PD-1, leu-12, TCR, TLRI, TLR2, TLR3, TLR4, TLR6, NKG2D, CCR, CCRI, CCR2, CCR4, CCR6, and CCR7.

Item 22. The composition of Item 21, wherein the cell surface antigen of a T-cell is an activated T antigen.

Item 23. The composition of Item 22, wherein the pan-T antigen is selected from the group consisting of CD4, CD69, CD71, CD25 and CD8.

Item 24. The composition of any one of Items 1-23, wherein the delivery vehicle is selected from the group consisting of a lipid carrier, a liposome, a lipid nanoparticle, and a micelle.

Item 25. The composition of Item 24, wherein the delivery vehicle is an ionizable lipid nanoparticle.

Item 26. The composition of Item 25, wherein the lipid nanoparticle comprises a PEG-lipid conjugated to the targeting domain.

Item 27. The composition of Item 26, wherein the at least one agent is encapsulated in the lipid nanoparticle.

Item 28. The composition of any one of Items 1-27, wherein the targeting domain is selected from the group consisting of a nucleic acid molecule, a peptide, an antibody, and a small molecule.

Item 29. The composition of Item 28, wherein the targeting domain is an antibody.

Item 30. The composition of Item 28, wherein the targeting domain is an anti-CD4 antibody.

Item 31. The composition of Item 28, wherein the targeting domain is a peptide.

Item 32. The composition of Item 28, wherein the targeting domain is a IL1R1 binding antagonist.

Item 33. The method of Item 28, wherein the composition is administered by a delivery route selected from the group consisting of intradermal, subcutaneous, intracranial, inhalation, intranasal, oral, peroral, and intramuscular.

Item 34. A method of treating or preventing inflammation or a disease or disorder associated with inflammation in a subject in need thereof, the method comprising administering to the subject the composition of any one of Items 1-33, wherein the composition is suitable for administration to a human subject in need of anti-inflammatory treatment.

Item 35. A dosage form comprising an ionizable lipid carrier and a nucleoside modified nucleic acid molecule encoding at least one agent for expressing “FoxP3” and a therapeutic agent according to any of the preceding Items for use in a method of treatment, amelioration, mitigation, slowing, arresting, reversing or prevention of a condition selected from the group consisting of: mitochondrial diseases/disorders, metabolic disorders, neurodegenerative diseases, polyglutamine diseases, anticoagulation and antithrombotic conditions, allergies and respiratory conditions, autoimmune diseases, vision impairment, dyslipidemia, hyperlipidemia, diabetes, metabolic syndrome, inflammation, sepsis, apoptosis, autoimmunity, neurodegeneration, including Alzheimers, Parkinson's, Huntington's, oxidative stress, hypercholesterolemia, atherosclerosis, cardiovascular disease (CVD), steatohepatitis (fatty liver disease), pancreatitis, renal lipid deposition, depression, and/or for reduce hCRP levels.

Item 36. The composition of Item 1, wherein the composition is a unit dosage form having a dosage of 15 micrograms to 800 micrograms of the encapsulated nucleotide for a dose of up to 2.5 mg/kg in a human subject.

Item 37. The method of Item 34, for use in a method of treatment, amelioration, mitigation, slowing, arresting or reversing or prevention of a condition selected from the group consisting of: mitochondrial diseases/disorders, metabolic disorders, neurodegenerative diseases, polyglutamine diseases, anticoagulation and antithrombotic conditions, allergies and respiratory conditions, autoimmune diseases, vision impairment, dyslipidemia, hyperlipidemia, diabetes, metabolic syndrome, inflammation, sepsis, apoptosis, autoimmunity, oxidative stress, hypercholesterolemia, atherosclerosis, cardiovascular disease (CVD), steatohepatitis (fatty liver disease), pancreatitis, renal lipid deposition, and fatty liver disease, depression, neurodegeneration, preferably Parkinson's and Alzheimer's, and cancer, preferably pancreatic cancer.

What is claimed is:

1. A composition comprising at least one nanoparticle conjugated to a targeting domain that specifically binds to a cell surface antigen of a T cell, a progenitor cell or a precursor to a T cell, wherein the nanoparticle contains a nucleoside-modified RNA molecule comprising a coding sequence for a human forkhead box P3 (FOXP3) polypeptide.

2. The composition of claim 1, wherein the RNA is a circular RNA, and wherein an internal ribosome entry site (IRES) sequence is operably linked to the coding sequence.

3. The composition of claim 2, wherein the circular RNA is synthesized from an expression vector comprising self-splicing introns, a 5′ spacer, a 3′ untranslated region (UTR), and an IRES.

4. The composition of claim 2, wherein the circular RNA comprises at least 5% N6-methyladenosine (m⁶A) residues.

5. The composition of claim 1, wherein the human FOXP3 polypeptide comprises an amino acid sequence at least 95% identical to any one of SEQ ID NOS: 8-11.

6. The composition of claim 1, wherein the nucleoside-modified RNA molecule comprises any one of SEQ ID NOS: 1-7.

7. The composition of claim 1, wherein the nucleoside-modified RNA molecule further comprises a polyA tail.

8. The composition of claim 1, wherein the nucleoside-modified RNA molecule further comprises at least one 1-methylpseudouridine.

9. The composition of claim 1 wherein the nanoparticle is selected from the group consisting of a lipid carrier, a liposome, a lipid nanoparticle, and a micelle.

10. The composition of claim 1 wherein the nanoparticle is an ionizable lipid nanoparticle.

11. The composition of claim 1, wherein the nanoparticle comprises a PEG-lipid conjugated to the targeting domain.

12. The composition of claim 1, wherein the targeting domain is selected from the group consisting of a nucleic acid molecule, a peptide, an antibody, and a small molecule.

13. The composition of claim 1, wherein the targeting domain is an anti-CD4 antibody.

14. The composition of claim 1, wherein the targeting domain binds IL1R1.

15. The composition of claim 1, wherein the cell surface antigen of the T cell is selected from the group consisting of CD1, CD2, CD3, CD4, CD5, CD7, CD8, CD16, CD25, CD26, CD27, CD28, CD30, CD38, CD39, CD40L, CD44, CD45, CD62L, CD69, CD71, CD73, CD80, CD83, CD86, CD95, CD103, CD119, CD126, CD150, CD152, CD153, CD154, CD161, CD183, CD223, CD254, CD275, CD45RA, CXCR3, CXCR5, FasL, IL18RI, CTLA-4, OX40, GITR, LAG3, ICOS, PD-1, leu-12, TCR, TLRI, TLR2, TLR3, TLR4, TLR6, NKG2D, CCR, CCRI, CCR2, CCR4, CCR6, and CCR7.

16. The composition of any one of claims 1-15, wherein the nanoparticle further contains at least a second agent.

17. The composition of claim 16, wherein the second agent is selected from the group consisting of a therapeutic agent, a stabilizing agent, an imaging agent, diagnostic agent, a contrast agent, a labeling agent and a detection agent.

18. The composition of any one of claims 1-17, wherein the second agent comprises a nucleoside modified nucleic acid molecule encoding a stabilizing agent to stabilize FOXP3.

19. The composition of claim 18, wherein the stabilizing agent is selected from the group consisting of IKZF2, PP1, NLK, OGT, OGA, SIRT1, RORγt, USP7, USP21, RNF31, TRAF6, PRMT1, PRMT5, NFAT, LAG-3, GITR, NRP1, c-REL, ALPK1, CREB, STAT5, SMAD3, RXR, ICOS, PHD3, FOXO1, IL-2R, IDO, TIGIT, GARP, CD98, CD28, CD73 and CD39.

20. The composition of any one of claims 1-19, wherein the nanoparticle further contains a nucleoside modified nucleic acid molecule encoding a human IKZF2 Helios polypeptide.

21. The composition of any one of claims 1-20, wherein the nanoparticle further contains a nucleoside modified RNA encoding a modified human IKZF2 Helios polypeptide at least 95% identical to SEQ ID NO: 16.

22. The composition of any one of claims 1-21, wherein the second agent comprises a nucleoside modified nucleic acid molecule encoding a therapeutic agent to target inflammation.

23. The composition of any one of claims 1-22, wherein the nanoparticle further contains a nucleoside modified RNA encoding a chimeric antigen receptor (CAR).

24. The composition of claim 23, wherein the nucleoside modified RNA comprises SEQ ID NO: 19, 20, 21, or 22.

25. The composition of claim 24, wherein the CAR is expressed in combination with at least one RNA molecule encoding at least one protein with a secretory signal.

26. The composition of any one of claims 1-25, wherein the second agent comprises a receptor linked to a downstream effector or at least one component for gene editing.

27. The composition of claim 26, wherein the at least one component for gene editing is a Cas9 mRNA, a guide RNA, or both of a Cas9 mRNA and a guide RNA.

28. A circular RNA comprising at least a first IRES operably linked to a first coding sequence and a second IRES operably linked to a second coding sequence.

29. The circular RNA of claim 28, wherein the first IRES operably linked to the first coding sequence and the second IRES operably linked to the second coding sequence are separated by an intron.

30. The circular RNA of claim 28, wherein the circular RNA comprises at least 5% N6-methyladenosine (m⁶A) residues.

31. The circular RNA of claim 28, wherein the first coding sequence or second coding sequence or both the first and second coding sequence encodes a human FOXP3 polypeptide at least 95% identical to SEQ ID NOS: 8-11.

32. The circular RNA of any one of claims 28-31, wherein the first coding sequence or second coding sequence or both the first and second coding sequence comprises any one of SEQ ID NOS: 1-7.

33. The circular RNA of any one of claims 28-32 wherein the first coding sequence or second coding sequence or both the first and second coding sequence comprises a nucleoside modified RNA encoding a modified human IKZF2 Helios polypeptide at least 95% identical SEQ ID NO: 16.

34. The circular RNA of any one of claims 28-33, wherein one of the first coding sequence and the second coding sequence encodes a human FOXP3 polypeptide at least 95% identical to SEQ ID NOS: 8-11 and one of the first coding sequence and the second coding sequence comprises a nucleoside modified RNA encoding a modified human IKZF2 Helios polypeptide at least 95% identical to SEQ ID NO: 16.

35. The circular RNA of any one of claims 28-34, wherein the first coding sequence encodes the human FOXP3 polypeptide and the second coding sequence encodes the human Helios polypeptide.

36. The circular RNA of any one of claims 28-34, wherein the first coding sequence encodes the human Helios polypeptide and the second coding sequence encodes the human FOXP3 polypeptide

37. The circular RNA of claim 36, wherein the second coding sequence comprises any one of SEQ ID NOS: 1-7.

38. The circular RNA of claim 35, wherein the second coding sequence encodes a human Helios polypeptide and comprises one of SEQ ID NOS: 12-15.

39. The circular RNA of any one of claims 28-38, wherein the first IRES comprises any one of SEQ ID NOs: 23-62.

40. The circular RNA of any one of claims 28-39, wherein the second IRES comprises any one of SEQ ID NOs: 23-62.

41. The circular RNA of claim 28, further comprising a third IRES operably linked to a third coding sequence.

42. A nucleoside-modified RNA molecule encoding FOXP3, wherein the nucleoside modified RNA molecule has a sequence comprising Formula I, 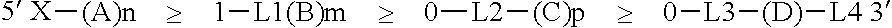
wherein,
at least one of regions A, B, or C is positionally modified;
at least one of the regions A, B or C is polynucleotide encoding the human FOXP3 polypeptide
at least one of the regions A, B or C is a polynucleotide encoding at least one therapeutic agent
n is an integer between 1 and 5;
m is an integer between 0 and 5;
p is an integer between 0 and 5;
X is a UTR between 1 base and 1 kilobase in size;
D is an optional region of linked nucleosides;
L1, L2 and L3 are independently optional linker moieties, said linker moieties being either nucleic acid based, or non-nucleic acid based; and
L4 is an optional conjugate or an optional linker moiety, said linker moiety being either nucleic acid based, or non-nucleic acid based.

43. The nucleoside-modified RNA molecule of claim 42, wherein the region A encodes the human FOXP3 polypeptide, optionally wherein the human FOXP3 polypeptide is at least 95% identical to one of SEQ ID NOS: 8-11.

44. The nucleoside-modified RNA molecule of claim 42, wherein the region A comprises any one of SEQ ID NOS: 1-7.

45. The nucleoside-modified RNA molecule of claim 42, wherein the region A encodes a human Helios polypeptide, comprising a nucleoside modified RNA encoding a modified human IKZF2 Helios polypeptide at least 95% identical to one of SEQ ID NOS: 12-15.

46. The nucleoside-modified RNA molecule of claim 42, wherein the region B encodes the human FOXP3 polypeptide, optionally wherein the human FOXP3 polypeptide is at least 95% identical to one of SEQ ID NOS: 8-11.

47. The nucleoside-modified RNA molecule of claim 42, wherein the region B comprises any one of SEQ ID NOS: 1-7.

48. The nucleoside-modified RNA molecule of claim 42, wherein the region B encodes a human Helios polypeptide, comprising a nucleoside modified RNA encoding a modified human IKZF2 Helios polypeptide at least 95% identical to one of SEQ ID NOS: 12-15.

49. The nucleoside-modified RNA molecule of claim 42, wherein the region C encodes the human FOXP3 polypeptide, optionally wherein the human FOXP3 polypeptide is at least 95% identical to one of SEQ ID NOS: 8-11.

50. The nucleoside-modified RNA molecule of claim 42, wherein the region C encodes a human Helios polypeptide, optionally wherein the human Helios polypeptide is at least 95% identical to one of SEQ ID NOS: 1-7.

51. The nucleoside-modified RNA molecule of claim 42, wherein the region C encodes a human Helios polypeptide, comprising a nucleoside modified RNA encoding a modified human IKZF2 Helios polypeptide at least 95% identical to one of SEQ ID NOS: 12-15.

52. The nucleoside-modified RNA molecule of claim 42, wherein the RNA is a circular RNA.

53. The nucleoside-modified RNA molecule of claim 42, wherein an IRES is operably linked to at least one, two or all three of the regions A, B, or C.

54. The nucleoside-modified RNA molecule of any one of claims 42-54, wherein an intron is present between regions A and B or regions B and C or between both regions A and B and regions B and C.

55. A method of treating or preventing inflammation or a disease or disorder associated with inflammation in a subject in need thereof, the method comprising administering to the subject the composition of any one of claims 1-27, the circular RNA of any one of claims 28-41, or the nucleoside-modified RNA molecule of any one of claims 42-53.

56. The method of claim 55, wherein the composition is administered by a delivery route selected from the group consisting of intradermal, subcutaneous, intracranial, inhalation, intranasal, oral, peroral, and intramuscular.

57. The method of claim 55, wherein the disease or disorder is selected from the group consisting of: an age-related disease or disorder, mitochondrial disease or disorder, metabolic disorder, neurodegenerative disease, polyglutamine disease, anticoagulation condition, antithrombotic condition, allergy, respiratory condition, autoimmune disease, vision impairment, dyslipidemia, hyperlipidemia, diabetes, metabolic syndrome, inflammation, sepsis, apoptosis, autoimmunity, neurodegeneration, Alzheimer's disease, Parkinson's disease, Huntington's disease, oxidative stress, hypercholesterolemia, atherosclerosis, cardiovascular disease (CVD), steatohepatitis (fatty liver disease), pancreatitis, renal lipid deposition, depression, an elevated hCRP level, and cancer.

58. The method of claim 55, wherein the composition is a unit dosage form having a dosage of 15 micrograms to 800 micrograms of the RNA molecule for a dose of up to 2.5 mg/kg in a human subject.


SEQUENCE LISTING (72 sequences)

SEQ ID NO: 1 - DNA, 2264 bp, Homo sapiens
  source 1..2264: mol_type = genomic DNA
agtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctcccca

SEQ ID NO: 2 - DNA, 1296 bp, Homo sapiens
  source 1..1296: mol_type = genomic DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 3 - DNA, 1299 bp, Homo sapiens
  source 1..1299: mol_type = genomic DNA
atgggccccaacccccgtccgggcaaaccctctgctccatcactcgcgctgggcccttcgcctggagcttcccccagttggcgggccgctcccaaggccagcgatttgctgggtgcccggggacccggaggtacgttccagggtcgcgacctgagaggcggcgcgcatgcctcttcgtcctccctcaatcccatgcctccaagccagctccaactgcccaccctgcctctggtgatggtcgccccatctggggctcgcctgggtccgcttcctcatctgcaagccctgctgcaggacaggcctcacttcatgcaccagctgagcaccgtggacgcccacgcccgcacccccgtgctgcaggtgcaccccctggaatctccggccatgatctctctgactccacctaccacagccaccggcgttttcagcctgaaagcccgtcctggcctgcctcctggcatcaacgtggcctccctcgagtgggtctcgcgggaaccggcgcttctgtgcacttttcccaatccttccgctcctcggaaggattccacactgagcgccgtgcctcagagtagctaccccttgctggccaacggcgtgtgcaagtggcccgggtgcgagaaggtgttcgaggagcccgaggacttcctgaagcactgtcaggccgaccacctgctggacgagaagggcagagcccagtgcctgcttcagcgcgagatggtgcagagcttggaacagcagctggtattagaaaaagaaaagctgtctgccatgcaggcccatctggctggcaagatggccctgaccaaggcaagctccgtggcttcgagcgacaagggcagctgttgtatcgtggccgccggatctcagggccctgtggtccctgcttggtctggcccacgcgaggcacccgactccctgttcgccgtgagaagacacctgtggggcagccacggcaacagcacctttccagagtttctgcacaacatggattatttcaagttccacaacatgagacctcccttcacctacgccacgctgatcagatgggccatcctggaggcccctgaaaagcagagaaccctgaacgagatctaccactggttcactcggatgtttgcgttcttccgcaatcaccctgctacctggaagaacgccattcgccacaacctgagcctgcacaagtgctttgtcagagtggagagcgagaaaggcgctgtgtggaccgttgatgagctggagttcagaaagaagaggagccagagaccatccaggtgtagcaaccccacacctggcccctga

SEQ ID NO: 4 - DNA, 1296 bp, Homo sapiens
  source 1..1296: mol_type = genomic DNA
atgggacccaatcccagacccgggaagcctagcgccccgtctcttgccttggggccctctcccggtgcctcacctagttggagagccgcgcctaaggcttctgatctgctgggcgctagaggccctggggggacgttccagggccgggacctgagaggcggagcccacgcctccagttcctccctgaacccaatgcctccatcgcagctgcagctgcccacactgcccttagtgatggtggctcctagcggagctcggctgggacccctgcctcatctgcaagccctactgcaggacaggccgcactttatgcaccagctgtccaccgtcgacgcacatgccaggacccctgtgctccaagtgcaccctctggagagtcctgctatgatcagcctgactccgcctaccacagccacaggcgtgttctccctgaaggccagacccggcctgcctcctggcatcaacgtggcttccctggagtgggtctctcgggagcccgctctgctgtgcactttccctaaccctagcgccccgagaaaggacagcacactgagcgccgttcctcagagctcgtaccccctgctggccaacggagtttgtaaatggcccgggtgcgagaaagtgttcgaagagccggaggacttcttgaagcactgtcaggccgatcacctgctcgacgaaaagggacgtgcccagtgcctgttgcagcgggagatggtgcagtccctagaacagcagctcgtgctggaaaaggaaaagctgagcgccatgcaggcccatctggcaggtaagatggcccttacaaaagcctcttccgtggccagtagcgacaaaggctcctgctgcatcgtcgctgctggctcccagggcccggtggtgcctgcctggtctggcccacgagaggctcctgatagcctctttgctgtgcggcgccatctctggggcagccacggcaattccaccttccccgagttcctgcacaacatggattatttcaagtttcacaacatgcgtccccctttcacctacgctacactgatccggtgggccatcctggaagcacctgagaaacagagaacactgaacgagatctaccactggttcacccggatgtttgcgttcttccgcaaccaccctgctacctggaagaacgctattcgccacaatctgtctctgcacaaatgctttgtccgagtagagagcgagaagggcgccgtgtggaccgtggatgaactggagttcagaaagaagagatcccagagaccatcgaggtgtagcaacccaactccgggcccc

SEQ ID NO: 5 - RNA, 1296 nt, Homo sapiens
  source 1..1296: mol_type = genomic RNA
atgggacccaatcccagacccgggaagcctagcgccccgtctcttgccttggggccctctcccggtgcctcacctagttggagagccgcgcctaaggcttctgatctgctgggcgctagaggccctggggggacgttccagggccgggacctgagaggcggagcccacgcctccagttcctccctgaacccaatgcctccatcgcagctgcagctgcccacactgcccttagtgatggtggctcctagcggagctcggctgggacccctgcctcatctgcaagccctactgcaggacaggccgcactttatgcaccagctgtccaccgtcgacgcacatgccaggacccctgtgctccaagtgcaccctctggagagtcctgctatgatcagcctgactccgcctaccacagccacaggcgtgttctccctgaaggccagacccggcctgcctcctggcatcaacgtggcttccctggagtgggtctctcgggagcccgctctgctgtgcactttccctaaccctagcgccccgagaaaggacagcacactgagcgccgttcctcagagctcgtaccccctgctggccaacggagtttgtaaatggcccgggtgcgagaaagtgttcgaagagccggaggacttcttgaagcactgtcaggccgatcacctgctcgacgaaaagggacgtgcccagtgcctgttgcagcgggagatggtgcagtccctagaacagcagctcgtgctggaaaaggaaaagctgagcgccatgcaggcccatctggcaggtaagatggcccttacaaaagcctcttccgtggccagtagcgacaaaggctcctgctgcatcgtcgctgctggctcccagggcccggtggtgcctgcctggtctggcccacgagaggctcctgatagcctctttgctgtgcggcgccatctctggggcagccacggcaattccaccttccccgagttcctgcacaacatggattatttcaagtttcacaacatgcgtccccctttcacctacgctacactgatccggtgggccatcctggaagcacctgagaaacagagaacactgaacgagatctaccactggttcacccggatgtttgcgttcttccgcaaccaccctgctacctggaagaacgctattcgccacaatctgtctctgcacaaatgctttgtccgagtagagagcgagaagggcgccgtgtggaccgtggatgaactggagttcagaaagaagagatcccagagaccatcgaggtgtagcaacccaactccgggcccc

SEQ ID NO: 6 - DNA, 2159 bp, Homo sapiens
  source 1..2159: mol_type = genomic DNA
agtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctcccca

SEQ ID NO: 7 - DNA, 1191 bp, Homo sapiens
  source 1..1191: mol_type = genomic DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 8 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 9 - protein, 396 aa, Homo sapiens
MPNPRPGKPSAPSLALGPSPGASPSWRAAPKASDLLGARGPGGTFQGRDLRGGAHASSSSLNPMPPSQLQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 10 - protein, 456 aa, Homo sapiens
MPNPRPGKPSAPSLALGPSPGASPSWRAAPKASDLLGARGPGGTFQGRDLRGGAHASSSSLNPMPPSQLQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKVSSSEVAVTGMASSAIAAQSGQAWVWAHRHIGEERDVGCWWWLLASEVDAHLLPVPGLPQNAIRHNLSLHKCFVRVESEKGAVWTVDELEFRKKRSQRPSRCSNPTPGP

SEQ ID NO: 11 - protein, 404 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 12 - DNA, 1503 bp, Homo sapiens
  source 1..1503: mol_type = genomic DNA
atggaaacagaggctattgatggctatataacgtgtgacaatgagctttcacccgaaagggagcactccaatatggcaattgacctcacctcaagcacacccaatggacagcatgcctcaccaagtcacatgacaagcacaaattcagtaaagctagaaatgcagagtgatgaagagtgtgacaggaaacccctgagccgtgaagatgagatcaggggccatgatgagggtagcagcctagaagaacccctaattgagagcagcgaggtggctgacaacaggaaagtccaggagcttcaaggcgagggaggaatccggcttccgaatggtgaacgccccttccactgtaaccagtgtggagcttcttttactcagaagggcaaccttctgagacacataaagttacactctggagagaagccgttcaaatgtcctttctgtagctacgcctgtagaagaagggacgccctcacaggacacctcaggacccattctgtgggtaaacctcacaagtgcaactactgtggacgaagctacaagcagcgcagttcactggaggagcacaaggaacgctgccacaactatctccagaatgtcagcatggaggctgctgggcaggtcatgagtcaccatgtacctcctatggaagattgtaaggaacaagagcctattatggacaacaatatttctctggtgccttttgagagacctgctgtcatagagaagctcacggggaatatgggaaaacgtaaaagctccactccacaaaagtttgtgggggaaaagctcatgcgattcagctacccagatattcactttgatatgaacttaacatatgagaaggaggctgagctgatgcagtctcatatgatggaccaagccatcaacaatgcaatcacctaccttggagctgaggcccttcaccctctgatgcagcacccgccaagcacaatcgctgaagtggccccagttataagctcagcttattctcaggtctatcatccaaataggatagaaagacccattagcagggaaactgctgatagtcatgaaaacaacatggatggccccatctctctcatcagaccaaagagtcgaccccaggaaagagaggcctctcccagcaatagctgcctggattccactgactcagaaagcagccatgatgaccaccagtcctaccaaggacaccctgccttaaatcccaagaggaaacaaagcccagcttacatgaaggaggatgtcaaagctttggatactaccaaggctcctaagggctctctgaaggacatctacaaggtcttcaatggagaaggagaacagattagggccttcaagtgtgagcactgccgagtccttttcctagaccatgtcatgtacaccattcacatgggttgccatggctaccgggacccactggaatgcaacatctgtggctacagaagccaggaccgttatgagttttcatcacacattgttcgaggggagcacacattccactag

SEQ ID NO: 13 - DNA, 1503 bp, Homo sapiens
  source 1..1503: mol_type = genomic DNA
atggaaaccgaggctatcgacggctacatcacctgcgacaacgagctttctcctgagagagaacactcaaatatggccatcgacctgacgtctagcactccgaacggacagcacgcgagccccagccatatgaccagcaccaacagcgttaagctggagatgcagagcgatgaagagtgtgataggaagcctctgagtcgcgaggacgagatccggggacacgacgagggctcctcgttggaagaacctcttattgagtctagcgaggtggcggacaacagaaaagtgcaggagctgcagggcgagggcggcattcgcctacctaacggtgagcggcctttccactgtaaccagtgtggcgctagcttcacccagaaaggcaacctgttgcgccacatcaaactgcactctggcgagaagccgtttaaatgtcccttctgcagctacgcctgccggcgccgcgacgccctgaccggccacttgagaacacacagcgtgggcaagcctcacaagtgcaactactgcggcagaagctacaagcaaagatcttcccttgaggagcacaaagagaggtgtcataactacctgcagaatgtgtccatggaagcagccggccaagtaatgagccaccacgtcccacccatggaagactgcaaggagcaggagcccatcatggataacaatatctctctggtgcccttcgagcgtccagccgtgatcgagaaactcactggtaatatgggaaagcgcaagagctctacacctcagaagttcgtgggggagaagctgatgcgcttttcataccccgacatccatttcgatatgaacctgacctatgagaaagaagctgagctgatgcagagtcatatgatggaccaggccatcaacaacgccattacctacctgggcgccgaggccctgcaccccctgatgcagcaccctccatccacgatcgccgaagtcgcccctgtgatctccagcgcctacagccaagtctaccaccccaaccggatcgaacggccgatttccagggagacagccgacagccacgagaacaacatggacgggcccatctctctgatccggcccaagtcccgccctcaggagagggaagcttcccccagcaactcatgcctggacagtaccgactctgagagcagccatgatgaccaccagagctatcagggacatcctgctctcaatccaaagcgcaagcagtccccagcgtacatgaaggaagatgtgaaggctctggatacaaccaaggctcctaagggctccctgaaggacatctacaaggtgttcaacggcgaaggcgaacagatcagagccttcaagtgcgagcactgcagagtgctgttcctggaccacgtgatgtacaccatccacatgggctgccacggttacagagatccactggagtgtaacatctgcggctatcgttctcaggacaggtacgagttcagctcgcacatcgtgcggggtgagcatacctttcactga

SEQ ID NO: 14 - DNA, 1503 bp, Homo sapiens
  source 1..1503: mol_type = genomic DNA
atggaaacggaggctattgacggctacatcacctgcgacaacgaactatctcctgaaagagagcactccaatatggcgatcgacctaaccagctccacccctaacggacagcatgcctctccgtcccacatgaccagcaccaacagcgtcaaactggagatgcagtccgacgaggagtgtgaccgcaagcccctgtctcgcgaggacgagataagaggtcatgacgagggatctagcctcgaggaaccactcattgagagctccgaggtggccgacaatcggaaggtgcaggaactacaaggcgaaggcggcatccgtctgcctaatggcgagagacctttccactgtaaccagtgtggcgcgagcttcacccagaagggcaatctgctgagacacatcaagctgcactctggtgagaagccttttaaatgcccattctgctcttacgcctgccgacggcgcgacgccctgaccggccacctccgcacccactctgtgggcaaacctcacaagtgcaactactgcggccgctcctacaagcagagaagcagcttggaagagcacaaggagcgctgtcacaactatctgcagaacgtgtccatggaagccgccggccaggtgatgagccaccacgtgccgcccatggaagattgcaaggagcaggagcccatcatggacaacaacatctcactggtccctttcgagagaccggcggtgattgagaagctgacaggaaacatgggcaagcgcaaaagctcgacgccccagaagttcgtcggcgagaagctgatgcgtttcagctatcccgacatccacttcgacatgaacctgacttacgagaaagaggctgaactgatgcaaagtcacatgatggaccaggccatcaataacgccatcacttacctgggcgccgaggccctgcaccccctgatgcagcatcccccgtctaccatcgccgaggtggcccccgtcattagcagcgcctactcccaggtgtaccaccccaaccgcatcgagcggcccatctctcgtgagactgcggattctcacgagaacaatatggatggtcccatcagcctcatcagacctaagagccgccctcaagagagagaggcctccccaagcaacagctgcctggattctaccgacagcgaatcgagccacgatgaccaccagagctatcagggtcaccctgccctcaacccgaagagaaagcagagccccgcctacatgaaggaagacgtgaaggccctggacaccaccaaggcccctaagggctccctgaaggacatctacaaggtgttcaacggcgagggcgagcaaatccgcgcattcaagtgcgagcactgtagagtgctgttcctggaccacgtgatgtacaccatccatatgggctgtcatggctaccgcgacccacttgagtgcaacatctgtggctacagatctcaggatcgctacgagttttcgagccacatcgtgcgcggagagcacacgtttcactga

SEQ ID NO: 15 - RNA, 1503 nt, Homo sapiens
  source 1..1503: mol_type = genomic RNA
atggaaacggaggctattgacggctacatcacctgcgacaacgaactatctcctgaaagagagcactccaatatggcgatcgacctaaccagctccacccctaacggacagcatgcctctccgtcccacatgaccagcaccaacagcgtcaaactggagatgcagtccgacgaggagtgtgaccgcaagcccctgtctcgcgaggacgagataagaggtcatgacgagggatctagcctcgaggaaccactcattgagagctccgaggtggccgacaatcggaaggtgcaggaactacaaggcgaaggcggcatccgtctgcctaatggcgagagacctttccactgtaaccagtgtggcgcgagcttcacccagaagggcaatctgctgagacacatcaagctgcactctggtgagaagccttttaaatgcccattctgctcttacgcctgccgacggcgcgacgccctgaccggccacctccgcacccactctgtgggcaaacctcacaagtgcaactactgcggccgctcctacaagcagagaagcagcttggaagagcacaaggagcgctgtcacaactatctgcagaacgtgtccatggaagccgccggccaggtgatgagccaccacgtgccgcccatggaagattgcaaggagcaggagcccatcatggacaacaacatctcactggtccctttcgagagaccggcggtgattgagaagctgacaggaaacatgggcaagcgcaaaagctcgacgccccagaagttcgtcggcgagaagctgatgcgtttcagctatcccgacatccacttcgacatgaacctgacttacgagaaagaggctgaactgatgcaaagtcacatgatggaccaggccatcaataacgccatcacttacctgggcgccgaggccctgcaccccctgatgcagcatcccccgtctaccatcgccgaggtggcccccgtcattagcagcgcctactcccaggtgtaccaccccaaccgcatcgagcggcccatctctcgtgagactgcggattctcacgagaacaatatggatggtcccatcagcctcatcagacctaagagccgccctcaagagagagaggcctccccaagcaacagctgcctggattctaccgacagcgaatcgagccacgatgaccaccagagctatcagggtcaccctgccctcaacccgaagagaaagcagagccccgcctacatgaaggaagacgtgaaggccctggacaccaccaaggcccctaagggctccctgaaggacatctacaaggtgttcaacggcgagggcgagcaaatccgcgcattcaagtgcgagcactgtagagtgctgttcctggaccacgtgatgtacaccatccatatgggctgtcatggctaccgcgacccacttgagtgcaacatctgtggctacagatctcaggatcgctacgagttttcgagccacatcgtgcgcggagagcacacgtttcactga

SEQ ID NO: 16 - protein, 500 aa, Homo sapiens
METEAIDGYITCDNELSPEREHSNMAIDLTSSTPNGQHASPSHMTSTNSVKLEMQSDEECDRKPLSREDEIRGHDEGSSLEEPLIESSEVADNRKVQELQGEGGIRLPNGERPFHCNQCGASFTQKGNLLRHIKLHSGEKPFKCPFCSYACRRRDALTGHLRTHSVGKPHKCNYCGRSYKQRSSLEEHKERCHNYLQNVSMEAAGQVMSHHVPPMEDCKEQEPIMDNNISLVPFERPAVIEKLTGNMGKRKSSTPQKFVGEKLMRFSYPDIHFDMNLTYEKEAELMQSHMMDQAINNAITYLGAEALHPLMQHPPSTIAEVAPVISSAYSQVYHPNRIERPISRETADSHENNMDGPISLIRPKSRPQEREASPSNSCLDSTDSESSHDDHQSYQGHPALNPKRKQSPAYMKEDVKALDTTKAPKGSLKDIYKVFNGEGEQIRAFKCEHCRVLFLDHVMYTIHMGCHGYRDPLECNICGYRSQDRYEFSSHIVRGEHTFH

SEQ ID NO: 17 - DNA, 1506 bp, synthetic construct
  source 1..1506: mol_type = other DNA
atggctagccccctgacacgcttcctgtccctgaacctgctgctgctgggagagagcatcatcctgggctccggcgaagccgaacagaaactgatttctgaggaagacctggacgtgcagctgcaagagtctgggcctgggctggtgaagcctagccagagcctgtctctaacctgcagcgtcacaggctactccatcacaagcggctactactggacctggatccgccagtttccgggcaacaagctggaatggatggggtcgattggctacgacggcaccaactactacaaccccagtctcaagaacagaatcagcatcacccgtgacaccagcaagaaccagttcttcttgaagctgcattccgtgaccaccgaggataccgctacctattactgcgccaagcggggcatcaccactggcgactattggggccagggaaccacactcacagttagctctggcgggggaggctccggtggcggcggtagcggaggtggtgggtcccaggccgtggtgacgcaggagagcgccctgacgacatctcccggcgagaccgtgaccctgacctgtcgcagcagcaccggcgccgtcactacctccaactacgcgaattgggtgcaggaaaaacccgaccacctgttcatcggactgataggtgggaccaataagcgtacacctggcgtaccagctcggttttctggttcccttattggggacaaggccgctctgaccatcactggcacacagactgaggacgaggccatctacttctgcgctctgtggtacagcaaccacctggtgttcggcggcggcaccaagctgaccgtcctgtcgaccacaaccaaacctgtgctgagaactccgtcgccagtgcaccccacgggcaccagccagcctcagagacctgaggattgtcgccctagagggtctgtgaaaggcacgggactggacttcgcctgcgacatctacatctgggcccctctggctggtatctgtgtggccctgctgctctcgctgatcatcaccctgatctgctacaacagcagacgaaaccggctcttgcagagcgactacatgaacatgacccccagaaggcctggcctaactcgcaagccctatcagccttacgcgccggcccgcgactttgcggcctaccggccaagagccaagttcagccggagagcagaaacagccgccaacctgcaggaccccaaccagctgtacaacgagctaaacctcggcagaagagaggagtacgacgtgctggagaagaagcgcgctcgcgacccagagatgggcggtaagcagcagcgccgtcggaatcctcaagagggcgtgtacaacgccctccagaaggataagatggccgaggcctatagcgagatcggcacaaagggcgagagaaggcgcggcaaaggccacgatggcctgtaccagggcctgtccaccgccaccaaggatacctacgatgccctgcacatgcagacgcttgcaccccggtga

SEQ ID NO: 18 - protein, 501 aa, synthetic construct
MASPLTRFLSLNLLLLGESIILGSGEAEQKLISEEDLDVQLQESGPGLVKPSQSLSLTCSVTGYSITSGYYWTWIRQFPGNKLEWMGSIGYDGTNYYNPSLKNRISITRDTSKNQFFLKLHSVTTEDTATYYCAKRGITTGDYWGQGTTLTVSSGGGGSGGGGSGGGGSQAVVTQESALTTSPGETVTLTCRSSTGAVTTSNYANWVQEKPDHLFIGLIGGTNKRTPGVPARFSGSLIGDKAALTITGTQTEDEAIYFCALWYSNHLVFGGGTKLTVLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRRAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 19 - DNA, 4884 bp, synthetic construct
  source 1..4884: mol_type = other DNA
atagtaatagccgccaccatgggacccaatcccagacccgggaagcctagcgccccgtctcttgccttggggccctctcccggtgcctcacctagttggagagccgcgcctaaggcttctgatctgctgggcgctagaggccctggggggacgttccagggccgggacctgagaggcggagcccacgcctccagttcctccctgaacccaatgcctccatcgcagctgcagctgcccacactgcccttagtgatggtggctcctagcggagctcggctgggacccctgcctcatctgcaagccctactgcaggacaggccgcactttatgcaccagctgtccaccgtcgacgcacatgccaggacccctgtgctccaagtgcaccctctggagagtcctgctatgatcagcctgactccgcctaccacagccacaggcgtgttctccctgaaggccagacccggcctgcctcctggcatcaacgtggcttccctggagtgggtctctcgggagcccgctctgctgtgcactttccctaaccctagcgccccgagaaaggacagcacactgagcgccgttcctcagagctcgtaccccctgctggccaacggagtttgtaaatggcccgggtgcgagaaagtgttcgaagagccggaggacttcttgaagcactgtcaggccgatcacctgctcgacgaaaagggacgtgcccagtgcctgttgcagcgggagatggtgcagtccctagaacagcagctcgtgctggaaaaggaaaagctgagcgccatgcaggcccatctggcaggtaagatggcccttacaaaagcctcttccgtggccagtagcgacaaaggctcctgctgcatcgtcgctgctggctcccagggcccggtggtgcctgcctggtctggcccacgagaggctcctgatagcctctttgctgtgcggcgccatctctggggcagccacggcaattccaccttccccgagttcctgcacaacatggattatttcaagtttcacaacatgcgtccccctttcacctacgctacactgatccggtgggccatcctggaagcacctgagaaacagagaacactgaacgagatctaccactggttcacccggatgtttgcgttcttccgcaaccaccctgctacctggaagaacgctattcgccacaatctgtctctgcacaaatgctttgtccgagtagagagcgagaagggcgccgtgtggaccgtggatgaactggagttcagaaagaagagatcccagagaccatcgaggtgtagcaacccaactccgggccccgggtcgggcgctaccaacttcagcctgctgaagcaggccggcgatgtggaagaaaaccctgggcccatggaaacggaggctattgacggctacatcacctgcgacaacgaactatctcctgaaagagagcactccaatatggcgatcgacctaaccagctccacccctaacggacagcatgcctctccgtcccacatgaccagcaccaacagcgtcaaactggagatgcagtccgacgaggagtgtgaccgcaagcccctgtctcgcgaggacgagataagaggtcatgacgagggatctagcctcgaggaaccactcattgagagctccgaggtggccgacaatcggaaggtgcaggaactacaaggcgaaggcggcatccgtctgcctaatggcgagagacctttccactgtaaccagtgtggcgcgagcttcacccagaagggcaatctgctgagacacatcaagctgcactctggtgagaagccttttaaatgcccattctgctcttacgcctgccgacggcgcgacgccctgaccggccacctccgcacccactctgtgggcaaacctcacaagtgcaactactgcggccgctcctacaagcagagaagcagcttggaagagcacaaggagcgctgtcacaactatctgcagaacgtgtccatggaagccgccggccaggtgatgagccaccacgtgccgcccatggaagattgcaaggagcaggagcccatcatggacaacaacatctcactggtccctttcgagagaccggcggtgattgagaagctgacaggaaacatgggcaagcgcaaaagctcgacgccccagaagttcgtcggcgagaagctgatgcgtttcagctatcccgacatccacttcgacatgaacctgacttacgagaaagaggctgaactgatgcaaagtcacatgatggaccaggccatcaataacgccatcacttacctgggcgccgaggccctgcaccccctgatgcagcatcccccgtctaccatcgccgaggtggcccccgtcattagcagcgcctactcccaggtgtaccaccccaaccgcatcgagcggcccatctctcgtgagactgcggattctcacgagaacaatatggatggtcccatcagcctcatcagacctaagagccgccctcaagagagagaggcctccccaagcaacagctgcctggattctaccgacagcgaatcgagccacgatgaccaccagagctatcagggtcaccctgccctcaacccgaagagaaagcagagccccgcctacatgaaggaagacgtgaaggccctggacaccaccaaggcccctaagggctccctgaaggacatctacaaggtgttcaacggcgagggcgagcaaatccgcgcattcaagtgcgagcactgtagagtgctgttcctggaccacgtgatgtacaccatccatatgggctgtcatggctaccgcgacccacttgagtgcaacatctgtggctacagatctcaggatcgctacgagttttcgagccacatcgtgcgcggagagcacacgtttcactgattaacaacaagagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataacatagcatacatagccgccaccatggcttcccccctgacccgcttcctgtctctgaacctgctactcctcggagagtccatcatcctgggctctggcgaggcagaacagaaactgatttctgaggaagatctggatgtgcagctgcaagaatctgggcccggcctggtgaagccctctcagagcttgtcactgacctgttccgtgaccggatacagcatcacctccggctactattggacctggatccggcagtttcctggcaacaagctggagtggatgggatccattggctatgatggcaccaactactacaatccatctcttaagaaccgcatctctatcaccagagatacatccaagaaccagttctttctgaagctgcacagcgtcaccaccgaggacaccgccacttactactgcgccaagcgcggcatcaccactggcgactactggggccagggtacaaccctgaccgtgtccagcggcgggggcgggtccggaggcggaggatccggcggcggtggcagccaggccgtggtgacgcaagagagcgctctgaccaccagccccggcgagacagtgaccctgacctgtagaagcagcaccggtgcggtaacgacaagcaactacgccaactgggtccaggagaagcccgaccatctgttcatcgggctgatcggtggcaccaacaagcggactccgggtgtgcctgctcgtttcagcgggtcgttaattggcgacaaggccgcgctcaccattacaggtacccagaccgaagacgaggctatctacttctgtgccctgtggtacagcaaccacctggtgttcggcgggggcaccaagctaacagtgctgtccaccacgaccaagcctgtcctgagaacacctagccctgtgcaccccacgggcactagccaacctcagagacccgaggactgccgaccccgtggatctgttaagggcaccggcctggacttcgcctgcgatatctacatctgggcccctctggccggcatctgcgtggccctcctgctgagcctgatcatcactctgatctgctacaacagcagacggaatcgtctgctgcagagcgattacatgaatatgacccctaggcggcctggccttacgagaaaaccctatcagccatacgctcctgcccgcgactttgcggcctaccggccgagagccaaattctcccgccgagccgagactgcagccaacctgcaggaccccaaccagctctacaacgagctgaacctgggcagacgcgaggagtacgacgtgctggaaaagaagagggctcgggaccctgagatgggcggcaagcagcagagaagacggaatccacaggagggcgtgtacaacgccttgcagaaggacaagatggccgaggcttattccgagatcggtacaaaaggtgagagaaggcgcggcaaaggccacgacggcctctaccagggcctgagcacagccaccaaggacacctacgacgccctgcacatgcagacactggcgccgcgctga

SEQ ID NO: 20 - RNA, 4884 nt, synthetic construct
  source 1..4884: mol_type = other RNA
atagtaatagccgccaccatgggacccaatcccagacccgggaagcctagcgccccgtctcttgccttggggccctctcccggtgcctcacctagttggagagccgcgcctaaggcttctgatctgctgggcgctagaggccctggggggacgttccagggccgggacctgagaggcggagcccacgcctccagttcctccctgaacccaatgcctccatcgcagctgcagctgcccacactgcccttagtgatggtggctcctagcggagctcggctgggacccctgcctcatctgcaagccctactgcaggacaggccgcactttatgcaccagctgtccaccgtcgacgcacatgccaggacccctgtgctccaagtgcaccctctggagagtcctgctatgatcagcctgactccgcctaccacagccacaggcgtgttctccctgaaggccagacccggcctgcctcctggcatcaacgtggcttccctggagtgggtctctcgggagcccgctctgctgtgcactttccctaaccctagcgccccgagaaaggacagcacactgagcgccgttcctcagagctcgtaccccctgctggccaacggagtttgtaaatggcccgggtgcgagaaagtgttcgaagagccggaggacttcttgaagcactgtcaggccgatcacctgctcgacgaaaagggacgtgcccagtgcctgttgcagcgggagatggtgcagtccctagaacagcagctcgtgctggaaaaggaaaagctgagcgccatgcaggcccatctggcaggtaagatggcccttacaaaagcctcttccgtggccagtagcgacaaaggctcctgctgcatcgtcgctgctggctcccagggcccggtggtgcctgcctggtctggcccacgagaggctcctgatagcctctttgctgtgcggcgccatctctggggcagccacggcaattccaccttccccgagttcctgcacaacatggattatttcaagtttcacaacatgcgtccccctttcacctacgctacactgatccggtgggccatcctggaagcacctgagaaacagagaacactgaacgagatctaccactggttcacccggatgtttgcgttcttccgcaaccaccctgctacctggaagaacgctattcgccacaatctgtctctgcacaaatgctttgtccgagtagagagcgagaagggcgccgtgtggaccgtggatgaactggagttcagaaagaagagatcccagagaccatcgaggtgtagcaacccaactccgggccccgggtcgggcgctaccaacttcagcctgctgaagcaggccggcgatgtggaagaaaaccctgggcccatggaaacggaggctattgacggctacatcacctgcgacaacgaactatctcctgaaagagagcactccaatatggcgatcgacctaaccagctccacccctaacggacagcatgcctctccgtcccacatgaccagcaccaacagcgtcaaactggagatgcagtccgacgaggagtgtgaccgcaagcccctgtctcgcgaggacgagataagaggtcatgacgagggatctagcctcgaggaaccactcattgagagctccgaggtggccgacaatcggaaggtgcaggaactacaaggcgaaggcggcatccgtctgcctaatggcgagagacctttccactgtaaccagtgtggcgcgagcttcacccagaagggcaatctgctgagacacatcaagctgcactctggtgagaagccttttaaatgcccattctgctcttacgcctgccgacggcgcgacgccctgaccggccacctccgcacccactctgtgggcaaacctcacaagtgcaactactgcggccgctcctacaagcagagaagcagcttggaagagcacaaggagcgctgtcacaactatctgcagaacgtgtccatggaagccgccggccaggtgatgagccaccacgtgccgcccatggaagattgcaaggagcaggagcccatcatggacaacaacatctcactggtccctttcgagagaccggcggtgattgagaagctgacaggaaacatgggcaagcgcaaaagctcgacgccccagaagttcgtcggcgagaagctgatgcgtttcagctatcccgacatccacttcgacatgaacctgacttacgagaaagaggctgaactgatgcaaagtcacatgatggaccaggccatcaataacgccatcacttacctgggcgccgaggccctgcaccccctgatgcagcatcccccgtctaccatcgccgaggtggcccccgtcattagcagcgcctactcccaggtgtaccaccccaaccgcatcgagcggcccatctctcgtgagactgcggattctcacgagaacaatatggatggtcccatcagcctcatcagacctaagagccgccctcaagagagagaggcctccccaagcaacagctgcctggattctaccgacagcgaatcgagccacgatgaccaccagagctatcagggtcaccctgccctcaacccgaagagaaagcagagccccgcctacatgaaggaagacgtgaaggccctggacaccaccaaggcccctaagggctccctgaaggacatctacaaggtgttcaacggcgagggcgagcaaatccgcgcattcaagtgcgagcactgtagagtgctgttcctggaccacgtgatgtacaccatccatatgggctgtcatggctaccgcgacccacttgagtgcaacatctgtggctacagatctcaggatcgctacgagttttcgagccacatcgtgcgcggagagcacacgtttcactgattaacaacaagagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataacatagcatacatagccgccaccatggcttcccccctgacccgcttcctgtctctgaacctgctactcctcggagagtccatcatcctgggctctggcgaggcagaacagaaactgatttctgaggaagatctggatgtgcagctgcaagaatctgggcccggcctggtgaagccctctcagagcttgtcactgacctgttccgtgaccggatacagcatcacctccggctactattggacctggatccggcagtttcctggcaacaagctggagtggatgggatccattggctatgatggcaccaactactacaatccatctcttaagaaccgcatctctatcaccagagatacatccaagaaccagttctttctgaagctgcacagcgtcaccaccgaggacaccgccacttactactgcgccaagcgcggcatcaccactggcgactactggggccagggtacaaccctgaccgtgtccagcggcgggggcgggtccggaggcggaggatccggcggcggtggcagccaggccgtggtgacgcaagagagcgctctgaccaccagccccggcgagacagtgaccctgacctgtagaagcagcaccggtgcggtaacgacaagcaactacgccaactgggtccaggagaagcccgaccatctgttcatcgggctgatcggtggcaccaacaagcggactccgggtgtgcctgctcgtttcagcgggtcgttaattggcgacaaggccgcgctcaccattacaggtacccagaccgaagacgaggctatctacttctgtgccctgtggtacagcaaccacctggtgttcggcgggggcaccaagctaacagtgctgtccaccacgaccaagcctgtcctgagaacacctagccctgtgcaccccacgggcactagccaacctcagagacccgaggactgccgaccccgtggatctgttaagggcaccggcctggacttcgcctgcgatatctacatctgggcccctctggccggcatctgcgtggccctcctgctgagcctgatcatcactctgatctgctacaacagcagacggaatcgtctgctgcagagcgattacatgaatatgacccctaggcggcctggccttacgagaaaaccctatcagccatacgctcctgcccgcgactttgcggcctaccggccgagagccaaattctcccgccgagccgagactgcagccaacctgcaggaccccaaccagctctacaacgagctgaacctgggcagacgcgaggagtacgacgtgctggaaaagaagagggctcgggaccctgagatgggcggcaagcagcagagaagacggaatccacaggagggcgtgtacaacgccttgcagaaggacaagatggccgaggcttattccgagatcggtacaaaaggtgagagaaggcgcggcaaaggccacgacggcctctaccagggcctgagcacagccaccaaggacacctacgacgccctgcacatgcagacactggcgccgcgctga

SEQ ID NO: 21 - DNA, 4881 bp, synthetic construct
  source 1..4881: mol_type = other DNA
atagtaatagccgccaccatggaaaccgaggccatcgatggctacatcacctgcgacaacgagctgagcccggagagagagcactcaaatatggccattgacctgaccagcagcacacctaatggtcagcacgcctctccgtctcatatgacctccaccaactccgtaaagctggagatgcagagcgatgaggagtgtgatcgcaagcctctgtcgcgggaagacgagatccggggccatgacgaaggcagctccctggaagagcctttgatcgagagctctgaagtcgcggacaaccgcaaagtgcaggagctgcagggggagggcggcatcaggctgcccaacggtgagcgaccgttccactgtaatcagtgtggggcttccttcacccagaagggtaacctgctgcgccacatcaagctgcacagcggcgagaaacccttcaagtgcccattctgctcttatgcctgcagacgacgcgacgctctgaccggccacctccgcacccacagcgtgggcaagccccacaagtgcaactactgcgggcgttcctacaagcagagatcttctctggaagaacacaaggagcggtgccacaactacctgcagaacgtgagcatggaggctgcgggacaggtgatgagccaccacgtgccacctatggaagattgcaaggaacaggagcccatcatggataacaacatctccctcgtgcctttcgagcgccctgccgtgattgagaaacttaccggcaacatgggcaagcgcaagtcctctactccgcagaagttcgtgggggagaagctgatgcggttcagctaccccgacatccatttcgacatgaacctgacctacgagaaagaggccgagctgatgcagagccacatgatggaccaggctatcaacaacgccattacctatctgggcgctgaggccctgcaccccctgatgcagcacccccctagcacaatcgccgaggtggctccggtgatctcatccgcctacagccaagtgtaccaccctaacagaatcgaacggcccatctcccgggagaccgccgacagccacgagaacaacatggacggccctatatccctgatccgccctaagagccgccctcaggagagagaggctagcccatctaattcatgcctggattctaccgactccgagagtagccacgatgaccaccagagctaccagggccaccccgctctgaacccaaagcggaagcagagccccgcctacatgaaggaggatgtgaaggccctagacaccaccaaggccccgaaagggtccctgaaagacatctacaaggtgtttaacggcgagggcgagcaaatcagagcttttaagtgcgagcactgccgggtgctgttcctggatcatgtgatgtacaccatccacatgggttgtcacggctatagagacccactggaatgtaacatctgtggctacagatcgcaagacagatatgagttcagcagccacatcgtgcgcggcgaacataccttccatggctccggcgctaccaacttctccctactgaagcaggctggcgatgtggaagagaatccagggccgatgcctaatcctcgacccggcaaaccttccgcaccgtctctggccctgggacccagccctggagcttctccaagttggcgggccgcgcccaaggcttcggacctgttgggcgcgagaggaccgggcggcaccttccagggccgggatctgcgcgggggagcgcacgccagcagctcctcgctgaaccccatgcctccatcgcagctgcaattgccaaccctgcccctggtgatggtggcccctagcggtgcccgcctgggccctctgcctcatcttcaggccctgctccaggacaggcctcattttatgcaccagctgagtacggttgacgcccatgcaagaacacctgtgttacaggtccaccctctggagtcccccgccatgatctctcttaccccgcctacaactgctacgggtgtgttcagtctgaaggcccgtcctggcctacccccaggcatcaacgtggctagtttggaatgggttagccgggaaccagccctgctgtgcactttccccaatccaagcgctcctagaaaggactccacactgtctgccgtccctcagtctagctaccctctgctggccaacggcgtgtgcaaatggcctggatgcgagaaagtgtttgaggagcctgaggacttcctgaagcactgtcaggccgaccacctgctcgacgagaagggccgcgcccagtgcctgctccagcgcgagatggtgcagagcctggaacagcagctggtgctcgaaaaagagaaacttagcgccatgcaggcccatctggcaggaaagatggctctcacaaaggcaagcagcgtggcctcctctgacaaggggagctgttgtattgtcgccgccggctcacagggaccggtggtccccgcttggtctggcccccgcgaagcccccgactccctgttcgccgtacgtcgacacctttggggctctcacggcaactccacatttcctgaattcctgcacaacatggactacttcaaatttcacaacatgagaccacccttcacctacgccacgctgatcagatgggccatcctggaggcgcccgagaagcagcggactctgaacgagatctaccactggttcacacgcatgtttgcgttcttccggaaccaccccgcaacctggaagaacgccatcagacacaacctgagcctccacaaatgtttcgtcagagtggagagcgagaagggcgccgtgtggaccgtggacgagctggaattcagaaagaagcggagccaaagaccttcgaggtgtagtaaccccacccccggaccttgattaacaacaagagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataacatagcatacatagccgccaccatggcctccccactgaccagattcttgtctctcaacctgctgctgctgggcgaaagcatcatcctgggctccggagaggctgaacagaagctgatcagcgaggaagacctggacgtgcagctgcaagagtctggacctggtctagtgaagccttcccagagcctgtctctgacctgtagcgtgaccggctatagcatcacctcgggctactattggacctggatccgccagtttcctggcaacaaactggagtggatgggaagtatcggatacgacggcaccaactactacaaccctagcctgaagaaccggatctctatcacccgcgacacttccaagaatcagttcttcctgaaactgcacagcgttaccaccgaggacactgccacctactactgcgccaaacgcggtatcaccacaggtgattattggggccagggcaccacactgactgtgtcttctggcggagggggttccggagggggaggttccggcgggggcggcagccaggccgtggtgacccaggagagcgccctgaccaccagccctggcgagacagtaacactgacctgcagatcctcgaccggcgcggtcaccacatctaattacgccaattgggtccaagagaagcccgaccatctgttcatcggcctcattggcggcaccaacaagcggactcctggcgtgcctgcacgattttcgggttctctgattggcgacaaggccgccctcacaattactggcacccagactgaggacgaggcgatctacttctgtgctctgtggtacagcaaccacctggtgttcggcggtggaaccaagctgaccgtgctgagcaccacaaccaagccggtcctgagaacaccttctcctgtgcaccccacggggacctcccagcctcagcgcccggaagattgcagaccaaggggcagtgtgaaaggcaccggtcttgactttgcctgcgacatctacatctgggcccctctggccggcatctgtgtggccctgctgctttccctcatcatcaccctcatctgctacaacagccgccggaaccgcctgctgcaaagcgactacatgaacatgacccccagacggcccggcctcacaagaaagccctatcagccatatgccccggcgcgggacttcgccgcttacaggcccagagccaagttcagcagacgcgctgagaccgcggccaacctgcaggaccccaaccagctatacaacgagctgaatctcggcagacgcgaggagtacgatgtgctggaaaagaagcgcgcgagagatcctgagatgggcggcaagcagcagagacgtcgtaacccccaggagggcgtgtacaacgccttgcagaaggacaagatggctgaagcctactccgagatcgggacaaagggcgagcggcgtagaggcaaaggccacgatggcctgtaccagggcctcagcacagctaccaaggatacatacgacgccctgcacatgcagacgttggctcctcgttga

SEQ ID NO: 22 - RNA, 4881 nt, synthetic construct
  source 1..4881: mol_type = other RNA
atagtaatagccgccaccatggaaaccgaggccatcgatggctacatcacctgcgacaacgagctgagcccggagagagagcactcaaatatggccattgacctgaccagcagcacacctaatggtcagcacgcctctccgtctcatatgacctccaccaactccgtaaagctggagatgcagagcgatgaggagtgtgatcgcaagcctctgtcgcgggaagacgagatccggggccatgacgaaggcagctccctggaagagcctttgatcgagagctctgaagtcgcggacaaccgcaaagtgcaggagctgcagggggagggcggcatcaggctgcccaacggtgagcgaccgttccactgtaatcagtgtggggcttccttcacccagaagggtaacctgctgcgccacatcaagctgcacagcggcgagaaacccttcaagtgcccattctgctcttatgcctgcagacgacgcgacgctctgaccggccacctccgcacccacagcgtgggcaagccccacaagtgcaactactgcgggcgttcctacaagcagagatcttctctggaagaacacaaggagcggtgccacaactacctgcagaacgtgagcatggaggctgcgggacaggtgatgagccaccacgtgccacctatggaagattgcaaggaacaggagcccatcatggataacaacatctccctcgtgcctttcgagcgccctgccgtgattgagaaacttaccggcaacatgggcaagcgcaagtcctctactccgcagaagttcgtgggggagaagctgatgcggttcagctaccccgacatccatttcgacatgaacctgacctacgagaaagaggccgagctgatgcagagccacatgatggaccaggctatcaacaacgccattacctatctgggcgctgaggccctgcaccccctgatgcagcacccccctagcacaatcgccgaggtggctccggtgatctcatccgcctacagccaagtgtaccaccctaacagaatcgaacggcccatctcccgggagaccgccgacagccacgagaacaacatggacggccctatatccctgatccgccctaagagccgccctcaggagagagaggctagcccatctaattcatgcctggattctaccgactccgagagtagccacgatgaccaccagagctaccagggccaccccgctctgaacccaaagcggaagcagagccccgcctacatgaaggaggatgtgaaggccctagacaccaccaaggccccgaaagggtccctgaaagacatctacaaggtgtttaacggcgagggcgagcaaatcagagcttttaagtgcgagcactgccgggtgctgttcctggatcatgtgatgtacaccatccacatgggttgtcacggctatagagacccactggaatgtaacatctgtggctacagatcgcaagacagatatgagttcagcagccacatcgtgcgcggcgaacataccttccatggctccggcgctaccaacttctccctactgaagcaggctggcgatgtggaagagaatccagggccgatgcctaatcctcgacccggcaaaccttccgcaccgtctctggccctgggacccagccctggagcttctccaagttggcgggccgcgcccaaggcttcggacctgttgggcgcgagaggaccgggcggcaccttccagggccgggatctgcgcgggggagcgcacgccagcagctcctcgctgaaccccatgcctccatcgcagctgcaattgccaaccctgcccctggtgatggtggcccctagcggtgcccgcctgggccctctgcctcatcttcaggccctgctccaggacaggcctcattttatgcaccagctgagtacggttgacgcccatgcaagaacacctgtgttacaggtccaccctctggagtcccccgccatgatctctcttaccccgcctacaactgctacgggtgtgttcagtctgaaggcccgtcctggcctacccccaggcatcaacgtggctagtttggaatgggttagccgggaaccagccctgctgtgcactttccccaatccaagcgctcctagaaaggactccacactgtctgccgtccctcagtctagctaccctctgctggccaacggcgtgtgcaaatggcctggatgcgagaaagtgtttgaggagcctgaggacttcctgaagcactgtcaggccgaccacctgctcgacgagaagggccgcgcccagtgcctgctccagcgcgagatggtgcagagcctggaacagcagctggtgctcgaaaaagagaaacttagcgccatgcaggcccatctggcaggaaagatggctctcacaaaggcaagcagcgtggcctcctctgacaaggggagctgttgtattgtcgccgccggctcacagggaccggtggtccccgcttggtctggcccccgcgaagcccccgactccctgttcgccgtacgtcgacacctttggggctctcacggcaactccacatttcctgaattcctgcacaacatggactacttcaaatttcacaacatgagaccacccttcacctacgccacgctgatcagatgggccatcctggaggcgcccgagaagcagcggactctgaacgagatctaccactggttcacacgcatgtttgcgttcttccggaaccaccccgcaacctggaagaacgccatcagacacaacctgagcctccacaaatgtttcgtcagagtggagagcgagaagggcgccgtgtggaccgtggacgagctggaattcagaaagaagcggagccaaagaccttcgaggtgtagtaaccccacccccggaccttgattaacaacaagagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataacatagcatacatagccgccaccatggcctccccactgaccagattcttgtctctcaacctgctgctgctgggcgaaagcatcatcctgggctccggagaggctgaacagaagctgatcagcgaggaagacctggacgtgcagctgcaagagtctggacctggtctagtgaagccttcccagagcctgtctctgacctgtagcgtgaccggctatagcatcacctcgggctactattggacctggatccgccagtttcctggcaacaaactggagtggatgggaagtatcggatacgacggcaccaactactacaaccctagcctgaagaaccggatctctatcacccgcgacacttccaagaatcagttcttcctgaaactgcacagcgttaccaccgaggacactgccacctactactgcgccaaacgcggtatcaccacaggtgattattggggccagggcaccacactgactgtgtcttctggcggagggggttccggagggggaggttccggcgggggcggcagccaggccgtggtgacccaggagagcgccctgaccaccagccctggcgagacagtaacactgacctgcagatcctcgaccggcgcggtcaccacatctaattacgccaattgggtccaagagaagcccgaccatctgttcatcggcctcattggcggcaccaacaagcggactcctggcgtgcctgcacgattttcgggttctctgattggcgacaaggccgccctcacaattactggcacccagactgaggacgaggcgatctacttctgtgctctgtggtacagcaaccacctggtgttcggcggtggaaccaagctgaccgtgctgagcaccacaaccaagccggtcctgagaacaccttctcctgtgcaccccacggggacctcccagcctcagcgcccggaagattgcagaccaaggggcagtgtgaaaggcaccggtcttgactttgcctgcgacatctacatctgggcccctctggccggcatctgtgtggccctgctgctttccctcatcatcaccctcatctgctacaacagccgccggaaccgcctgctgcaaagcgactacatgaacatgacccccagacggcccggcctcacaagaaagccctatcagccatatgccccggcgcgggacttcgccgcttacaggcccagagccaagttcagcagacgcgctgagaccgcggccaacctgcaggaccccaaccagctatacaacgagctgaatctcggcagacgcgaggagtacgatgtgctggaaaagaagcgcgcgagagatcctgagatgggcggcaagcagcagagacgtcgtaacccccaggagggcgtgtacaacgccttgcagaaggacaagatggctgaagcctactccgagatcgggacaaagggcgagcggcgtagaggcaaaggccacgatggcctgtaccagggcctcagcacagctaccaaggatacatacgacgccctgcacatgcagacgttggctcctcgttga

SEQ ID NO: 23 - DNA, 587 bp, synthetic construct
  source 1..587: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 24 - DNA, 653 bp, synthetic construct
  source 1..653: mol_type = other DNA
gagtgttcccacccaacaggcccactgggtgttgtactctggtattacggtacctttgtacgcctattttatttcccccccctttttgaaacttagaagttaataataaacacgctcactaggtgcactacatccagtagtgtaatgagcaagcacttctgtcttccccgggagggatatatggtacgctgtgcaaacggcggaaattaatcctaccgttaaccgcccacctactccgagaagcctagtacctaattggatttatcaatggagttgcgctcagcaggtgaccctgacctgccagctccggctgatggacctgggctttccccacaggcgactgtggcccaggtcgcgtggcggccggcccacccccctgggtgggacgccttgataatgacaaggtgggaagagcctattgggctagctggtttcctccggcctcctgaatgcggctaaccttaaccccagagcatatggtagcaacccagctactagtatgtcataatgcgtaagtctgggatgggaccgactactttggagagtccgtgtttctattgtttctttaatcaatcttatggtgacaatttatagtgccctgagtattgattggttgttgcttttgacaattattgagacatcacatagacata

SEQ ID NO: 25 - DNA, 265 bp, synthetic construct
  source 1..265: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 26 - DNA, 383 bp, synthetic construct
  source 1..383: mol_type = other DNA
gccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaac

SEQ ID NO: 27 - DNA, 742 bp, synthetic construct
  source 1..742: mol_type = other DNA
ttaaaacagcctgtgggttgttcccacccgcagggcccactgggcgccagcactctggtatcacggtacctttgtgcgcctgttttaaaaccctctccccaatttgaaacttagaagcaatacacctcgatcaatagtaggcatgacacgccagccatgtcttgatcaagcacttctgtttccccggactgagtatcaataaactgcttgcgcggtcgaaggagaaaacgtccgttacccgactaactacttcgagaaacccagtaacaccatggaaattgcggagtgtttcactcagcacattcccagtgtagatcaggtcgatgagtcaccgcattccccacgggtgaccgtggcggtggctgcgctggcggcctgcccatggggcaacccatgggacgcttcaatatggacatggtgtgaagagtctattgagctagttagtagtcctccggcccctgaatgcggctaatcctaactgcggagcacgtgcctccattccagggggtggcgtgtcgtaacgggcaactctgcagcggaaccgactactttgggtgtccgtgtttcttttaattttatactggctgcttatggtgacaattgaaagattgttgccatatagctattggattggccatccggtatccaacagagcaattgtgtacctttttgttggatttgtaccacttaccagaacaagttttcatacactgtgttacattattagactaaacacagaaaa

SEQ ID NO: 28 - DNA, 744 bp, synthetic construct
  source 1..744: mol_type = other DNA
ttaaaacagctctggggttgtacccaccccagaggcccacgtggcggctagtactccggtattacggtacccttgtacgcctgttttatactccctccctcgtaacttagaagcacaaaaccaagttcaatagaagggggtgcaaaccagtaccaccacgaacaagcatttctgtttccccggtgatgttgtatagactgcccgcgcggttgaaagcaacggtaccgttacccgctcaagtacttcgagaagcctagtattaccttggaatcttcgatgcgttgcgttcagcactcgaccctggagtgtagcttaggctgatgagtctggacgtccctcaccggtgacggtggtccaggctgcgttggcggcctacctatggctaacgccataggacgctagttgtgaacaaggtgtgaagagcctattgagctacttgagagtcctccggcccctgaatgcggctaatcctaaccatggagcaggcggtcacagaccagtgactagcttgtcgtaatgcgcaagtctatggcggaaccgactactttgggtgtccgtgtttccttttatttttattatggctgcttatggtgacaatcattgattgttatcataaagcgaattggattggccatccggtgaaagcgagacttactatttacttacttgttggacttaccacacttaatacatttattctaggtgtcacttgtatagcaattagaatcaaacagttgcatcata

SEQ ID NO: 29 - DNA, 743 bp, synthetic construct
  source 1..743: mol_type = other DNA
ttaaaacagcctgtgggttgttcccacccacagggcccactgggcgctagcacactggtatcacggtacctttgtgcgcctgtttgacttaccctccccaaacgcaacttagaagcacaacttaaatggtcaataggcggctcagtatgccaactgagtctcgatcaagcacttctgttaccccggactgagtaccaataggctgctcacgcggctgaaggggaaaccgttcgttacccgactaactacttcgagaaacctagtaccaccatgaaagttgcgcagcgtttcgctccgcacaaccccagtgtagatcaggtcgatgagtcaccgcaaaccccacgggcgaccgtggcggtggctgcgctggcggcctgcccatggggcaactcatgggacgcttcaatactgacatggtgcgaagagtctattgagctagttggtagtcctccggcccctgaatgcggctaatcctaactgcggagcagatacccacgcaccagtgggcagtctgtcgtaatgggcaactctgcagcggaaccgactactttgggtgtccgtgtttccttttgttcttatactggctacttatggtgacaattgagagattgtaaccatattgctattggattggccacccggcgacgaatagaacagttgcttacctgtttgttagtctcgtatcactgaactacaaatccttaaacaccctttaatttcgtcataacgctcaatacgttaaa

SEQ ID NO: 30 - DNA, 600 bp, synthetic construct
  source 1..600: mol_type = other DNA
atccaggttgttcccacctggatttcccacagggagtggtactctgttattacggtaactttgtacgccagttttatctcccttcccccatgtaacttagaagtttttcacaaagaccaatagccggtaatcagccagattactgaaggtcaagcacttctgtttccccggtcaatgttgatatgctccaacagggcaaaaacaactgcgatcgttaaccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaatgtgtatctagtcgtaatgagcaattgcgggatgggaccaactactttgggtgtccgtgtttcactttttcctttatatttgcttatggtgacaatatatacaatatatatattggcacc

SEQ ID NO: 31 - DNA, 612 bp, synthetic construct
  source 1..612: mol_type = other DNA
ttaaagctggatcatggttgttcccaccatgattacccacgcggtgcagtggtcttgtattacggtacatttccataccagttttatacaccccaccccgaaactcatagaagtttgtacacaatgaccaataggtggtggccatccaggtcgctaatggtcaagcacttctgtttccccggcacccttgtatacgcttcacccgaggcgaaaaatgaggttgtcgttatccgcaaagtgcctacgaaaagcctagtaacactttgaaaacccatggttggtcgctcagctgtttacccaacagtagacctggcagatgaggctagacattccccaccagcgatggtggtctagcctgcgtggctgcctgcacaccctgccgggtgtgaagccagaaagtggacaaggtgtgaagagcctattgtgctcactttgagtcctccggcccctgaatgtggctaaccctaaccccgtagctgttgcatgtaacccaacatgtatgcagtcgtaatgggcaactatgggatgggaccaactactttgggtgtccgtgtttcctgttttactttttcattgcttatggtgacaattgtatctgatacacttgttacc

SEQ ID NO: 32 - DNA, 618 bp, synthetic construct
  source 1..618: mol_type = other DNA
ttaaaactggatacaggttgttcccacctgtatcacccaagtggtgtggtgctcttgtatttcggtacgtttgcacgccagtttgctaccccttcccttttacgtaacttagaagtttacacaaagaccaataggcggtggtaaatccataccactaacggtcaaatacttctgtttccccggcatgcgaggaataggctccaaaagggctgaagccactagtgtcgttatccgcattggtactacgcaaagcctagtattaccttgaaaatttcttggctggtcgctccaccagataccccactggtagacctggcagatgaggcaggacttaccccactggcgacagtggtcctgcctgcgtggctgcctgcacaccccttacggggtgtgaagcccagaaacagacaaggtgtgaagagccccgtgtgctactagtgagtcctccggcccctgaatgcggctaatcttaccccacagctgttgcacgcaaaccagcgtgtatgcagtcgtaatgagcaattgtgggatggaaccgactactttgggtgtccgtgtttctttttattcctattatttgcttatggtgacaatattgatattatcagtgttgtcatc

SEQ ID NO: 33 - DNA, 741 bp, synthetic construct
  source 1..741: mol_type = other DNA
ttaaaacagcctgtgggttgttcccacccacaggcccattgggcgctagcactctggtatcacggtacctttgtgcgcctgttttacatcccctccccaaattgtaatttagaagtttcacacaccgatcattagcaagcgtggcacaccagccatgttttgatcaagcacttctgttaccccggactgagtatcaatagaccgctaacgcggttgaaggagaaaacgttcgttacccggccaactacttcgaaaaacctagtaacaccatggaagttgcggagtgtttcgctcagcactaccccagtgtagatcaggtcgatgagtcaccgcgttccccacgggcgaccgtggcggtggctgcgttggcggcctgcctacggggaaacccgtaggacgctctaatacagacatggtgcgaagagtctattgagctagttggtaatcctccggcccctgaatgcggctaatcctaactgcggagcacataccctcaaaccagggggcagtgtgtcgtaacgggcaactctgcagcggaaccgactactttgggtgtccgtgtttcattttattcctatactggctgcttatggtgacaattgacaggttgttaccatatagttattggattggccatccggtgactaacagagcaattatatatctctttgttgggtttataccacttagcttgaaagaggttaaaacactacatctcatcattaaactaaatacaacaaa

SEQ ID NO: 34 - DNA, 747 bp, synthetic construct
  source 1..747: mol_type = other DNA
ttaaaacagctctggggttgttcccaccccagaggcccacgtggcggccagtacactggtatcgcggtacctttgtacgcctgttttatactcccttcccccgtaacttagaagcacaacgtccaagttcaatagaagggggtacaaaccagtaccaccacgaacaagcacttctgttcccccggtgaggctgtataggctgtttccacggctaaaagcggctgatccgttatccgctcatgtacttcgagaagcctagtatcaccttggaatcttcgatgcgttgcgctcaacactcaaccccagagtgtagcttaggtcgatgagtctggacgttcctcaccggcgacggtggtccaggctgcgttggcggcctacctgtggcccaaagccacaggacgctagttgtgaacaaggtgtgaagagcctattgagctacctgagagtcctccggcccctgaatgcggctaatcctaaccacggagcaggcagtggcaatccagcgaccagcctgtcgtaacgcgcaagttcgtggcggaaccgactactttgggtgtccgtgtttccttttatttttacaatggctgcttatggtgacaattattgatagttatcataaagcaaattggattggccatccggtgagaatttgattattaaattactctcttgttgggattgctcctttgaaatcctgtgcactcacacctattggaattacctcattgttgagatattattaccact

SEQ ID NO: 35 - DNA, 618 bp, synthetic construct
  source 1..618: mol_type = other DNA
tttaaacagcggatgggttccccaccatccgacccactgggtgtagtactctggtattttgtacctttgtacgcctgtttctcccctacctcccaacctaaacaatcctggtaacttagaagacttaaatcatcgtacaataggtgctgtcacatccagtgacggctagtacaagcacttctgtttccccggagcggagtataaatggccaccgctgtcaaaagctcttaaccgttatccgccaattaactacgcaacggctagtaacatcttgttatttttagggcgttcgatcaggtgagtaaacccctcactagtctggtcgatgaggctgagaattccccacgggcgaccgtgtctcagcctgcgtggcggccagcccagctaatgctgggacgccttaattgtgacatggtgtgaagacccacgtgtgcttaattgtgagtcctccggcccctgaatgcggctaacctaaaccctggagccttgagacacaatccagtgttggcaaggtcgtaatgagtaattccgggacgggaccgactactttgggtgtccgtgtttccttttattttcaaattgttcttatggtcacaatataagtaatatattgtgatc

SEQ ID NO: 36 - DNA, 743 bp, synthetic construct
  source 1..743: mol_type = other DNA
ttaaaacagcctgtgggttgtacccacccacagggcccactgggcgctagcactctggtattacggtacccttgtgcgcctgttttatataccccgccccaagcaaacgctagatgtaacgcacttatgatcaatagcaggcgtggcactccagccacgttatgatcaagcacttctgtctccccggaccgagtatcaatagactgctcacgcggtcgaaggagaaaacgttcgttacccgaccagctacttcgagaaacctagtaactccatggaggttgcagagtgtttcgttcagcacttcccccgtgtagatcaggctgatgagtcaccgcgttcctcacgggcgaccgtggcggtggctgcgttggcggcctgcctatgggtatacccataggacgctctaatactgacatggtgcgaagagtctattgagctagttggtagtcctccggcccctgaatgcggctaatcctaactgcggagcacatacccccaacccagggggcagtgtgtcgtaacgggtaactctgcagcggaaccgactactttgggtgtccgtgtttccttttattcttacattggctgcttatggtgacaattgagagattgttaccatatagctattggattggccatccagtgactaacagagctattatttacctttttgttggcttcgtatcacttggtttaaaagaggttagtactttatattgcattatattactaaacacgagaaa

SEQ ID NO: 37 - DNA, 751 bp, synthetic construct
  source 1..751: mol_type = other DNA
ttaaaacagcctgtgggttgtacccacccacagggcccactgggcgctagcacactggtatcacggtacctttgtgcgcctgttttatacccccttcccgcaaccgcaaatttagaagcaaagctaacccgatcgatagcggatgcgcatgccagccgcattttgatcaagtacttctgtttccccggaccgagtatcaatagactgctcacgcggttgaaggagaaaacgtccgttacccgaccaactacttcgagaaacctagtaacatcatgaatgttgcagggcgtttcgatcagcacgaccctggtgtagatcaggctgatgagtcaccgcattccccacgggtgaccgtggcggtggctgcgttggcggcctgcctatggggtgacccataggacgctctaatacggacatggtgcgaagagtctattgagctagttggtagtcctccggcccctgaatgcggttaatcctaactgcggacgacatacccctaatccaaggggcagtgtgtcgtaacgggcaactctgcagcggaaccgactactttgggtgtccgtgtttccttttatttttatactggctgcttatggtgacaatctcagagttgttaccatatagctattggattggccatccggtgagcaacagagctgtcatttatcagtttgttggctttatacctctaaatcacacggttttttttttttggaacgcttgtattcatcttaaccctcaataaggcaaa

SEQ ID NO: 38 - DNA, 751 bp, synthetic construct; identical to SEQ ID NO: 37
  source 1..751: mol_type = other DNA

SEQ ID NO: 39 - DNA, 232 bp, synthetic construct
  source 1..232: mol_type = other DNA
ttctagcactagtaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctacatttcaagataatctagccacc

SEQ ID NO: 40 - DNA, 618 bp, synthetic construct
  source 1..618: mol_type = other DNA
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatc

SEQ ID NO: 41 - DNA, 632 bp, synthetic construct
  source 1..632: mol_type = other DNA
ttaaaacagcgggtgggtatcccaccacccgacccactgggtgttgtacactggtattttgtacctttgtatgcctgtttgcacctccccaccccttccaattacccttacccgaattgtattatgcggtaacattagaagaagtgaacacagtgcaataggacgtatcacatccagtgatataaagcacaagcaattcttgttccccggagctggatatagactgctaacgtggttgaaggtccttaaccgttatccgccaaccaactacgacacggctagtaatatcatgtttgtctttgagcgttcgatcaggtgaattccccattcactagtttggtcgatgaggctgagaactccccacaggtgactgtgtctcagcctgcgtggcggccaacccagccacggctgggacgcccactgatagacatggtgtgaagacccaattgtgcttggttgtgactcctccggcccctgaatgcggctaacctcaaccccggagccttgtagtgtaagccaacacatacaaggtcgtaatgggcaactctgggacgggaccgactactttgggtgtccgtgtttcctttatttttatcttttgtgtcttatggttacaagtattgattgtaacc

SEQ ID NO: 42 - DNA, 812 bp, synthetic construct
  source 1..812: mol_type = other DNA
ttaaaacagcctgggggttgtacccacccctggggcccacgcggcgccagtactctggtacgctagtacctttgtacgcctgttttccccacccttaaataaattaagattaccactgctgtggggagtagtccgactccgcaccgatacgtcgcaccagtgaactggttcgcttagaacctttgcacggagcagctggtatccccccccgtaacttagaagcctggacaaaccgaccaatagaggcgttgtagccagcagcgcaacggtcaagcacttctgtttccccggcgccatgcgtcgttacccgcccggcctactgcgggaagcctagtaggaccccaatcggacgcgcggttgcgttcagccacaaccccagtggtagctctgaggaatgggactcgcctaccccccacagcaatgtggtggcttgtccgcgtgtgtcctcgggttcgctcgttgagcgatcaccgcaactctgagtaaggtctcaagagcctactgcgctaggtcggttctcctccggagccgtgaatgctgctaatcccaacctccgagcgtgtgcgcacaacccagtgttgctacgtcgtaatgcgtaagttggaggcggaacagactactttcggtactccgtgtttccttaatttttgttcattattacatggtgacattgactgacatttgtgaatttgcccgcctgcccttgaacatagctctctattacttgattgcatttcacaaaacaacccaactcactgttgaacttgttgacttcgcagtctacttgaacttacagtacaatcacattcaca

SEQ ID NO: 43 - DNA, 743 bp, synthetic construct
  source 1..743: mol_type = other DNA
ttaaaacagcctgtgggttgttcccacccacagggcccactgggcgttagcacactggtatcacggtacctttgtgcgcctgttttatactcccccccctaaggaaactttagaagcaaagcaattgtgatcaatagtgggtatggcacaccagtcatatcttgatcaagcacttctgttcccccggacttagtaccaatagactgctcaagcggttgaaggggaaaacgttcgttatccggccaactacttcgagaaacctagtagcaccatgaaagttgcggagtgtttcgctcagcacttcccccgtgtagatcaggctgatgagtcaccgtattccccacgggcgaccgtgacggtggctgcgttggcggcctgcccatggggtaacccatgggacgctctaaaacagacacggtgcgaagagtctattgagctagttggtagtcctccggcccctgaatgcggccaatcctaactgcggagcacatactcccaatccagggagcagtgtgtcgtaatgggtaactctgcagcggaaccgactactttgggtgtccgtgtttccttttattctcacattgactgcttatggtgacaattgaaagattgttaccatatagctattggattggtcatccggtgagcaatagagctattgtttatcaatttgttggatttgtaccactcaacttttctgttttgagaacactcaactacatcttactgctaaacacatcaaa

SEQ ID NO: 44 - DNA, 614 bp, synthetic construct
  source 1..614: mol_type = other DNA
ttaaaactgggtccaggttgttcccacctggatctcctattgggagttgtactctattattccggtaattttgtacgccagttttatctccccttccccaattgcaacttagaagtttatcaatatgaccaataggcggtagttagccaaactaccagaggtcaagcacttctgtttccccggtcaaagttgatatgctccaacagggcaaaaacaactgagatcgttatccgcaaagtgcctacgcaaagcctagtaacacctttgaagatttatggttggtcgttccgctatttcccatagtagacctggcagatgaggctagaaattccccactggcgacagtgttctagcctgcgtggctgcctgcgcaccccttgggtgcgaagccatacattggacaaggtgtgaagagccccgtgtgctcactttgagtcctccggcccctgaatgtggctaaccttaaccctgcagctagtgcatgtaatccaacatgttgctagtcgtaatgagcaattgcgggacgggaccaactactttgggtgtccgtgtttcactttttccttttaatattgcttatggtgacaatatatataaacatatatattgacacc

SEQ ID NO: 45 - DNA, 742 bp, synthetic construct
  source 1..742: mol_type = other DNA
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcacc

SEQ ID NO: 46 - DNA, 742 bp, synthetic construct
  source 1..742: mol_type = other DNA
ttaaaacagcctgtgggttgcacccactcacagggcccactgggcgcaagcactctggtacctcggtacctttgtgcgcctgttttacaccccccccccagtgaaacttagaagcagcaaaccacgatcaatagcgggcataacgctccagttatgtcttgatcaagcacttctgtttccccggactgagtatcaatagactgctcgcgcggttgaaggagaaaacgttcgttatccggctagctacttcgggaaacctagtaacaccatgaaagttgcggagagcttcgttcagcactcccccagtgtagatcaggtcgatgagtcaccgcattccccacgggcgaccgtggcggtggctgcgttggcggcctgcccatggggtaacccatggggcgctctaatacggacatggtgtgaagagtctactgagctagttagtagtcctccggcccctgaatgcggctaatcccaactgcggagcacacgcccacaagccagcgggtagcgtgtcgtaacgggtaactctgcagcggaaccgactactttgggtgtccgtgtttctttttatctttatactggctgcttatggtgacaattaaagaattgttaccatatagctattggattggccatccggtgtgcaacagagcaattgtttacctattcattggtttcgtaccattaaccttgaagtctgtgaccacccttaactacatcttgacccttaacacagctaaac

SEQ ID NO: 47 - DNA, 611 bp, synthetic construct
  source 1..611: mol_type = other DNA
ttaaaactggatctgggttgttcccactcagatctcccacgtggagtagtacactattattacggtaatcttgtacgccagttttataatccccttacccaagtaacttagaagataaacacaaagaccaataggagatgatcatccagatcatcataggtcaagcacttctgtttccccggtcaaggttgatatgctctaacagggcaaaaacagctgagatcgttatccgcaaagcgcctacgcaaagcctagtaactatctggaagttgcttggttggtcgctccgccatatcccatggtagacctggcagatgaggctagaaattccccactggtgacagtgttctagcctgcgtggctgcctgcacaccctctgggtgtgaaaccaagtaatggacagggtgtgaagagccccgtgtgctcgctttgagtcctccggcccctgaatgtggctaaccttaaccctgcagctagggcacacaatccagtgtgtatctagtcgtaatgagcaattgcgggatgggaccaactactttgggtgtccgtgtttcttgtttttcttttatgtttgcttatggtgacaatatatagtaatatatattggcatc

SEQ ID NO: 48 - DNA, 666 bp, synthetic construct
  source 1..666: mol_type = other DNA
ttaaaatagctgctgggttgttcccatccagcaggcccactgggcgtgatactctggtattgcggtacctttgtatgcctattttacctcccttccccctgaaacttagaagaaagaaacaaacacgctcactaggtacactgcatccagcagtgtaatgagcaagcacttctgtttccccggaagggatatatggtacgctgtgcaaacggcggaaattaatcctaccgttaaccgcccatctactccgagaagcctagtacctaattgaacttatcaatggagttgcgctcagcaggtgaccctgacctgccagctccggctgatggacctgggcatcccccacaggcgactgtggcccaggtcgcgtggcggccggcccactcttttgagtgggacgccttgataatgacaaggtgggaagagcctattgggctagctggtttcctccggcctcctgaatgcggctaaccctaaccccggagcatatggtggcaacccagccactggtatgtcgtaacgcgtaagtctgggatgggaccgactactttggagagtccgtgtttccattattttctttattgaatcttatggtgacaaattgggagttactcgggtgacgattgattattacttctgaaaacgtagagatatagaatcaacaca

SEQ ID NO: 49 - DNA, 714 bp, synthetic construct
  source 1..714: mol_type = other DNA
ttaaaacagcctgtgggttgttcccaccccagaggcccacgtggcggccagtactctggtatcacggtacctttgtacgcctgttttatatccccttcccccgcaacttagaagaaaacaaatcaagttcactaggagggggtacaaaccagtaccaccacgaacaagcacttctgtttccccggtgatgtcgtatagactgtaaccacggttgaaaacgattgatccgttatccgctcttgtacttcgaaaagcccagtatcaccttggaatcttcgatgcgttgcgctcagcactcaaccccagagtgtagcttaggtcgatgagtctggacactcctcaccggcgacggtggtccaggctgcgttggcggcctacctgtggtccaaagccacaggacgctagttgtgaacaaggtgtgaagagcctattgagctacaagagaatcctccggcccctgaatgcggctaatcctaaccacggagcaagggtacacaaaccagtgtatatcttgtcgtaacgcgcaagtctgtggcggaaccgactactttgggtgtccgtgtttccttttgtttttatcatggctgcttatggtgacaatctaagattgttatcatatagctgttggattggccatccggtaatttattgagatttgagcatttgcttgtttcttcaacaatttcacctattcattgcatttcagcagtcaaa

SEQ ID NO: 50 - DNA, 722 bp, synthetic construct
  source 1..722: mol_type = other DNA
ttaaaatagcctgtgggttgctcccacccacagggcccaagtggcgtagtacttggtattccggtacctttgtacacctatttacaaaccctaccccttgtaaccttagaagcaattatttaaccgctcactagggggtgtgctatccaagcacatcaagagcaagcacttctgtctccccgggaggggctaatggtacgctgtgcccacggcggaaatgagccctaccgttaaccggcagtctacttcgggaagcccagtaactacattgaaactttgaggcgttacactcagcacataaccccaatgtgtagttctggtcgatgagccttggcatcccccacaggcgactgtggccaaggctgcgttggcggccagcctgcggaccaaaagtccgtaggacgcctaattgtggacatggtgtgaagagcctactgagctagactgtagtcctccggcccctgaatgcggctaatcctaaccctggagcatccgcgtgcaacccagtacgtagggtgtcgtaatgcgtaagtctgggatggaaccgactactttgggtgtccgtgtttcttgtttttcatactgggtcgcttatggttacaactaattgttgtaatcattggcagtgcgcgctgaccacgcgattattgatatttccatttgttggatactccaatagtgtcaactcatatacacaacttttaccactgatcaagataaaa

SEQ ID NO: 51 - DNA, 747 bp, synthetic construct
  source 1..747: mol_type = other DNA
ttaaaacagctctggggttgctcccaccccagaggcccacgtggcggccagtactctggtattgcggtacctttgtacgcctgttttatactccctccccccgtgcaactttagaagaaatccacaaagttcaatagagggggtgcaaaccagcaccaccacgaacaagcacttctgtttccccggtgatgtcgtataagctgtacccacggctgaaggcgacggatccgttatccgcttgagtacttcgagaagcctagtattaccttggaatcttcgacgcgttgcgctcagcactctaccccgagtgtagcttaggtcgatgagtctgggcacgccccaccggcgacggtggcccaggctgcgttggcggcctacccatggctatcaccatgggacgctagttgtgaacaaggtgtgaagagcctattgagctacccaagagtcctccggcccctgaatgcggctaatcctaaccacggagcaagtgtcctcaacccaggggatggcttgtcgtaacgcgaaagtctgtggcggaaccgactactttgggtgtccgtgtttccttttatttttatgtatggctgcttatggtgacaatcaaaggttgttaccataaagcaatttggattggccatccggtgagaatcaaacatattatctacctgtttgttgggttttcttcttttacttgaacaatacctctaataataactgctatattgtcaataagacattatcatcaca

SEQ ID NO: 52 - DNA, 607 bp, synthetic construct
  source 1..607: mol_type = other DNA
ttaaaacagctgtgtggttgttcccaccaccaggcacactgtgcgttgtacactgggattccggtcactttgtacgcctgtttgctatcccccccaacttatgtaatttagaagatgtacacaacgcccattaggatgcggccaaaccaggtccgcttagggcaagcacttctgtttccccgggtgtgtgaatagactctaacagggttgaagctgtagcactcgttatccgcgcaactactacgcgaatgttagtagcatcctgtgttgcatttgggatttcgctccgcagaaaaccccatctgtagattagggcaatgaggctacacataccccactggcgacagtggtgtagcctgcgtggtgccctacccaggccatcttggcctgggattccacttacaagacagggtgtgaaggcactagtgtgctagttgtgagtcctccggcccctgaatgcggctaatcttaaccccgtagcccccgcaagtaaaccaacttgtaggtggtcgtaatgagtaattacgggatggaaccgactactttgggtgtccgtgtttccttttattctttatatttgcatcctatggttacaacataagtaatc

SEQ ID NO: 53 - DNA, 611 bp, synthetic construct
  source 1..611: mol_type = other DNA
ttaaaactgaatccagattgttcccatctggatttcctacatggagttgtactctattattccggtaattttgtacgccagttttatcaccccttcccccgtaacttagaagtttgaaacaaaagaccaataggaggtaactatccaagttactataggtcaagcacttctgtttccccggtcaaagttgatatgctccaccagggcaaaaacaattgagatcgttatccgcaaagtgcctacgcaaagcctagtagtatcttgaaaagcatgtggttggttgctccgctgtaccccacagtaaacctggcagatgaggctagaagttccccactggtgacagtgttctagcctgcgtggctgcctgcgcactctttgagtgcgaagccatatgtttgacaaggtgtgaagagccccgtgtgctcactttgagtcctccggcccctgaatgtggctaaccttaaccctgcagctagtgcatgcaatccagcatgtggctagtcgtaatgagcaattgcgggatgggaccaactactttgggtgtccgtgtttcacttttttccttttataattgcttatggtgacaatatatagtgatatatattgacacc

SEQ ID NO: 54 - DNA, 630 bp, synthetic construct
  source 1..630: mol_type = other DNA
tttaaacagcggatgggtatcccaccatccgacccactgggtgtagtactctggtattttgtacctttgtacgcctgttgttcctaatgtacccaccctaaaacttcctacccaagtaacgttagaagtttcatcaacaagtacaataggaagcatcacatccagtggtgttttgtacaagcacttctgtttccccggagcgaggtataggctgtacccactgccgaaagcctttaaccgttatccgccaaccaactacgtaaaagctagtatcatcatgttttaaaataggcgttcgatcaggtggtacccccctccactagtttggtcgatgaggctaggaactccccacgggtgaccgtgtcctagcctgcgtggcggccaacccagcttctgctgggacgcctttttatggacatggtgtgaagactcgcatgtgcttggttgtgactcctccggcccctgaatgcggctaaccttaaccccggagccctgtgttgcaatccagtaacattagggtcgtaatgagcaatttcgggacgggaccgactactttgggtgtccgtgtttctcatttttcttattattgtcttatggtcacagcatatatataacgtatatactgtgatc

SEQ ID NO: 55 - DNA, 625 bp, synthetic construct
  source 1..625: mol_type = other DNA
ttaaaacagcggatgggtttcccaccatccgacccactgggtgtagtgctctggtattttgtacctttgcacgcctgtttcccatttgtacccttccttaatctccttcccccgtaacgttagaagttttggaattttaaagtacaataggaagcgccacatccagtggtgttgcgtacaaatacttctgtttacccggagcgaggtataggttgtacccacggccaaaagcctttaaccgttatccgccaatcaactacgtaacggctagtatcatcttgcttttgatttggtgttcgatcaggtggtatcccccactagtctggtcgatgaggctaggaattccccacgggcgaccgtgtcctagcctgcgtggcggccagcccagcttttgctgggacgccttttcaaagacatggtgtgaagacctgcatgtgcttggttgtgagtcctccggcccctgaatgcggctaaccttaaccctggagcccagcagcataatccaatgttgtttgggtcgtaatgagcaattccgggacgggaccgactactttgggtgtccgtgtttccttttattcttacattgtcttatggtcacagcatatatattatatatactgtgatc

SEQ ID NO: 56 - DNA, 627 bp, synthetic construct
  source 1..627: mol_type = other DNA
ttaaaacagcggatgggtatcccaccatccggcccactgggtgtagtactctggtacattgtacctttgtacgcctgttttccccctcttgtacccgcccttcaagctccttgcccaagtaacgttagaagtttgaacattggtacaataggaagcatcacatccagtggtgtactgtacaaacacttctgttgccccggagcgaggtatagatggtccccaccgtcaaaagcctttaaccgttatccgccaatcaactacgtaatggctagtagcaccttggatttaagttggcgttcgatcaggtggtaacccccactagtttggtcgatgaggctaggaattccccacgggtgaccgtgtcctagcctgcgtggcggccaacccagcatccgctgggacgccaatttaatgacatggtgtgaagacctgcatgtgcttgattgtgagtcctccggcccctgaatgcggctaaccctaaccccggagccttgcagcacaatccagtgttgttaaggtcgtaatgagcaattctgggatgggaccgactactttgggtgtccgtgtttcttatttttcttgaatttttcttatggtcacagcatatatacattatatactgtgatc

SEQ ID NO: 57 - DNA, 626 bp, synthetic construct
  source 1..626: mol_type = other DNA
ttaaaactgggtgtgggttgttcccacccacaccacccaatgggtgttgtactctgttattccggtaactttgtacgccagtttttccctcccctccccatccttttacgtaacttagaagttttaaatacaagaccaatagtaggcaactctccaggttgtctaaggtcaagcacttctgtttccccggttgatgttgatatgctccaacagggcaaaaacaacagataccgttatccgcaaagtgcctacacagagcttagtaggattctgaaagatctttggttggtcgttcagctgcatacccagcagtagaccttgcagatgaggctggacattccccactggtaacagtggtccagcctgcgtggctgcctgcgcacctctcatgaggtgtgaagccaaagatcggacagggtgtgaagagccgcgtgtgctcactttgagtcctccggcccctgaatgcggctaaccttaaacctgcagccatggctcataagccaatgagtttatggtcgtaacgagtaattgcgggatgggaccgactactttgggtgtccgtgtttcactttttcctttattaattgcttatggtgacaatatatatattgatatatattggcatc

SEQ ID NO: 58 - DNA, 743 bp, synthetic construct
  source 1..743: mol_type = other DNA
ttaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaa

SEQ ID NO: 59 - DNA, 544 bp, synthetic construct
  source 1..544: mol_type = other DNA
ttgtttgatgccagttttatctccccccccaattgcaacttagaagatgtacacaaagaccaataggcagtggtcacccagaccactgaaggtcaagcacttctgtctccccggtcaaagttgatatgctccaacagggcaaaaacaacttagatcgttatccgcaaagtgcctacgcaaagcttagtaccatctttgagagcctatggttggtcgctccactgaaccccacagtagacctggcagatgaggctagaagacccccactggcgacagtgttctagcctgcgtggctgcctgcacacccttacgggtgtgaagccatatgtttgacaaggtgcgaagagccccgtgtgctcactttgagtcctccggcccctgaatgtggctaaccttaaccctgcagctagtgcacacaagccagtgtgttgctagtcgtaatgagcaattgcgggatgggaccaactactttgggtgtccgtgtttcactttttacctttattttgcttatggtgacaatatatatagtatatatattggcacc

SEQ ID NO: 60 - DNA, 460 bp, synthetic construct
  source 1..460: mol_type = other DNA
cttgtaaaaacaactttgatgccttgaatatataatgattcattataacaattatgcatagattttaataatctgcatattttatgctttcatgtttttcctaattaatgatttgacatggttaataattataatatattctgcatcacagtttacatatttatgtaaaataagcatttaaaaattattagttttattctgcctgcttaaatattactttcctcaaaaagagaaaacaaaaatgctagattttactttatgacttgaatgatgtggtaatgtcgaactctagtatttagaattagaatgtttcttagcggtcgtgtagttatttttatgtcataagtggataatttgttagctcctataacaaaagtctgttgcttgtgtttcacattttggatttcctaatataatgttctctttttagaaaaggtggacaagtcctattttcaagaga

SEQ ID NO: 61 - DNA, 625 bp, synthetic construct
  source 1..625: mol_type = other DNA
ttaaaacagcggatgggtttcccaccatccgacccactgggtgtagtgctctggtattttgtacctttgcacgcctgtttcccctttgtacccatcctgaatttcctccctctgcaacgttagaagtttgtgaaattaaaagtacaataggaagcatcacatccagtggtgttcagtacaagcactcctgtttccccggagcgaggtataggttgtacccacgaccgaaagcctttaaccgttatccgccaatcaactacataacggctagtatcatcatgtttttgatctggcgttcgatcaggtggtttcccccactagtctggtcgatgaggctaggatttccccacgggcgaccgtgtcctagcctgcgtggcggccagcccagcttatgctgggacgcctttttaaagacatggtgtgaagacctgcatgtgcttgattgtgagtcctccggcccctgaatgcggctaaccttaaccctggagcccgacagcataatccaatgttgtttgggtcgtaatgagcaattccgggatgggaccgactactttgggtgtccgtgtttctttttattcttatattgtcttatggtcacagcatatatagtatatatactgtgatc

SEQ ID NO: 62 - DNA, 584 bp, synthetic construct
  source 1..584: mol_type = other DNA
tactctggtattatgtacctttgtacgcctgtttcttccctacaaccccttcctaaaactcccacccatgaaacgttagaagcttgacatttaagtacaataggtggcaccacatccagtggtgtctacgtacaagcacttctgtttccccggagcgaggtataggctgtacccactgccaaaagcctttaaccgttatccgccaaccaactacgtaacagctagtatcatcttgttcttcactggacgttcgatcaggtggattccccctccactagtttggtcgatgaggctaggagctccccacgggtgaccgtgtcctagcctgcgtggcggccaacccagctcatgctgggacgcccttttaaggacatggtgtgaagactcgcatgtgcttggttgtgagtcctccggcccctgaatgcggctaaccttaaccctggagccttatgccacgaaccagtggttgtaaggtcgtaacgagcaattccgggatgggaccgactactttgggtgtccgtgtttcctatttttctttatattgtcttatggtcacagcatatatataagtatatactgtgatc

SEQ ID NO: 63 - DNA, 2059 bp, synthetic construct
  source 1..2059: mol_type = other DNA
taatacgactcactataggggggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaaacaaaaaaaaaaaataattgactaatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctaggagtactgagctcgtttagtgaaccgtcagatcgcctggagacgccatccacgctgttttgacctccatagaagacaccgggaccgatccagcctccgcggccccgaattcaccatgggctctgtgtctaaaggagaagaagacaacatggcgagtctgccggcgacacatgagctgcacatctttggatccatcaacggtgtcgactttgacatggttggccaaggcaccggaaatccaaatgatggctatgaggagctgaacctgaagagtacgaaaggagacctgcagttctccccctggatcctggtgcctcacattggctatggcttccaccagtatctgccctacccagacggcatgagccctttccaggctgccatggtggatggatcaggctaccaggtgcacaggaccatgcagtttgaggatggggcttcgttgactgtgaactacagatacacatatgaagggtctcatattaaaggagaggcccaggtgaaggggacaggcttccctgctgacggtcctgtcatgaccaacagcctgactgcagctgactggtgccgcagcaaaaagacgtaccccaatgacaagaccatcataagcactttcaagtggagctacaccactgggaatggcaagaggtaccgctccactgcccgtaccacctatacctttgccaagccaatggctgccaattacctgaagaaccagcccatgtacgtcttccggaagacagagttgaagcacagcaaaactgaactcaacttcaaagagtggcagaaggccttcacagatgtgatggggatggatgagctctacaaatgagctggagcctcggtggccatgcttcttgccccttgggcctccccccagcccctcctccccttcctgcacccgtacccccgtggtctttgaataaagtctgactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 64 - DNA, 1915 bp, synthetic construct
  source 1..1915: mol_type = other DNA
taatacgactcactataggggggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaaacaaaaaaaaaaaataattgactaatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatggcttcatcggaagacgtcatcaaagaattcatgaggttcaaggttcgcatggagggcagtgtgaacggccatgagtttgaaatcgagggtgagggggagggccgcccctacgaagggacacagaccgccaaactaaaggtcaccaaagggggcccgttgccctttgcctgggacatcttatccccccagttccagtacgggagcaaggcctatgtaaaacaccctgctgacattccagactaccttaaactgagtttcccagaaggcttcaagtgggagagggtgatgaactttgaggatggcggagtggtgacagtgactcaagactcctccctgcaggatggggagttcatctacaaagtgaagctccgcggcactaacttcccttctgatggcccggtcatgcagaagaaaaccatgggctgggaagccagcacggagagaatgtaccctgaggacggtgccctgaaaggtgagataaagatgcggctgaagttgaaggacggagggcactatgatgctgaggtcaagaccacctatatggcgaagaagcctgtccagctgccaggagcctacaagacagacattaaacttgatatcacctctcacaatgaagactataccattgtggagcagtatgagcgtgcagagggacggcacagcactggggcgtgagctggagcctcggtggccatgcttcttgccccttgggcctccccccagcccctcctccccttcctgcacccgtacccccgtggtctttgaataaagtctgactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 65 - DNA, 3733 bp, synthetic construct
  source 1..3733: mol_type = other DNA
taatacgactcactataggggggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaaacaaaaaaaaaaaataattgactaatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctaggagtactgagctcgtttagtgaaccgtcagatcgcctggagacgccatccacgctgttttgacctccatagaagacaccgggaccgatccagcctccgcggccccgaattcaccatgggctctgtgtctaaaggagaagaagacaacatggcgagtctgccggcgacacatgagctgcacatctttggatccatcaacggtgtcgactttgacatggttggccaaggcaccggaaatccaaatgatggctatgaggagctgaacctgaagagtacgaaaggagacctgcagttctccccctggatcctggtgcctcacattggctatggcttccaccagtatctgccctacccagacggcatgagccctttccaggctgccatggtggatggatcaggctaccaggtgcacaggaccatgcagtttgaggatggggcttcgttgactgtgaactacagatacacatatgaagggtctcatattaaaggagaggcccaggtgaaggggacaggcttccctgctgacggtcctgtcatgaccaacagcctgactgcagctgactggtgccgcagcaaaaagacgtaccccaatgacaagaccatcataagcactttcaagtggagctacaccactgggaatggcaagaggtaccgctccactgcccgtaccacctatacctttgccaagccaatggctgccaattacctgaagaaccagcccatgtacgtcttccggaagacagagttgaagcacagcaaaactgaactcaacttcaaagagtggcagaaggccttcacagatgtgatggggatggatgagctctacaaatgatcgaggttaattaatgagcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctcccccccccccttttccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatggcttcatcggaagacgtcatcaaagaattcatgaggttcaaggttcgcatggagggcagtgtgaacggccatgagtttgaaatcgagggtgagggggagggccgcccctacgaagggacacagaccgccaaactaaaggtcaccaaagggggcccgttgccctttgcctgggacatcttatccccccagttccagtacgggagcaaggcctatgtaaaacaccctgctgacattccagactaccttaaactgagtttcccagaaggcttcaagtgggagagggtgatgaactttgaggatggcggagtggtgacagtgactcaagactcctccctgcaggatggggagttcatctacaaagtgaagctccgcggcactaacttcccttctgatggcccggtcatgcagaagaaaaccatgggctgggaagccagcacggagagaatgtaccctgaggacggtgccctgaaaggtgagataaagatgcggctgaagttgaaggacggagggcactatgatgctgaggtcaagaccacctatatggcgaagaagcctgtccagctgccaggagcctacaagacagacattaaacttgatatcacctctcacaatgaagactataccattgtggagcagtatgagcgtgcagagggacggcacagcactggggcgtgagctggagcctcggtggccatgcttcttgccccttgggcctccccccagcccctcctccccttcctgcacccgtacccccgtggtctttgaataaagtctgactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 66 - DNA, 4120 bp, synthetic construct
  source 1..4120: mol_type = other DNA
taatacgactcactataggggggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaaacaaaaaaaaaaaataattgactaatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctaggagtactgagctcgtttagtgaaccgtcagatcgcctggagacgccatccacgctgttttgacctccatagaagacaccgggaccgatccagcctccgcggccccgaattcaccatgccaaacccccgtcctgccaagcccatggctccttctcttgcactgggaccctctcctggggtgcttccatcctggaaaacagcccccaaaggctcggaattactcggcactcgaggctctggagggcctttccagggcagagatctccgcagtggtgctcacacaagtagtagcttaaatccactcccgcccagccagctacaactgcccactgttccactggtgatggtcgcacccagtggtgccaggttgggcccctcaccacatttgcaagcacttctgcaggacaggcctcacttcatgcaccagctgtcgacggtggacgctcatgcccagacacctgtccttcaggttcgccccctggacaaccctgctatgatctctctaccgcccccatcagcagccacaggagtcttctctctcaaagctcggccagggctgcctccaggaattaatgttgcttcccttgagtgggtatcacgtgagcccgccttgctgtgtacatttcctcggtcaggaactccccggaaagacagcaacttgttagctgcacctcaaggctcctatccactgcttgccaacggtgtctgcaagtggcctggctgtgagaaggtgtttgaagagcctgaggagttcctgaagcactgccaggctgaccacctgctggatgaaaaagggaaagctcagtgtctgctccaaagggaagtggtgcagagcttggagcagcagttggaactggaaaaggagaaacttggtgccatgcaagcccatctcgcaggcaagatggccttagccaaagcaccgagcgtggcctccatggacaagtcctcctgctgcattgtagccaccagtacccagggaagcgtactgccagcgtggagcgcgccgcgggaagcacctgatgggggcctgtttgccgtcagacgccatctgtggggtagccatgggaattcttccttcccagaattcttccacaacatggattacttcaagtatcataatatgaggccacccttcacctatgccactctcatccgctgggcaatattggaagccccagagagacagaggacgctgaatgagatttaccactggtttaccaggatgtttgcatactttagaaaccacccagctacctggaagaatgccatccgacataacctctccctgcacaagtgctttgttcgagtggagtcagaaaaaggagctgtgtggacagtggatgagtttgagttcagaaagaagagatcccagcggcccaacaagtgtagcaatccatgtccatgataatcgaggttaattaatgagcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctcccccccccccttttccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtgagctggagcctcggtggccatgcttcttgccccttgggcctccccccagcccctcctccccttcctgcacccgtacccccgtggtctttgaataaagtctgactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 67 - DNA, 2234 bp, synthetic construct
  source 1..2234: mol_type = other DNA
taatacgactcactataggggggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaaacaaaaaaaaaaaataattgactaattaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaagcttgccacaacccgggatcctctaggagtactgagctcgtttagtgaaccgtcagatcgcctggagacgccatccacgctgttttgacctccatagaagacaccgggaccgatccagcctccgcggccccgaattcaccatgggctctgtgtctaaaggagaagaagacaacatggcgagtctgccggcgacacatgagctgcacatctttggatccatcaacggtgtcgactttgacatggttggccaaggcaccggaaatccaaatgatggctatgaggagctgaacctgaagagtacgaaaggagacctgcagttctccccctggatcctggtgcctcacattggctatggcttccaccagtatctgccctacccagacggcatgagccctttccaggctgccatggtggatggatcaggctaccaggtgcacaggaccatgcagtttgaggatggggcttcgttgactgtgaactacagatacacatatgaagggtctcatattaaaggagaggcccaggtgaaggggacaggcttccctgctgacggtcctgtcatgaccaacagcctgactgcagctgactggtgccgcagcaaaaagacgtaccccaatgacaagaccatcataagcactttcaagtggagctacaccactgggaatggcaagaggtaccgctccactgcccgtaccacctatacctttgccaagccaatggctgccaattacctgaagaaccagcccatgtacgtcttccggaagacagagttgaagcacagcaaaactgaactcaacttcaaagagtggcagaaggccttcacagatgtgatggggatggatgagctctacaaatgagctggagcctcggtggccatgcttcttgccccttgggcctccccccagcccctcctccccttcctgcacccgtacccccgtggtctttgaataaagtctgactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 68 - DNA, 2090 bp, synthetic construct
  source 1..2090: mol_type = other DNA
taatacgactcactataggggggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaaacaaaaaaaaaaaataattgactaattaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaagcttgccacaacccgggataattcctgcagccaatatggcttcatcggaagacgtcatcaaagaattcatgaggttcaaggttcgcatggagggcagtgtgaacggccatgagtttgaaatcgagggtgagggggagggccgcccctacgaagggacacagaccgccaaactaaaggtcaccaaagggggcccgttgccctttgcctgggacatcttatccccccagttccagtacgggagcaaggcctatgtaaaacaccctgctgacattccagactaccttaaactgagtttcccagaaggcttcaagtgggagagggtgatgaactttgaggatggcggagtggtgacagtgactcaagactcctccctgcaggatggggagttcatctacaaagtgaagctccgcggcactaacttcccttctgatggcccggtcatgcagaagaaaaccatgggctgggaagccagcacggagagaatgtaccctgaggacggtgccctgaaaggtgagataaagatgcggctgaagttgaaggacggagggcactatgatgctgaggtcaagaccacctatatggcgaagaagcctgtccagctgccaggagcctacaagacagacattaaacttgatatcacctctcacaatgaagactataccattgtggagcagtatgagcgtgcagagggacggcacagcactggggcgtgagctggagcctcggtggccatgcttcttgccccttgggcctccccccagcccctcctccccttcctgcacccgtacccccgtggtctttgaataaagtctgactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 69 - DNA, 4083 bp, synthetic construct
  source 1..4083: mol_type = other DNA
taatacgactcactataggggggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaaacaaaaaaaaaaaataattgactaattaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaagcttgccacaacccgggatcctctaggagtactgagctcgtttagtgaaccgtcagatcgcctggagacgccatccacgctgttttgacctccatagaagacaccgggaccgatccagcctccgcggccccgaattcaccatgggctctgtgtctaaaggagaagaagacaacatggcgagtctgccggcgacacatgagctgcacatctttggatccatcaacggtgtcgactttgacatggttggccaaggcaccggaaatccaaatgatggctatgaggagctgaacctgaagagtacgaaaggagacctgcagttctccccctggatcctggtgcctcacattggctatggcttccaccagtatctgccctacccagacggcatgagccctttccaggctgccatggtggatggatcaggctaccaggtgcacaggaccatgcagtttgaggatggggcttcgttgactgtgaactacagatacacatatgaagggtctcatattaaaggagaggcccaggtgaaggggacaggcttccctgctgacggtcctgtcatgaccaacagcctgactgcagctgactggtgccgcagcaaaaagacgtaccccaatgacaagaccatcataagcactttcaagtggagctacaccactgggaatggcaagaggtaccgctccactgcccgtaccacctatacctttgccaagccaatggctgccaattacctgaagaaccagcccatgtacgtcttccggaagacagagttgaagcacagcaaaactgaactcaacttcaaagagtggcagaaggccttcacagatgtgatggggatggatgagctctacaaatgatcgaggttaattaatgagcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccccccccccctttttaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaagcttgccacaacccgggataattcctgcagccaatatggcttcatcggaagacgtcatcaaagaattcatgaggttcaaggttcgcatggagggcagtgtgaacggccatgagtttgaaatcgagggtgagggggagggccgcccctacgaagggacacagaccgccaaactaaaggtcaccaaagggggcccgttgccctttgcctgggacatcttatccccccagttccagtacgggagcaaggcctatgtaaaacaccctgctgacattccagactaccttaaactgagtttcccagaaggcttcaagtgggagagggtgatgaactttgaggatggcggagtggtgacagtgactcaagactcctccctgcaggatggggagttcatctacaaagtgaagctccgcggcactaacttcccttctgatggcccggtcatgcagaagaaaaccatgggctgggaagccagcacggagagaatgtaccctgaggacggtgccctgaaaggtgagataaagatgcggctgaagttgaaggacggagggcactatgatgctgaggtcaagaccacctatatggcgaagaagcctgtccagctgccaggagcctacaagacagacattaaacttgatatcacctctcacaatgaagactataccattgtggagcagtatgagcgtgcagagggacggcacagcactggggcgtgagctggagcctcggtggccatgcttcttgccccttgggcctccccccagcccctcctccccttcctgcacccgtacccccgtggtctttgaataaagtctgactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 70 - DNA, 4470 bp, synthetic construct
  source 1..4470: mol_type = other DNA
taatacgactcactataggggggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaaacaaaaaaaaaaaataattgactaattaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaagcttgccacaacccgggatcctctaggagtactgagctcgtttagtgaaccgtcagatcgcctggagacgccatccacgctgttttgacctccatagaagacaccgggaccgatccagcctccgcggccccgaattcaccatgccaaacccccgtcctgccaagcccatggctccttctcttgcactgggaccctctcctggggtgcttccatcctggaaaacagcccccaaaggctcggaattactcggcactcgaggctctggagggcctttccagggcagagatctccgcagtggtgctcacacaagtagtagcttaaatccactcccgcccagccagctacaactgcccactgttccactggtgatggtcgcacccagtggtgccaggttgggcccctcaccacatttgcaagcacttctgcaggacaggcctcacttcatgcaccagctgtcgacggtggacgctcatgcccagacacctgtccttcaggttcgccccctggacaaccctgctatgatctctctaccgcccccatcagcagccacaggagtcttctctctcaaagctcggccagggctgcctccaggaattaatgttgcttcccttgagtgggtatcacgtgagcccgccttgctgtgtacatttcctcggtcaggaactccccggaaagacagcaacttgttagctgcacctcaaggctcctatccactgcttgccaacggtgtctgcaagtggcctggctgtgagaaggtgtttgaagagcctgaggagttcctgaagcactgccaggctgaccacctgctggatgaaaaagggaaagctcagtgtctgctccaaagggaagtggtgcagagcttggagcagcagttggaactggaaaaggagaaacttggtgccatgcaagcccatctcgcaggcaagatggccttagccaaagcaccgagcgtggcctccatggacaagtcctcctgctgcattgtagccaccagtacccagggaagcgtactgccagcgtggagcgcgccgcgggaagcacctgatgggggcctgtttgccgtcagacgccatctgtggggtagccatgggaattcttccttcccagaattcttccacaacatggattacttcaagtatcataatatgaggccacccttcacctatgccactctcatccgctgggcaatattggaagccccagagagacagaggacgctgaatgagatttaccactggtttaccaggatgtttgcatactttagaaaccacccagctacctggaagaatgccatccgacataacctctccctgcacaagtgctttgttcgagtggagtcagaaaaaggagctgtgtggacagtggatgagtttgagttcagaaagaagagatcccagcggcccaacaagtgtagcaatccatgtccatgataatcgaggttaattaatgagcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccccccccccctttttaaaacagcctgtgggttgttcccacccgcagggcccactgggcgctagcacactggtatcccggtacccttgtgcgcctgttttatataccctcccccttatgtaacttagaagtatgattcaaacggtcgacaggcggctcagtgcaccaactgagtcatgaccaagcacttctgttaccccggactgagtatcaataagctgttcacacggctgaaggagaaaacgttcgttacccggccaattacttcgagaaacctagtaccaccatgaaggttgcgcagtgtttcgctccacacaaccccagtgtagatcaggtcgatgagtcaccgcattccccacgggcgaccgtggcggtggctgcgttggcggcctgcccatggggcaacccatgggacgcttcaatactgacatggtgtgaagagtctattgagctaattggtagtcctccggcccctgaatgcggctaatcccaactgtggagcagatactcacaaaccagtgagcggtctgtcgtaacgggcaactccgcagcggaaccgactactttgggtgtccgtgtttctttttattcttacattggctgcttatggtgacaattgacaaattgttaccatatagctattggattggccatccggtgacaaacagagctattgtttacttgtttgttggtttcataccattaaattacaaggtcttagaaactctcaactttattttgacactcaatacagcaaagcttgccacaacccgggataattcctgcagccaatatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtgagctggagcctcggtggccatgcttcttgccccttgggcctccccccagcccctcctccccttcctgcacccgtacccccgtggtctttgaataaagtctgactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 71 - protein, 5 aa, synthetic construct
GGGGS

SEQ ID NO: 72 - protein, 4 aa, synthetic construct
AGAG